(19) United States
(12) Patent Application Publication
(10) Pub. No.: US 2022/0144960 A1
(43) Pub. Date: May 12, 2022

(54) CD30-BINDING MOIETIES, CHIMERIC ANTIGEN RECEPTORS, AND USES THEREOF

(71) Applicant: NANJING LEGEND BIOTECH CO., LTD., Nanjing, Jiangsu Province (CN)

(72) Inventors: Shuai Yang, Nanjing (CN); Sujuan Wang, Nanjing (CN); Chenyu Shu, Nanjing (CN); Yuanyuan Peng, Nanjing (CN); Chen Hu, Nanjing City (CN); Shu Wu, Nanjing (CN)

(21) Appl. No.: 17/312,497

(22) PCT Filed: Dec. 26, 2019
(86) PCT No.: PCT/CN2019/128613

§ 371 Date: Jun. 10, 2021

(30) Foreign Application Priority Data
Dec. 26, 2018 (CN) PCT/CN2018/123977

(51) Int. Cl. C07K 16/28; A61K 35/17; A61P 35/00; C07K 14/725; C12N 5/0783

(52) U.S. Cl. CPC C07K 16/2878; A61K 35/17; A61K 2039/505; C07K 14/7051; C12N 5/0636; A61P 35/00

(57) ABSTRACT

CD30-binding moieties, chimeric antigen receptors (CARs) having these CD30-binding moieties, and uses thereof are provided. Polynucleotides encoding the CD30-binding moieties and CARs, compositions comprising CD30-binding moieties and CARs, genetically modified immune cells having a chimeric antigen receptor for use in adoptive cell therapy for treating CD30-expressing cancer or tumor in a subject in need thereof are also provided herein.

This application claims priority benefits of International Patent Application No. PCT/CN2018/123977 filed on Dec. 26, 2018, the contents of which are incorporated herein by reference in their entirety.

### FIELD

The present disclosure relates to the fields of molecular biology, cell biology, and cancer biology. In particular, provided herein include CD30-binding moieties, chimeric antigen receptors (CARs) comprising such CD30-binding moieties (“CD30 CARs”), genetically engineered immune cells expressing such CD30 CARs, and uses thereof in treating CD30-expressing tumors or cancers.

### BACKGROUND

CD30, also commonly known as Ki-1 or TNFRSF8, is a member of the tumor necrosis factor receptor superfamily. The human CD30 is expressed transiently at low levels on intrafollicular and perifollicular T and B cell blasts in lymphoid tissues, and is specifically upregulated on certain hematopoietic malignancies, including anaplastic large cell lymphoma and Hodgkin lymphoma, among others.

CD30 is a marker for, for example, the malignant cells in Hodgkin's disease (HD) and a subset of non-Hodgkin's (NHL) lymphomas, such as anaplastic large cell lymphoma (ALCL). Current antibody therapies targeting CD30, however, have only had limited success. Thus, additional CD30-targeting therapeutic options represent unmet needs. The compositions and methods provided herein meet these needs and provide other relative advantages.

### BRIEF SUMMARY OF THE INVENTION

Provided herein is a binding moiety that specifically binds CD30, comprising a single domain antibody comprising: (i) a CDR1 comprising an amino acid sequence selected from the group consisting of SEQ ID NOs:87-95; (ii) a CDR2 comprising an amino acid sequence selected from the group consisting of SEQ ID NOs:100-106; and (iii) a CDR3 comprising an amino acid sequence selected from the group consisting of SEQ ID NOs:111-120; or a variant thereof comprising up to 3 amino acid substitutions in each of CDR1, CDR2, and CDR3.

In some embodiments, the CD30-binding moiety provided herein comprises a single domain antibody comprising a CDR1 comprising SEQ ID NO:87; a CDR2 comprising SEQ ID NO:100; and a CDR3 comprising SEQ ID NO:111. In some embodiments, the CD30-binding moiety provided herein comprises a single domain antibody comprising a CDR1 comprising SEQ ID NO:87; a CDR2 comprising SEQ ID NO:100; and a CDR3 comprising SEQ ID NO:112. In some embodiments, the CD30-binding moiety provided herein comprises a single domain antibody comprising a CDR1 comprising SEQ ID NO:88; a CDR2 comprising SEQ ID NO:101; and a CDR3 comprising SEQ ID NO:113. In some embodiments, the CD30-binding moiety provided herein comprises a single domain antibody comprising a CDR1 comprising SEQ ID NO:89; a CDR2 comprising SEQ ID NO:102; and a CDR3 comprising SEQ ID NO:114. In some embodiments, the CD30-binding moiety provided herein comprises a single domain antibody comprising a CDR1 comprising SEQ ID NO:90; a CDR2 comprising SEQ ID NO:103; and a CDR3 comprising SEQ ID NO:115. In some embodiments, the CD30-binding moiety provided herein comprises a single domain antibody comprising a CDR1 comprising SEQ ID NO:91; a CDR2 comprising SEQ ID NO:104; and a CDR3 comprising SEQ ID NO:116. In some embodiments, the CD30-binding moiety provided herein comprises a single domain antibody comprising a CDR1 comprising SEQ ID NO:92; a CDR2 comprising SEQ ID NO:105; and a CDR3 comprising SEQ ID NO:117. In some embodiments, the CD30-binding moiety provided herein comprises a single domain antibody comprising a CDR1 comprising SEQ ID NO:93; a CDR2 comprising SEQ ID NO:106; and a CDR3 comprising SEQ ID NO:118. In some embodiments, the CD30-binding moiety provided herein comprises a single domain antibody comprising a CDR1 comprising SEQ ID NO:94; a CDR2 comprising SEQ ID NO:103; and a CDR3 comprising SEQ ID NO:119. In some embodiments, the CD30-binding moiety provided herein comprises a single domain antibody comprising a CDR1 comprising SEQ ID NO:95; a CDR2 comprising SEQ ID NO:103; and a CDR3 comprising SEQ ID NO:120. In some embodiments, provided herein are variants of these CD30-binding moieties comprising up to about 5 amino acid substitutions (e.g. one, two, three, four or five amino acid substitutions) in the CDRs.

In some embodiments, the CD30-binding moiety provided herein comprises a single domain antibody having an amino acid sequence that is at least 90%, 95%, or 99% identical to an amino acid sequence selected from the group consisting of SEQ ID NOs:9-54 and 199.

In some embodiments, the CD30-binding moiety provided herein comprises a single domain antibody having an amino acid sequence selected from the group consisting of SEQ ID NOs:9-54 and 199.

In some embodiments, the CD30-binding moiety provided herein comprises a single domain antibody comprising a CDR1, CDR2, and CDR3 from a binding moiety comprising a single domain having an amino acid sequence selected from the group consisting of SEQ ID NOs:9-54 and 199.

In some embodiments, the CD30-binding moiety provided herein specifically binds human CD30, rhesus CD30, or both.

In some embodiments, the CD30-binding moiety provided herein specifically binds the cysteine rich domain 6 (CRD6) of CD30 (SEQ ID NO:8), or the cysteine rich domain 1 (CRD1) of CD30 (SEQ ID NO:3).

In some embodiments, the CD30-binding moiety provided herein comprises an antibody or antigen-binding fragment thereof, or an extracellular domain of a receptor.

In some embodiments, the CD30-binding moiety provided herein comprises an antibody or antigen-binding fragment thereof selected from the group consisting of a single domain antibody (sdAb), a heavy chain antibody (HCAb), a Fab, a Fab′, a F(ab′)₂, a Fv, a scFv, a (scFv)₂, an IgG1 antibody, an IgG2 antibody, an IgG3 antibody, and an IgG4 antibody.

In some embodiments, the CD30-binding moiety provided herein comprises a camel antibody or antigen-binding fragment thereof, a chimeric antibody or antigen-binding fragment thereof, a humanized antibody or antigen-binding fragment thereof, or a human antibody or antigen-binding fragment thereof.

In some embodiments, the CD30-binding moiety provided herein comprises a sdAb.

In some embodiments, the CD30-binding moiety provided herein comprises a HCAb that comprises a sdAb fused with human IgG1 hinge and Fc region.

In some embodiments, the CD30-binding moiety provided herein comprises a monovalent sdAb.

In some embodiments, the CD30-binding moiety provided herein comprises a first sdAb, a linker, and a second sdAb, from N-terminus to C-terminus. In some embodiments, the first and second sdAbs recognize different epitopes on CD30. In some embodiments, the first and second sdAbs recognize the same epitope on CD30. In some embodiments, the second sdAb is a tandem repeat of the first sdAb.

In some embodiments, the CD30-binding moiety provided herein comprises a first sdAb, a linker, and a second sdAb, from N-terminus to C-terminus, wherein the first and second sdAbs each having an amino acid sequence selected from the group consisting of SEQ ID NOs:9-54 and 199.

In some embodiments, the CD30-binding moiety provided herein comprises a first sdAb, a linker, and a second sdAb, wherein the linker has an amino acid sequence comprising or consisting of SEQ ID NO:55, 56, 57, 202, 203 or 204.

In some embodiments, provided herein is a CD30-binding moiety comprising an antibody or an antigen-binding fragment thereof comprising: (a) a heavy chain variable region (VH) comprising (i) a VH CDR1 comprising SEQ ID NO:96, 97, or 98; (ii) a VH CDR2 comprising SEQ ID NO:107, 108, or 109; and (iii) a VH CDR3 comprising SEQ ID NO:121, 122, or 123; and (b) a light chain variable region (VL) comprising (i) a VL CDR1 comprising SEQ ID NO:99; (ii) a VL CDR2 comprising SEQ ID NO:110; and (iii) a VL CDR3 comprising SEQ ID NO:124, 125, or 126; or a variant thereof comprising up to 3 amino acid substitutions in each of VH CDR1, VH CDR2, VH CDR3, VL CDR1, VL CDR2, and VL CDR3.

In some embodiments, the CD30-binding moiety provided herein comprises an antibody or an antigen-binding fragment thereof comprising (a) a VH comprising a VH CDR1 comprising SEQ ID NO:96, a VH CDR2 comprising SEQ ID NO:107, and a VH CDR3 comprising SEQ ID NO:121; and/or (b) a VL comprising a VL CDR1 comprising SEQ ID NO:99, a VL CDR2 comprising SEQ ID NO:110, and a VL CDR3 comprising SEQ ID NO:124; or a variant thereof comprising up to 5 amino acid substitutions in VH CDRs and up to 5 amino acid substitutions in VL CDRs.

In some embodiments, the CD30-binding moiety provided herein comprises an antibody or an antigen-binding fragment thereof comprising (a) a VH comprising a VH CDR1 comprising SEQ ID NO:97, a VH CDR2 comprising SEQ ID NO:108, and a VH CDR3 comprising SEQ ID NO:122; and/or (b) a VL comprising a VL CDR1 comprising SEQ ID NO:99, a VL CDR2 comprising SEQ ID NO:110, and a VL CDR3 comprising SEQ ID NO:125; or a variant thereof comprising up to 5 amino acid substitutions in VH CDRs and up to 5 amino acid substitutions in VL CDRs.

In some embodiments, the CD30-binding moiety provided herein comprises an antibody or an antigen-binding fragment thereof comprising (a) a VH comprising a VH CDR1 comprising SEQ ID NO:98, a VH CDR2 comprising SEQ ID NO:109, and a VH CDR3 comprising SEQ ID NO:123; and/or (b) a VL comprising a VL CDR1 comprising SEQ ID NO:99, a VL CDR2 comprising SEQ ID NO:110, and a VL CDR3 comprising SEQ ID NO:126; or a variant thereof comprising up to 5 amino acid substitutions in VH CDRs and up to 5 amino acid substitutions in VL CDRs.

In some embodiments, the CD30-binding moiety provided herein comprises an antibody or an antigen-binding fragment thereof comprising (1) a VH comprising an amino acid sequence having 90%, 95%, 99% or 100% identity to SEQ ID NO: 218, and/or a VL comprising an amino acid sequence having 90%, 95%, 99% or 100% identity to SEQ ID NO: 219; (2) a VH comprising an amino acid sequence having 90%, 95%, 99% or 100% identity to SEQ ID NO:220, and/or a VL comprising an amino acid sequence having 90%, 95%, 99% or 100% identity to SEQ ID NO:221; or (3) a VH comprising an amino acid sequence having 90%, 95%, 99% or 100% identity to SEQ ID NO: 222, and/or a VL comprising an amino acid sequence having 90%, 95%, 99% or 100% identity to SEQ ID NO:223.

In some embodiments, the VH and VL of the CD30-binding moiety provided herein are connected by a linker. In some embodiments, the linker has an amino acid sequence comprising or consisting of SEQ ID NO:55, 56, 57, 202, 203 or 204.

In some embodiments, the CD30-binding moiety provided herein comprises a single chain variable fragment (scFv) comprising an amino acid sequence that is at least 90%, 95%, 99% identical to SEQ ID NO:58, 59, or 60. In some embodiments, the CD30-binding moiety provided herein comprises a single chain variable fragment (scFv) comprising an amino acid sequence comprising SEQ ID NO:58, 59, or 60.

In some embodiments, provided herein is a CD30-binding moiety comprising a VH CDR1, a VH CDR2, a VH CDR3, a VL CDR1, a VL CDR2, and a VL CDR3 from a binding moiety comprising a single chain variable fragment (scFv) having an amino acid sequence comprising SEQ ID: 58, 59, or 60. In some embodiments, provided herein is a CD30-binding moiety comprising a VH and a VL from a binding moiety comprising a single chain variable fragment (scFv) having an amino acid sequence comprising SEQ ID: 58, 59, or 60.

In some embodiments, the CD30-binding moiety provided herein specifically binds human CD30, rhesus CD30, or both.

In some embodiments, the CD30-binding moiety provided herein comprises an antibody or antigen-binding fragment thereof, or an extracellular domain of a receptor.

In some embodiments, the CD30-binding moiety provided herein comprises an antibody or antigen-binding fragment thereof selected from the group consisting of a single domain antibody (sdAb), a heavy chain antibody (HCAb), a Fab, a Fab′, a F(ab′)₂, a Fv, a scFv, a (scFv)₂, an IgG1 antibody, an IgG2 antibody, an IgG3 antibody, and an IgG4 antibody.

In some embodiments, the CD30-binding moiety provided herein comprises a camel antibody or antigen-binding fragment thereof, a chimeric antibody or antigen-binding fragment thereof, a humanized antibody or antigen-binding fragment thereof, or a human antibody or antigen-binding fragment thereof.

In some embodiments, the CD30-binding moiety provided herein comprises a sdAb, a HCAb, a Fab, a Fab′, a F(ab′)₂, a Fv, a scFv, a (scFv)₂, an IgG1 antibody, an IgG1 antibody, an IgG2 antibody, or an IgG3 antibody. In some embodiments, the CD30-binding moiety provided herein comprises a scFv.

In some embodiments, the CD30-binding moiety provided herein has a binding affinity (K_{D}) to CD30 that is between 10.0 pM and 500.0 nM, 100.0 pM and 200.0 nM, or 1.0 nM and 200.0 nM. In some embodiments, the K_{D} is between 3.0 nM and 170.0 nM.

Provided herein are also a polynucleotide encoding the CD30-binding moiety disclosed herein. Provided herein are also a vector comprising the polynucleotide disclosed herein. In some embodiments, the vector is a viral vector.

Provided herein is also a CD30 CAR comprising, from N-terminus to C-terminus: (a) a CD30-binding moiety disclosed herein; (b) a transmembrane domain; and (c) a cytoplasmic domain. In some embodiments, the CD30-binding moiety is a CD30-binding scFv or a CD30-binding sdAb described herein.

Provided herein are also a CAR that specifically binds CD30 (“CD30 CAR”), comprising, from N-terminus to C-terminus: (a) a bivalent CD30-binding moiety comprising a first anti-CD30 sdAb and a second anti-CD30 sdAb; (b) a transmembrane domain; and (c) a cytoplasmic domain. The first anti-CD30 sdAb and the second anti-CD30 sdAb may be identical or different, linked by a linker. If the two sdAbs are different, they may bind the same or different epitopes.

In some embodiments, the transmembrane domain of the CD30 CARs provided herein comprises CD8a transmembrane region (having an amino acid sequence of e.g., SEQ ID NO: 63) or CD28 transmembrane region.

In some embodiments, the cytoplasmic domain of the CD30 CARs provided herein comprises at least one signaling domain selected from the group consisting of CD3ζ, FcRγ, FcRβ, CD3γ, CD3δ, CD3ε, CDS, CD22, CD79a, CD79b, and CD66d.

In some embodiments, the cytoplasmic domain of the CD30 CARs provided herein comprises at least one costimulatory domains selected from the group consisting of CD28, 4-1BB (CD137), CD27, OX40, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

In some embodiments, the cytoplasmic domain of the CD30 CARs provided herein comprises a CD3ζ signaling domain (having an amino acid sequence of e.g., SEQ ID NO: 65) and a 4-1BB costimulatory domain (having an amino acid sequence of e.g., SEQ ID NO: 64). In some embodiments, the cytoplasmic domain of the CD30 CARs provided herein comprises a CD3ζ signaling domain and a CD28 costimulatory domain (having an amino acid sequence of e.g., SEQ ID NO: 129).

In some embodiments, the CD30 CARs provided herein further comprise a CD8a hinge (having an amino acid sequence of e.g., SEQ ID NO: 62) between the CD30-binding moiety and CD8a transmembrane domain (having an amino acid sequence of e.g., SEQ ID NO: 63).

In some embodiments, the CD30 CARs provided herein further comprise a CD28 hinge (having an amino acid sequence of e.g., SEQ ID NO: 127) between the CD30-binding moiety and CD28 transmembrane domain (having an amino acid sequence of e.g., SEQ ID NO: 128).

In some embodiments, the CD30 CARs provided herein further comprises a leader sequence (having an amino acid sequence of e.g., SEQ ID NO: 61) at the N-terminus.

In some embodiments, provided herein are CD30 CARs having an amino acid sequence selected from the group consisting of SEQ ID NOs: 70-86, 182-194, 201 and 208-211.

In some embodiments, the CD30 CARs provided herein are conjugated to a factor selected from the group consisting of: (i) C—C chemokine receptor type 4 (CCR4), (ii) dominant negative transforming growth factor beta receptor II (dnTGFβRII), and (iii) a chimeric switch programmed death 1 receptor (PD1CD28). In some embodiments, CCR4 comprises SEQ ID NO:67. In some embodiments, dnTGFβRII comprises SEQ ID NO:68. In some embodiments, PD1CD28 comprises SEQ ID NO:69.

In some embodiments, the CD30 CARs provided herein are conjugated to the C-terminus of the factor.

In some embodiments, the CD30 CARs provided herein are conjugated to the N-terminus of the factor.

In some embodiments, the CD30 CARs provided herein are conjugated to the factor via a 2A linker selected from the group consisting of P2A, T2A, E2A and F2A.

In some embodiments, the CD30 CARs provided herein are conjugated to a first factor and a second factor, each selected from the group consisting of: CCR4, PD1CD28 and dnTGFβRII. In some embodiments, the CD30 CAR provided herein comprises an amino acid sequence selected form the group consisting of SEQ ID NO: 195-198, 205-207, and 212-215.

In some embodiments, the CD30 CARs provided herein are conjugated to dnTGFβRII. In some embodiments, the CD30 CAR provided herein comprises an amino acid sequence selected form the group consisting of SEQ ID NO: 195, 196, 205-207, and 212-215.

In some embodiments, the CD30 CARs provided herein are conjugated to the C-terminus of the first factor, and the N-terminus of the second factor.

Provided herein are also polynucleotides encoding the CD30 CARs provided herein.

Provided herein are also vector comprising the polynucleotide provided herein. In some embodiments, the vector is a viral vector. Provided herein are also host cells comprising the polynucleotides disclosed herein or the vectors disclosed herein.

Provided herein are also cells that recombinantly express the CD30 CARs provided herein. In some embodiments, the cell is a T cell. In some embodiments, the T cell is selected from the group consisting of a cytotoxic T cell, a helper T cell, a natural killer T cell, and a γδT cell.

In some embodiments, provided herein are populations of cells comprising at least two of the cells disclosed herein.

In some embodiments, provided herein are pharmaceutical compositions comprising a therapeutically effective amount of the population of cells disclosed herein, and a pharmaceutically acceptable carrier.

Provided herein are methods of treating CD30-expressing tumor or cancer in a subject in need thereof, comprising administering to the subject a therapeutically effective amount of the pharmaceutical composition disclosed herein. In some embodiments, the CD30-expressing tumor is a lymphoma, an embryonal carcinoma (EC) or a testicular germ cell tumor (TGCT).

In some embodiments, provided herein are methods of treating lymphoma in a subject in need thereof, comprising administering to the subject a therapeutically effective amount of the pharmaceutical composition disclosed herein. In some embodiments, the lymphoma is a B-cell lymphoma. In some embodiments, the B-cell lymphoma is diffuse large B cell lymphoma (DLBCL), primary mediastinal B-cell lymphoma (PMBL), Hodgkin lymphoma (HL), non-Hodgkin lymphoma, mediastinal gray zone lymphoma, or nodular sclerosis HL. In some embodiments, the lymphoma is T-cell lymphoma. In some embodiments, the T-cell lymphoma is anaplastic large cell lymphoma (ALCL), peripheral T cell lymphoma not otherwise specified (PTCL-NOS), or angioimmunoblastic T cell lymphoma (AITL).

In some embodiments, the population of host cells is autologous to the subject.

In some embodiments, the methods provided herein further comprise obtaining T cells from the subject.

In some embodiments, the methods provided herein further comprise administering an additional therapy to the subject.

In some embodiments, the subject is a human.

### BRIEF DESCRIPTION OF THE DRAWINGS

FIG. 1. Immune response of immunized camel against recombinant human CD30 (FIG. 1A) and recombinant rhesus CD30 proteins (FIG. 1B).

FIG. 2. Binding of selected sdAbs to CD30 fragments by ELISA.

FIG. 3. In vitro cytotoxicity of selected CAR constructs assayed by LDH method. 5F11 was used as positive control.

FIG. 4. In vivo efficacy of AS48542bbz, AS48542-28z and positive control 5F11bbz. FIG. 4A depicts the tumor growth inhibition of CAR T cells on HH tumor. FIG. 4B depicts the body weight of mice after treatment by negative controls and CAR T cells.

FIG. 5. In vitro cytotoxicity of biparatopic and tandem repeat CAR constructs on CD30 high-expression MJ cell line (A) and CD30 low-expression H9 cell line (B) assayed by LDH method.

FIG. 6. In vitro cytotoxicity of humanized CAR constructs on CD30 high-expression MJ cell line (E:T=1:1) and CD30 low-expression H9 cell line (E:T=2:1) by LDH method. 5F11bbz was used as positive control.

FIG. 7. In vitro cytotoxicity of humanized tandem-repeat and biparatopic CAR T cells on CD30 high-expression MJ cell line (E:T=1:1) and CD30 low-expression H9 cell line (E:T=2:1) by LDH method. 5F11bbz was used as positive control.

FIG. 8. In vitro cytotoxicity of humanized tandem-repeat and biparatopic CAR T cells on CD30 high-expression MJ cell line (E:T=0.2:1) and CD30 low-expression H9 cell line (E:T=0.2:1) by FACS method. 5F11bbz was used as positive control.

FIG. 9. In vivo efficacy of AS48542VH5bbz, AS48542VH5dil-bbz, AS47863VH4dil-bbz, AS53574VH7-AS47863VH4bbz and positive control 5F11bbz CAR T cells. FIG. 9A depicts the tumor growth inhibition of CAR T cells on HH tumor. FIG. 9B depicts the body weight of mice after treatment by negative controls and CAR T cells.

FIG. 10. In vitro cytotoxicity of armored CAR T cells on CD30 high-expression MJ cell line (E:T=0.1:1) by FACS method.

FIG. 11. Schematic representation of chimeric antigen receptors in a cell membrane. A naked CD30 CAR includes a CD30-binding moiety (or CD30-binding domain, or target binding domain), transmembrane domain, and cytoplasmic domains, which include the signaling domain of CD28 or 4-1BB and the the signaling domain of CD3ζ (top left). CD30 CARs can be co-expressed with C—C chemokine receptor type 4 (CCR4) (top right), dominant negative transforming growth factor beta receptor II (dnTGFβRII) (bottom right), and a chimeric switch programmed death 1 receptor (PD1CD28) (bottom left).

FIG. 12. Schematic representation of chimeric antigen receptor proteins in a cell membrane. CD30 CARs can be co-expressed with CCR4 and dnTGFβRII.

FIG. 13. L540 cell lysis after 6 rounds of co-incubation with armored and unarmored, single-binder and tanden-repeat, -bbz and -28z CAR T cells. 5F11bbz CAR T was used as positive control.

FIG. 14. T cell proliferation after 6 rounds of co-incubation L540 cells at E:T ratio of 1:3. 5F11bbz CAR T was used as positive control.

FIG. 15. In vivo efficacy of armored and unarmored AS48542VH5bbz and AS47863VH4dil-bbz CAR T cells. FIG. 15A depicts the tumor growth inhibition of CAR T cells on HH tumor. FIG. 15B depicts the body weight of mice after treatment by negative controls and CAR T cells.

### DETAILED DESCRIPTION

The present disclosure provides novel binding moieties, including antibodies that specifically bind CD30. Further, the present disclosure also provides chimeric antigen receptors (CARs) that comprise such binding moieties that specifically bind CD30, as well as engineered T cells and populations of T cells that recombinantly express a CAR (CAR T cells) that specifically binds CD30. Pharmaceutical compositions comprising a therapeutically effective amount of such CAR T cells are also disclosed herein as well as methods for treating CD30-expressing tumor or cancer by administering a therapeutically effective amount of such pharmaceutical compositions.

### 1. DEFINITIONS

Unless otherwise defined herein, technical and scientific terms used in the present description have the meanings that are commonly understood by those of ordinary skill in the art.

The articles “a” and “an” as used herein refer to one or to more than one (i.e., to at least one) of the grammatical object of the article. By way of example, “an antibody” means one antibody or more than one antibody.

The term “binding moiety” as used herein refers to a molecule or a portion of a molecule which binds a target molecule (e.g., CD30). A binding moiety can comprise a protein, peptide, nucleic acid, carbohydrate, lipid, or small molecular weight compound. In some embodiments, the binding moiety comprises an antibody. In some embodiments, a binding moiety comprises an antigen-binding fragment of an antibody. In some embodiments, a binding moiety comprises a small molecular weight component. The binding moiety can also be an antibody or an antigen-binding fragment thereof. In some embodiments, a binding moiety comprises the ligand-binding domain of a receptor. In some embodiments, a binding moiety comprises the extracelluar domain of a transmembrane receptor. The binding moiety can also be the ligand-binding domain of a receptor, or the extracelluar domain of a transmembrane receptor. A binding moiety can be monovalent, which means that it contains one binding site that specifically interacts with the target molecule. A binding moiety can also be bivalent, meaning that it contains two binding sites that specifically interact with the target molecule. A binding moiety can also be multivalent, meaning that is contains multiple binding sites that specifically interact with the target molecule. A bivalent binding moiety or multivalent binding moiety can interact with one or more epitopes on a single target molecule, in which case they are also referred to as “biparatopic antibodies” or “multiparatopic antibodies.” A bivalent binding moiety or multivalent binding moiety can also interact with two or more target molecules, in which case they are also referred to as “bispecific antibodies” or “multispecific antibodies.”

The term “binding affinity” as used herein generally refers to the strength of noncovalent interactions between a binding moiety and a target molecule. The interaction between a binding moiety and a target molecule is a reversible process, and the binding affinity is a measure of the dynamic equilibrium of the ratio of the dissociation rate (k_{off} or k_{d}) to the association rate (kₒₙ or kₐ) and typically reported as the equilibrium dissociation constant (K_{D}). A variety of methods of measuring binding affinity are known in the art, any of which can be used for purposes of the present disclosure. In one embodiment, the “K_{D}” or “K_{D} value” is measured by assays known in the art, for example by a binding assay. The K_{D} may be measured in a radiolabeled antigen binding assay (RIA) (Chen, et al., (1999) J. Mol Biol 293:865-881). The K_{D} or K_{D} value may also be measured by using surface plasmon resonance assays by Biacore, using, for example, a BIAcore™-2000 or a BIAcore™-3000 BIAcore, Inc., Piscataway, N.J.), or by biolayer interferometry using, for example, the OctetQK384 system (ForteBio, Menlo Park, Calif.). Avidity is commonly applied to antibody interactions in which multiple antigen-binding sites simultaneously interact with the target antigenic epitopes, and therefore refers to the accumulated strength of multiple affinities. IgM usually has low affinity but high avidity as it has 10 weak binding sites for antigen, enabling its effective antigen binding.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g. antibody) that specifically binds a target molecule (e.g. antigen) can be identified, for example, by immunoassays, ELISAs, SPR (e.g., Biacore), or other techniques known to those of skill in the art. Typically a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety that specifically binds a first target may or may not specifically bind a second target. As such, “specific binding” does not necessarily require (although it can include) exclusive binding, i.e., binding to a single target. Thus, a binding moiety can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding fragments” of intact antibodies.

The term “antigen-binding fragment” as used in connection with an antibody refers to a portion of an intact antibody and refers to the antigenic determining variable regions of an intact antibody. Examples of antibody fragments include, but are not limited to, Fab, Fab′, F(ab′)₂, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), single variable domain of heavy chain antibodies (VHH), and multispecific antibodies formed from antibody fragments.

The term “variable region” of an antibody as used herein refers to the variable region of an antibody light chain, or the variable region of an antibody heavy chain, either alone or in combination. In naturally occurring heavy chain only antibodies, the term “variable region” refers to the heavy chain variable region, also termed as VHH fragment. Generally, the heavy or light chain variable region, or the VHH fragment, may consist of four framework regions (FR) and three complementarity determining regions (CDRs), also known as “hypervariable regions.” The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (Kabat et al., 1991, Sequences of Proteins of Immunological Interest (5 ed.). Bethesda, Md.: National Institutes of Health), and (2) an approach based on crystallographic studies of antigen-antibody complexes (Al-Lazikani et al., 1997, J. Mol. Biol., 273(4):927-48). In addition, combinations of these two approaches are used in the art and can be used to determine CDRs.

The term “single domain antibody” or “sdAb”, as used herein, refers to an antibody consisting of a single variable region having three CDRs, which alone is capable of binding to an antigen without pairing with a corresponding CDR-containing polypeptide. The single domain antibody includes the VHH fragment from or derived from a camelid heavy chain only antibody, and can be fused to a heavy chain constant region as needed.

The term “camelid antibody”, as used herein, is intended to include antibodies having variable regions in which both the framework and CDR regions are derived from camelid germline heavy chain antibody sequences. Furthermore, if the antibody contains a constant region, the constant region also is derived from camelid germline heavy chain antibody sequences. The camelid antibodies of the invention can include amino acid residues not encoded by camelid germline heavy chain antibody sequences (e.g., mutations introduced by random or site-specific mutagenesis in vitro or by somatic mutation in vivo). However, the term “camelid antibody”, as used herein, is not intended to include antibodies in which CDR sequences derived from the germline of another mammalian species have been grafted onto camelid framework sequences.

The term “single chain variable fragment” or “scFv” refers to a fusion protein of the heavy chain variable region and light chain variable region of immunoglobulins, connected with a short linker peptide of ten to twenty-five amino acids. The linker is usually rich in glycine for flexibility, as well as serine or threonine for solubility. The scFv retains the specificity of the original immunoglobulin. The scFvs can be linkered by linkers of different lengths to form di-scFvs, diabodies, tri-scFvs, triabodies, or tetrabodies, which may show specificity to one or more antigens.

The term “chimeric antibody” refers to an antibody made by combining genetic material from a nonhuman source with genetic material from a human being. Or more generally, a chimetic antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “human antibody” as used herein refers to an antibody produced by a human or an antibody having an amino acid sequence corresponding to an antibody produced by a human made using any of the techniques known in the art.

The terms “epitope” and “antigenic determinant” are used interchangeably herein an refer to the site on the surface of a target molecule to which a binding moiety binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g. a polypeptide) contributing to an epitope may be contiguous amino acids of the polypeptide or the epitope may come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “linker” or “linker region” as used herein refers to a molecular sequence that connects two molecules or two sequences on the same molecule. In some embodiments, the linker is a peptide linker. Preferably, linkers do not adversely affect the expression, secretion, or bioactivity of the polypeptides. In addition, linkers are preferably not antigenic and do not elicit an immune response. In some embodiments, the linker can be an endogenous amino acid sequence, an exogenous amino acid sequence (e.g., GS-rich sequence), or a non-peptide chemical linker.

The terms “polypeptide,” “peptide,” and “protein” as used interchangeably herein refer to polymers of amino acids of any length, which can be linear or branched. It can include unnatural or modified amino acids, or be interrupted by non-amino acids. A polypeptide, peptide, or protein, can also be modified with, for example, disulfide bond formation, glycosylation, lipidation, acetylation, phosphorylation, or any other manipulation or modification.

The terms “polynucleotide” and “nucleic acid” as used interchangeably herein refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The terms “identical” or percent “identity” as used herein in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “amino acid substitution,” as used herein, refers to the replacement of one amino acid residue with another in a polypeptide sequence. A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “variant” as used herein in relation to a binding moiety (e.g. an antibody) having a polypeptide with particular sequence features (the “reference binding moiety”) refers to a different binding moiety having a polypeptide comprising one or more (such as, for example, about 1 to about 25, about 1 to about 20, about 1 to about 15, about 1 to about 10, or about 1 to about 5) amino acid sequence substitutions, deletions, and/or additions as compared to the reference binding moiety. An anti-CD30-binding moiety variant or anti-CD30 antibody variant at least retains specific binding to CD30. In some embodiments, a binding moiety variant can result from one or more (such as, for example, about 1 to about 25, about 1 to about 20, about 1 to about 15, about 1 to about 10, or about 1 to about 5) changes to an amino acid sequence of a reference binding moiety. Also by way of example, a variant of an anti-CD30 antibody can result from one or more (such as, for example, about 1 to about 25, about 1 to about 20, about 1 to about 15, about 1 to about 10, or about 1 to about 5) changes to an amino acid sequence of a reference anti-CD30 antibody. The changes to an amino acid sequence can be amino acid substitutions. In some embodiments, the changes to an amino acid sequence can be conservative amino acid substitutions. In some embodiments, an anti-CD30-binding moiety variant or anti-CD30 antibody variant can result from one or more (such as, for example, about 1 to about 25, about 1 to about 20, about 1 to about 15, about 1 to about 10, or about 1 to about 5) amino acid substitutions in the VH or VL regions or subregions, such as one or more CDRs. In some embodiments, an anti-CD30-binding moiety variant or anti-CD30 antibody variant can result from one, up to two, up to three, up to four, or up to five amino acid substitutions in each of the VH or VL region. In some embodiments, an anti-CD30-binding moiety variant or anti-CD30 antibody variant can result from one, up to two, up to three, up to four, or up to five amino acid substitutions in each of the CDRs region.

The term “vector” refers to a substance that is used to carry or include a nucleic acid sequences, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. It is understood by those skilled in the art that the nucleic acid molecules are expressed in a sufficient amount to produce a desired product (e.g. an anti-CD30 CAR as described herein), and it is further understood that expression levels can be optimized to obtain sufficient expression using methods well known in the art.

The term “chimeric antigen receptor” or “CAR” as used herein refers to an artificially constructed hybrid protein or polypeptide containing a binding moiety (e.g. an antibody) linked to immune cell (e.g. T cell) signaling or activation domains. In some embodiments, CARs are synthetic receptors that retarget T cells to tumor surface antigens (Sadelain et al., Nat. Rev. Cancer 3(1):35-45 (2003); Sadelain et al., Cancer Discovery 3(4):388-398 (2013)). CARs can provide both antigen binding and immune cell activation functions onto an immune cell such as a T cell. CARs have the ability to redirect T-cell specificity and reactivity toward a selected target in a non-MHC-restricted manner, exploiting the antigen-binding properties of monoclonal antibodies. The non-MHC-restricted antigen recognition can give T-cells expressing CARs the ability to recognize an antigen independent of antigen processing, thus bypassing a mechanism of tumor escape.

The term “subject” refers to any animal (e.g., a mammal), including, but not limited to, humans, non-human primates, canines, felines, rodents, and the like, which is to be the recipient of a particular treatment. In some embodiments, a subject is a human A “subject” can be a patient with a particular disease. In some embodiments, a subject is a patient having a CD-30 expressing cancer or tumor.

The term “treat” as used herein in connection with a disease or a condition, or a subject having a disease or a condition refers to an action that suppresses, eliminates, reduces, and/or ameliorates a symptom, the severity of the symptom, and/or the frequency of the symptom associated with the disease or disorder being treated. When used in reference to a cancer or tumor, the term “treat” refers to an action that reduces the severity of the cancer or tumor, or retards or slows the progression of the cancer or tumor, including (a) inhibiting the growth, or arresting development of the cancer or tumor, or (b) causing regression of the cancer or tumor, or (c) delaying, ameliorating or minimizing one or more symptoms associated with the presence of the cancer or tumor.

The term “administer,” “administering,” or “administration” as used herein refers to the act of delivering, or causing to be delivered, a therapeutic or a pharmaceutical composition to the body of a subject by a method described herein or otherwise known in the art. The therapeutic can be a compound, a polypeptide, a cell, or a population of cells. Administering a therapeutic or a pharmaceutical composition includes prescribing a therapeutic or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The term “therapeutically effective amount” as used herein refers to an amount of a compound, polypeptide, cell, formulation, material, or composition, as described herein sufficient to provide a therapeutic benefit in the treatment of the disease or disorder or to delay or minimize one or more symptoms associated with the disease or disorder. The disease or disorder can be a CD-30 expressing cancer or tumor.

As used herein, the term “carrier” include “pharmaceutically acceptable carriers,” excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete or incomplete)), excipient, or vehicle with which therapeutic is administered. Such carriers, including pharmaceutical carriers, can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences (1990) Mack Publishing Co., Easton, Pa. Compositions, including pharmaceutical compounds, may contain a prophylactically or therapeutically effective amount of an anti-beta klotho antibody, for example, in isolated or purified form, together with a suitable amount of carrier so as to provide the form for proper administration to the subject (e.g., patient). The formulation should suit the mode of administration.

The term “autologous” as used herein refers to any material derived from the same individual to which it is later to be re-introduced into the individual.

The term “allogeneic” as used herein refers to a graft derived from a different animal of the same species.

### 2. CD30-BINDING MOIETIES

CD30, also commonly known as Ki-1 or TNFRSF8, is a member of the tumor necrosis factor receptor superfamily. The human CD30 is expressed transiently at low levels on intrafollicular and perifollicular T and B cell blasts in lymphoid tissues, but is specifically upregulated on certain hematopoietic malignancies, including anaplastic large cell lymphoma and Hodgkin lymphoma, among others.

The human CD30 protein has 595 amino acids. Representative amino acid sequences for full length human CD30 can be found at GenBank Nos: AAA51947.1 or CAC16652.1. CD30 exists as a 120 kDa membrane glycoprotein chain, with the extracellular domain (ECD) containing binding sites for CD30 ligand, and the cytoplasmic domain playing a crucial role in signal transduction. Representative amino acid sequences for the extracellular domain (ECD) of human CD30 and rhesus CD30 are provided herein as SEQ ID NO:1 and SEQ ID NO:2, respectively. The human CD30 ECD can be further divided into six cysteine-rich domains (CRDs) of approximately 40 amino acids each, which are: CRD1 (F19-Q68, SEQ ID NO:3), CRD2 (R66-E107, SEQ ID NO:4), CRD3 (E107-5153, SEQ ID NO:5), CRD4 (E150-Q243, SEQ ID NO:6), CRD5 (R241-E282, SEQ ID NO:7) and CRD6 (E282-K379, SEQ ID NO:8).

The present disclosure provides binding moieties that specifically bind CD30. In some embodiments, a CD30-binding moiety specifically binds a fragment of CD30. In some embodiments, a CD30-binding moiety specifically binds the ECD of CD30. In some embodiments, a CD30-binding moiety specifically binds the CRD1 of CD30. In some embodiments, a CD30-binding moiety specifically binds the CRD2 of CD30. In some embodiments, a CD30-binding moiety specifically binds the CRD3 of CD30. In some embodiments, a CD30-binding moiety specifically binds the CRD4 of CD30. In some embodiments, a CD30-binding moiety specifically binds the CRD5 of CD30. In some embodiments, a CD30-binding moiety specifically binds the CRD6 of CD30. In some embodiments, a CD30-binding moiety specifically binds at least two of the CRD1, CRD2, CRD3, CRD4, CRD5, and CRD6 domains of CD30. In some embodiments, a CD30-binding moiety specifically binds at least CRD1 and CRD6 domains of CD30. In some embodiments, a CD30-binding moiety specifically binds at least three of the CRD1, CRD2, CRD3, CRD4, CRD5, and CRD6 domains of CD30. In some embodiments, a CD30-binding moiety specifically binds at least four of the CRD1, CRD2, CRD3, CRD4, CRD5, and CRD6 domains of CD30. In some embodiments, a CD30-binding moiety specifically binds at least five of the CRD1, CRD2, CRD3, CRD4, CRD5, and CRD6 domains of CD30. In some embodiments, a CD30-binding moiety specifically binds all six of the CRD1, CRD2, CRD3, CRD4, CRD5, and CRD6 domains of CD30. In some embodiments, a CD30-binding moiety specifically binds an epitope on CD30. In some embodiments, a CD30-binding moiety specifically binds a linear epitope on CD30. In some embodiments, a CD30-binding moiety specifically binds a conformational epitope on CD30. In some embodiments, a CD30-binding moiety specifically binds human CD30. In some embodiments, a CD30-binding moiety specifically binds rhesus CD30. In some embodiments, a CD30-binding moiety specifically binds human CD30 and rhesus CD30.

In some embodiments, a CD30-binding moiety specifically binds within amino acids 19-68 of human CD30. In some embodiments, a CD30-binding moiety specifically binds within amino acids 66-107 of human CD30. In some embodiments, a CD30-binding moiety specifically binds within amino acids 107-153 of human CD30. In some embodiments, a CD30-binding moiety specifically binds within amino acids 150-243 of human CD30. In some embodiments, a CD30-binding moiety specifically binds within amino acids 241-282 of human CD30. In some embodiments, a CD30-binding moiety specifically binds within amino acids 282-379 of human CD30.

In some embodiments, the CD30-binding moiety specifically binds an epitope comprising amino acids within SEQ ID NO:3. In some embodiments, the CD30-binding moiety specifically binds an epitope comprising amino acids within SEQ ID NO:4. In some embodiments, the CD30-binding moiety specifically binds an epitope comprising amino acids within SEQ ID NO:5. In some embodiments, the CD30-binding moiety specifically binds an epitope comprising amino acids within SEQ ID NO:6. In some embodiments, the CD30-binding moiety specifically binds an epitope comprising amino acids within SEQ ID NO:7. In some embodiments, the CD30-binding moiety specifically binds an epitope comprising amino acids within SEQ ID NO:8. In some embodiments, the CD30-binding moiety specifically binds at least one amino acid within SEQ ID NO:3. In some embodiments, the CD30-binding moiety specifically binds at least one amino acid within SEQ ID NO:4. In some embodiments, the CD30-binding moiety specifically binds at least one amino acid within SEQ ID NO:5. In some embodiments, the CD30-binding moiety specifically binds at least one amino acid within SEQ ID NO:6. In some embodiments, the CD30-binding moiety specifically binds at least one amino acid within SEQ ID NO:7. In some embodiments, the CD30-binding moiety specifically binds at least one amino acid within SEQ ID NO:8.

In some embodiments, the binding moiety comprises a ligand-binding domain of a receptor. In some embodiments, the binding moiety is a ligand-binding domain of a receptor. In some embodiments, the binding moiety comprises an ECD of a transmembrane receptor. In some embodiments, the binding moiety is an ECD of a transmembrane receptor.

In some embodiments, a CD30-binding moiety comprises an antibody (including an antigen-binding fragment thereof). In some embodiments, a CD30-binding moiety comprises an antigen-binding fragment of an antibody. In some embodiments, a CD30-binding moiety is an antibody. In some embodiments, the antibody is an IgA, IgD, IgE, IgG, or IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, a CD30-binding moiety comprises a single domain antibody (sdAb). In some embodiments, a CD30-binding moiety comprises a heavy chain antibody (HCAb). In some embodiments, a CD30-binding moiety comprises a Fab. In some embodiments, the antibody is a Fab′. In some embodiments, a CD30-binding moiety comprises a F(ab′)₂. In some embodiments, a CD30-binding moiety comprises a Fv. In some embodiments, a CD30-binding moiety comprises a scFv. In some embodiments a CD30-binding moiety comprises a disulfide-linked scFv [(scFv)₂]. In some embodiments, a CD30-binding moiety comprises a diabody (dAb).

In some embodiments, a CD30-binding moiety comprises comprises a sdAb. Exemplary sdAbs include, but are not limited to, naturally occurring sdAbs, recombinant sdAbs derived from conventional four-chain antibodies, engineered single domain scaffolds other than those derived from antibodies. sdAbs can be derived from any species including, but not limited to mouse, human, camel, llama, fish, shark, goat, rabbit, and bovine. In some embodiments, the binding moiety comprises a HCAb. In some embodiments, the HCAb comprises a sdAb that is fused with a Fc region. In some embodiments, the HCAb comprises a sdAb that is fused with a human IgG1 hinge and Fc region.

In some embodiments, a CD30-binding moiety comprises a recombinant antibody. In some embodiments, a CD30-binding moiety comprises a monoclonal antibody. In some embodiments, a CD30-binding moiety comprises a polyclonal antibody. In some embodiments, a CD30-binding moiety comprises a camelid (e.g., camels, dromedary and llamas) antibody. In some embodiments, a CD30-binding moiety comprises a camel antibody. In some embodiments, a CD30-binding moiety comprises a chimeric antibody. In some embodiments, a CD30-binding moiety comprises a humanized antibody. In some embodiments, a CD30-binding moiety comprises a human antibody.

In some embodiments, a CD30-binding moiety comprises a bispecific binding moiety or a multispecific binding moiety.

In some embodiments, a CD30-binding moiety comprises a monovalent binding moiety. In some embodiments, a CD30-binding moiety (e.g. antibody) comprises a monospecific binding moiety. In some embodiments, a CD30-binding moiety (e.g. antibody) comprises a bivalent binding moiety. In some embodiments, the bivalent binding moiety comprises two antibodies. In some embodiments, the bivalent binding moiety comprises a first antibody and a second antibody. In some embodiments, the first antibody and the second antibody are connected by a linker. In some embodiments, a CD30-binding moiety (e.g. antibody) comprises a first antibody, a linker and a second antibody, from N-terminus to C-terminus. In some embodiments, the second antibody is a tandem repeat of the first antibody. In some embodiments, the first antibody and the second antibody recognize different epitopes on CD30. In some embodiments, the first antibody and the second antibody recognize the same epitope on CD30.

The antibody can be selected from the group consisting of a single domain antibody (sdAb), a heavy chain antibody (HCAb), a Fab, a Fab′, a F(ab′)₂, a Fv, a scFv, a (scFv)₂, an IgG1 antibody, an IgG2 antibody, an IgG3 antibody, and an IgG4 antibody.

In some embodiments, a bivalent CD30-binding moiety comprises a first sdAb and a second sdAb. In some embodiments, the first sdAb and a second sdAb are connected by a linker. In some embodiments, a bivalent CD30-binding moiety comprises a first sdAb, a linker and a second sdAb, from N-terminus to C-terminus. In some embodiments, the second sdAb is a tandem repeat of the first sdAb. In some embodiments, the first sdAb and the second sdAb recognize different epitopes on CD30. In some embodiments, the first sdAb and the second sdAb recognize the same epitope on CD30.

In some embodiments, the antibody is isolated. In some embodiments, the antibody is substantially pure.

In some embodiments, a CD30-binding moiety is a monoclonal antibody. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable domains or CDRs of a desired species. Screening of phage libraries can be accomplished by various techniques known in the art.

In addition to normal heavy and light chain antibodies, camelids (e.g., camels, dromedary and llamas) generate single domain antibodies (sdAbs) comprising a single monomeric heavy chain. In some embodiments, the binding moieties disclosed herein comprise a sdAb derived from camelid. These are coded for by a distinct set of VH segments, referred to as VHH genes. Methods are known in the art for achieving high affinity binding with camelid-derived sdAbs and are similar to those for conventional antibodies. A non-limiting example of such a method is hypermutation of the variable region and selection of the cells expressing such high affinity antibodies (affinity maturation).

In certain embodiments, the binding moieties comprise one or more sdAbs that are recombinant, CDR-grafted, humanized, camelized, de-immunized, and/or in vitro generated (e.g., selected by phage display). Techniques for generating antibodies and sdAb, and modifying them recombinantly are known in the art.

In addition to the use of display libraries, the specified antigen (e.g. recombinant CD30 or an epitope thereof) can be used to immunize a non-human animal, e.g., a rodent or camelid. In certain embodiments, camelid antigen-binding fragments (e.g., sdAbs) can be generated and isolated using methods known in the art and/or disclosed herein. In some embodiments, a camel can be immunized with an antigen (e.g., recombinant CD30 or an epitope thereof).

In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a rhesus protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody and their sequence is determined using standard techniques. The isolated polynucleotides encoding the heavy and light chains are then cloned into suitable expression vectors that produce the monoclonal antibodies when transfected into host cells such as E. coli, simian COS cells, Chinese hamster ovary (CHO) cells, or myeloma cells that do not otherwise produce immunoglobulin proteins.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, a CD30-binding moiety is a humanized antibody. Various methods for generating humanized antibodies are known in the art. In some embodiments, a humanized antibody comprises one or more amino acid residues that have been introduced into its sequence from a source that is non-human. In some embodiments, humanization is performed by substituting one or more non-human CDR sequences for the corresponding CDR sequences of a human antibody. In some embodiments, the humanized antibodies are constructed by substituting all three CDRs of a non-human antibody (e.g., a camelid VHH) for the corresponding CDRs of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region are used for generating humanized antibodies can be made based on a variety of factors and by a variety of methods known in the art. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

In some embodiments, a CD30-binding moiety is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequence and determination of CDRs.

The specific CDR sequences defined herein are generally based on a combination of Kabat and Chothia definitions (Exemplary system). However, it will be understood that reference to a heavy chain CDR or CDRs and/or a light chain CDR or CDRs of a specific antibody will encompass all CDR definitions as known to those of skill in the art.

The amino acid sequences and/or sequence ID numbers of camelid and humanized sdAbs (or V_{H}Hs) and the corresponding CDRs of the disclosure are summarized in Table 1, and those of CDRs, VHs, VLs and scFvs of exemplary 4-chain antibodies are listed in Table 2.

TABLE 1
Amino acid sequences and/or
amino acid sequence ID numbers of CDRs and sdAbs

| Antibody ID | CDR1 | CDR2 | CDR3 | sdAb |
|---|---|---|---|---|
| AS47863 | GSTFGDSDMG | IISSDGRTYYVDSV | DLRQYCRDGR | SEQ ID NO: 9 |
| AS47863VH4 | (SEQ ID NO: 87) | KG | CCGY | SEQ ID NO: 19 |
| AS47863VH5 | | (SEQ ID NO: 100) | (SEQ ID NO: 111) | SEQ ID NO: 20 |
| AS47863VH11 | | | | SEQ ID NO: 21 |
| AS47863VH12 | | | | SEQ ID NO: 22 |
| AS48433 | GSTFGDSDMG | IISSDGRTYYVDSV | DLRLNCRDGR | SEQ ID NO: 10 |
| AS48433VH4 | (SEQ ID NO: 87) | KG | CCGY | SEQ ID NO: 23 |
| AS48433VH5 | | (SEQ ID NO: 100) | (SEQ ID NO: 112) | SEQ ID NO: 24 |
| AS48433VH11 | | | | SEQ ID NO: 25 |
| AS48433VH12 | | | | SEQ ID NO: 26 |
| AS48463 | GFTFANSDMG | IISSHGGTTYYVDS | DPRSNCRGGYC | SEQ ID NO: 11 |
| AS48463VH4 | (SEQ ID NO: 88) | VKG | CGY | SEQ ID NO: 27 |
| AS48463VH11 | | (SEQ ID NO: 101) | (SEQ ID NO: 113) | SEQ ID NO: 28 |
| AS48481 | GFTFADSAMG | IIRTDGTTYYGDS | DRETSFIGGSW | SEQ ID NO: 12 |
| AS48481VH5 | (SEQ ID NO: 89) | AKG | CVAKY | SEQ ID NO: 29 |
| AS48481VH6 | | (SEQ ID NO: 102) | (SEQ ID NO: 114) | SEQ ID NO: 30 |
| AS48481VH13 | | | | SEQ ID NO: 31 |
| AS48481VH14 | | | | SEQ ID NO: 32 |
| AS48508 | RFTFDGPDMA | IISADGRTYYTDS | DPRRNCRGGY | SEQ ID NO: 13 |
| AS48508VH4 | (SEQ ID NO: 90) | VKG | CCGN | SEQ ID NO: 33 |
| AS48508VH5 | | (SEQ ID NO: 103) | (SEQ ID NO: 115) | SEQ ID NO: 34 |
| AS48508VH11 | | | | SEQ ID NO: 35 |
| AS48508VH12 | | | | SEQ ID NO: 36 |
| AS48542 | AFTFDGPDMA | IISADGRTYYADS | DPRKNCRGGY | SEQ ID NO: 14 |
| AS48542VH5 | (SEQ ID NO: 91) | VKG | CCAN | SEQ ID NO: 37 |
| AS48542VH12 | | (SEQ ID NO: 104) | (SEQ ID NO: 116) | SEQ ID NO: 38 |
| AS53445 | GYIFCMG | TIYTGGDSTYYDD | GGQECYLTNW | SEQ ID NO: 15 |
| AS53445VH4 | (SEQ ID NO: 92) | SVKG | VSY | SEQ ID NO: 39 |
| AS53445VH11 | | (SEQ ID NO: 105) | (SEQ ID NO: 117) | SEQ ID NO: 40 |
| AS53574 | GYIYSSNCMG | RIHTGSGSTYYAD | GRVVLGAVVC | SEQ ID NO: 16 |
| AS53574VH4 | (SEQ ID NO: 93) | SVKG | TNEY | SEQ ID NO: 41 |
| AS53574VH5 | | (SEQ ID NO: 106) | (SEQ ID NO: 118) | SEQ ID NO: 42 |
| AS53574VH6 | | | | SEQ ID NO: 43 |
| AS53574VH11 | | | | SEQ ID NO: 44 |
| AS53574VH12 | | | | SEQ ID NO: 45 |
| AS53574VH13 | | | | SEQ ID NO: 46 |
| AS53574VH7 | | | | SEQ ID NO: 199 |
| AS53750 | GFTDDGPDMA | IISADGRTYYTDS | DPRRNCRGGD | SEQ ID NO: 17 |
| AS53750VH4 | (SEQ ID NO: 94) | VKG | CCGN | SEQ ID NO: 47 |
| AS53750VH5 | | (SEQ ID NO: 103) | (SEQ ID NO: 119) | SEQ ID NO: 48 |
| AS53750VH11 | | | | SEQ ID NO: 49 |
| AS53750VH12 | | | | SEQ ID NO: 50 |
| AS54233 | GFTFDGPDMA | IISADGRTYYTDS | DPRRNCRGNC | SEQ ID NO: 18 |
| AS54233VH4 | (SEQ ID NO: 95) | VKG | CGN | SEQ ID NO: 51 |
| AS54233VH5 | | (SEQ ID NO: 103) | (SEQ ID NO: 120) | SEQ ID NO: 52 |
| AS54233VH11 | | | | SEQ ID NO: 53 |
| AS54233VH12 | | | | SEQ ID NO: 54 |

TABLE 2
Amino acid sequences and/or
sequence ID numbers of CDRs, VHs, VLs and scFvs

| Antibody | | | | | |
|---|---|---|---|---|---|
| ID | VH/VL | CDR1 | CDR2 | CDR3 | scFv |
| AS57911 | VH (SEQ ID | GFNISSSYIH | YISSYYSYTYYADSVKG | GYPYGMDY | SEQ ID |
| | NO: 218) | (SEQ ID NO: 98) | (SEQ ID NO: 109) | (SEQ ID NO: 123) | NO: 58 |
| | VL (SEQ ID | RASQSVSSAVA | SASSLYS | QQSHALIT | |
| | NO: 219) | (SEQ ID NO: 99) | (SEQ ID NO: 110) | (SEQ ID NO: 126) | |
| AS57659 | VH (SEQ ID | GFNIYSYYIH | SIYSSYSSTYYADSVKG | SWFSYPGLDY | SEQ ID |
| | NO: 220) | (SEQ ID NO: 96) | (SEQ ID NO: 107) | (SEQ ID NO: 121) | NO: 59 |
| | VL (SEQ ID | RASQSVSSAVA | SASSLYS | QQPYYLIT | |
| | NO: 221) | (SEQ ID NO: 99) | (SEQ ID NO: 110) | (SEQ ID NO: 124) | |
| AS57765 | VH (SEQ ID | GFNIYYSYMH | YIYPYSGSTSYADSVKG | PAVHWHGYGGGYYYGLDY | SEQ ID |
| | NO: 222) | (SEQ ID NO: 97) | (SEQ ID NO: 108) | (SEQ ID NO: 122) | NO: 60 |
| | VL (SEQ ID | RASQSVSSAVA | SASSLYS | QQAYYSLIT | |
| | NO: 223) | (SEQ ID NO: 99) | (SEQ ID NO: 110) | (SEQ ID NO: 125) | |

In some embodiments, a CD30-binding moiety comprises an antibody. In some embodiments, a CD30-binding moiety comprises a sdAb. In some embodiments, a CD30-binding moiety comprises an antibody having CDR1, CDR2 and/or CDR3 from an antibody described herein. In some embodiments, a CD30-binding moiety comprises an antibody having CDR1, CDR2 and CDR3 from an antibody described herein. In some embodiments, a CD30-binding moiety comprises a humanized version of an antibody described herein. In some embodiments, a CD30-binding moiety comprises a variant of an anti-CD30 antibody described herein. In some embodiments, a variant of the anti-CD30 antibody comprises one to thirty conservative amino acid substitutions. In some embodiments, a variant of the anti-CD30 antibody comprises one to twenty-five conservative amino acid substitutions. In some embodiments, a variant of the anti-CD30 antibody comprises one to twenty conservative amino acid substitutions. In some embodiments, a variant of the anti-CD30 antibody comprises one to fifteen conservative amino acid substitutions. In some embodiments, a variant of the anti-CD30 antibody comprises one to ten conservative amino acid substitution(s). In some embodiments, a variant of the anti-CD30 antibody comprises one to five conservative amino acid substitution(s). In some embodiments, a variant of the anti-CD30 antibody comprises one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, a CD30-binding moiety comprises: (a) a CDR1 comprising SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, or a variant thereof comprising 1, 2, 3, or 4 amino acid substitutions; (b) a CDR2 comprising SEQ ID NO:100, SEQ ID NO:101, SEQ ID NO:102, SEQ ID NO:103, SEQ ID NO:104, SEQ ID NO:105, SEQ ID NO:106, or a variant thereof comprising 1, 2, 3, or 4 amino acid substitutions; and/or (c) a CDR3 comprising SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, or a variant thereof comprising 1, 2, 3, or 4 amino acid substitutions. In some embodiments, a CDR (CDR1, CDR2, and/or CDR3) comprises one amino acid substitution. In some embodiments, a CDR (CDR1, CDR2, and/or CDR3) comprises two amino acid substitutions. In some embodiments, a CDR (CDR1, CDR2, and/or CDR3) comprises three amino acid substitutions. In some embodiments, a CDR (CDR1, CDR2, and/or CDR3) comprises four amino acid substitutions. In some embodiments, the one or more amino acid substitutions are conservative substitutions. In some embodiments, the one or more substitutions are made as part of a humanization process. In some embodiments, the one or more substitutions are made as part of a germline humanization process. In some embodiments, the one or more substitutions are made as part of an affinity maturation process. In some embodiments, the one or more substitutions are made as part of an optimization process.

In some embodiments, a CD30-binding moiety comprises: a CDR1 comprising SEQ ID NO:87, a CDR2 comprising SEQ ID NO:100, and/or a CDR3 comprising SEQ ID NO:111. In some embodiments, a CD30-binding moiety comprises: a CDR1 comprising SEQ ID NO:87, a CDR2 comprising SEQ ID NO:100, and a CDR3 comprising SEQ ID NO:111. In some embodiments, a CD30-binding moiety comprises: a CDR1 comprising SEQ ID NO: 87, or a variant thereof comprising 1, 2, 3, or 4 amino acid substitutions; a CDR2 comprising SEQ ID NO:100, or a variant thereof comprising 1, 2, 3, or 4 amino acid substitutions; a CDR3 comprising SEQ ID NO:111, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:9. In some embodiments, a CD30-binding moiety comprises a tandem repeat of an antibody having a CDR1, CDR2, and CDR3 from a sdAb having the amino acid sequence of SEQ ID NO:9.

In some embodiments, a CD30-binding moiety comprises an amino acid sequence that is at least about 80% sequence identity to SEQ ID NO:9. In some embodiments, a CD30-binding moiety comprises an amino acid sequence that is at least about 85%, at least about 90%, at least about 95%, at least about 97%, or at least about 99% sequence identity to SEQ ID NO:9. In some embodiments, a CD30-binding moiety comprises an amino acid sequence that is at least about 85% sequence identity to SEQ ID NO:9. In some embodiments, a CD30-binding moiety comprises an amino acid sequence that is at least about 90% sequence identity to SEQ ID NO:9. In some embodiments, a CD30-binding moiety comprises an amino acid sequence that is at least about 95% sequence identity to SEQ ID NO:9. In some embodiments, a CD30-binding moiety comprises an amino acid sequence that is at least about 97% sequence identity to SEQ ID NO:9. In some embodiments, a CD30-binding moiety comprises an amino acid sequence that is at least about 99% sequence identity to SEQ ID NO:9. In some embodiments, a CD30-binding moiety comprises an amino acid sequence that is SEQ ID NO:9. In some embodiments, a CD30-binding moiety comprises an amino acid sequence that is SEQ ID NO:19. In some embodiments, a CD30-binding moiety comprises an amino acid sequence that is SEQ ID NO:20. In some embodiments, a CD30-binding moiety comprises an amino acid sequence that is SEQ ID NO:21. In some embodiments, a CD30-binding moiety comprises an amino acid sequence that is SEQ ID NO:22.

In some embodiments, a CD30-binding moiety comprises a CDR1, CDR2, and/or CDR3 from antibody AS47863, a humanized version thereof, or variants thereof. In some embodiments, a CD30-binding moiety comprises a CDR1, CDR2, and CDR3 from antibody AS47863. In some embodiments, a CD30-binding moiety comprises a humanized version of antibody AS47863. In some embodiments, a CD30-binding moiety comprises a variant of antibody AS47863. In some embodiments, a CD30-binding moiety comprises antibody AS47863 (SEQ ID NO:9). In some embodiments, a CD30-binding moiety comprises antibody AS47863VH4 (SEQ ID NO:19). In some embodiments, a CD30-binding moiety comprises antibody AS47863VH5 (SEQ ID NO:20). In some embodiments, a CD30-binding moiety comprises antibody AS47863VH11 (SEQ ID NO:21). In some embodiments, a CD30-binding moiety comprises antibody AS47863VH12 (SEQ ID NO:22). In some embodiments, a CD30-binding moiety comprises a tandem repeat of antibody AS47863, AS47863VH4, AS47863VH5, AS47863VH11, or AS47863VH12.

In some embodiments, a binding moiety competes for binding to CD30 with a reference antibody, wherein the reference antibody comprises a CDR1 comprising SEQ ID NO:87, a CDR2 comprising SEQ ID NO:100, and a CDR3 comprising SEQ ID NO:111. In some embodiments, a binding moiety competes for binding to CD30 with a reference antibody, wherein the reference antibody is AS47863.

In some embodiments, a CD30-binding moiety comprises: a CDR1 comprising SEQ ID NO:87, a CDR2 comprising SEQ ID NO:100, and/or a CDR3 comprising SEQ ID NO:112. In some embodiments, a CD30-binding moiety comprises: a CDR1 comprising SEQ ID NO:87, a CDR2 comprising SEQ ID NO:100, and a CDR3 comprising SEQ ID NO:112. In some embodiments, a CD30-binding moiety comprises: a CDR1 comprising SEQ ID NO: 87, or a variant thereof comprising 1, 2, 3, or 4 amino acid substitutions; a CDR2 comprising SEQ ID NO:100, or a variant thereof comprising 1, 2, 3, or 4 amino acid substitutions; a CDR3 comprising SEQ ID NO:112, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:10. In some embodiments, a CD30-binding moiety comprises a tandem repeat of an antibody having a CDR1, CDR2, and CDR3 from a sdAb having the amino acid sequence of SEQ ID NO:10.

In some embodiments, a CD30-binding moiety comprises an amino acid sequence that is at least about 80% sequence identity to SEQ ID NO:10. In some embodiments, a CD30-binding moiety comprises an amino acid sequence that is at least about 85%, at least about 90%, at least about 95%, at least about 97%, or at least about 99% sequence identity to SEQ ID NO:10. In some embodiments, a CD30-binding moiety comprises an amino acid sequence that is at least about 85% sequence identity to SEQ ID NO:10. In some embodiments, a CD30-binding moiety comprises an amino acid sequence that is at least about 90% sequence identity to SEQ ID NO:10. In some embodiments, a CD30-binding moiety comprises an amino acid sequence that is at least about 95% sequence identity to SEQ ID NO:10. In some embodiments, a CD30-binding moiety comprises an amino acid sequence that is at least about 97% sequence identity to SEQ ID NO:10. In some embodiments, a CD30-binding moiety comprises an amino acid sequence that is at least about 99% sequence identity to SEQ ID NO:10. In some embodiments, a CD30-binding moiety comprises an amino acid sequence that is SEQ ID NO:10. In some embodiments, a CD30-binding moiety comprises an amino acid sequence that is SEQ ID NO:23. In some embodiments, a CD30-binding moiety comprises an amino acid sequence that is SEQ ID NO:24. In some embodiments, a CD30-binding moiety comprises an amino acid sequence that is SEQ ID NO:25. In some embodiments, a CD30-binding moiety comprises an amino acid sequence that is SEQ ID NO:26.

In some embodiments, a CD30-binding moiety comprises a CDR1, CDR2, and/or CDR3 from antibody AS48433, a humanized version thereof, or variants thereof. In some embodiments, a CD30-binding moiety comprises a CDR1, CDR2, and CDR3 from antibody AS48433. In some embodiments, a CD30-binding moiety comprises a humanized version of antibody AS48433. In some embodiments, a CD30-binding moiety comprises a variant of antibody AS48433. In some embodiments, a CD30-binding moiety comprises antibody AS48433 (SEQ ID NO:10). In some embodiments, a CD30-binding moiety comprises antibody AS48433VH4 (SEQ ID NO:23). In some embodiments, a CD30-binding moiety comprises antibody AS48433VH5 (SEQ ID NO:24). In some embodiments, a CD30-binding moiety comprises antibody AS48433VH11 (SEQ ID NO:25). In some embodiments, a CD30-binding moiety comprises antibody AS48433VH12 (SEQ ID NO:26). In some embodiments, a CD30-binding moiety comprises a tandem repeat of antibody AS48433, AS48433VH4, AS48433VH5, AS48433VH11, or AS48433VH12.

In some embodiments, a binding moiety competes for binding to CD30 with a reference antibody, wherein the reference antibody comprises a CDR1 comprising SEQ ID NO:87, a CDR2 comprising SEQ ID NO:100, and a CDR3 comprising SEQ ID NO:112. In some embodiments, a binding moiety competes for binding to CD30 with a reference antibody, wherein the reference antibody is AS48433.

In some embodiments, a CD30-binding moiety comprises: a CDR1 comprising SEQ ID NO:88, a CDR2 comprising SEQ ID NO:101, and/or a CDR3 comprising SEQ ID NO:113. In some embodiments, a CD30-binding moiety comprises: a CDR1 comprising SEQ ID NO:88, a CDR2 comprising SEQ ID NO:101, and a CDR3 comprising SEQ ID NO:113. In some embodiments, a CD30-binding moiety comprises: a CDR1 comprising SEQ ID NO: 88, or a variant thereof comprising 1, 2, 3, or 4 amino acid substitutions; a CDR2 comprising SEQ ID NO:101, or a variant thereof comprising 1, 2, 3, or 4 amino acid substitutions; a CDR3 comprising SEQ ID NO:113, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:11. In some embodiments, a CD30-binding moiety comprises a tandem repeat of an antibody having a CDR1, CDR2, and CDR3 from a sdAb having the amino acid sequence of SEQ ID NO:11.

In some embodiments, a CD30-binding moiety comprises an amino acid sequence that is at least about 80% sequence identity to SEQ ID NO:11. In some embodiments, a CD30-binding moiety comprises an amino acid sequence that is at least about 85%, at least about 90%, at least about 95%, at least about 97%, or at least about 99% sequence identity to SEQ ID NO:11. In some embodiments, a CD30-binding moiety comprises an amino acid sequence that is at least about 85% sequence identity to SEQ ID NO:11. In some embodiments, a CD30-binding moiety comprises an amino acid sequence that is at least about 90% sequence identity to SEQ ID NO:11. In some embodiments, a CD30-binding moiety comprises an amino acid sequence that is at least about 95% sequence identity to SEQ ID NO:11. In some embodiments, a CD30-binding moiety comprises an amino acid sequence that is at least about 97% sequence identity to SEQ ID NO:11. In some embodiments, a CD30-binding moiety comprises an amino acid sequence that is at least about 99% sequence identity to SEQ ID NO:11. In some embodiments, a CD30-binding moiety comprises an amino acid sequence that is SEQ ID NO:11. In some embodiments, a CD30-binding moiety comprises an amino acid sequence that is SEQ ID NO:27. In some embodiments, a CD30-binding moiety comprises an amino acid sequence that is SEQ ID NO:28.

In some embodiments, a CD30-binding moiety comprises a CDR1, CDR2, and/or CDR3 from antibody AS48463, a humanized version thereof, or variants thereof. In some embodiments, a CD30-binding moiety comprises a CDR1, CDR2, and CDR3 from antibody AS48463. In some embodiments, a CD30-binding moiety comprises a humanized version of antibody AS48463. In some embodiments, a CD30-binding moiety comprises a variant of antibody AS48463. In some embodiments, a CD30-binding moiety comprises antibody AS48463 (SEQ ID NO:11). In some embodiments, a CD30-binding moiety comprises antibody AS48463VH4 (SEQ ID NO:27). In some embodiments, a CD30-binding moiety comprises antibody AS48463VH11 (SEQ ID NO:28). In some embodiments, a CD30-binding moiety comprises a tandem repeat of antibody AS48463, AS48463VH4, or AS48463VH11.

In some embodiments, a binding moiety competes for binding to CD30 with a reference antibody, wherein the reference antibody comprises a CDR1 comprising SEQ ID NO:88, a CDR2 comprising SEQ ID NO:101, and a CDR3 comprising SEQ ID NO:113. In some embodiments, a binding moiety competes for binding to CD30 with a reference antibody, wherein the reference antibody is AS48463.

In some embodiments, a CD30-binding moiety comprises: a CDR1 comprising SEQ ID NO:89, a CDR2 comprising SEQ ID NO:102, and/or a CDR3 comprising SEQ ID NO:114. In some embodiments, a CD30-binding moiety comprises: a CDR1 comprising SEQ ID NO:89, a CDR2 comprising SEQ ID NO:102, and a CDR3 comprising SEQ ID NO:114. In some embodiments, a CD30-binding moiety comprises: a CDR1 comprising SEQ ID NO: 89, or a variant thereof comprising 1, 2, 3, or 4 amino acid substitutions; a CDR2 comprising SEQ ID NO:102, or a variant thereof comprising 1, 2, 3, or 4 amino acid substitutions; a CDR3 comprising SEQ ID NO:114, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:12. In some embodiments, a CD30-binding moiety comprises a tandem repeat of an antibody having a CDR1, CDR2, and CDR3 from a sdAb having the amino acid sequence of SEQ ID NO:12.

In some embodiments, a CD30-binding moiety comprises an amino acid sequence that is at least about 80% sequence identity to SEQ ID NO:12. In some embodiments, a CD30-binding moiety comprises an amino acid sequence that is at least about 85%, at least about 90%, at least about 95%, at least about 97%, or at least about 99% sequence identity to SEQ ID NO:12. In some embodiments, a CD30-binding moiety comprises an amino acid sequence that is at least about 85% sequence identity to SEQ ID NO:12. In some embodiments, a CD30-binding moiety comprises an amino acid sequence that is at least about 90% sequence identity to SEQ ID NO:12. In some embodiments, a CD30-binding moiety comprises an amino acid sequence that is at least about 95% sequence identity to SEQ ID NO:12. In some embodiments, a CD30-binding moiety comprises an amino acid sequence that is at least about 97% sequence identity to SEQ ID NO:12. In some embodiments, a CD30-binding moiety comprises an amino acid sequence that is at least about 99% sequence identity to SEQ ID NO:12. In some embodiments, a CD30-binding moiety comprises an amino acid sequence that is SEQ ID NO:12. In some embodiments, a CD30-binding moiety comprises an amino acid sequence that is SEQ ID NO:29. In some embodiments, a CD30-binding moiety comprises an amino acid sequence that is SEQ ID NO:30. In some embodiments, a CD30-binding moiety comprises an amino acid sequence that is SEQ ID NO:31. In some embodiments, a CD30-binding moiety comprises an amino acid sequence that is SEQ ID NO:32.

In some embodiments, a CD30-binding moiety comprises a CDR1, CDR2, and/or CDR3 from antibody AS48481, a humanized version thereof, or variants thereof. In some embodiments, a CD30-binding moiety comprises a CDR1, CDR2, and CDR3 from antibody AS48481. In some embodiments, a CD30-binding moiety comprises a humanized version of antibody AS48481. In some embodiments, a CD30-binding moiety comprises a variant of antibody AS48481. In some embodiments, a CD30-binding moiety comprises antibody AS48481 (SEQ ID NO:12). In some embodiments, a CD30-binding moiety comprises antibody AS48481VH5 (SEQ ID NO:29). In some embodiments, a CD30-binding moiety comprises antibody AS48481VH6 (SEQ ID NO:30). In some embodiments, a CD30-binding moiety comprises antibody AS48481VH13 (SEQ ID NO:31). In some embodiments, a CD30-binding moiety comprises antibody AS48481VH14 (SEQ ID NO:32). In some embodiments, a CD30-binding moiety comprises a tandem repeat of antibody AS48481, AS48481VH5, AS48481VH6, AS48481VH13, or AS48481VH14.

In some embodiments, a binding moiety competes for binding to CD30 with a reference antibody, wherein the reference antibody comprises a CDR1 comprising SEQ ID NO:89, a CDR2 comprising SEQ ID NO:102, and a CDR3 comprising SEQ ID NO:114. In some embodiments, a binding moiety competes for binding to CD30 with a reference antibody, wherein the reference antibody is AS48481.

In some embodiments, a CD30-binding moiety comprises: a CDR1 comprising SEQ ID NO:90, a CDR2 comprising SEQ ID NO:103, and/or a CDR3 comprising SEQ ID NO:115. In some embodiments, a CD30-binding moiety comprises: a CDR1 comprising SEQ ID NO:90, a CDR2 comprising SEQ ID NO:103, and a CDR3 comprising SEQ ID NO:115. In some embodiments, a CD30-binding moiety comprises: a CDR1 comprising SEQ ID NO: 90, or a variant thereof comprising 1, 2, 3, or 4 amino acid substitutions; a CDR2 comprising SEQ ID NO:103, or a variant thereof comprising 1, 2, 3, or 4 amino acid substitutions; a CDR3 comprising SEQ ID NO:115, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:13. In some embodiments, a CD30-binding moiety comprises a tandem repeat of an antibody having a CDR1, CDR2, and CDR3 from a sdAb having the amino acid sequence of SEQ ID NO:13.

In some embodiments, a CD30-binding moiety comprises an amino acid sequence that is at least about 80% sequence identity to SEQ ID NO:13. In some embodiments, a CD30-binding moiety comprises an amino acid sequence that is at least about 85%, at least about 90%, at least about 95%, at least about 97%, or at least about 99% sequence identity to SEQ ID NO:13. In some embodiments, a CD30-binding moiety comprises an amino acid sequence that is at least about 85% sequence identity to SEQ ID NO:13. In some embodiments, a CD30-binding moiety comprises an amino acid sequence that is at least about 90% sequence identity to SEQ ID NO:13. In some embodiments, a CD30-binding moiety comprises an amino acid sequence that is at least about 95% sequence identity to SEQ ID NO:13. In some embodiments, a CD30-binding moiety comprises an amino acid sequence that is at least about 97% sequence identity to SEQ ID NO:13. In some embodiments, a CD30-binding moiety comprises an amino acid sequence that is at least about 99% sequence identity to SEQ ID NO:13. In some embodiments, a CD30-binding moiety comprises an amino acid sequence that is SEQ ID NO:13. In some embodiments, a CD30-binding moiety comprises an amino acid sequence that is SEQ ID NO:33. In some embodiments, a CD30-binding moiety comprises an amino acid sequence that is SEQ ID NO:34. In some embodiments, a CD30-binding moiety comprises an amino acid sequence that is SEQ ID NO:35. In some embodiments, a CD30-binding moiety comprises an amino acid sequence that is SEQ ID NO:36.

In some embodiments, a CD30-binding moiety comprises a CDR1, CDR2, and/or CDR3 from antibody AS48508, a humanized version thereof, or variants thereof. In some embodiments, a CD30-binding moiety comprises a CDR1, CDR2, and CDR3 from antibody AS48508. In some embodiments, a CD30-binding moiety comprises a humanized version of antibody AS48508. In some embodiments, a CD30-binding moiety comprises a variant of antibody AS48508. In some embodiments, a CD30-binding moiety comprises antibody AS48508 (SEQ ID NO:13). In some embodiments, a CD30-binding moiety comprises antibody AS48508VH4 (SEQ ID NO:33). In some embodiments, a CD30-binding moiety comprises antibody AS48508VH5 (SEQ ID NO:34). In some embodiments, a CD30-binding moiety comprises antibody AS48508VH11 (SEQ ID NO:35). In some embodiments, a CD30-binding moiety comprises antibody AS48508VH12 (SEQ ID NO:36). In some embodiments, a CD30-binding moiety comprises a tandem repeat of antibody AS48508, AS48508VH4, AS48508VH5, AS48508VH11, or AS48508VH12.

In some embodiments, a binding moiety competes for binding to CD30 with a reference antibody, wherein the reference antibody comprises a CDR1 comprising SEQ ID NO:90, a CDR2 comprising SEQ ID NO:103, and a CDR3 comprising SEQ ID NO:115. In some embodiments, a binding moiety competes for binding to CD30 with a reference antibody, wherein the reference antibody is AS48508.

In some embodiments, a CD30-binding moiety comprises: a CDR1 comprising SEQ ID NO:91, a CDR2 comprising SEQ ID NO:104, and/or a CDR3 comprising SEQ ID NO:116. In some embodiments, a CD30-binding moiety comprises: a CDR1 comprising SEQ ID NO:91, a CDR2 comprising SEQ ID NO:104, and a CDR3 comprising SEQ ID NO:116. In some embodiments, a CD30-binding moiety comprises: a CDR1 comprising SEQ ID NO: 91, or a variant thereof comprising 1, 2, 3, or 4 amino acid substitutions; a CDR2 comprising SEQ ID NO:104, or a variant thereof comprising 1, 2, 3, or 4 amino acid substitutions; a CDR3 comprising SEQ ID NO:116, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:14. In some embodiments, a CD30-binding moiety comprises a tandem repeat of an antibody having a CDR1, CDR2, and CDR3 from a sdAb having the amino acid sequence of SEQ ID NO:14.

In some embodiments, a CD30-binding moiety comprises an amino acid sequence that is at least about 80% sequence identity to SEQ ID NO:14. In some embodiments, a CD30-binding moiety comprises an amino acid sequence that is at least about 85%, at least about 90%, at least about 95%, at least about 97%, or at least about 99% sequence identity to SEQ ID NO:14. In some embodiments, a CD30-binding moiety comprises an amino acid sequence that is at least about 85% sequence identity to SEQ ID NO:14. In some embodiments, a CD30-binding moiety comprises an amino acid sequence that is at least about 90% sequence identity to SEQ ID NO:14. In some embodiments, a CD30-binding moiety comprises an amino acid sequence that is at least about 95% sequence identity to SEQ ID NO:14. In some embodiments, a CD30-binding moiety comprises an amino acid sequence that is at least about 97% sequence identity to SEQ ID NO:14. In some embodiments, a CD30-binding moiety comprises an amino acid sequence that is at least about 99% sequence identity to SEQ ID NO:14. In some embodiments, a CD30-binding moiety comprises an amino acid sequence that is SEQ ID NO:14. In some embodiments, a CD30-binding moiety comprises an amino acid sequence that is SEQ ID NO:37. In some embodiments, a CD30-binding moiety comprises an amino acid sequence that is SEQ ID NO:38.

In some embodiments, a CD30-binding moiety comprises a CDR1, CDR2, and/or CDR3 from antibody AS48542, a humanized version thereof, or variants thereof. In some embodiments, a CD30-binding moiety comprises a CDR1, CDR2, and CDR3 from antibody AS48542. In some embodiments, a CD30-binding moiety comprises a humanized version of antibody AS48542. In some embodiments, a CD30-binding moiety comprises a variant of antibody AS48542. In some embodiments, a CD30-binding moiety comprises antibody AS48542. In some embodiments, a CD30-binding moiety comprises antibody AS48542VH5. In some embodiments, a CD30-binding moiety comprises antibody AS48542VH12. In some embodiments, a CD30-binding moiety comprises a tandem repeat of antibody AS48542, AS48542VH5, or AS48542VH12.

In some embodiments, a binding moiety competes for binding to CD30 with a reference antibody, wherein the reference antibody comprises a CDR1 comprising SEQ ID NO:91, a CDR2 comprising SEQ ID NO:104, and a CDR3 comprising SEQ ID NO:116. In some embodiments, a binding moiety competes for binding to CD30 with a reference antibody, wherein the reference antibody is AS48542.

In some embodiments, a CD30-binding moiety comprises: a CDR1 comprising SEQ ID NO:92, a CDR2 comprising SEQ ID NO:105, and/or a CDR3 comprising SEQ ID NO:117. In some embodiments, a CD30-binding moiety comprises: a CDR1 comprising SEQ ID NO:92, a CDR2 comprising SEQ ID NO:105, and a CDR3 comprising SEQ ID NO:117. In some embodiments, a CD30-binding moiety comprises: a CDR1 comprising SEQ ID NO: 92, or a variant thereof comprising 1, 2, 3, or 4 amino acid substitutions; a CDR2 comprising SEQ ID NO:105, or a variant thereof comprising 1, 2, 3, or 4 amino acid substitutions; a CDR3 comprising SEQ ID NO:117, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:15. In some embodiments, a CD30-binding moiety comprises a tandem repeat of an antibody having a CDR1, CDR2, and CDR3 from a sdAb having the amino acid sequence of SEQ ID NO:15.

In some embodiments, a CD30-binding moiety comprises an amino acid sequence that is at least about 80% sequence identity to SEQ ID NO:15. In some embodiments, a CD30-binding moiety comprises an amino acid sequence that is at least about 85%, at least about 90%, at least about 95%, at least about 97%, or at least about 99% sequence identity to SEQ ID NO:15. In some embodiments, a CD30-binding moiety comprises an amino acid sequence that is at least about 85% sequence identity to SEQ ID NO:15. In some embodiments, a CD30-binding moiety comprises an amino acid sequence that is at least about 90% sequence identity to SEQ ID NO:15. In some embodiments, a CD30-binding moiety comprises an amino acid sequence that is at least about 95% sequence identity to SEQ ID NO:15. In some embodiments, a CD30-binding moiety comprises an amino acid sequence that is at least about 97% sequence identity to SEQ ID NO:15. In some embodiments, a CD30-binding moiety comprises an amino acid sequence that is at least about 99% sequence identity to SEQ ID NO:15. In some embodiments, a CD30-binding moiety comprises an amino acid sequence that is SEQ ID NO:15. In some embodiments, a CD30-binding moiety comprises an amino acid sequence that is SEQ ID NO:39. In some embodiments, a CD30-binding moiety comprises an amino acid sequence that is SEQ ID NO:40.

In some embodiments, a CD30-binding moiety comprises a CDR1, CDR2, and/or CDR3 from antibody AS53445, a humanized version thereof, or variants thereof. In some embodiments, a CD30-binding moiety comprises a CDR1, CDR2, and CDR3 from antibody AS53445. In some embodiments, a CD30-binding moiety comprises a humanized version of antibody AS53445. In some embodiments, a CD30-binding moiety comprises a variant of antibody AS53445. In some embodiments, a CD30-binding moiety comprises antibody AS53445 (SEQ ID NO:15). In some embodiments, a CD30-binding moiety comprises antibody AS53445VH4 (SEQ ID NO:39). In some embodiments, a CD30-binding moiety comprises antibody AS53445VH11 (SEQ ID NO:40). In some embodiments, a CD30-binding moiety comprises a tandem repeat of antibody AS53445, AS53445VH4 or AS53445VH11.

In some embodiments, a binding moiety competes for binding to CD30 with a reference antibody, wherein the reference antibody comprises a CDR1 comprising SEQ ID NO:92, a CDR2 comprising SEQ ID NO:105, and a CDR3 comprising SEQ ID NO:117. In some embodiments, a binding moiety competes for binding to CD30 with a reference antibody, wherein the reference antibody is AS53445.

In some embodiments, a CD30-binding moiety comprises: a CDR1 comprising SEQ ID NO:93, a CDR2 comprising SEQ ID NO:106, and/or a CDR3 comprising SEQ ID NO:118. In some embodiments, a CD30-binding moiety comprises: a CDR1 comprising SEQ ID NO:93, a CDR2 comprising SEQ ID NO:106, and a CDR3 comprising SEQ ID NO:118. In some embodiments, a CD30-binding moiety comprises: a CDR1 comprising SEQ ID NO: 93, or a variant thereof comprising 1, 2, 3, or 4 amino acid substitutions; a CDR2 comprising SEQ ID NO:106, or a variant thereof comprising 1, 2, 3, or 4 amino acid substitutions; a CDR3 comprising SEQ ID NO:118, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:16. In some embodiments, a CD30-binding moiety comprises a tandem repeat of an antibody having a CDR1, CDR2, and CDR3 from a sdAb having the amino acid sequence of SEQ ID NO:16.

In some embodiments, a CD30-binding moiety comprises an amino acid sequence that is at least about 80% sequence identity to SEQ ID NO:16. In some embodiments, a CD30-binding moiety comprises an amino acid sequence that is at least about 85%, at least about 90%, at least about 95%, at least about 97%, or at least about 99% sequence identity to SEQ ID NO:16. In some embodiments, a CD30-binding moiety comprises an amino acid sequence that is at least about 85% sequence identity to SEQ ID NO:16. In some embodiments, a CD30-binding moiety comprises an amino acid sequence that is at least about 90% sequence identity to SEQ ID NO:16. In some embodiments, a CD30-binding moiety comprises an amino acid sequence that is at least about 95% sequence identity to SEQ ID NO:16. In some embodiments, a CD30-binding moiety comprises an amino acid sequence that is at least about 97% sequence identity to SEQ ID NO:16. In some embodiments, a CD30-binding moiety comprises an amino acid sequence that is at least about 99% sequence identity to SEQ ID NO:16. In some embodiments, a CD30-binding moiety comprises an amino acid sequence that is SEQ ID NO:16. In some embodiments, a CD30-binding moiety comprises an amino acid sequence that is SEQ ID NO:41. In some embodiments, a CD30-binding moiety comprises an amino acid sequence that is SEQ ID NO:42. In some embodiments, a CD30-binding moiety comprises an amino acid sequence that is SEQ ID NO:43. In some embodiments, a CD30-binding moiety comprises an amino acid sequence that is SEQ ID NO:199. In some embodiments, a CD30-binding moiety comprises an amino acid sequence that is SEQ ID NO:44. In some embodiments, a CD30-binding moiety comprises an amino acid sequence that is SEQ ID NO:45. In some embodiments, a CD30-binding moiety comprises an amino acid sequence that is SEQ ID NO:46.

In some embodiments, a CD30-binding moiety comprises a CDR1, CDR2, and/or CDR3 from antibody AS53574, a humanized version thereof, or variants thereof. In some embodiments, a CD30-binding moiety comprises a CDR1, CDR2, and CDR3 from antibody AS53574. In some embodiments, a CD30-binding moiety comprises a humanized version of antibody AS53574. In some embodiments, a CD30-binding moiety comprises a variant of antibody AS53574. In some embodiments, a CD30-binding moiety comprises antibody AS53574 (SEQ ID NO:16). In some embodiments, a CD30-binding moiety comprises antibody AS53574VH4 (SEQ ID NO:41). In some embodiments, a CD30-binding moiety comprises antibody AS53574VH5 (SEQ ID NO:42). In some embodiments, a CD30-binding moiety comprises antibody AS53574VH6 (SEQ ID NO:43). In some embodiments, a CD30-binding moiety comprises antibody AS53574VH7 (SEQ ID NO:199). In some embodiments, a CD30-binding moiety comprises antibody AS53574VH11 (SEQ ID NO:44). In some embodiments, a CD30-binding moiety comprises antibody AS53574VH12 (SEQ ID NO:45). In some embodiments, a CD30-binding moiety comprises antibody AS53574VH13 (SEQ ID NO:46). In some embodiments, a CD30-binding moiety comprises a tandem repeat of antibody AS53574, AS53574VH4, AS53574VH5, AS53574VH6, AS53574VH7, AS53574VH11, AS53574VH12, or AS53574VH13.

In some embodiments, a binding moiety competes for binding to CD30 with a reference antibody, wherein the reference antibody comprises a CDR1 comprising SEQ ID NO:93, a CDR2 comprising SEQ ID NO:106, and a CDR3 comprising SEQ ID NO:118. In some embodiments, a binding moiety competes for binding to CD30 with a reference antibody, wherein the reference antibody is AS53574.

In some embodiments, a CD30-binding moiety comprises: a CDR1 comprising SEQ ID NO:94, a CDR2 comprising SEQ ID NO:103, and/or a CDR3 comprising SEQ ID NO:119. In some embodiments, a CD30-binding moiety comprises: a CDR1 comprising SEQ ID NO:94, a CDR2 comprising SEQ ID NO:103, and a CDR3 comprising SEQ ID NO:119. In some embodiments, a CD30-binding moiety comprises: a CDR1 comprising SEQ ID NO: 94, or a variant thereof comprising 1, 2, 3, or 4 amino acid substitutions; a CDR2 comprising SEQ ID NO:103, or a variant thereof comprising 1, 2, 3, or 4 amino acid substitutions; a CDR3 comprising SEQ ID NO:119, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:17. In some embodiments, a CD30-binding moiety comprises a tandem repeat of an antibody having a CDR1, CDR2, and CDR3 from a sdAb having the amino acid sequence of SEQ ID NO:17.

In some embodiments, a CD30-binding moiety comprises an amino acid sequence that is at least about 80% sequence identity to SEQ ID NO:17. In some embodiments, a CD30-binding moiety comprises an amino acid sequence that is at least about 85%, at least about 90%, at least about 95%, at least about 97%, or at least about 99% sequence identity to SEQ ID NO:17. In some embodiments, a CD30-binding moiety comprises an amino acid sequence that is at least about 85% sequence identity to SEQ ID NO:17. In some embodiments, a CD30-binding moiety comprises an amino acid sequence that is at least about 90% sequence identity to SEQ ID NO:17. In some embodiments, a CD30-binding moiety comprises an amino acid sequence that is at least about 95% sequence identity to SEQ ID NO:17. In some embodiments, a CD30-binding moiety comprises an amino acid sequence that is at least about 97% sequence identity to SEQ ID NO:17. In some embodiments, a CD30-binding moiety comprises an amino acid sequence that is at least about 99% sequence identity to SEQ ID NO:17. In some embodiments, a CD30-binding moiety comprises an amino acid sequence that is SEQ ID NO:17. In some embodiments, a CD30-binding moiety comprises an amino acid sequence that is SEQ ID NO:47. In some embodiments, a CD30-binding moiety comprises an amino acid sequence that is SEQ ID NO:48. In some embodiments, a CD30-binding moiety comprises an amino acid sequence that is SEQ ID NO:49. In some embodiments, a CD30-binding moiety comprises an amino acid sequence that is SEQ ID NO:50.

In some embodiments, a CD30-binding moiety comprises a CDR1, CDR2, and/or CDR3 from antibody AS53750, a humanized version thereof, or variants thereof. In some embodiments, a CD30-binding moiety comprises a CDR1, CDR2, and CDR3 from antibody AS53750. In some embodiments, a CD30-binding moiety comprises a humanized version of antibody AS53750. In some embodiments, a CD30-binding moiety comprises a variant of antibody AS53750. In some embodiments, a CD30-binding moiety comprises antibody AS53750 (SEQ ID NO:17). In some embodiments, a CD30-binding moiety comprises antibody AS53750VH4 (SEQ ID NO:47). In some embodiments, a CD30-binding moiety comprises antibody AS53750VH5 (SEQ ID NO:48). In some embodiments, a CD30-binding moiety comprises antibody AS53750VH11 (SEQ ID NO:49). In some embodiments, a CD30-binding moiety comprises antibody AS53750VH12 (SEQ ID NO:50). In some embodiments, a CD30-binding moiety comprises a tandem repeat of antibody AS53750, AS53750VH4, AS53750VH5, AS53750VH11, or AS53750VH12.

In some embodiments, a binding moiety competes for binding to CD30 with a reference antibody, wherein the reference antibody comprises a CDR1 comprising SEQ ID NO:94, a CDR2 comprising SEQ ID NO:103, and a CDR3 comprising SEQ ID NO:119. In some embodiments, a binding moiety competes for binding to CD30 with a reference antibody, wherein the reference antibody is AS53750.

In some embodiments, a CD30-binding moiety comprises: a CDR1 comprising SEQ ID NO:95, a CDR2 comprising SEQ ID NO:103, and/or a CDR3 comprising SEQ ID NO:120. In some embodiments, a CD30-binding moiety comprises: a CDR1 comprising SEQ ID NO:95, a CDR2 comprising SEQ ID NO:103, and a CDR3 comprising SEQ ID NO:120. In some embodiments, a CD30-binding moiety comprises: a CDR1 comprising SEQ ID NO: 95, or a variant thereof comprising 1, 2, 3, or 4 amino acid substitutions; a CDR2 comprising SEQ ID NO:103, or a variant thereof comprising 1, 2, 3, or 4 amino acid substitutions; a CDR3 comprising SEQ ID NO:120, or a variant thereof comprising 1, 2, 3, or 4 amino acid substitutions. In some embodiments, a CD30-binding moiety comprises a CDR1, CDR2, and CDR3 from a sdAb having the amino acid sequence of SEQ ID NO:18. In some embodiments, a CD30-binding moiety comprises a tandem repeat of an antibody having a CDR1, CDR2, and CDR3 from a sdAb having the amino acid sequence of SEQ ID NO:18.

In some embodiments, a CD30-binding moiety comprises an amino acid sequence that is at least about 80% sequence identity to SEQ ID NO:18. In some embodiments, a CD30-binding moiety comprises an amino acid sequence that is at least about 85%, at least about 90%, at least about 95%, at least about 97%, or at least about 99% sequence identity to SEQ ID NO:18. In some embodiments, a CD30-binding moiety comprises an amino acid sequence that is at least about 85% sequence identity to SEQ ID NO:18. In some embodiments, a CD30-binding moiety comprises an amino acid sequence that is at least about 90% sequence identity to SEQ ID NO:18. In some embodiments, a CD30-binding moiety comprises an amino acid sequence that is at least about 95% sequence identity to SEQ ID NO:18. In some embodiments, a CD30-binding moiety comprises an amino acid sequence that is at least about 97% sequence identity to SEQ ID NO:18. In some embodiments, a CD30-binding moiety comprises an amino acid sequence that is at least about 99% sequence identity to SEQ ID NO:18. In some embodiments, a CD30-binding moiety comprises an amino acid sequence that is SEQ ID NO:18. In some embodiments, a CD30-binding moiety comprises an amino acid sequence that is SEQ ID NO:51. In some embodiments, a CD30-binding moiety comprises an amino acid sequence that is SEQ ID NO:52. In some embodiments, a CD30-binding moiety comprises an amino acid sequence that is SEQ ID NO:53. In some embodiments, a CD30-binding moiety comprises an amino acid sequence that is SEQ ID NO:54.

In some embodiments, a CD30-binding moiety comprises a CDR1, CDR2, and/or CDR3 from antibody AS54233, a humanized version thereof, or variants thereof. In some embodiments, a CD30-binding moiety comprises a CDR1, CDR2, and CDR3 from antibody AS54233. In some embodiments, a CD30-binding moiety comprises a humanized version of antibody AS54233. In some embodiments, a CD30-binding moiety comprises a variant of antibody AS54233. In some embodiments, a CD30-binding moiety comprises antibody AS54233 (SEQ ID NO:18). In some embodiments, a CD30-binding moiety comprises antibody AS54233VH4 (SEQ ID NO:51). In some embodiments, a CD30-binding moiety comprises antibody AS54233VH5 (SEQ ID NO:52). In some embodiments, a CD30-binding moiety comprises antibody AS54233VH11 (SEQ ID NO:53). In some embodiments, a CD30-binding moiety comprises antibody AS54233VH12 (SEQ ID NO:54). In some embodiments, a CD30-binding moiety comprises a tandem repeat of antibody AS54233, AS54233VH4, AS54233VH5, AS54233VH11, or AS54233VH12.

In some embodiments, a binding moiety competes for binding to CD30 with a reference antibody, wherein the reference antibody comprises a CDR1 comprising SEQ ID NO:95, a CDR2 comprising SEQ ID NO:103, and a CDR3 comprising SEQ ID NO:120. In some embodiments, a binding moiety competes for binding to CD30 with a reference antibody, wherein the reference antibody is AS54233.

In some embodiments, a CD30-binding moiety is monovalent and comprises one antibody described herein. In some embodiments, a CD30-binding moiety is bivalent and comprises two antibodies described herein, each comprising (i) a CDR1 comprising an amino acid sequence selected from the group consisting of SEQ ID NOs:87-95; (ii) a CDR2 comprising an amino acid sequence selected from the group consisting of SEQ ID NOs:100-106; and (iii) a CDR3 comprising an amino acid sequence selected from the group consisting of SEQ ID NOs:111-120; or a variant thereof comprising up to 3 amino acid substitutions in each of CDR1, CDR2, and CDR3.

In some embodiment, a CD30-binding moiety comprises, from N-terminus to C-terminus, a first antibody, a linker, and a second antibody. In some embodiment, the first antibody and the second antibody recognized different epitopes of CD30 (e.g. CRD1 and CRD6). In certain embodiments, a CD30-binding moiety comprises a monovalent sdAb. In some embodiments, a CD30-binding moiety comprises two sdAbs described herein, each comprising (i) a CDR1 comprising an amino acid sequence selected from the group consisting of SEQ ID NOs:87-95; (ii) a CDR2 comprising an amino acid sequence selected from the group consisting of SEQ ID NOs:100-106; and (iii) a CDR3 comprising an amino acid sequence selected from the group consisting of SEQ ID NOs:111-120; or a variant thereof comprising up to 3 amino acid substitutions in each of CDR1, CDR2, and CDR3. In some embodiments, a CD30-binding moiety comprises a first sdAb, a linker, and a second sdAb, from N-terminus to C-terminus.

In some embodiments, a CD30-binding moiety comprises a first and second sdAbs, wherein each sdAb comprises a CDR1, CDR2, and CDR3 from antibody AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, or AS54233VH12. In some embodiments, a CD30-binding moiety comprises a first and second sdAbs, wherein each sdAb has an amino acid sequence selected from the group consisting of SEQ ID NOs:9-54 and 199.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863 and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS47863, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS47863, AS47863VH4, AS47863VH5, AS47863VH11, and AS47863VH12, and wherein the second sdAb is selected from the group consisting of AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48433, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS48433, AS48433VH4, AS48433VH5, AS48433VH11, and AS48433VH12, and wherein the second sdAb is selected from the group consisting of AS47863, AS47863VH4, AS47863VH5, AS47863VH11, AS47863VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48463, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS48463, AS48463VH4 and AS48463VH11, and wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48481, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS48481, AS48481VH5, AS48481VH6, AS48481VH13, and AS48481VH14, and wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48508, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS48508, AS48508VH4, AS48508VH5, AS48508VH11, and AS48508VH12, and wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS48542, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS48542, AS48542VH5, and AS48542VH12, and wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53445, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS53445, AS53445VH4, and AS53445VH11, wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53574, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS53574, AS53574VH4, AS53574VH5, AS53574VH6, AS53574VH7, AS53574VH11, AS53574VH12, and AS53574VH13, wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53750, AS53750VH4, AS53750VH5, AS53750VH11, AS53750VH12, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb comprises a CDR1, CDR2, and CDR3 from antibody AS54233. In some embodiments, a CD30-binding moiety comprises, from N-terminus to C-terminus, a first sdAb, a linker, and a second sdAb, wherein the first sdAb comprises a CDR1, CDR2, and CDR3 from antibody AS53750, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS53750, AS53750VH4, AS53750VH5, AS53750VH11, and AS53750VH12, wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS54233, AS54233VH4, AS54233VH5, AS54233VH11, and AS54233VH12.

In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS47863. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS48433. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS48463. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS48481. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS48508. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS48542. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS53445. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS53574. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb comprises a CDR1, CDR2, and CDR3 from antibody AS53750. In some embodiments, a CD30-binding moiety comprises, from N-terminus to C-terminus, a first sdAb, a linker, and a second sdAb, wherein the first sdAb comprises a CDR1, CDR2, and CDR3 from antibody AS54233, and wherein the second sdAb is tandem repeat of the first sdAb. In some embodiments, a CD30-binding moiety comprises, from N-terminus to C-terminus, a first sdAb, a linker, and a second sdAb, wherein the first sdAb is selected from the group consisting of AS54233VH4, AS54233VH5, AS54233VH11, and AS54233VH12, wherein the second sdAb is selected from the group consisting of AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS54233, AS53750, AS53750VH4, AS53750VH5, AS53750VH11, and AS53750VH12.

The linker that connects the first and second antibodies in a bivalent binding moiety can be any linker known in the art. For instance, the linker can be a biocompatible polymer with a length of 1 to 100 atoms. In some embodiments, the linker can be a peptide of between about 1 and about 50 amino acids. In some embodiments, the linker can be a peptide of between about 1 and about 5, about 5 and about 10, about 10 and about 15, about 15 and about 20, about 20 and about 30, about 40, or about 40 and about 50 amino acids. Exemplary linkers that can be used include Gly-Ser repeats, for example, (Gly)₄-Ser repeats of at one, two, three, four, five, six, seven or more repeats. In embodiments, the linker has the following sequences: (Gly)₄-Ser-(Gly)₃-Ser (SEQ ID NO:202) or ((Gly)₄-Ser)n, where n is 4, 5, or 6 (SEQ ID NO:203). In some embodiments, the linker has an amino acid sequence comprising or consisting of SEQ ID NO:55. In some embodiments, the linker has an amino acid sequence comprising or consisting of SEQ ID NO:56. In some embodiments, the linker has an amino acid sequence comprising or consisting of SEQ ID NO:57. In some embodiments, the linker has an amino acid sequence comprising or consisting of SEQ ID NO:202. In some embodiments, the linker has an amino acid sequence comprising or consisting of SEQ ID NO:203. In some embodiments, wherein the CD30-binding moiety comprises a first sdAb, a linker, and a second sdAb, from N-terminus to C-terminus, the linker has an amino acid sequence comprising or consisting of SEQ ID NO:55, SEQ ID NO:56, SEQ ID NO:57, SEQ ID NO:202, or SEQ ID NO:203.

In some embodiments, a CD30-binding moiety comprises a heavy chain variable region (VH) and a light chain variable region (VL). The CD30-binding moiety may be an antibody that comprises VH CDRs and VL CDRs described in Table 2.

In some embodiments, the VH and VL of the CD30-binding moiety are connected by a linker. The linker that connects the VH and VL of the CD30-binding moiety can be any linker known in the art. For instance, the linker can be a biocompatible polymer with a length of 1 to 100 atoms. In some embodiments, the linker can be a peptide of between about 1 and about 50 amino acids. In some embodiments, the linker can be a peptide of between about 1 and about 5, about 5 and about 10, about 10 and about 15, about 15 and about 20, about 20 and about 30, about 40, or about 40 and about 50 amino acids. Exemplary linkers that can be used include Gly-Ser repeats, for example, (Gly)₄-Ser repeats of at one, two, three, four, five, six, seven or more repeats. In embodiments, the linker has the following sequences: (Gly)₄-Ser-(Gly)₃-Ser (SEQ ID NO:202) or ((Gly)₄-Ser)ₙ, where n is 4, 5, or 6 (SEQ ID NO:203). In some embodiments, the linker has an amino acid sequence comprising or consisting of SEQ ID NO:55. In some embodiments, the linker has an amino acid sequence comprising or consisting of SEQ ID NO:56. In some embodiments, the linker has an amino acid sequence comprising or consisting of SEQ ID NO:57. In some embodiments, the linker has an amino acid sequence comprising or consisting of SEQ ID NO:202. In some embodiments, the linker has an amino acid sequence comprising or consisting of SEQ ID NO:203. In some embodiments, wherein the CD30-binding moiety comprises a VH, a linker, and a VL, the linker has an amino acid sequence comprising or consisting of SEQ ID NO:55, SEQ ID NO:56, SEQ ID NO:57, SEQ ID NO:202, or SEQ ID NO:203.

In some embodiments, a CD30-binding moiety having a VH and a VL comprises a sdAb, a HCAb, a Fab, a Fab′, a F(ab′)₂, a Fv, a scFv, a (scFv)₂, an IgG1 antibody, an IgG2 antibody, an IgG3 antibody, or an IgG4 antibody. In some embodiment, a CD30-binding moiety comprises a scFv (i.e., a truncated Fab fragment including the variable domain of an antibody heavy chain (VH) linked to a variable domain of a light antibody (VL) chain). In some embodiments, a VH of a scFv and a VL of a scFv is linked via a peptide. ScFv can be generated using routine recombinant DNA technology techniques known in the art.

In some embodiments, a CD30-binding moiety comprises a VH CDR1, CDR2, and CDR3 and/or a VL CDR1, CDR2, and CDR3 from an antibody described herein. In some embodiments, a CD30-binding moiety comprises a humanized version of an antibody described herein. In some embodiments, a CD30-binding moiety comprises a variant of an antibody described herein.

In some embodiments, a CD30-binding moiety comprises a VH CDR1, CDR2, and CDR3 and/or a VL CDR1, CDR2, and CDR3 from antibody AS57659, a humanized version thereof, or variants thereof. In some embodiments, a CD30-binding moiety comprises a VH CDR1, a VH CDR2, and a VH CDR3 from antibody AS57659. In other embodiments, a CD30-binding moiety comprises a VL CDR1, a VL CDR2, and a VL CDR3 from antibody AS57659. In certain embodiments, a CD30-binding moiety comprises a VH CDR1, a VH CDR2, a VH CDR3, a VL CDR1, a VL CDR2, and a VL CDR3 from antibody AS57659. In some embodiments, a CD30-binding moiety comprises a variant of antibody AS57659. In some embodiments, a CD30-binding moiety comprises antibody AS57659.

In some embodiments, a CD30-binding moiety comprises a VH CDR1, CDR2, and CDR3 and/or a VL CDR1, CDR2, and CDR3 from antibody AS57765, a humanized version thereof, or variants thereof. In some embodiments, a CD30-binding moiety comprises a VH CDR1, a VH CDR2, and a VH CDR3 from antibody AS57765. In other embodiments, a CD30-binding moiety comprises a VL CDR1, a VL CDR2, and a VL CDR3 from antibody AS57765. In certain embodiments, a CD30-binding moiety comprises a VH CDR1, a VH CDR2, a VH CDR3, a VL CDR1, a VL CDR2, and a VL CDR3 from antibody AS57765. In some embodiments, a CD30-binding moiety comprises a variant of antibody AS57765. In some embodiments, a CD30-binding moiety comprises antibody AS57765.

In some embodiments, a CD30-binding moiety comprises a VH CDR1, CDR2, and CDR3 and/or a VL CDR1, CDR2, and CDR3 from antibody AS57911, a humanized version thereof, or variants thereof. In some embodiments, a CD30-binding moiety comprises a VH CDR1, a VH CDR2, and a VH CDR3 from antibody AS57911. In other embodiments, a CD30-binding moiety comprises a VL CDR1, a VL CDR2, and a VL CDR3 from antibody AS57911. In certain embodiments, a CD30-binding moiety comprises a VH CDR1, a VH CDR2, a VH CDR3, a VL CDR1, a VL CDR2, and a VL CDR3 from antibody AS57911. In some embodiments, a CD30-binding moiety comprises a variant of antibody AS57911. In some embodiments, a CD30-binding moiety comprises antibody AS57911.

In some embodiments, a CD30-binding moiety comprises an antibody. In some embodiments, a CD30-binding moiety comprises a scFv. In some embodiments, a variant of an anti-CD30 antibody described herein comprises one to thirty conservative amino acid substitutions. In some embodiments, a variant of the anti-CD30 antibody comprises one to twenty-five conservative amino acid substitutions. In some embodiments, a variant of the anti-CD30 antibody comprises one to twenty conservative amino acid substitutions. In some embodiments, a variant of the anti-CD30 antibody comprises one to fifteen conservative amino acid substitutions. In some embodiments, a variant of the anti-CD30 antibody comprises one to ten conservative amino acid substitution(s). In some embodiments, a variant of the anti-CD30 antibody comprises one to five conservative amino acid substitution(s). In some embodiments, a variant of the anti-CD30 antibody comprises one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, a CD30-binding moiety comprises: (a) a VH CDR1 comprising SEQ ID NO:96, SEQ ID NO:97, or SEQ ID NO:98; a VH CDR2 comprising SEQ ID NO:107, SEQ ID NO:108, or SEQ ID NO:109; and a VH CDR3 comprising SEQ ID NO:121, SEQ ID NO:122, or SEQ ID NO:123; and/or (b) a VL CDR1 comprising SEQ ID NO:99; a VL CDR2 comprising SEQ ID NO:110; and a VL CDR3 comprising SEQ ID NO:124, SEQ ID NO:125, or SEQ ID NO:126; or a variant thereof comprising 1, 2, 3, or 4 amino acid substitutions in each of VH CDR1, VH CDR2, VH CDR3, VL CDR1, VL CDR2 and VL CDR3. In some embodiments, a CD30-binding moiety comprises a VH CDR1 comprising SEQ ID NO:96, SEQ ID NO:97, or SEQ ID NO:98; a VH CDR2 comprising SEQ ID NO:107, SEQ ID NO:108, or SEQ ID NO:109; and/or a VH CDR3 comprising SEQ ID NO:121, SEQ ID NO:122, or SEQ ID NO:123; or a variant thereof comprising 1, 2, 3, or 4 amino acid substitutions in each of VH CDR1, VH CDR2, VH CDR3. In some embodiments, a CD30-binding moiety comprises a VL CDR1 comprising SEQ ID NO:99; a VL CDR2 comprising SEQ ID NO:110; and/or a VL CDR3 comprising SEQ ID NO:124, SEQ ID NO:125, or SEQ ID NO:126; or a variant thereof comprising 1, 2, 3, or 4 amino acid substitutions in each of VL CDR1, VL CDR2 and VL CDR3. In some embodiments, a CD30-binding moiety comprises: (a) a VH CDR1 comprising SEQ ID NO:96, SEQ ID NO:97, or SEQ ID NO:98; a VH CDR2 comprising SEQ ID NO:107, SEQ ID NO:108, or SEQ ID NO:109; and a VH CDR3 comprising SEQ ID NO:121, SEQ ID NO:122, or SEQ ID NO:123; and (b) a VL CDR1 comprising SEQ ID NO:99; a VL CDR2 comprising SEQ ID NO:110; and a VL CDR3 comprising SEQ ID NO:124, SEQ ID NO:125, or SEQ ID NO:126; or a variant thereof comprising 1, 2, 3, or 4 amino acid substitutions in each of VH CDR1, VH CDR2, VH CDR3, VL CDR1, VL CDR2 and VL CDR3.

In some embodiments, a CD30-binding moiety comprises a heavy chain variable region that has at least about 80% sequence identity to the heavy chain variable region from scFv AS57659, AS57765, or AS57911. In some embodiments, a CD30-binding moiety comprises a heavy chain variable region that has at least about 85%, at least about 90%, at least about 95%, at least about 97%, or at least about 99% sequence identity to the heavy chain variable region from scFv AS57659, AS57765, or AS57911. In some embodiments, a CD30-binding moiety comprises a heavy chain variable region that is identical to the heavy chain variable region from scFv AS57659, AS57765, or AS57911. In some embodiments, a CD30-binding moiety comprises a light chain variable region that has at least about 80% sequence identity to the light chain variable region from scFv AS57659, AS57765, or AS57911. In some embodiments, a CD30-binding moiety comprises a light chain variable region that has at least about 85%, at least about 90%, at least about 95%, at least about 97%, or at least about 99% sequence identity to the light chain variable region from scFv AS57659, AS57765, or AS57911. In some embodiments, a CD30-binding moiety comprises a light chain variable region that is identical to the light chain variable region from scFv AS57659, AS57765, or AS57911. In some embodiments, a CD30-binding moiety comprises a heavy chain variable region and a light chain variable region that has at least about 80% sequence identity to the heavy chain variable region and the light chain variable region from scFv AS57659, AS57765, or AS57911. In some embodiments, a CD30-binding moiety comprises a heavy chain variable region and a light chain variable region that has at least about 85%, at least about 90%, at least about 95%, at least about 97%, or at least about 99% sequence identity to the heavy chain variable region and the light chain variable region from scFv AS57659, AS57765, or AS57911. In some embodiments, a CD30-binding moiety comprises a heavy chain variable region and a light chain variable region that are identical to the heavy chain variable region and the light chain variable region from scFv AS57659, AS57765, or AS57911.

In some embodiments, a CD30-binding moiety comprises a scFv having at least about 80% sequence identity to SEQ ID NO:58, SEQ ID NO:59, or SEQ ID NO:60. In some embodiments, a CD30-binding moiety comprises a scFv having at least about 85%, at least about 90%, at least about 95%, at least about 97%, or at least about 99% sequence identity to SEQ ID NO:58, SEQ ID NO:59, or SEQ ID NO:60.

In some embodiments, a CD30-binding moiety comprises: (a) a VH CDR1 comprising SEQ ID NO:96, a VH CDR2 comprising SEQ ID NO:107, and a VH CDR3 comprising SEQ ID NO:121; or a variant thereof comprising 1, 2, 3, 4, or 5 amino acid substitutions in VH CDRs; and/or (b) a VL CDR1 comprising SEQ ID NO:99, a VL CDR2 comprising SEQ ID NO:110, and a VL CDR3 comprising SEQ ID NO:124; or a variant thereof comprising 1, 2, 3, 4, or 5 amino acid substitutions in the VL CDRs. In some embodiments, the variant comprises one amino acid substitution in VH CDRs, VL CDRs, or each. In some embodiments, the variant comprises up to two amino acid substitutions in VH CDRs, VL CDRs, or each. In some embodiments, the variant comprises up to three amino acid substitutions in VH CDRs, VL CDRs, or each. In some embodiments, the variant comprises up to four amino acid substitutions in VH CDRs, VL CDRs, or each. In some embodiments, the variant comprises up to five amino acid substitutions in VH CDRs, VL CDRs, or each.

In some embodiments, a CD30-binding moiety comprises a VH CDR1 comprising SEQ ID NO:96, a VH CDR2 comprising SEQ ID NO:107, and a VH CDR3 comprising SEQ ID NO:121, or a variant thereof comprising 1, 2, 3, or 4 amino acid substitutions in each of VH CDR1, VH CDR2, and VH CDR3. In some embodiments, a CD30-binding moiety comprises a VL CDR1 comprising SEQ ID NO:99, a VL CDR2 comprising SEQ ID NO:110, and a VL CDR3 comprising SEQ ID NO:124; or a variant thereof comprising 1, 2, 3, or 4 amino acid substitutions in each of VL CDR1, VL CDR2 and VL CDR3. In some embodiments, a CD30-binding moiety comprises: (a) a VH CDR1 comprising SEQ ID NO:96, a VH CDR2 comprising SEQ ID NO:107, and a VH CDR3 comprising SEQ ID NO:121; and (b) a VL CDR1 comprising SEQ ID NO:99, a VL CDR2 comprising SEQ ID NO:110, and a VL CDR3 comprising SEQ ID NO:124; or a variant thereof comprising 1, 2, 3, or 4 amino acid substitutions in each of VH CDR1, VH CDR2, VH CDR3, VL CDR1, VL CDR2 and VL CDR3.

In some embodiments, a CD30-binding moiety comprises a VH CDR1, VH CDR2, and VH CDR3 from a scFv having the amino acid sequence of SEQ ID NO:59. In some embodiments, a CD30-binding moiety comprises a VL CDR1, VL CDR2, and VL CDR3 from a scFv having the amino acid sequence of SEQ ID NO: 59. In some embodiments, a CD30-binding moiety comprises a VH CDR1, VH CDR2, VH CDR3, VL CDR1, VL CDR2, and VL CDR3 from a scFv having the amino acid sequence of SEQ ID NO: 59.

In some embodiments, a CD30-binding moiety comprises a heavy chain variable region that has at least about 80% sequence identity to the heavy chain variable region from scFv AS57659. In some embodiments, a CD30-binding moiety comprises a heavy chain variable region that has at least about 85%, at least about 90%, at least about 95%, at least about 97%, or at least about 99% sequence identity to the heavy chain variable region from scFv AS57659. In some embodiments, a CD30-binding moiety comprises a heavy chain variable region that is identical to the heavy chain variable region from scFv AS57659. In some embodiments, a CD30-binding moiety comprises a light chain variable region that has at least about 80% sequence identity to the light chain variable region from scFv AS57659. In some embodiments, a CD30-binding moiety comprises a light chain variable region that has at least about 85%, at least about 90%, at least about 95%, at least about 97%, or at least about 99% sequence identity to the light chain variable region from scFv AS57659. In some embodiments, a CD30-binding moiety comprises a light chain variable region that is identical to the light chain variable region from scFv AS57659. In some embodiments, a CD30-binding moiety comprises a heavy chain variable region and a light chain variable region that has at least about 80% sequence identity to the heavy chain variable region and the light chain variable region from scFv AS57659. In some embodiments, a CD30-binding moiety comprises a heavy chain variable region and a light chain variable region that has at least about 85%, at least about 90%, at least about 95%, at least about 97%, or at least about 99% sequence identity to the heavy chain variable region and the light chain variable region from scFv AS57659. In some embodiments, a CD30-binding moiety comprises a heavy chain variable region and a light chain variable region that are identical to the heavy chain variable region and the light chain variable region from scFv AS57659.

In some embodiments, a CD30-binding moiety comprises a scFv having at least about 80% sequence identity to SEQ ID NO:59. In some embodiments, a CD30-binding moiety comprises a scFv having at least about 85%, at least about 90%, at least about 95%, at least about 97%, or at least about 99% sequence identity to SEQ ID NO:59. In some embodiments, a CD30-binding moiety comprises a scFv having an amino acid sequence that is identical to SEQ ID NO: 59.

In some embodiments, an antibody competes for binding to CD30 with a reference antibody, wherein the reference antibody comprises: (a) a VH CDR1 comprising SEQ ID NO:96, a VH CDR2 comprising SEQ ID NO:107, and a VH CDR3 comprising SEQ ID NO:121; and (b) a VL CDR1 comprising SEQ ID NO:99, a VL CDR2 comprising SEQ ID NO:110, and a VL CDR3 comprising SEQ ID NO:124. In some embodiments, an antibody competes for binding to CD30 with a reference antibody, wherein the reference antibody is scFv AS57659.

In some embodiments, a CD30-binding moiety comprises: (a) a VH CDR1 comprising SEQ ID NO:97, a VH CDR2 comprising SEQ ID NO:108, and a VH CDR3 comprising SEQ ID NO:122; or a variant thereof comprising 1, 2, 3, 4, or 5 amino acid substitutions in VH CDRs; and/or (b) a VL CDR1 comprising SEQ ID NO:99, a VL CDR2 comprising SEQ ID NO:110, and a VL CDR3 comprising SEQ ID NO:125; or a variant thereof comprising 1, 2, 3, 4, or 5 amino acid substitutions in the VL CDRs. In some embodiments, the variant comprises one amino acid substitution in VH CDRs, VL CDRs, or each. In some embodiments, the variant comprises up to two amino acid substitutions in VH CDRs, VL CDRs, or each. In some embodiments, the variant comprises up to three amino acid substitutions in VH CDRs, VL CDRs, or each. In some embodiments, the variant comprises up to four amino acid substitutions in VH CDRs, VL CDRs, or each. In some embodiments, the variant comprises up to five amino acid substitutions in VH CDRs, VL CDRs, or each.

In some embodiments, a CD30-binding moiety comprises a VH CDR1 comprising SEQ ID NO:97, a VH CDR2 comprising SEQ ID NO:108, and a VH CDR3 comprising SEQ ID NO:122, or a variant thereof comprising 1, 2, 3, or 4 amino acid substitutions in each of VH CDR1, VH CDR2, and VH CDR3. In some embodiments, a CD30-binding moiety comprises a VL CDR1 comprising SEQ ID NO:99, a VL CDR2 comprising SEQ ID NO:110, and a VL CDR3 comprising SEQ ID NO:125; or a variant thereof comprising 1, 2, 3, or 4 amino acid substitutions in each of VL CDR1, VL CDR2 and VL CDR3. In some embodiments, a CD30-binding moiety comprises: (a) a VH CDR1 comprising SEQ ID NO:97, a VH CDR2 comprising SEQ ID NO:108, and a VH CDR3 comprising SEQ ID NO:122; and (b) a VL CDR1 comprising SEQ ID NO:99, a VL CDR2 comprising SEQ ID NO:110, and a VL CDR3 comprising SEQ ID NO:125; or a variant thereof comprising 1, 2, 3, or 4 amino acid substitutions in each of VH CDR1, VH CDR2, VH CDR3, VL CDR1, VL CDR2 and VL CDR3.

In some embodiments, a CD30-binding moiety comprises a VH CDR1, VH CDR2, and VH CDR3 from a scFv having the amino acid sequence of SEQ ID NO:60. In some embodiments, a CD30-binding moiety comprises a VL CDR1, VL CDR2, and VL CDR3 from a scFv having the amino acid sequence of SEQ ID NO:60. In some embodiments, a CD30-binding moiety comprises a VH CDR1, VH CDR2, VH CDR3, VL CDR1, VL CDR2, and VL CDR3 from a scFv having the amino acid sequence of SEQ ID NO:60.

In some embodiments, a CD30-binding moiety comprises a heavy chain variable region that has at least about 80% sequence identity to the heavy chain variable region from scFv AS57765. In some embodiments, a CD30-binding moiety comprises a heavy chain variable region that has at least about 85%, at least about 90%, at least about 95%, at least about 97%, or at least about 99% sequence identity to the heavy chain variable region from scFv AS57765. In some embodiments, a CD30-binding moiety comprises a heavy chain variable region that is identical to the heavy chain variable region from scFv AS57765. In some embodiments, a CD30-binding moiety comprises a light chain variable region that has at least about 80% sequence identity to the light chain variable region from scFv AS57765. In some embodiments, a CD30-binding moiety comprises a light chain variable region that has at least about 85%, at least about 90%, at least about 95%, at least about 97%, or at least about 99% sequence identity to the light chain variable region from scFv AS57765. In some embodiments, a CD30-binding moiety comprises a light chain variable region that is identical to the light chain variable region from scFv AS57765. In some embodiments, a CD30-binding moiety comprises a heavy chain variable region and a light chain variable region that has at least about 80% sequence identity to the heavy chain variable region and the light chain variable region from scFv AS57765. In some embodiments, a CD30-binding moiety comprises a heavy chain variable region and a light chain variable region that has at least about 85%, at least about 90%, at least about 95%, at least about 97%, or at least about 99% sequence identity to the heavy chain variable region and the light chain variable region from scFv AS57765. In some embodiments, a CD30-binding moiety comprises a heavy chain variable region and a light chain variable region that are identical to the heavy chain variable region and the light chain variable region from scFv AS57765.

In some embodiments, a CD30-binding moiety comprises a scFv having at least about 80% sequence identity to SEQ ID NO:60. In some embodiments, a CD30-binding moiety comprises a scFv having at least about 85%, at least about 90%, at least about 95%, at least about 97%, or at least about 99% sequence identity to SEQ ID NO:60. In some embodiments, a CD30-binding moiety comprises a scFv having an amino acid sequence that is identical to SEQ ID NO:60.

In some embodiments, a CD30-binding moiety comprises: (a) a VH CDR1 comprising SEQ ID NO:98, a VH CDR2 comprising SEQ ID NO:109, and a VH CDR3 comprising SEQ ID NO:123; or a variant thereof comprising 1, 2, 3, 4, or 5 amino acid substitutions in VH CDRs; and/or (b) a VL CDR1 comprising SEQ ID NO:99, a VL CDR2 comprising SEQ ID NO:110, and a VL CDR3 comprising SEQ ID NO:126; or a variant thereof comprising 1, 2, 3, 4, or 5 amino acid substitutions in the VL CDRs. In some embodiments, the variant comprises one amino acid substitution in VH CDRs, VL CDRs, or each. In some embodiments, the variant comprises up to two amino acid substitutions in VH CDRs, VL CDRs, or each. In some embodiments, the variant comprises up to three amino acid substitutions in VH CDRs, VL CDRs, or each. In some embodiments, the variant comprises up to four amino acid substitutions in VH CDRs, VL CDRs, or each. In some embodiments, the variant comprises up to five amino acid substitutions in VH CDRs, VL CDRs, or each.

In some embodiments, a CD30-binding moiety comprises a VH CDR1 comprising SEQ ID NO:98, a VH CDR2 comprising SEQ ID NO:109, and a VH CDR3 comprising SEQ ID NO:123, or a variant thereof comprising 1, 2, 3, or 4 amino acid substitutions in each of VH CDR1, VH CDR2, and VH CDR3. In some embodiments, a CD30-binding moiety comprises a VL CDR1 comprising SEQ ID NO:99, a VL CDR2 comprising SEQ ID NO:110, and a VL CDR3 comprising SEQ ID NO:126; or a variant thereof comprising 1, 2, 3, or 4 amino acid substitutions in each of VL CDR1, VL CDR2 and VL CDR3. In some embodiments, a CD30-binding moiety comprises: (a) a VH CDR1 comprising SEQ ID NO:98, a VH CDR2 comprising SEQ ID NO:109, and a VH CDR3 comprising SEQ ID NO:123; and (b) a VL CDR1 comprising SEQ ID NO:99, a VL CDR2 comprising SEQ ID NO:110, and a VL CDR3 comprising SEQ ID NO:126; or a variant thereof comprising 1, 2, 3, or 4 amino acid substitutions in each of VH CDR1, VH CDR2, VH CDR3, VL CDR1, VL CDR2 and VL CDR3.

In some embodiments, a CD30-binding moiety comprises a VH CDR1, VH CDR2, and VH CDR3 from a scFv having the amino acid sequence of SEQ ID NO:58. In some embodiments, a CD30-binding moiety comprises a VL CDR1, VL CDR2, and VL CDR3 from a scFv having the amino acid sequence of SEQ ID NO:58. In some embodiments, a CD30-binding moiety comprises a VH CDR1, VH CDR2, VH CDR3, VL CDR1, VL CDR2, and VL CDR3 from a scFv having the amino acid sequence of SEQ ID NO:58.

In some embodiments, an antibody competes for binding to CD30 with a reference antibody, wherein the reference antibody comprises: (a) a VH CDR1 comprising SEQ ID NO:97, a VH CDR2 comprising SEQ ID NO:108, and a VH CDR3 comprising SEQ ID NO:122; and (b) a VL CDR1 comprising SEQ ID NO:99, a VL CDR2 comprising SEQ ID NO:110, and a VL CDR3 comprising SEQ ID NO:125. In some embodiments, an antibody competes for binding to CD30 with a reference antibody, wherein the reference antibody is scFv AS57765.

In some embodiments, a CD30-binding moiety comprises a heavy chain variable region that has at least about 80% sequence identity to the heavy chain variable region from scFv AS57911. In some embodiments, a CD30-binding moiety comprises a heavy chain variable region that has at least about 85%, at least about 90%, at least about 95%, at least about 97%, or at least about 99% sequence identity to the heavy chain variable region from scFv AS57911. In some embodiments, a CD30-binding moiety comprises a heavy chain variable region that is identical to the heavy chain variable region from scFv AS57911. In some embodiments, a CD30-binding moiety comprises a light chain variable region that has at least about 80% sequence identity to the light chain variable region from scFv AS57911. In some embodiments, a CD30-binding moiety comprises a light chain variable region that has at least about 85%, at least about 90%, at least about 95%, at least about 97%, or at least about 99% sequence identity to the light chain variable region from scFv AS57911. In some embodiments, a CD30-binding moiety comprises a light chain variable region that is identical to the light chain variable region from scFv AS57911. In some embodiments, a CD30-binding moiety comprises a heavy chain variable region and a light chain variable region that has at least about 80% sequence identity to the heavy chain variable region and the light chain variable region from scFv AS57911. In some embodiments, a CD30-binding moiety comprises a heavy chain variable region and a light chain variable region that has at least about 85%, at least about 90%, at least about 95%, at least about 97%, or at least about 99% sequence identity to the heavy chain variable region and the light chain variable region from scFv AS57911. In some embodiments, a CD30-binding moiety comprises a heavy chain variable region and a light chain variable region that are identical to the heavy chain variable region and the light chain variable region from scFv AS57911.

In some embodiments, a CD30-binding moiety comprises a scFv having at least about 80% sequence identity to SEQ ID NO:58. In some embodiments, a CD30-binding moiety comprises a scFv having at least about 85%, at least about 90%, at least about 95%, at least about 97%, or at least about 99% sequence identity to SEQ ID NO:58. In some embodiments, a CD30-binding moiety comprises a scFv having an amino acid sequence that is identical to SEQ ID NO:58.

In some embodiments, an antibody competes for binding to CD30 with a reference antibody, wherein the reference antibody comprises: (a) a VH CDR1 comprising SEQ ID NO:98, a VH CDR2 comprising SEQ ID NO:109, and a VH CDR3 comprising SEQ ID NO:123; and (b) a VL CDR1 comprising SEQ ID NO:99, a VL CDR2 comprising SEQ ID NO:110, and a VL CDR3 comprising SEQ ID NO:126. In some embodiments, an antibody competes for binding to CD30 with a reference antibody, wherein the reference antibody is scFv AS57911.

In some embodiments, a CD30-binding moiety described herein comprises one or more of antibody constant regions. In some embodiments, a CD30-binding moiety comprises one or more of the three heavy chain constant regions (CH1, CH2 or CH3) and/or to the light chain constant region (CL). In some embodiments, the heavy chain constant region comprises at least one human constant region. In some embodiments, the heavy chain constant region comprises more than one human constant region. In some embodiments, a CD30-binding moiety having at least one constant region further comprises additions, deletions, or substitutions of one or more amino acids in one or more regions.

It is known in the art that the constant region(s) of an antibody mediates several effector functions and these effector functions can vary depending on the isotype of the antibody. For example, binding of the C1 component of complement to the Fc region of IgG antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, a CD30-binding moiety comprises a Fc region. The amino acid sequences of the Fc region of human IgG1, IgG2, IgG3, and IgG4 are known to those of ordinary skill in the art. In some cases, Fc regions with amino acid variations have been identified in native antibodies. In some embodiments, a CD30-binding moiety comprises a variant Fc region that is engineered with substitutions at specific amino acid positions as compared to a native Fc region. In some embodiments, the Fc region is fused via a hinge. The hinge can be an IgG1 hinge, an IgG2 hinge, or an IgG3 hinge. In some embodiments, a CD30-binding moiety comprises a HCAb which comprises a sdAb fused with a Fc region via an IgG1 hinge.

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant may be determined by systematically making insertions, deletions, or substitutions in the sequence and testing the resulting variant proteins for activity as compared to the parent protein.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

In some embodiments, a variant of a CD30-binding moiety disclosed herein can retain the ability to recognize a target (e.g., CD30) to a similar extent, the same extent, or to a higher extent, as the parent binding moiety. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent binding moiety. In some embodiments, the variant can have an amino acid sequence that is at least about 80%, about 85%, about 90%, about 91%, about 92%, about 93%, about 94%, about 95%, about 96%, about 97%, about 98%, about 99% or more identical to an amino acid of SEQ ID NO:9-54, or 58-60.

In certain embodiments, a variant of a CD30-binding moiety comprises the amino acid sequence of the parent a CD30-binding moiety with one or more conservative amino acid substitution. Conservative amino acid substitutions are known in the art, and include amino acid substitutions in which one amino acid having certain physical and/or chemical properties is exchanged for another amino acid that has the same or similar chemical or physical properties.

In some embodiments, a variant of a CD30-binding moiety comprises the amino acid sequence of the parent binding moiety with one or more non-conservative amino acid substitutions. In some embodiments, a variant of a CD30-binding moiety comprises the amino acid sequence of the parent binding moiety with one or more non-conservative amino acid substitution, wherein the one or more non-conservative amino acid substitutions do not interfere with or inhibit one or more biological activities of the variant (e.g., CD30 binding). In certain embodiments, the one or more conservative amino acid substitutions and/or the one or more non-conservative amino acid substitutions can enhance a biological activity of the variant, such that the biological activity of the functional variant is increased as compared to the parent binding moiety.

In some embodiments, the function variant can have 1, 2, 3, 4, or 5 amino acid substitutions in the CDRs (e.g., CDR1, CDR2, and CDR3) of the binding moiety.

In some embodiments, an antigen-binding fragment can be modified naturally or by intervention. As a non-limiting example, an antigen-binding fragment can be modified through disulfide bond formation, glycosylation, lipidation, acetylation, phosphorylation, or any other manipulation or modification, such as conjugation with a labeling component. The antigen-binding fragments of embodiments of the invention can comprise one or more analogs of an amino acid (including, for example, unnatural amino acids), as well as other modifications known in the art. It is understood that, because the antigen-binding fragments of this invention can be based upon antibodies or other members of the immunoglobulin superfamily, in some embodiments, the polypeptides can occur as single chains.

In some embodiments, an antibody of the present disclosure is “deimmunized”. The deimmunization of antibodies generally consists of introducing specific amino acid mutations (e.g., substitutions, deletions, additions) that result in removal of predicted T-cell epitopes without significantly reducing the binding affinity or other desired characteristics of the antibody.

The variant antibodies or polypeptides described herein may be generated using methods known in the art, including but not limited to, site-directed mutagenesis, alanine scanning mutagenesis, and PCR mutagenesis.

The binding affinity of a CD30-binding moiety to CD30 is a reversible process, and can be measured as an equilibrium dissociation constant (K_{D}). K_{D} is the ratio of the dissociation rate to the association rate (kₒₙ). The lower the K_{D} of an antibody, the higher the affinity of the antibody for its target. In some embodiments, affinity is measured using SPR technology in a Biacore system. In some embodiments, a CD30-binding moiety (e.g., an antibody) binds CD30 with a K_{D} of about 500 nM to 1 μM, about 200 nM to about 500 nM, about 100 nM to about 200 nM, about 50 nM to about 100 nM, about 20 nM to 50 nM, about 10 nM to about 20 nM, about 5 nM to about 10 nM, about 2 nM to 5 nM, about 1 nM to about 2 nM, about 500 pM to about 1 nM, about 200 pM to 500 pM, about 100 pM to about 200 pM, about 50 pM to about 100 pM, about 20 pM to 50 pM, about 10 pM to about 20 pM, about 5 pM to about 10 pM, or about 2 pM to 5 pM.

In some embodiments, a CD30-binding moiety binds CD30 with a K_{D} of about 200 nM to about 500 nM. In some embodiments, a CD30-binding moiety binds CD30 with a K_{D} of about 50 nM to about 200 nM. In some embodiments, a CD30-binding moiety binds CD30 with a K_{D} of about 20 nM to 50 nM. In some embodiments, a CD30-binding moiety binds CD30 with a K_{D} of about 5 nM to about 20 nM. In some embodiments, a CD30-binding moiety binds CD30 with a K_{D} of about 2 nM to 5 nM. In some embodiments, a CD30-binding moiety binds CD30 with a K_{D} of about 500 pM to about 2 nM. In some embodiments, a CD30-binding moiety binds CD30 with a K_{D} of about 200 pM to 500 pM. In some embodiments, a CD30-binding moiety binds CD30 with a K_{D} of about 50 pM to about 200 pM. In some embodiments, a CD30-binding moiety binds CD30 with a K_{D} of about 20 pM to about 50 pM. In some embodiments, a CD30-binding moiety binds CD30 with a K_{D} of about 5 pM to about 20 pM.

In some embodiments, a CD30-binding moiety binds CD30 with a K_{D} of about 5 pM to about 500 nM. In some embodiments, a CD30-binding moiety binds CD30 with a K_{D} of about 200 pM to 200 nM. In some embodiments, a CD30-binding moiety binds CD30 with a K_{D} of about 2 nM to 200 nM.

In some embodiments, provided herein are polynucleotides comprising polynucleotides encoding that encode a polypeptide (i.e., a CD30-binding moiety) described herein. The term “polynucleotides that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotide comprises a polynucleotide (e.g., a nucleotide sequence) encoding a polypeptide comprising an amino acid sequence selected from the group consisting of: SEQ ID NOs:9-54, 58-60 and 199. In some embodiments, the polynucleotide comprises a polynucleotide encoding a polypeptide comprising an amino acid sequence of SEQ ID NO:9. In some embodiments, the polynucleotide comprises a polynucleotide encoding a polypeptide comprising an amino acid sequence of SEQ ID NO:10. In some embodiments, the polynucleotide comprises a polynucleotide encoding a polypeptide comprising an amino acid sequence of SEQ ID NO:11. In some embodiments, the polynucleotide comprises a polynucleotide encoding a polypeptide comprising an amino acid sequence of SEQ ID NO:12. In some embodiments, the polynucleotide comprises a polynucleotide encoding a polypeptide comprising an amino acid sequence of SEQ ID NO:13. In some embodiments, the polynucleotide comprises a polynucleotide encoding a polypeptide comprising an amino acid sequence of SEQ ID NO:14. In some embodiments, the polynucleotide comprises a polynucleotide encoding a polypeptide comprising an amino acid sequence of SEQ ID NO:15. In some embodiments, the polynucleotide comprises a polynucleotide encoding a polypeptide comprising an amino acid sequence of SEQ ID NO:16. In some embodiments, the polynucleotide comprises a polynucleotide encoding a polypeptide comprising an amino acid sequence of SEQ ID NO:17. In some embodiments, the polynucleotide comprises a polynucleotide encoding a polypeptide comprising an amino acid sequence of SEQ ID NO:18. In some embodiments, the polynucleotide comprises a polynucleotide encoding a polypeptide comprising an amino acid sequence of SEQ ID NO:19. In some embodiments, the polynucleotide comprises a polynucleotide encoding a polypeptide comprising an amino acid sequence of SEQ ID NO:20. In some embodiments, the polynucleotide comprises a polynucleotide encoding a polypeptide comprising an amino acid sequence of SEQ ID NO:21. In some embodiments, the polynucleotide comprises a polynucleotide encoding a polypeptide comprising an amino acid sequence of SEQ ID NO:22. In some embodiments, the polynucleotide comprises a polynucleotide encoding a polypeptide comprising an amino acid sequence of SEQ ID NO:23. In some embodiments, the polynucleotide comprises a polynucleotide encoding a polypeptide comprising an amino acid sequence of SEQ ID NO:24. In some embodiments, the polynucleotide comprises a polynucleotide encoding a polypeptide comprising an amino acid sequence of SEQ ID NO:25. In some embodiments, the polynucleotide comprises a polynucleotide encoding a polypeptide comprising an amino acid sequence of SEQ ID NO:26. In some embodiments, the polynucleotide comprises a polynucleotide encoding a polypeptide comprising an amino acid sequence of SEQ ID NO:27. In some embodiments, the polynucleotide comprises a polynucleotide encoding a polypeptide comprising an amino acid sequence of SEQ ID NO:28. In some embodiments, the polynucleotide comprises a polynucleotide encoding a polypeptide comprising an amino acid sequence of SEQ ID NO:29. In some embodiments, the polynucleotide comprises a polynucleotide encoding a polypeptide comprising an amino acid sequence of SEQ ID NO:30. In some embodiments, the polynucleotide comprises a polynucleotide encoding a polypeptide comprising an amino acid sequence of SEQ ID NO:31. In some embodiments, the polynucleotide comprises a polynucleotide encoding a polypeptide comprising an amino acid sequence of SEQ ID NO:32. In some embodiments, the polynucleotide comprises a polynucleotide encoding a polypeptide comprising an amino acid sequence of SEQ ID NO:33. In some embodiments, the polynucleotide comprises a polynucleotide encoding a polypeptide comprising an amino acid sequence of SEQ ID NO:34. In some embodiments, the polynucleotide comprises a polynucleotide encoding a polypeptide comprising an amino acid sequence of SEQ ID NO:35. In some embodiments, the polynucleotide comprises a polynucleotide encoding a polypeptide comprising an amino acid sequence of SEQ ID NO:36. In some embodiments, the polynucleotide comprises a polynucleotide encoding a polypeptide comprising an amino acid sequence of SEQ ID NO:37. In some embodiments, the polynucleotide comprises a polynucleotide encoding a polypeptide comprising an amino acid sequence of SEQ ID NO:38. In some embodiments, the polynucleotide comprises a polynucleotide encoding a polypeptide comprising an amino acid sequence of SEQ ID NO:39. In some embodiments, the polynucleotide comprises a polynucleotide encoding a polypeptide comprising an amino acid sequence of SEQ ID NO:40. In some embodiments, the polynucleotide comprises a polynucleotide encoding a polypeptide comprising an amino acid sequence of SEQ ID NO:41. In some embodiments, the polynucleotide comprises a polynucleotide encoding a polypeptide comprising an amino acid sequence of SEQ ID NO:42. In some embodiments, the polynucleotide comprises a polynucleotide encoding a polypeptide comprising an amino acid sequence of SEQ ID NO:43. In some embodiments, the polynucleotide comprises a polynucleotide encoding a polypeptide comprising an amino acid sequence of SEQ ID NO:199. In some embodiments, the polynucleotide comprises a polynucleotide encoding a polypeptide comprising an amino acid sequence of SEQ ID NO:44. In some embodiments, the polynucleotide comprises a polynucleotide encoding a polypeptide comprising an amino acid sequence of SEQ ID NO:45. In some embodiments, the polynucleotide comprises a polynucleotide encoding a polypeptide comprising an amino acid sequence of SEQ ID NO:46. In some embodiments, the polynucleotide comprises a polynucleotide encoding a polypeptide comprising an amino acid sequence of SEQ ID NO:47. In some embodiments, the polynucleotide comprises a polynucleotide encoding a polypeptide comprising an amino acid sequence of SEQ ID NO:48. In some embodiments, the polynucleotide comprises a polynucleotide encoding a polypeptide comprising an amino acid sequence of SEQ ID NO:49. In some embodiments, the polynucleotide comprises a polynucleotide encoding a polypeptide comprising an amino acid sequence of SEQ ID NO:50. In some embodiments, the polynucleotide comprises a polynucleotide encoding a polypeptide comprising an amino acid sequence of SEQ ID NO:51. In some embodiments, the polynucleotide comprises a polynucleotide encoding a polypeptide comprising an amino acid sequence of SEQ ID NO:52. In some embodiments, the polynucleotide comprises a polynucleotide encoding a polypeptide comprising an amino acid sequence of SEQ ID NO:53. In some embodiments, the polynucleotide comprises a polynucleotide encoding a polypeptide comprising an amino acid sequence of SEQ ID NO:54. In some embodiments, the polynucleotide comprises a polynucleotide encoding a polypeptide comprising an amino acid sequence of SEQ ID NO:58. In some embodiments, the polynucleotide comprises a polynucleotide encoding a polypeptide comprising an amino acid sequence of SEQ ID NO:59. In some embodiments, the polynucleotide comprises a polynucleotide encoding a polypeptide comprising an amino acid sequence of SEQ ID NO:60.

In some embodiments, the polynucleotide comprises a polynucleotide (e.g., a nucleotide sequence) encoding a polypeptide comprising more than one amino acid sequence selected from the group consisting of: SEQ ID NOs:9-54, 58-60 and 199. In some embodiments, the polynucleotide comprises a polynucleotide selected from the group consisting of SEQ ID NOs: 130-175, 179-181 and 200. In some embodiments, the polynucleotide comprises SEQ ID NO: 130. In some embodiments, the polynucleotide comprises SEQ ID NO: 131. In some embodiments, the polynucleotide comprises SEQ ID NO: 132. In some embodiments, the polynucleotide comprises SEQ ID NO: 133. In some embodiments, the polynucleotide comprises SEQ ID NO: 134. In some embodiments, the polynucleotide comprises SEQ ID NO: 135. In some embodiments, the polynucleotide comprises SEQ ID NO: 136. In some embodiments, the polynucleotide comprises SEQ ID NO: 137. In some embodiments, the polynucleotide comprises SEQ ID NO: 138. In some embodiments, the polynucleotide comprises SEQ ID NO: 139. In some embodiments, the polynucleotide comprises SEQ ID NO: 140. In some embodiments, the polynucleotide comprises SEQ ID NO: 141. In some embodiments, the polynucleotide comprises SEQ ID NO: 142. In some embodiments, the polynucleotide comprises SEQ ID NO: 143. In some embodiments, the polynucleotide comprises SEQ ID NO: 144. In some embodiments, the polynucleotide comprises SEQ ID NO: 145. In some embodiments, the polynucleotide comprises SEQ ID NO: 146. In some embodiments, the polynucleotide comprises SEQ ID NO: 147. In some embodiments, the polynucleotide comprises SEQ ID NO: 148. In some embodiments, the polynucleotide comprises SEQ ID NO: 149. In some embodiments, the polynucleotide comprises SEQ ID NO: 150. In some embodiments, the polynucleotide comprises SEQ ID NO: 151. In some embodiments, the polynucleotide comprises SEQ ID NO: 152. In some embodiments, the polynucleotide comprises SEQ ID NO: 153. In some embodiments, the polynucleotide comprises SEQ ID NO: 154. In some embodiments, the polynucleotide comprises SEQ ID NO: 155. In some embodiments, the polynucleotide comprises SEQ ID NO: 156. In some embodiments, the polynucleotide comprises SEQ ID NO: 157. In some embodiments, the polynucleotide comprises SEQ ID NO: 158. In some embodiments, the polynucleotide comprises SEQ ID NO: 160. In some embodiments, the polynucleotide comprises SEQ ID NO: 161. In some embodiments, the polynucleotide comprises SEQ ID NO: 162. In some embodiments, the polynucleotide comprises SEQ ID NO: 163. In some embodiments, the polynucleotide comprises SEQ ID NO: 164. In some embodiments, the polynucleotide comprises SEQ ID NO: 200. In some embodiments, the polynucleotide comprises SEQ ID NO: 165. In some embodiments, the polynucleotide comprises SEQ ID NO: 166. In some embodiments, the polynucleotide comprises SEQ ID NO: 167. In some embodiments, the polynucleotide comprises SEQ ID NO: 168. In some embodiments, the polynucleotide comprises SEQ ID NO: 169. In some embodiments, the polynucleotide comprises SEQ ID NO: 170. In some embodiments, the polynucleotide comprises SEQ ID NO: 171. In some embodiments, the polynucleotide comprises SEQ ID NO: 172. In some embodiments, the polynucleotide comprises SEQ ID NO: 173. In some embodiments, the polynucleotide comprises SEQ ID NO: 174. In some embodiments, the polynucleotide comprises SEQ ID NO: 179. In some embodiments, the polynucleotide comprises SEQ ID NO: 180. In some embodiments, the polynucleotide comprises SEQ ID NO: 181.

The present disclosure also provides variants of the polynucleotides described herein, wherein the variant encodes, for example, fragments, analogs, and/or derivatives of a CD30-binding moiety described herein. In some embodiments, the present disclosure provides a polynucleotide comprising a polynucleotide having a nucleotide sequence at least about 80% identical, at least about 85% identical, at least about 90% identical, at least about 95% identical, at least about 96% identical, at least 97% identical, at least 98% identical, or at least 99% identical to a polynucleotide sequence encoding a polypeptide described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises a polynucleotide having a nucleotide sequence at least about 80% identical, at least about 85% identical, at least about 90% identical, at least about 95% identical, at least about 96% identical, at least 97% identical, at least 98% identical, or at least 99% identical to a polynucleotide encoding an amino acid sequence selected from the group consisting of: SEQ ID NOs:9-54, 58-60 and 199. Also provided is a polynucleotide that comprises a polynucleotide that hybridizes to a polynucleotide encoding an amino acid sequence selected from the group consisting of: SEQ ID NOs: SEQ ID NOs:9-54, 58-60 and 199. In some embodiments, a polynucleotide comprises a polynucleotide that is at least about 80% identical, at least about 85% identical, at least about 90% identical, at least about 95% identical, at least about 96% identical, at least 97% identical, at least 98% identical, or at least 99% identical to a polynucleotide selected from the group consisting of: 130-175, 179-181 and 200. Also provided is a polynucleotide that comprises a polynucleotide that hybridizes to a polynucleotide selected from the group consisting of: 130-175, 179-181 and 200. In some embodiments, the hybridization is under conditions of high stringency as is known to those skilled in the art.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker may be used in conjunction with other markers or tags.

In some embodiments, a polynucleotide is isolated. In some embodiments, a polynucleotide is substantially pure.

Vectors and cells comprising the polynucleotides described herein are also provided. In some embodiments, an expression vector comprises a polynucleotide encoding a CD30-binding moiety described herein. In some embodiments, an expression vector comprises a polynucleotide molecule encoding a polypeptide that is part of a CD30-binding moiety described herein. In some embodiments, a host cell comprises an expression vector comprising a polynucleotide molecule encoding a CD30-binding moiety described herein. In some embodiments, a host cell comprises an expression vector comprising a polynucleotide molecule encoding a polypeptide that is part of a CD30-binding moiety described herein. In some embodiments, a host cell comprises a polynucleotide encoding a CD30-binding moiety described herein.

The CD30-binding moieties described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press; Borrebaeck (ed.) (1995) Antibody Engineering, Second Edition, Oxford University Press; Lo (ed.) (2006) Antibody Engineering: Methods and Protocols (Methods in Molecular Biology); Vol. 248, Humana Press, Inc; each of which is incorporated herein by reference in its entirety.

The CD30-binding moieties described herein can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge Science 269:202 (1995); Merrifield, Methods Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

The CD30-binding moieties described herein that comprise antibody can be prepared using a wide variety of techniques known in the art including the use of hybridoma and recombinant technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981), each of which is incorporated herein by reference in its entirety. Other methods of producing the cobinders are also known in the art.

In some embodiments, a recombinant expression vector is used to amplify and express DNA encoding a CD30-binding moiety. For example, a recombinant expression vector can be a replicable DNA construct that includes synthetic or cDNA-derived DNA fragments encoding a polypeptide chain of a CD30-binding moiety, such as an anti-CD30 antibody operatively linked to suitable transcriptional and/or translational regulatory elements derived from mammalian, microbial, viral or insect genes. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, a CD30-binding moiety (e.g., an antibody) of the present disclosure is expressed from one or more vectors. Suitable host cells for expression of a CD30-binding moiety (e.g., an antibody) or a CD30 protein or fragment thereof to use as an antigen or immunogen include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the CD30-binding moieties described herein. In some embodiments, the cells produce the CD30-binding moieties described herein. In some embodiments, the cells produce an antibody. In some embodiments, the cells produce an antibody that binds human CD30. In some embodiments, the cells produce an antibody that binds rhesus CD30. In some embodiments, the cells produce an antibody that binds human CD30 and rhesus CD30. In some embodiments, the cells produce antibody AS47863. In some embodiments, the cells produce a humanized version of antibody AS47863, referred to as AS47863VH4, AS47863VH5, AS47863VH11, or AS47863VH12. In some embodiments, the cells produce antibody AS48433. In some embodiments, the cells produce a humanized version of antibody AS48433, referred to as AS48433VH4, AS48433VH5, AS48433VH11, or AS48433VH12. In some embodiments, the cells produce an antibody designated AS48463. In some embodiments, the cells produce a humanized version of antibody designated AS48463, referred to as AS48463VH4, or AS48463VH11. In some embodiments, the cells produce an antibody designated AS48481. In some embodiments, the cells produce a humanized version of antibody designated AS48481, referred to as AS48481VH5, AS48481VH6, AS48481VH13, or AS48481VH14. In some embodiments, the cells produce an antibody designated AS48508. In some embodiments, the cells produce a humanized version of antibody designated AS48508, referred to as AS48508VH4, AS48508VH5, AS48508VH11, or AS48508VH12. In some embodiments, the cells produce an antibody designated AS48542. In some embodiments, the cells produce a humanized version of antibody designated AS48542, referred to as AS48542VH5 or AS48542VH12. In some embodiments, the cells produce an antibody designated AS53445. In some embodiments, the cells produce a humanized version of antibody designated AS53445, referred to as AS53445VH4 or AS53445VH11. In some embodiments, the cells produce an antibody designated AS53574. In some embodiments, the cells produce a humanized version of antibody designated AS53574, referred to as AS53574VH4, AS53574VH5, AS53574VH6, AS53574VH7, AS53574VH11, AS53574VH12 or AS53574VH13. In some embodiments, the cells produce antibody AS53750. In some embodiments, the cells produce a humanized version of antibody designated AS53750, referred to as AS53750VH4, AS53750VH5, AS53750VH11, or AS53750VH12. In some embodiments, the cells produce antibody AS54233. In some embodiments, the cells produce a humanized version of antibody designated AS54233, referred to as AS54233VH4, AS54233VH5, AS54233VH11, or AS54233VH12. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell.

CD30-binding moieties (e.g., antibodies) of the present disclosure can be analyzed for their physical, chemical and/or biological properties by various methods known in the art. In some embodiments, an anti-CD30 antibody is tested for its ability to bind CD30 (e.g., human CD30 and/or rhesus CD30). Binding assays include, but are not limited to, SPR (e.g., Biacore), ELISA, and FACS. In addition, antibodies may be evaluated for solubility, stability, thermostability, viscosity, expression levels, expression quality, and/or purification efficiency.

Epitope mapping is a method of identifying the binding site, region, or epitope on a target protein where an antibody (or other binding moiety) binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, CD30-binding moieties (e.g., antibodies) described herein are characterized by assays including, but not limited to, N-terminal sequencing, amino acid analysis, HPLC, mass spectrometry, ion exchange chromatography, and papain digestion.

In some embodiments, a CD30-binding moiety comprises conjugates comprising an anti-CD30 antibody described herein. In some embodiments, an anti-CD30 antibody is conjugated to a cytotoxic agent or moiety. In some embodiments, an anti-CD30 antibody is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC 1065.

In some embodiments, a CD30-binding moiety (e.g., an antibody) described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

A CD30-binding moiety (e.g., an antibody) described herein may be attached to a solid support. Such solid supports include, but are not limited to, glass, cellulose, polyacrylamide, nylon, polystyrene, polyvinyl chloride, or polypropylene. In some embodiments, an immobilized anti-CD30 antibody is used in an immunoassay. In some embodiments, an immobilized anti-CD30 antibody is used in purification of the target antigen (e.g., human CD30 or rhesus CD30).

### 3. THE CD30 CARS

In one aspect, provided herein are chimeric antigen receptors (CARs) that specifically bind CD30 (“CD30 CAR”). In some embodiments, the CD30 CARs comprise, from N-terminus to C-terminus, a CD30-binding moiety, a transmembrane (TM) domain, and a cytoplasmic domain. The CD30-binding moiety can be any CD30-binding moiety can be any CD30-binding moiety described herein or a variant thereof. In some embodiments, the CD30-binding moiety comprises an anti-CD30 antibody described herein or a variant thereof. In some embodiments, the CD30-binding moiety comprises two anti-CD30 antibodies described herein or variants thereof. In certain embodiments, the CD30-binding moiety comprises a sdAb disclosed herein or a variant thereof. In some embodiments, the CD30-binding moiety comprises a HCAb which comprise a sdAb fused with human IgG1 hinge and Fc region. In certain embodiments, the CD30-binding moiety comprises a scFv disclosed herein or a variant thereof. In certain embodiments, the CD30-binding moiety comprises a tandem repeat of a scFv disclosed herein or a variant thereof. In some embodiments, the CD30-binding moiety comprises an antigen-binding fragment comprising a sdAb, a HCAb, a Fab, a Fab′, a F(ab′)₂, a Fv, a scFv, a (scFv)₂, an IgG1 antibody, an IgG2 antibody, an IgG3 antibody, or an IgG4 antibody comprising the CDRs (e.g. CDR1, CDR2, and CDR3) described herein or variants thereof.

In some embodiments, a CD30 CAR comprise, from N-terminus to C-terminus, a CD30-binding moiety that specifically binds CRD6 of CD30 (e.g., SEQ ID NO:8), a transmembrane domain, and a cytoplasmic domain. In some embodiments, a CD30 CAR comprise, from N-terminus to C-terminus, an antigen-binding fragment that specifically binds CRD1 of CD30 (e.g., SEQ ID NO:3), a transmembrane domain, and a cytoplasmic domain. In some embodiments, a CD30 CAR comprise, from N-terminus to C-terminus, a CD30-binding moiety that specifically binds CRD6 of CD30 (e.g., SEQ ID NO:8) and CRD1 of CD30 (e.g., SEQ ID NO:3), a transmembrane domain, and a cytoplasmic domain.

In some embodiments, CARs provided herein have a CD30-binding moiety that comprises (i) a CDR1 comprising an amino acid sequence selected from the group consisting of SEQ ID NOs:87-95; (ii) a CDR2 comprising an amino acid sequence selected from the group consisting of SEQ ID NOs:100-106; and (iii) a CDR3 comprising an amino acid sequence selected from the group consisting of SEQ ID NOs:111-120; or a variant thereof comprising up to 3 amino acid substitutions in each of CDR1, CDR2, and CDR3.

In some embodiments, CARs provided herein have a CD30-binding moiety that comprises (1) a CDR1 comprising SEQ ID NO:87; a CDR2 comprising SEQ ID NO:100; and a CDR3 comprising SEQ ID NO:111; (2) a CDR1 comprising SEQ ID NO:87; a CDR2 comprising SEQ ID NO:100; and a CDR3 comprising SEQ ID NO:112; (3) a CDR1 comprising SEQ ID NO:88; a CDR2 comprising SEQ ID NO:101; and a CDR3 comprising SEQ ID NO:113; (4) a CDR1 comprising SEQ ID NO:89; a CDR2 comprising SEQ ID NO:102; and a CDR3 comprising SEQ ID NO:114; (5) a CDR1 comprising SEQ ID NO:90; a CDR2 comprising SEQ ID NO:103; and a CDR3 comprising SEQ ID NO:115; (6) a CDR1 comprising SEQ ID NO:91; a CDR2 comprising SEQ ID NO:104; and a CDR3 comprising SEQ ID NO:116; (7) a CDR1 comprising SEQ ID NO:92; a CDR2 comprising SEQ ID NO:105; and a CDR3 comprising SEQ ID NO:117; (8) a CDR1 comprising SEQ ID NO:93; a CDR2 comprising SEQ ID NO:106; and a CDR3 comprising SEQ ID NO:118; (9) a CDR1 comprising SEQ ID NO:94; a CDR2 comprising SEQ ID NO:103; and a CDR3 comprising SEQ ID NO:119; or (10) a CDR1 comprising SEQ ID NO:95; a CDR2 comprising SEQ ID NO:103; and a CDR3 comprising SEQ ID NO:120; or a variant thereof comprising up to about 5 amino acid substitutions in the CDRs.

In some embodiments, CARs provided herein have a CD30-binding moiety that comprises an amino acid sequence selected from the group consisting of SEQ ID NOs:9-54, 199, or a variant thereof.

In some embodiments, CARs provided herein have a CD30-binding moiety that is a sdAb. In some embodiments, CARs provided herein have a CD30-binding moiety that is a sdAb designated as AS47863, AS47863VH4, AS47863VH5, AS47863VH11, AS47863VH12, AS48433, AS48433VH4, AS48433VH5, AS48433VH11, AS48433VH12, AS48463, AS48463VH4, AS48463VH11, AS48481, AS48481VH5, AS48481VH6, AS48481VH13, AS48481VH14, AS48508, AS48508VH4, AS48508VH5, AS48508VH11, AS48508VH12, AS48542, AS48542VH5, AS48542VH12, AS53445, AS53445VH4, AS53445VH11, AS53574, AS53574VH4, AS53574VH5, AS53574VH6, AS53574VH7, AS53574VH11, AS53574VH12, AS53574VH13, AS53750, AS53750VH4, AS53750VH5, AS53750VH11, AS53750VH12, AS54233, AS54233VH4, AS54233VH5, AS54233VH11, or AS54233VH12. In some embodiments, CARs provided herein have a CD30-binding moiety that is AS47863. In some embodiments, CARs provided herein have a CD30-binding moiety that is AS47863VH4. In some embodiments, CARs provided herein have a CD30-binding moiety that is AS47863VH5. In some embodiments, CARs provided herein have a CD30-binding moiety that is AS47863VH11. In some embodiments, CARs provided herein have a CD30-binding moiety that is AS47863VH12. In some embodiments, CARs provided herein have a CD30-binding moiety that is AS48433. In some embodiments, CARs provided herein have a CD30-binding moiety that is AS48433VH4. In some embodiments, CARs provided herein have a CD30-binding moiety that is AS48433VH5. In some embodiments, CARs provided herein have a CD30-binding moiety that is AS48433VH11. In some embodiments, CARs provided herein have a CD30-binding moiety that is AS48433VH12. In some embodiments, CARs provided herein have a CD30-binding moiety that is AS48463. In some embodiments, CARs provided herein have a CD30-binding moiety that is AS48463VH4. In some embodiments, CARs provided herein have a CD30-binding moiety that is AS48463VH11. In some embodiments, CARs provided herein have a CD30-binding moiety that is AS48481. In some embodiments, CARs provided herein have a CD30-binding moiety that is AS48481VH5. In some embodiments, CARs provided herein have a CD30-binding moiety that is AS48481VH6. In some embodiments, CARs provided herein have a CD30-binding moiety that is AS48481VH13. In some embodiments, CARs provided herein have a CD30-binding moiety that is AS48481VH14. In some embodiments, CARs provided herein have a CD30-binding moiety that is AS48508. In some embodiments, CARs provided herein have a CD30-binding moiety that is AS48508VH4. In some embodiments, CARs provided herein have a CD30-binding moiety that is AS48508VH5. In some embodiments, CARs provided herein have a CD30-binding moiety that is AS48508VH11. In some embodiments, CARs provided herein have a CD30-binding moiety that is AS48508VH12. In some embodiments, CARs provided herein have a CD30-binding moiety that is AS48542. In some embodiments, CARs provided herein have a CD30-binding moiety that is AS48542VH5. In some embodiments, CARs provided herein have a CD30-binding moiety that is AS48542VH12. In some embodiments, CARs provided herein have a CD30-binding moiety that is AS53445. In some embodiments, CARs provided herein have a CD30-binding moiety that is AS53445VH4. In some embodiments, CARs provided herein have a CD30-binding moiety that is AS53445VH11. In some embodiments, CARs provided herein have a CD30-binding moiety that is AS53574. In some embodiments, CARs provided herein have a CD30-binding moiety that is AS53574VH4. In some embodiments, CARs provided herein have a CD30-binding moiety that is AS53574VH5. In some embodiments, CARs provided herein have a CD30-binding moiety that is AS53574VH6. In some embodiments, CARs provided herein have a CD30-binding moiety that is AS53574VH11. In some embodiments, CARs provided herein have a CD30-binding moiety that is AS53574VH12. In some embodiments, CARs provided herein have a CD30-binding moiety that is AS53574VH13. In some embodiments, CARs provided herein have a CD30-binding moiety that is AS53750. In some embodiments, CARs provided herein have a CD30-binding moiety that is AS53750VH4. In some embodiments, CARs provided herein have a CD30-binding moiety that is AS53750VH5. In some embodiments, CARs provided herein have a CD30-binding moiety that is AS53750VH11. In some embodiments, CARs provided herein have a CD30-binding moiety that is AS53750VH12. In some embodiments, CARs provided herein have a CD30-binding moiety that is AS54233. In some embodiments, CARs provided herein have a CD30-binding moiety that is AS54233VH4. In some embodiments, CARs provided herein have a CD30-binding moiety that is AS54233VH5. In some embodiments, CARs provided herein have a CD30-binding moiety that is AS54233VH11. In some embodiments, CARs provided herein have a CD30-binding moiety that is AS54233VH12.

In some embodiments, CARs provided herein has a CD30-binding moiety comprising (a) a VH comprising (i) a VH CDR1 comprising SEQ ID NO:96, 97, or 98; (ii) a VH CDR2 comprising SEQ ID NO:107, 108, or 109, and (iii) a VH CDR3 comprising SEQ ID NO:121, 122, or 123; and/or (b) a VL comprising (i) a VL CDR1 comprising SEQ ID NO:99; (ii) a VL CDR2 comprising SEQ ID NO:110; and (iii) a VL CDR3 comprising SEQ ID NO:124, 125, or 126; or a variant thereof comprising up to about 3 amino acid substitutions in each of VH CDR1, VH CDR2, VH CDR3, VL CDR1, VL CDR2, and VL CDR3.

In some embodiments, CARs provided herein has a CD30-binding moiety comprising (a) a VH comprising a VH CDR1 comprising SEQ ID NO:96, a VH CDR2 comprising SEQ ID NO:107, and a VH CDR3 comprising SEQ ID NO:121; and/or (b) a VL comprising a VL CDR1 comprising SEQ ID NO:99, a VL CDR2 comprising SEQ ID NO:110, and a VL CDR3 comprising SEQ ID NO:124.

In some embodiments, CARs provided herein has a CD30-binding moiety comprising (a) a VH comprising a VH CDR1 comprising SEQ ID NO:97, a VH CDR2 comprising SEQ ID NO:108, and a VH CDR3 comprising SEQ ID NO:122; and/or (b) a VL comprising a VL CDR1 comprising SEQ ID NO:99, a VL CDR2 comprising SEQ ID NO:110, and a VL CDR3 comprising SEQ ID NO:125.

In some embodiments, CARs provided herein has a CD30-binding moiety comprising (a) a VH comprising a VH CDR1 comprising SEQ ID NO:98, a VH CDR2 comprising SEQ ID NO:109, and a VH CDR3 comprising SEQ ID NO:123; and/or (b) a VL comprising a VL CDR1 comprising SEQ ID NO:99, a VL CDR2 comprising SEQ ID NO:110, and a VL CDR3 comprising SEQ ID NO:126.

In some embodiments, CARs provided herein has a CD30-binding moiety comprising a scFv designated as AS57659, AS57765, or AS57911.

CARs provided herein can have a bivalent CD30-binding moiety. In some embodiments, the CARs provided herein comprises, from N-terminus to C-terminus, a first anti-CD30 antibody, a linker, a second anti-CD30 antibody, a transmembrane domain and a cytoplasmic domain. The first anti-CD30 antibody and second anti-CD3 antibody can bind different epitopes of CD30. The first anti-CD30 antibody and second anti-CD3 antibody can bind the same epitopes of CD30. The first anti-CD30 antibody and the second anti-CD30 antibody can be any anti-CD30 antibodies described herein or variants thereof. For example, the CARs provided herein comprises, from N-terminus to C-terminus, AS48542, a linker, AS53574, a transmembrane domain and a cytoplasmic domain. For another example, the CARs provided herein comprises, from N-terminus to C-terminus, AS53574, a linker, AS48542, a transmembrane domain and a cytoplasmic domain.

Based in part on the unexpected finding that a bivalent CD-30 binding moiety that comprises a tandem repeat of an anti-CD30 antibody confers improved cytotoxicity to T-cells, in some embodiments, CARs provided herein have a bivalent CD30-binding moiety that comprises a tandem repeat of an anti-CD30 antibody described herein or a variant thereof, a transmembrane domain and a cytoplasmic domain. In certain embodiments, the CD30-binding moiety comprises a tandem repeat of a sdAb disclosed herein or a variant thereof. For example, in some embodiments, CARs provided herein has a CD30-binding moiety comprising a tandem repeat of AS53574. In some embodiments, CARs provided herein has a CD30-binding moiety comprising a tandem repeat of AS48542.

In certain embodiments, the CD30-binding moiety of a CAR disclosed herein comprises a leader sequence (e.g., a leader sequence of amino acid sequence SEQ ID NO:61). Without being bound by theory, in certain embodiments, the leader sequence facilitates expression of the CAR on the surface of the cell, but the presence of the leader sequence in an expressed CAR may not be necessary for the CAR to function. In some embodiments, upon expression of the CAR on the cell surface, all or a portion of the leader sequence can be cleaved off from the CAR.

In some embodiments, the leader sequence is positioned at the N-terminus of the CD30-binding moiety. The leader sequence can comprise any suitable leader sequence known in the art. In some embodiments, the CD30-binding moiety of a CAR disclosed herein comprises a leader sequence comprising or consisting of SEQ ID NO: 61.

In some embodiments, a CAR disclosed herein comprises a hinge domain that connects the CD-30 binding moiety and the transmembrane domain. In some embodiments, the hinge domain is a human hinge. In some embodiments, the hinge domain comprises human CD8a hinge domain. In some embodiments, the hinge domain comprises or consists of the amino acid sequence of SEQ ID NO:62. In some embodiments, the hinge domain comprises human CD28 hinge domain. In some embodiments, the hinge domain comprises or consists of the amino acid sequence of SEQ ID NO:127.

CARs provided in the present disclosure comprise a CD30-binding moiety, transmembrane (TM) domain, and a cytoplasmic domain. In some embodiments, the transmembrane domain is a human transmembrane domain. In some embodiments, the transmembrane domain comprises human CD8a transmembrane domain. In some embodiments, the transmembrane domain comprises or consists of the amino acid sequence of SEQ ID NO:63. In some embodiments, the transmembrane domain comprises human CD28 transmembrane domain. In some embodiments, the transmembrane domain comprises or consists of the amino acid sequence of SEQ ID NO:128.

CARs provided in the present disclosure comprise a CD30-binding moiety, transmembrane domain, and a cytoplasmic domain. The cytoplasmic domain mediates the activation of T cells that express such CAR upon binding of CD30-expressing cells. In some embodiments, the cytoplasmic domain comprises one or more domains (e.g., signaling domains and/or costimulatory domains) In some embodiments, the cytoplasmic domain comprises a signaling domain. In some embodiments, the cytoplasmic domain comprises a costimulatory domain. In some embodiments, the cytoplasmic domain(s) is human domain(s) Generally speaking, the signaling domain (e g. CD3 zeta) can mediate downstream signaling during T cell activation, which can be derived from the intracellular signaling portion of the T cell receptor; the costimulatory domain can enhance cytokine production, which can be derived from the intracellular signaling domains of costimulatory proteins (e.g. CD28 and 4-1BB).

In some embodiments, CARs provided in the present disclosure comprise a CD30-binding moiety, transmembrane domain, and at least one signaling domain. The signaling domain can be any signaling domain known in the art as appropriate for mediating downstream signaling during T cell activation. In some embodiments, the signaling domain is derived from CD3ζ, FcRγ, FcRβ, CD3γ, CD3δ, CD3ε, CD5, CD22, CD79a, CD79b, CD66d, or any combination thereof. In some embodiments, a signaling domain is the signaling domain of CD3ζ, FcRγ, FcRβ, CD3γ, CD3δ, CD3ε, CD5, CD22, CD79a, CD79b, CD66d, or any combination thereof. In some embodiments, a signaling domain is the cytoplasmic portion of CD3ζ, FcRγ, FcRβ, CD3γ, CD3δ, CD3ε, CD5, CD22, CD79a, CD79b, CD66d, or any combination thereof. In some embodiments, the signaling domain can also be a variant of the native signaling domain that maintains its activity in mediating downstream signaling during T cell activation. In some embodiments, a CAR disclosed herein comprises at least one signaling domain. In some embodiments, a CAR disclosed herein comprises at least two signaling domains. In some embodiments, a CAR disclosed herein comprises at least three signaling domains. In some embodiments, a CAR disclosed herein comprises the signaling domain of CD3ζ or a variant thereof. In some embodiments, a CAR disclosed herein comprises the signaling domain of FcRγ or a variant thereof. In some embodiments, a CAR disclosed herein comprises the signaling domain of FcRβ or a variant thereof. In some embodiments, a CAR disclosed herein comprises the signaling domain of CD3γ or a variant thereof. In some embodiments, a CAR disclosed herein comprises the signaling domain of CD3δ or a variant thereof. In some embodiments, a CAR disclosed herein comprises the signaling domain of CD3ε or a variant thereof. In some embodiments, a CAR disclosed herein comprises the signaling domain of CD5 or a variant thereof. In some embodiments, a CAR disclosed herein comprises the signaling domain of CD22 or a variant thereof. In some embodiments, a CAR disclosed herein comprises the signaling domain of CD79a or a variant thereof. In some embodiments, a CAR disclosed herein comprises the signaling domain of CD79b or a variant thereof. In some embodiments, a CAR disclosed herein comprises the signaling domain of CD66d or a variant thereof.

In some embodiments, CARs provided in the present disclosure comprise a CD30-binding moiety, a transmembrane domain, and at least one costimulatory domain. The costimulatory domain can be any costimulatory domain known in the art as appropriate for enhancing cytokine production, T cell survival and proliferation, or other T cell functionality during T cell activation. In some embodiments, a CAR disclosed herein comprises at least one costimulatory domain. In some embodiments, a CAR disclosed herein comprises at least two costimulatory domains. In some embodiments, a CAR disclosed herein comprises at least three costimulatory domains. In some embodiments, the costimulatory domain can be derived from CD28, 4-1BB (CD137), OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, a ligand that specifically binds with CD83, or any combination thereof. In some embodiments, the costimulatory domain can be the signaling domain from CD28, 4-1BB (CD137), OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, a ligand that specifically binds with CD83, or any combination thereof. In some embodiments, the costimulatory domain can be the cytoplasmic portion of CD28, 4-1BB (CD137), OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, a ligand that specifically binds with CD83, or any combination thereof. In some embodiments, the costimulatory domain is the cytoplasmic domain of CD28, 4-1BB (CD137), OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, a ligand that specifically binds with CD83, or any combination thereof. In some embodiments, the signaling domain can also be a variant of the native costimulatory domain that maintains its activity in enhancing cytokine production, T cell survival and proliferation, or other T cell functionality during T cell activation.

In some embodiments, the costimulatory domain comprises the signaling domain of CD28 or a variant thereof. In some embodiments, the costimulatory domain comprises the signaling domain of 4-1BB (CD137) or a variant thereof. In some embodiments, the costimulatory domain comprises the signaling domain of CD27 or a variant thereof. In some embodiments, the costimulatory domain comprises the signaling domain of OX40 or a variant thereof. In some embodiments, the costimulatory domain comprises the signaling domain of CD40 or a variant thereof. In some embodiments, the costimulatory domain comprises the signaling domain of PD-1 or a variant thereof. In some embodiments, the costimulatory domain comprises the signaling domain of ICOS or a variant thereof. In some embodiments, the costimulatory domain comprises the signaling domain of lymphocyte function-associated antigen-1 (LFA-1) or a variant thereof. In some embodiments, the costimulatory domain comprises the signaling domain of CD2 or a variant thereof. In some embodiments, the costimulatory domain comprises the signaling domain of CD7 or a variant thereof. In some embodiments, the costimulatory domain comprises the signaling domain of LIGHT or a variant thereof. In some embodiments, the costimulatory domain comprises the signaling domain of NKG2C or a variant thereof. In some embodiments, the costimulatory domain comprises the signaling domain of B7-H3 or a variant thereof. In some embodiments, the costimulatory domain comprises the signaling domain of the T cell signaling domain of TNFRSF9 or a variant thereof. In some embodiments, the costimulatory domain comprises the signaling domain of TNFRSF4 or a variant thereof. In some embodiments, the costimulatory domain comprises the signaling domain of TNFRSF8 or a variant thereof. In some embodiments, the costimulatory domain comprises the T cell signaling domain of CD40LG or a variant thereof. In some embodiments, the costimulatory domain comprises the signaling domain of ITGB2 or a variant thereof. In some embodiments, the costimulatory domain comprises the signaling domain of KLRC2 or a variant thereof. In some embodiments, the costimulatory domain comprises the signaling domain of TNFRSF18 or a variant thereof. In some embodiments, the costimulatory domain comprises the signaling domain of TNFRSF14 or a variant thereof. In some embodiments, the costimulatory domain comprises the signaling domain of HAVCR1 or a variant thereof. In some embodiments, the costimulatory domain comprises the signaling domain of LGALS9 or a variant thereof. In some embodiments, the costimulatory domain comprises the signaling domain of CD83 or a variant thereof. In some embodiments, the costimulatory domain comprises the signaling domain of a ligand that specifically binds with CD83 or a variant thereof.

In some embodiments, the cytoplasmic domain of the CARs disclosed herein comprises a signaling domain and a costimulatory domain. In some embodiments, the cytoplasmic domain comprises, for N-terminus to C-terminus, a signaling domain and a costimulatory domain. In some embodiments, the cytoplasmic domain comprises, for N-terminus to C-terminus, a costimulatory domain and a signaling domain. The signaling domain can be any signaling domain disclosed herein or otherwise known in the art. The costimulatory domain can be any costimulatory domain disclosed herein or otherwise known in the art. For example, in some embodiments, a CAR disclosed herein comprises a cytoplasmic domain that comprises the signaling domains of CD3ζ and 4-1BB. In another embodiment, a CAR disclosed herein comprises a cytoplasmic domain that comprises the signaling domains of CD3ζ and CD28. In another embodiment, a CAR disclosed herein comprises a cytoplasmic domain that comprises the signaling domains of CD3, 4-1BB, and CD28. In some embodiments, the CD3ζ signaling domain comprises or consists of the amino acid sequence of SEQ ID NO:65. In some embodiments, the 4-1BB signaling domain comprises or consists of the amino acid sequence of SEQ ID NO:64. In certain embodiments, the CD28 signaling domain comprises or consists of amino acid sequence of SEQ ID NO:129.

In some embodiments, a CAR disclosed herein comprises, from N-terminus to the C-terminus, a leader sequence, a CD30-binding moiety, a hinge, a transmembrane region, and a cytoplasmic domain. In some embodiments, a CAR disclosed herein comprises, from N-terminus to the C-terminus, a leader sequence, a CD30-binding moiety, a hinge, a transmembrane, a costimulatory domain and a signaling domain.

In some embodiments, a CAR disclosed herein comprises, from N-terminus to the C-terminus, a leader sequence (e.g., SEQ ID NO: 61), a CD30-binding moiety (e.g., sdAbs or scFvs disclosed herein), a hinge (e.g., CD8a hinge or CD28 hinge), a transmembrane region (e.g., CD8a transmembrane region or CD28 transmembrane region), a costimulatory domain (e.g. the T cell signaling domain of 4-1BB, or CD28), and a signaling domain (e.g., the T cell signaling domain of CD3ζ).

In some embodiments, a CAR disclosed herein comprises, from N-terminus to the C-terminus, a leader sequence (SEQ ID NO: 61), target binding moiety (i.e. anti-CD30 sdAb or scFv), CD8a hinge (SEQ ID NO: 62), CD8a transmembrane (TM) region (SEQ ID NO: 63), the cytoplasmic portion of the 4-1BB (CD137) molecule (SEQ ID NO: 64), and the cytoplasmic portion of the CD3ζ molecule (SEQ ID NO: 65). These CARs were designated “[CD30-binding moiety]bbz.” For example, a CAR designated AS47863bbz comprises, from N-terminus to the C-terminus, a leader sequence (e.g., SEQ ID NO: 61), the sdAb antibody designated AS47863 (SEQ ID NO:9), CD8α hinge (SEQ ID NO: 62), CD8α transmembrane (TM) region (SEQ ID NO: 63), the cytoplasmic portion of the 4-1BB (CD137) molecule (SEQ ID NO: 64), and the cytoplasmic portion of the CD3ζ molecule (SEQ ID NO: 65). For another example, a CAR designated AS48542VH5bbz comprises, from N-terminus to the C-terminus, a leader sequence (e.g., SEQ ID NO: 61), the antibody designated AS48542VH5 (SEQ ID NO:37), CD8α hinge (SEQ ID NO: 62), CD8α transmembrane (TM) region (SEQ ID NO: 63), the cytoplasmic portion of the 4-1BB (CD137) molecule (SEQ ID NO: 64), and the cytoplasmic portion of the CD3ζ molecule (SEQ ID NO: 65).

In some embodiments, a CAR disclosed herein comprises, from N-terminus to the C-terminus, a leader sequence (SEQ ID NO: 61), target binding moiety (i.e. anti-CD30 sdAb or scFv), CD28 hinge (SEQ ID NO: 127), CD28 transmembrane (TM) region (SEQ ID NO: 128), the cytoplasmic portion of CD28 molecule (SEQ ID NO: 129), and the cytoplasmic portion of the CD3ζ molecule (SEQ ID NO: 65). These CARs were designated “[CD30-binding moiety]-28z.” For another example, a CAR designated AS48542-28z (SEQ ID NO: 201) comprises, from N-terminus to the C-terminus, a leader sequence (e.g., SEQ ID NO: 61), the antibody designated AS48542 (SEQ ID NO:14), CD28 hinge (SEQ ID NO: 127), CD28 transmembrane (TM) region (SEQ ID NO: 128), the cytoplasmic portion of CD28 molecule (SEQ ID NO: 129), and the cytoplasmic portion of the CD3ζ molecule (SEQ ID NO: 65).

Accordingly, in some embodiments, provided herein are CD30 CARs designated as AS47863bbz, AS48433bbz, AS48463bbz, AS48481bbz, AS48508bbz, AS48542bbz, AS53445bbz, AS53574bbz, AS53750bbz, AS54233bbz, AS57659bbz, AS57765bbz, or AS57911bbz. In some embodiments, provided herein is a CD30 CAR designated as AS47863bbz or a variant thereof. In some embodiments, a CD30 CAR designated AS47863bbz comprises an amino acid sequence of SEQ ID NO:70. In some embodiments, provided herein is a CD30 CAR designated as AS48433bbz or a variant thereof. In some embodiments, a CD30 CAR designated AS48433bbz comprises an amino acid sequence of SEQ ID NO:71. In some embodiments, provided herein is a CD30 CAR designated as AS48463bbz or a variant thereof. In some embodiments, a CD30 CAR designated AS48463bbz comprises an amino acid sequence of SEQ ID NO:72. In some embodiments, provided herein is a CD30 CAR designated as AS48481bbz or a variant thereof. In some embodiments, a CD30 CAR designated AS48481bbz comprises an amino acid sequence of SEQ ID NO:73. In some embodiments, provided herein is a CD30 CAR designated as AS48508bbz or a variant thereof. In some embodiments, a CD30 CAR designated AS48508bbz comprises an amino acid sequence of SEQ ID NO:74. In some embodiments, provided herein is a CD30 CAR designated as AS48542bbz or a variant thereof. In some embodiments, a CD30 CAR designated AS48542bbz comprises an amino acid sequence of SEQ ID NO:75. In some embodiments, provided herein is a CD30 CAR designated as AS53750bbz or a variant thereof. In some embodiments, a CD30 CAR designated AS53750bbz comprises an amino acid sequence of SEQ ID NO:76. In some embodiments, provided herein is a CD30 CAR designated as AS54233bbz or a variant thereof. In some embodiments, a CD30 CAR designated AS54233bbz comprises an amino acid sequence of SEQ ID NO:77. In some embodiments, provided herein is a CD30 CAR designated as AS53445bbz or a variant thereof. In some embodiments, a CD30 CAR designated AS53445bbz comprises an amino acid sequence of SEQ ID NO:78. In some embodiments, provided herein is a CD30 CAR designated as AS53574bbz or a variant thereof. In some embodiments, a CD30 CAR designated AS53574bbz comprises an amino acid sequence of SEQ ID NO:79. In some embodiments, provided herein is a CD30 CAR designated as AS57911bbz or a variant thereof. In some embodiments, a CD30 CAR designated AS57911bbz comprises an amino acid sequence of SEQ ID NO:80. In some embodiments, provided herein is a CD30 CAR designated as AS57659bbz or a variant thereof. In some embodiments, a CD30 CAR designated AS57659bbz comprises an amino acid sequence of SEQ ID NO:81. In some embodiments, provided herein is a CD30 CAR designated as AS57765bbz or a variant thereof. In some embodiments, a CD30 CAR designated AS57765bbz comprises an amino acid sequence of SEQ ID NO:82.

In some embodiments, provided herein are CD30 CARs designated as AS47863VH4bbz, AS47863VH5bbz, AS47863VH11bbz, AS47863VH12bbz, AS48433VH4bbz, AS48433VH5bbz, AS48433VH11bbz, AS48433VH12bbz, AS48463VH4bbz, AS48463VH11bbz, AS48481VH5bbz, AS48481VH6bbz, AS48481VH13bbz, AS48481VH14bbz, AS48508VH4bbz, AS48508VH5bbz, AS48508VH11bbz, AS48508VH12bbz, AS48542VH5bbz, AS48542VH12bbz, AS53445VH4bbz, AS53445VH11bbz, AS53574VH4bbz, AS53574VH5bbz, AS53574VH6bbz, AS53574VH7, AS53574VH11bbz, AS53574VH12bbz, AS53574VH13bbz, AS53750VH4bbz, AS53750VH5bbz, AS53750VH11bbz, AS53750VH12bbz, AS54233VH4bbz, AS54233VH5bbz, AS54233VH11bbz, or AS54233VH12bbz. In some embodiments, provided herein are CD30 CARs designated as AS47863VH4bbz or a variant thereof. In some embodiments, a CD30 CAR designated AS47863VH4bbz comprises an amino acid sequence of SEQ ID NO:184. In some embodiments, provided herein are CD30 CARs designated as AS47863VH5bbz or a variant thereof. In some embodiments, provided herein are CD30 CARs designated as AS47863VH11bbz or a variant thereof. In some embodiments, provided herein are CD30 CARs designated as AS47863VH12bbz or a variant thereof. In some embodiments, provided herein are CD30 CARs designated as AS48433VH4bbz or a variant thereof. In some embodiments, provided herein are CD30 CARs designated as AS48433VH5bbz or a variant thereof. In some embodiments, provided herein are CD30 CARs designated as AS48433VH11bbz or a variant thereof. In some embodiments, provided herein are CD30 CARs designated as AS48433VH12bbz or a variant thereof. In some embodiments, provided herein are CD30 CARs designated as AS48463VH4bbz or a variant thereof. In some embodiments, a CD30 CAR designated AS48463VH4bbz comprises an amino acid sequence of SEQ ID NO:183. In some embodiments, provided herein are CD30 CARs designated as AS48463VH11bbz or a variant thereof. In some embodiments, provided herein are CD30 CARs designated as AS48481VH5bbz or a variant thereof. In some embodiments, provided herein are CD30 CARs designated as AS48481VH6bbz or a variant thereof. In some embodiments, provided herein are CD30 CARs designated as AS48481VH13bbz or a variant thereof. In some embodiments, provided herein are CD30 CARs designated as AS48481VH14bbz or a variant thereof. In some embodiments, provided herein are CD30 CARs designated as AS48508VH4bbz or a variant thereof. In some embodiments, provided herein are CD30 CARs designated as AS48508VH5bbz or a variant thereof. In some embodiments, provided herein are CD30 CARs designated as AS48508VH11bbz or a variant thereof. In some embodiments, provided herein are CD30 CARs designated as AS48508VH12bbz or a variant thereof. In some embodiments, provided herein are CD30 CARs designated as AS48542VH5bbz or a variant thereof. In some embodiments, a CD30 CAR designated AS48542VH5bbz comprises an amino acid sequence of SEQ ID NO:182. In some embodiments, provided herein are CD30 CARs designated as AS48542VH12bbz or a variant thereof. In some embodiments, provided herein are CD30 CARs designated as AS53445VH4bbz or a variant thereof. In some embodiments, provided herein are CD30 CARs designated as AS53445VH11bbz or a variant thereof. In some embodiments, provided herein are CD30 CARs designated as AS53574VH4bbz or a variant thereof. In some embodiments, provided herein are CD30 CARs designated as AS53574VH5bbz or a variant thereof. In some embodiments, provided herein are CD30 CARs designated as AS53574VH6bbz or a variant thereof. In some embodiments, provided herein are CD30 CARs designated as AS53574VH7bbz or a variant thereof. In some embodiments, provided herein are CD30 CARs designated as AS53574VH11bbz or a variant thereof. In some embodiments, provided herein are CD30 CARs designated as AS53574VH12bbz or a variant thereof. In some embodiments, provided herein are CD30 CARs designated as AS53574VH13bbz or a variant thereof. In some embodiments, provided herein are CD30 CARs designated as AS53750VH4bbz or a variant thereof. In some embodiments, provided herein are CD30 CARs designated as AS53750VH5bbz or a variant thereof. In some embodiments, provided herein are CD30 CARs designated as AS53750VH11bbz or a variant thereof. In some embodiments, provided herein are CD30 CARs designated as AS53750VH12bbz or a variant thereof. In some embodiments, provided herein are CD30 CARs designated as AS54233VH4bbz or a variant thereof. In some embodiments, provided herein are CD30 CARs designated as AS54233VH5bbz or a variant thereof. In some embodiments, provided herein are CD30 CARs designated as AS54233VH11bbz or a variant thereof. In some embodiments, provided herein are CD30 CARs designated as AS54233VH12bbz or a variant thereof.

Accordingly, in some embodiments, provided herein are CD30 CARs designated as AS47863-28z, AS48433-28z, AS48463-28z, AS48481-28z, AS48508-28z, AS48542-28z, AS53445-28z, AS53574-28z, AS53750-28z, AS54233-28z, AS57659-28z, AS57765-28z, or AS57911-28z. In some embodiments, provided herein is a CD30 CAR designated as AS47863-28z or a variant thereof. In some embodiments, provided herein is a CD30 CAR designated as AS48433-28z or a variant thereof. In some embodiments, provided herein is a CD30 CAR designated as AS48463-28z or a variant thereof. In some embodiments, provided herein is a CD30 CAR designated as AS48481-28z or a variant thereof. In some embodiments, provided herein is a CD30 CAR designated as AS48508-28z or a variant thereof. In some embodiments, provided herein is a CD30 CAR designated as AS48542-28z or a variant thereof. In some embodiments, provided herein is a CD30 CAR designated as AS53750-28z or a variant thereof. In some embodiments, provided herein is a CD30 CAR designated as AS54233-28z or a variant thereof. In some embodiments, provided herein is a CD30 CAR designated as AS53445-28z or a variant thereof. In some embodiments, provided herein is a CD30 CAR designated as AS53574-28z or a variant thereof. In some embodiments, provided herein is a CD30 CAR designated as AS57911-28z or a variant thereof. In some embodiments, provided herein is a CD30 CAR designated as AS57659-28z or a variant thereof. In some embodiments, provided herein is a CD30 CAR designated as AS57765-28z or a variant thereof. In some embodiments, a CD30 CAR designated AS48542-28z comprises an amino acid sequence of SEQ ID NO:201.

In some embodiments, provided herein are CD30 CARs designated as AS47863VH4-28z, A547863VH5-28z, A547863VH11-28z, A547863VH12-28z, A548433VH4-28z, A548433VH5-28z, A548433VH11-28z, A548433VH12-28z, A548463VH4-28z, A548463VH11-28z, A548481VH5-28z, A548481VH6-28z, A548481VH13-28z, A548481VH14-28z, A548508VH4-28z, A548508VH5-28z, A548508VH11-28z, A548508VH12-28z, A548542VH5-28z, A548542VH12-28z, A553445VH4-28z, A553445VH11-28z, A553574VH4-28z, A553574VH5-28z, A553574VH6-28z, AS53574VH7, A553574VH11-28z, A553574VH12-28z, A553574VH13-28z, A553750VH4-28z, A553750VH5-28z, A553750VH11-28z, A553750VH12-28z, A554233VH4-28z, A554233VH5-28z, A554233VH11-28z, or AS54233VH12-28z. In some embodiments, provided herein are CD30 CARs designated as AS47863VH4-28z or a variant thereof. In some embodiments, provided herein are CD30 CARs designated as AS47863VH5-28z or a variant thereof. In some embodiments, provided herein are CD30 CARs designated as AS47863VH11-28z or a variant thereof. In some embodiments, provided herein are CD30 CARs designated as AS47863VH12-28z or a variant thereof. In some embodiments, provided herein are CD30 CARs designated as AS48433VH4-28z or a variant thereof. In some embodiments, provided herein are CD30 CARs designated as AS48433VH5-28z or a variant thereof. In some embodiments, provided herein are CD30 CARs designated as AS48433VH11-28z or a variant thereof. In some embodiments, provided herein are CD30 CARs designated as AS48433VH12-28z or a variant thereof. In some embodiments, provided herein are CD30 CARs designated as AS48463VH4-28z or a variant thereof. In some embodiments, provided herein are CD30 CARs designated as AS48463VH11-28z or a variant thereof. In some embodiments, provided herein are CD30 CARs designated as AS48481VH5-28z or a variant thereof. In some embodiments, provided herein are CD30 CARs designated as AS48481VH6-28z or a variant thereof. In some embodiments, provided herein are CD30 CARs designated as AS48481VH13-28z or a variant thereof. In some embodiments, provided herein are CD30 CARs designated as AS48481VH14-28z or a variant thereof. In some embodiments, provided herein are CD30 CARs designated as AS48508VH4-28z or a variant thereof. In some embodiments, provided herein are CD30 CARs designated as AS48508VH5-28z or a variant thereof. In some embodiments, provided herein are CD30 CARs designated as AS48508VH11-28z or a variant thereof. In some embodiments, provided herein are CD30 CARs designated as AS48508VH12-28z or a variant thereof. In some embodiments, provided herein are CD30 CARs designated as AS48542VH5-28z or a variant thereof. In some embodiments, provided herein are CD30 CARs designated as AS48542VH12-28z or a variant thereof. In some embodiments, provided herein are CD30 CARs designated as AS53445VH4-28z or a variant thereof. In some embodiments, provided herein are CD30 CARs designated as AS53445VH11-28z or a variant thereof. In some embodiments, provided herein are CD30 CARs designated as AS53574VH4-28z or a variant thereof. In some embodiments, provided herein are CD30 CARs designated as AS53574VH5-28z or a variant thereof. In some embodiments, provided herein are CD30 CARs designated as AS53574VH6-28z or a variant thereof. In some embodiments, provided herein are CD30 CARs designated as AS53574VH7-28z or a variant thereof. In some embodiments, provided herein are CD30 CARs designated as AS53574VH11-28z or a variant thereof. In some embodiments, provided herein are CD30 CARs designated as AS53574VH12-28z or a variant thereof. In some embodiments, provided herein are CD30 CARs designated as AS53574VH13-28z or a variant thereof. In some embodiments, provided herein are CD30 CARs designated as AS53750VH4-28z or a variant thereof. In some embodiments, provided herein are CD30 CARs designated as AS53750VH5-28z or a variant thereof. In some embodiments, provided herein are CD30 CARs designated as AS53750VH11-28z or a variant thereof. In some embodiments, provided herein are CD30 CARs designated as AS53750VH12-28z or a variant thereof. In some embodiments, provided herein are CD30 CARs designated as AS54233VH4-28z or a variant thereof. In some embodiments, provided herein are CD30 CARs designated as AS54233VH5-28z or a variant thereof. In some embodiments, provided herein are CD30 CARs designated as AS54233VH11-28z or a variant thereof. In some embodiments, provided herein are CD30 CARs designated as AS54233VH12-28z or a variant thereof. In some embodiments, a CD30 CAR designated AS48542VH5-28z comprises an amino acid sequence of SEQ ID NO:208. In some embodiments, a CD30 CAR designated AS47863VH4-28z comprises an amino acid sequence of SEQ ID NO:210.

In some embodiments, provided herein are CARs having a bivalent CD30-binding moiety. In some embodiments, provided herein are CARs having a biparatopic CD30-binding moiety. In some embodiments, a CAR disclosed herein comprises, from N-terminus to the C-terminus, a leader sequence (e.g., SEQ ID NO: 61), a first anti-CD30 sdAb, a linker, a second anti-CD30 sdAb, a hinge (e.g., CD8α hinge or CD28 hinge), a transmembrane region (e.g., CD8α transmembrane region or CD28 transmembrane region), a costimulatory domain (e.g., the T cell signaling domain of 4-1BB, or CD28), and a signaling domain (e.g., the T cell signaling domain of CD3ζ). In some embodiments, the second anti-CD30 sdAb is a tandem repeat of the first anti-CD30 sdAb. In some embodiments, the second anti-CD30 sdAb is different from the first anti-CD30 sdAb. In some embodiments, the second anti-CD30 sdAb and the first anti-CD30 sdAb bind different epitopes on CD30.

In some embodiments, a CAR disclosed herein comprises, from N-terminus to the C-terminus, a leader sequence (e.g., SEQ ID NO: 61), a first anti-CD30 sdAb, a linker, a second anti-CD30 sdAb, CD8α hinge (SEQ ID NO: 62), CD8α transmembrane (TM) region (SEQ ID NO: 63), the cytoplasmic portion of the 4-1BB (CD137) molecule (SEQ ID NO: 64), and the cytoplasmic portion of the CD3 molecule (SEQ ID NO: 65), wherein the second anti-CD30 sdAb is a tandem repeat of the first anti-CD30 sdAb. If the linker is a long (G4S)₃ linker (SEQ ID NO:56), such CARs are designated “[CD30-binding moiety]dil-bbz.” If the linker is a short G45 linker (SEQ ID NO:57), such CARs are designated “[CD30-binding moiety]dis-bbz.” For example, CARs designated as AS48542dis-bbz comprises a CD30-binding moiety comprising, from N-terminus to C-terminus, AS48542 (SEQ ID NO:14), a short G45 linker (SEQ ID NO:57), and AS48542 (SEQ ID NO:14). For another example, CARs designated as AS47863VH4dil-bbz comprises a CD30-binding moiety comprising, from N-terminus to C-terminus, AS47863VH4 (SEQ ID NO:19), a long G45 linker (SEQ ID NO:56), and AS47863VH4 (SEQ ID NO:19). All combinations and permutations of sdAbs and linkers are contemplated herein. For example, in some embodiments, provided herein is a CD30 CAR designated as AS48542dis-bbz, or a variant thereof. In some embodiments, the CD30 CAR designated as AS48542dis-bbz has an amino acid sequence of SEQ ID NO: 83. In some embodiments, provided herein is a CD30 CAR designated as AS48542dil-bbz, or a variant thereof. In some embodiments, the CD30 CAR designated as AS48542dil-bbz has an amino acid sequence of SEQ ID NO: 84. In some embodiments, provided herein is a CD30 CAR designated as AS48542VH5dil-bbz, or a variant thereof. In some embodiments, the CD30 CAR designated as AS48542 VH5dil-bbz has an amino acid sequence of SEQ ID NO: 186. In some embodiments, provided herein is a CD30 CAR designated as AS48463VH4dil-bbz, or a variant thereof. In some embodiments, the CD30 CAR designated as AS48463VH4dil-bbz has an amino acid sequence of SEQ ID NO: 187. In some embodiments, provided herein is a CD30 CAR designated as AS47863VH4dil-bbz, or a variant thereof. In some embodiments, the CD30 CAR designated as AS47863VH4dil-bbz has an amino acid sequence of SEQ ID NO: 188.

In some embodiments, a CAR disclosed herein comprises, from N-terminus to the C-terminus, a leader sequence (e.g., SEQ ID NO: 61), a first anti-CD30 sdAb, a linker, a second anti-CD30 sdAb, CD8α hinge (SEQ ID NO: 62), CD8α transmembrane (TM) region (SEQ ID NO: 63), the cytoplasmic portion of the 4-1BB (CD137) molecule (SEQ ID NO: 64), and the cytoplasmic portion of the CD3 molecule (SEQ ID NO: 65), wherein the second anti-CD30 sdAb differs from first anti-CD30 sdAb. Such CARs are designated “[CD30-binding moiety]bil-bbz” if the linker is a long (G4S)₃ linker (SEQ ID NO:56), or “[CD30-binding moiety]bis-bbz” if the linker is a short G4S linker (SEQ ID NO:57). For example, CARs designated as AS48542-AS53574bil-bbz comprises a CD30-binding moiety comprising, from N-terminus to C-terminus, a first sdAb, a long (G4S)₃ linker (SEQ ID NO:56), and a second sdAb, wherein the first sdAb is AS48542, and the second sdAb is AS53574. For another example, CARs designated as AS47863VH4-AS48463VH4bis-bbz comprises a CD30-binding moiety comprises, from N-terminus to C-terminus, a first sdAb, a short G45 linker (SEQ ID NO:57), and a second sdAb, wherein the first sdAb is AS47863VH4, and the second sdAb is AS48463VH4. All combinations and permutations of different first sdAbs, second sdAb, and linkers are contemplated herein. For example, in some embodiments, provided herein is a CD30 CAR designated as AS48542-AS53574bil-bbz, or a variant thereof. In some embodiments, the CD30 CAR designated as AS48542-AS53574bil-bbz comprises an amino acid sequence of SEQ ID NO:189. In some embodiments, provided herein is a CD30 CAR designated as AS48463VH4-AS53574VH7bil-bbz, or a variant thereof. In some embodiments, the CD30 CAR designated as AS48463VH4-AS53574VH7bil-bbz comprises an amino acid sequence of SEQ ID NO:190. In some embodiments, provided herein are CD30 CARs designated as AS47863VH4-AS53574VH7bil-bbz, or a variant thereof. In some embodiments, the CD30 CAR designated as AS47863VH4-AS53574VH7bil-bbz comprises an amino acid sequence of SEQ ID NO:191. In some embodiments, provided herein is a CD30 CAR designated as AS53574VH7-AS48542VH5bil-bbz, or a variant thereof. In some embodiments, the CD30 CAR designated as AS53574VH7-AS48542VH5bil-bbz comprises an amino acid sequence of SEQ ID NO:192. In some embodiments, provided herein are CD30 CARs designated as AS53574VH7-AS48463VH4bil-bbz, or a variant thereof. In some embodiments, the CD30 CAR designated as AS53574VH7-AS48463VH4bil-bbz comprises an amino acid sequence of SEQ ID NO:193. In some embodiments, provided herein is a CD30 CAR designated as AS53574VH7-AS47863VH4bil-bbz, or a variant thereof. In some embodiments, the CD30 CAR designated as AS53574VH7-AS47863VH4bil-bbz comprises an amino acid sequence of SEQ ID NO:194. In some embodiments, provided herein is a CD30 CAR designated as AS53574-AS48542bil-bbz, or a variant thereof. In some embodiments, the CD30 CAR designated as AS53574-AS48542bil-bbz comprises an amino acid sequence of SEQ ID NO:86. In some embodiments, provided herein is a CD30 CAR designated as AS48542-AS53574bil-bbz. In some embodiments, the CD30 CAR designated as AS48542-AS53574bil-bbz comprises an amino acid sequence of SEQ ID NO:85.

In some embodiments, a CAR disclosed herein comprises, from N-terminus to the C-terminus, a leader sequence (e.g., SEQ ID NO: 61), a first anti-CD30 sdAb, a linker, a second anti-CD30 sdAb, CD28 hinge (SEQ ID NO: 127), CD28 transmembrane (TM) region (SEQ ID NO: 128), the cytoplasmic portion of CD28 molecule (SEQ ID NO: 129), and the cytoplasmic portion of the CD3ζ molecule (SEQ ID NO: 65), wherein the second anti-CD30 sdAb is a tandem repeat of the first anti-CD30 sdAb. If the linker is a long (G4S)₃ linker (SEQ ID NO:56), such CARs are designated “[CD30-binding moiety]dil-28z.” If the linker is a short G4S linker (SEQ ID NO:57), such CARs are designated “[CD30-binding moiety]dis-28z.” For example, the CD30 CAR designated as AS48542VH5dil-28z comprises an amino acid sequence of SEQ ID NO: 209. The CD30 CAR designated as AS47863VH4dil-28z comprises an amino acid sequence of SEQ ID NO: 211.

In some embodiments, a CAR disclosed herein comprises, from N-terminus to the C-terminus, a leader sequence (e.g., SEQ ID NO: 61), a first anti-CD30 sdAb, a linker, a second anti-CD30 sdAb, CD28 hinge (SEQ ID NO: 127), CD28 transmembrane (TM) region (SEQ ID NO: 128), the cytoplasmic portion of CD28 molecule (SEQ ID NO: 129), and the cytoplasmic portion of the CD3ζ molecule (SEQ ID NO: 65), wherein the second anti-CD30 sdAb differs from first anti-CD30 sdAb. Such CARs are designated “[CD30-binding moiety]bil-28z” if the linker is a long (G4S)₃ linker (SEQ ID NO:56), or “[CD30-binding moiety]bis-28z” if the linker is a short G45 linker (SEQ ID NO:57).

In some embodiments, provided herein are CD30 CARs having an amino acid sequence that is at least 80% identical to SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:201, SEQ ID NO:208, SEQ ID NO:209, SEQ ID NO:210 or SEQ ID NO:211. In some embodiments, provided herein are CD30 CARs having an amino acid sequence that is at least 85%, at least 90%, at least 95%, at least 97%, or at least 99% identical to SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:201, SEQ ID NO:208, SEQ ID NO:209, SEQ ID NO:210 or SEQ ID NO:211. In some embodiments, a CAR has an amino acid sequence comprising SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:201, SEQ ID NO:208, SEQ ID NO:209, SEQ ID NO:210 or SEQ ID NO:211.

In some embodiments, a CD30 CAR has an amino acid sequence that is at least 80% identical to SEQ ID NO:70. In some embodiments, a CD30 CAR has an amino acid sequence that is at least 85% identical to SEQ ID NO:70. In some embodiments, a CD30 CAR has an amino acid sequence that is at least 90% identical to SEQ ID NO:70. In some embodiments, a CD30 CAR has an amino acid sequence that is at least 95% identical to SEQ ID NO:70. In some embodiments, a CD30 CAR has an amino acid sequence that is at least 97% identical to SEQ ID NO:70. In some embodiments, a CD30 CAR has an amino acid sequence that is at least 99% identical to SEQ ID NO:70. In some embodiments, a CAR has an amino acid sequence comprising SEQ ID NO:70.

In some embodiments, a CD30 CAR has an amino acid sequence that is at least 80% identical to SEQ ID NO:71. In some embodiments, a CD30 CAR has an amino acid sequence that is at least 85% identical to SEQ ID NO:71. In some embodiments, a CD30 CAR has an amino acid sequence that is at least 90% identical to SEQ ID NO:71. In some embodiments, a CD30 CAR has an amino acid sequence that is at least 95% identical to SEQ ID NO:71. In some embodiments, a CD30 CAR has an amino acid sequence that is at least 97% identical to SEQ ID NO:71. In some embodiments, a CD30 CAR has an amino acid sequence that is at least 99% identical to SEQ ID NO:71. In some embodiments, a CAR has an amino acid sequence comprising SEQ ID NO:71.

In some embodiments, a CD30 CAR has an amino acid sequence that is at least 80% identical to SEQ ID NO:72. In some embodiments, a CD30 CAR has an amino acid sequence that is at least 85% identical to SEQ ID NO:72. In some embodiments, a CD30 CAR has an amino acid sequence that is at least 90% identical to SEQ ID NO:72. In some embodiments, a CD30 CAR has an amino acid sequence that is at least 95% identical to SEQ ID NO:72. In some embodiments, a CD30 CAR has an amino acid sequence that is at least 97% identical to SEQ ID NO:72. In some embodiments, a CD30 CAR has an amino acid sequence that is at least 99% identical to SEQ ID NO:72. In some embodiments, a CAR has an amino acid sequence comprising SEQ ID NO:72.

In some embodiments, a CD30 CAR has an amino acid sequence that is at least 80% identical to SEQ ID NO:73. In some embodiments, a CD30 CAR has an amino acid sequence that is at least 85% identical to SEQ ID NO:73. In some embodiments, a CD30 CAR has an amino acid sequence that is at least 90% identical to SEQ ID NO:73. In some embodiments, a CD30 CAR has an amino acid sequence that is at least 95% identical to SEQ ID NO:73. In some embodiments, a CD30 CAR has an amino acid sequence that is at least 97% identical to SEQ ID NO:73. In some embodiments, a CD30 CAR has an amino acid sequence that is at least 99% identical to SEQ ID NO:73. In some embodiments, a CAR has an amino acid sequence comprising SEQ ID NO:73.

In some embodiments, a CD30 CAR has an amino acid sequence that is at least 80% identical to SEQ ID NO:74. In some embodiments, a CD30 CAR has an amino acid sequence that is at least 85% identical to SEQ ID NO:74. In some embodiments, a CD30 CAR has an amino acid sequence that is at least 90% identical to SEQ ID NO:74. In some embodiments, a CD30 CAR has an amino acid sequence that is at least 95% identical to SEQ ID NO:74. In some embodiments, a CD30 CAR has an amino acid sequence that is at least 97% identical to SEQ ID NO:74. In some embodiments, a CD30 CAR has an amino acid sequence that is at least 99% identical to SEQ ID NO:74. In some embodiments, a CAR has an amino acid sequence comprising SEQ ID NO:74.

In some embodiments, a CD30 CAR has an amino acid sequence that is at least 80% identical to SEQ ID NO:75. In some embodiments, a CD30 CAR has an amino acid sequence that is at least 85% identical to SEQ ID NO:75. In some embodiments, a CD30 CAR has an amino acid sequence that is at least 90% identical to SEQ ID NO:75. In some embodiments, a CD30 CAR has an amino acid sequence that is at least 95% identical to SEQ ID NO:75. In some embodiments, a CD30 CAR has an amino acid sequence that is at least 97% identical to SEQ ID NO:75. In some embodiments, a CD30 CAR has an amino acid sequence that is at least 99% identical to SEQ ID NO:75. In some embodiments, a CAR has an amino acid sequence comprising SEQ ID NO:75.

In some embodiments, a CD30 CAR has an amino acid sequence that is at least 80% identical to SEQ ID NO:76. In some embodiments, a CD30 CAR has an amino acid sequence that is at least 85% identical to SEQ ID NO:76. In some embodiments, a CD30 CAR has an amino acid sequence that is at least 90% identical to SEQ ID NO:76. In some embodiments, a CD30 CAR has an amino acid sequence that is at least 95% identical to SEQ ID NO:76. In some embodiments, a CD30 CAR has an amino acid sequence that is at least 97% identical to SEQ ID NO:76. In some embodiments, a CD30 CAR has an amino acid sequence that is at least 99% identical to SEQ ID NO:76. In some embodiments, a CAR has an amino acid sequence comprising SEQ ID NO:76.

In some embodiments, a CD30 CAR has an amino acid sequence that is at least 80% identical to SEQ ID NO:77. In some embodiments, a CD30 CAR has an amino acid sequence that is at least 85% identical to SEQ ID NO:77. In some embodiments, a CD30 CAR has an amino acid sequence that is at least 90% identical to SEQ ID NO:77. In some embodiments, a CD30 CAR has an amino acid sequence that is at least 95% identical to SEQ ID NO:77. In some embodiments, a CD30 CAR has an amino acid sequence that is at least 97% identical to SEQ ID NO:77. In some embodiments, a CD30 CAR has an amino acid sequence that is at least 99% identical to SEQ ID NO:77. In some embodiments, a CAR has an amino acid sequence comprising SEQ ID NO:77.

In some embodiments, a CD30 CAR has an amino acid sequence that is at least 80% identical to SEQ ID NO:78. In some embodiments, a CD30 CAR has an amino acid sequence that is at least 85% identical to SEQ ID NO:78. In some embodiments, a CD30 CAR has an amino acid sequence that is at least 90% identical to SEQ ID NO:78. In some embodiments, a CD30 CAR has an amino acid sequence that is at least 95% identical to SEQ ID NO:78. In some embodiments, a CD30 CAR has an amino acid sequence that is at least 97% identical to SEQ ID NO:78. In some embodiments, a CD30 CAR has an amino acid sequence that is at least 99% identical to SEQ ID NO:78. In some embodiments, a CAR has an amino acid sequence comprising SEQ ID NO:78.

In some embodiments, a CD30 CAR has an amino acid sequence that is at least 80% identical to SEQ ID NO:79. In some embodiments, a CD30 CAR has an amino acid sequence that is at least 85% identical to SEQ ID NO:79. In some embodiments, a CD30 CAR has an amino acid sequence that is at least 90% identical to SEQ ID NO:79. In some embodiments, a CD30 CAR has an amino acid sequence that is at least 95% identical to SEQ ID NO:79. In some embodiments, a CD30 CAR has an amino acid sequence that is at least 97% identical to SEQ ID NO:79. In some embodiments, a CD30 CAR has an amino acid sequence that is at least 99% identical to SEQ ID NO:79. In some embodiments, a CAR has an amino acid sequence comprising SEQ ID NO:79.

In some embodiments, a CD30 CAR has an amino acid sequence that is at least 80% identical to SEQ ID NO:80. In some embodiments, a CD30 CAR has an amino acid sequence that is at least 85% identical to SEQ ID NO:80. In some embodiments, a CD30 CAR has an amino acid sequence that is at least 90% identical to SEQ ID NO:80. In some embodiments, a CD30 CAR has an amino acid sequence that is at least 95% identical to SEQ ID NO:80. In some embodiments, a CD30 CAR has an amino acid sequence that is at least 97% identical to SEQ ID NO:80. In some embodiments, a CD30 CAR has an amino acid sequence that is at least 99% identical to SEQ ID NO:80. In some embodiments, a CAR has an amino acid sequence comprising SEQ ID NO:80.

In some embodiments, a CD30 CAR has an amino acid sequence that is at least 80% identical to SEQ ID NO:81. In some embodiments, a CD30 CAR has an amino acid sequence that is at least 85% identical to SEQ ID NO:81. In some embodiments, a CD30 CAR has an amino acid sequence that is at least 90% identical to SEQ ID NO:81. In some embodiments, a CD30 CAR has an amino acid sequence that is at least 95% identical to SEQ ID NO:81. In some embodiments, a CD30 CAR has an amino acid sequence that is at least 97% identical to SEQ ID NO:81. In some embodiments, a CD30 CAR has an amino acid sequence that is at least 99% identical to SEQ ID NO:81. In some embodiments, a CAR has an amino acid sequence comprising SEQ ID NO:81.

In some embodiments, a CD30 CAR has an amino acid sequence that is at least 80% identical to SEQ ID NO:82. In some embodiments, a CD30 CAR has an amino acid sequence that is at least 85% identical to SEQ ID NO:82. In some embodiments, a CD30 CAR has an amino acid sequence that is at least 90% identical to SEQ ID NO:82. In some embodiments, a CD30 CAR has an amino acid sequence that is at least 95% identical to SEQ ID NO:82. In some embodiments, a CD30 CAR has an amino acid sequence that is at least 97% identical to SEQ ID NO:82. In some embodiments, a CD30 CAR has an amino acid sequence that is at least 99% identical to SEQ ID NO:82. In some embodiments, a CAR has an amino acid sequence comprising SEQ ID NO:82.

In some embodiments, a CD30 CAR has an amino acid sequence that is at least 80% identical to SEQ ID NO:83. In some embodiments, a CD30 CAR has an amino acid sequence that is at least 85% identical to SEQ ID NO:83. In some embodiments, a CD30 CAR has an amino acid sequence that is at least 90% identical to SEQ ID NO:83. In some embodiments, a CD30 CAR has an amino acid sequence that is at least 95% identical to SEQ ID NO:83. In some embodiments, a CD30 CAR has an amino acid sequence that is at least 97% identical to SEQ ID NO:83. In some embodiments, a CD30 CAR has an amino acid sequence that is at least 99% identical to SEQ ID NO:83. In some embodiments, a CAR has an amino acid sequence comprising SEQ ID NO:83.

In some embodiments, a CD30 CAR has an amino acid sequence that is at least 80% identical to SEQ ID NO:84. In some embodiments, a CD30 CAR has an amino acid sequence that is at least 85% identical to SEQ ID NO:84. In some embodiments, a CD30 CAR has an amino acid sequence that is at least 90% identical to SEQ ID NO:84. In some embodiments, a CD30 CAR has an amino acid sequence that is at least 95% identical to SEQ ID NO:84. In some embodiments, a CD30 CAR has an amino acid sequence that is at least 97% identical to SEQ ID NO:84. In some embodiments, a CD30 CAR has an amino acid sequence that is at least 99% identical to SEQ ID NO:84. In some embodiments, a CAR has an amino acid sequence comprising SEQ ID NO:84.

In some embodiments, a CD30 CAR has an amino acid sequence that is at least 80% identical to SEQ ID NO:85. In some embodiments, a CD30 CAR has an amino acid sequence that is at least 85% identical to SEQ ID NO:85. In some embodiments, a CD30 CAR has an amino acid sequence that is at least 90% identical to SEQ ID NO:85. In some embodiments, a CD30 CAR has an amino acid sequence that is at least 95% identical to SEQ ID NO:85. In some embodiments, a CD30 CAR has an amino acid sequence that is at least 97% identical to SEQ ID NO:85. In some embodiments, a CD30 CAR has an amino acid sequence that is at least 99% identical to SEQ ID NO:85. In some embodiments, a CAR has an amino acid sequence comprising SEQ ID NO:85.

In some embodiments, a CD30 CAR has an amino acid sequence that is at least 80% identical to SEQ ID NO:86. In some embodiments, a CD30 CAR has an amino acid sequence that is at least 85% identical to SEQ ID NO:86. In some embodiments, a CD30 CAR has an amino acid sequence that is at least 90% identical to SEQ ID NO:86. In some embodiments, a CD30 CAR has an amino acid sequence that is at least 95% identical to SEQ ID NO:86. In some embodiments, a CD30 CAR has an amino acid sequence that is at least 97% identical to SEQ ID NO:86. In some embodiments, a CD30 CAR has an amino acid sequence that is at least 99% identical to SEQ ID NO:86. In some embodiments, a CAR has an amino acid sequence comprising SEQ ID NO:86.

In some embodiments, a CD30 CAR has an amino acid sequence that is at least 80% identical to SEQ ID NO:182. In some embodiments, a CD30 CAR has an amino acid sequence that is at least 85% identical to SEQ ID NO:182. In some embodiments, a CD30 CAR has an amino acid sequence that is at least 90% identical to SEQ ID NO:182. In some embodiments, a CD30 CAR has an amino acid sequence that is at least 95% identical to SEQ ID NO:182. In some embodiments, a CD30 CAR has an amino acid sequence that is at least 97% identical to SEQ ID NO:182. In some embodiments, a CD30 CAR has an amino acid sequence that is at least 99% identical to SEQ ID NO:182. In some embodiments, a CAR has an amino acid sequence comprising SEQ ID NO:182.

In some embodiments, a CD30 CAR has an amino acid sequence that is at least 80% identical to SEQ ID NO:183. In some embodiments, a CD30 CAR has an amino acid sequence that is at least 85% identical to SEQ ID NO:183. In some embodiments, a CD30 CAR has an amino acid sequence that is at least 90% identical to SEQ ID NO:183. In some embodiments, a CD30 CAR has an amino acid sequence that is at least 95% identical to SEQ ID NO:183. In some embodiments, a CD30 CAR has an amino acid sequence that is at least 97% identical to SEQ ID NO:183. In some embodiments, a CD30 CAR has an amino acid sequence that is at least 99% identical to SEQ ID NO:183. In some embodiments, a CAR has an amino acid sequence comprising SEQ ID NO:183.

In some embodiments, a CD30 CAR has an amino acid sequence that is at least 80% identical to SEQ ID NO:184. In some embodiments, a CD30 CAR has an amino acid sequence that is at least 85% identical to SEQ ID NO:184. In some embodiments, a CD30 CAR has an amino acid sequence that is at least 90% identical to SEQ ID NO:184. In some embodiments, a CD30 CAR has an amino acid sequence that is at least 95% identical to SEQ ID NO:184. In some embodiments, a CD30 CAR has an amino acid sequence that is at least 97% identical to SEQ ID NO:184. In some embodiments, a CD30 CAR has an amino acid sequence that is at least 99% identical to SEQ ID NO:184. In some embodiments, a CAR has an amino acid sequence comprising SEQ ID NO:184.

In some embodiments, a CD30 CAR has an amino acid sequence that is at least 80% identical to SEQ ID NO:185. In some embodiments, a CD30 CAR has an amino acid sequence that is at least 85% identical to SEQ ID NO:185. In some embodiments, a CD30 CAR has an amino acid sequence that is at least 90% identical to SEQ ID NO:185. In some embodiments, a CD30 CAR has an amino acid sequence that is at least 95% identical to SEQ ID NO:185. In some embodiments, a CD30 CAR has an amino acid sequence that is at least 97% identical to SEQ ID NO:185. In some embodiments, a CD30 CAR has an amino acid sequence that is at least 99% identical to SEQ ID NO:185. In some embodiments, a CAR has an amino acid sequence comprising SEQ ID NO:185.

In some embodiments, a CD30 CAR has an amino acid sequence that is at least 80% identical to SEQ ID NO:186. In some embodiments, a CD30 CAR has an amino acid sequence that is at least 85% identical to SEQ ID NO:186. In some embodiments, a CD30 CAR has an amino acid sequence that is at least 90% identical to SEQ ID NO:186. In some embodiments, a CD30 CAR has an amino acid sequence that is at least 95% identical to SEQ ID NO:186. In some embodiments, a CD30 CAR has an amino acid sequence that is at least 97% identical to SEQ ID NO:186. In some embodiments, a CD30 CAR has an amino acid sequence that is at least 99% identical to SEQ ID NO:186. In some embodiments, a CAR has an amino acid sequence comprising SEQ ID NO:186.

In some embodiments, a CD30 CAR has an amino acid sequence that is at least 80% identical to SEQ ID NO:187. In some embodiments, a CD30 CAR has an amino acid sequence that is at least 85% identical to SEQ ID NO:187. In some embodiments, a CD30 CAR has an amino acid sequence that is at least 90% identical to SEQ ID NO:187. In some embodiments, a CD30 CAR has an amino acid sequence that is at least 95% identical to SEQ ID NO:187. In some embodiments, a CD30 CAR has an amino acid sequence that is at least 97% identical to SEQ ID NO:187. In some embodiments, a CD30 CAR has an amino acid sequence that is at least 99% identical to SEQ ID NO:187. In some embodiments, a CAR has an amino acid sequence comprising SEQ ID NO:187.

In some embodiments, a CD30 CAR has an amino acid sequence that is at least 80% identical to SEQ ID NO:188. In some embodiments, a CD30 CAR has an amino acid sequence that is at least 85% identical to SEQ ID NO:188. In some embodiments, a CD30 CAR has an amino acid sequence that is at least 90% identical to SEQ ID NO:188. In some embodiments, a CD30 CAR has an amino acid sequence that is at least 95% identical to SEQ ID NO:188. In some embodiments, a CD30 CAR has an amino acid sequence that is at least 97% identical to SEQ ID NO:188. In some embodiments, a CD30 CAR has an amino acid sequence that is at least 99% identical to SEQ ID NO:188. In some embodiments, a CAR has an amino acid sequence comprising SEQ ID NO:188.

In some embodiments, a CD30 CAR has an amino acid sequence that is at least 80% identical to SEQ ID NO:189. In some embodiments, a CD30 CAR has an amino acid sequence that is at least 85% identical to SEQ ID NO:189. In some embodiments, a CD30 CAR has an amino acid sequence that is at least 90% identical to SEQ ID NO:189. In some embodiments, a CD30 CAR has an amino acid sequence that is at least 95% identical to SEQ ID NO:189. In some embodiments, a CD30 CAR has an amino acid sequence that is at least 97% identical to SEQ ID NO:189. In some embodiments, a CD30 CAR has an amino acid sequence that is at least 99% identical to SEQ ID NO:189. In some embodiments, a CAR has an amino acid sequence comprising SEQ ID NO:189.

In some embodiments, a CD30 CAR has an amino acid sequence that is at least 80% identical to SEQ ID NO:190. In some embodiments, a CD30 CAR has an amino acid sequence that is at least 85% identical to SEQ ID NO:190. In some embodiments, a CD30 CAR has an amino acid sequence that is at least 90% identical to SEQ ID NO:190. In some embodiments, a CD30 CAR has an amino acid sequence that is at least 95% identical to SEQ ID NO:190. In some embodiments, a CD30 CAR has an amino acid sequence that is at least 97% identical to SEQ ID NO:190. In some embodiments, a CD30 CAR has an amino acid sequence that is at least 99% identical to SEQ ID NO:190. In some embodiments, a CAR has an amino acid sequence comprising SEQ ID NO:190.

In some embodiments, a CD30 CAR has an amino acid sequence that is at least 80% identical to SEQ ID NO:191. In some embodiments, a CD30 CAR has an amino acid sequence that is at least 85% identical to SEQ ID NO:191. In some embodiments, a CD30 CAR has an amino acid sequence that is at least 90% identical to SEQ ID NO:191. In some embodiments, a CD30 CAR has an amino acid sequence that is at least 95% identical to SEQ ID NO:191. In some embodiments, a CD30 CAR has an amino acid sequence that is at least 97% identical to SEQ ID NO:191. In some embodiments, a CD30 CAR has an amino acid sequence that is at least 99% identical to SEQ ID NO:191. In some embodiments, a CAR has an amino acid sequence comprising SEQ ID NO:191.

In some embodiments, a CD30 CAR has an amino acid sequence that is at least 80% identical to SEQ ID NO:192. In some embodiments, a CD30 CAR has an amino acid sequence that is at least 85% identical to SEQ ID NO:192. In some embodiments, a CD30 CAR has an amino acid sequence that is at least 90% identical to SEQ ID NO:192. In some embodiments, a CD30 CAR has an amino acid sequence that is at least 95% identical to SEQ ID NO:192. In some embodiments, a CD30 CAR has an amino acid sequence that is at least 97% identical to SEQ ID NO:192. In some embodiments, a CD30 CAR has an amino acid sequence that is at least 99% identical to SEQ ID NO:192. In some embodiments, a CAR has an amino acid sequence comprising SEQ ID NO:192.

In some embodiments, a CD30 CAR has an amino acid sequence that is at least 80% identical to SEQ ID NO:193. In some embodiments, a CD30 CAR has an amino acid sequence that is at least 85% identical to SEQ ID NO:193. In some embodiments, a CD30 CAR has an amino acid sequence that is at least 90% identical to SEQ ID NO:193. In some embodiments, a CD30 CAR has an amino acid sequence that is at least 95% identical to SEQ ID NO:193. In some embodiments, a CD30 CAR has an amino acid sequence that is at least 97% identical to SEQ ID NO:193. In some embodiments, a CD30 CAR has an amino acid sequence that is at least 99% identical to SEQ ID NO:193. In some embodiments, a CAR has an amino acid sequence comprising SEQ ID NO:193.

In some embodiments, a CD30 CAR has an amino acid sequence that is at least 80% identical to SEQ ID NO:194. In some embodiments, a CD30 CAR has an amino acid sequence that is at least 85% identical to SEQ ID NO:194. In some embodiments, a CD30 CAR has an amino acid sequence that is at least 90% identical to SEQ ID NO:194. In some embodiments, a CD30 CAR has an amino acid sequence that is at least 95% identical to SEQ ID NO:194. In some embodiments, a CD30 CAR has an amino acid sequence that is at least 97% identical to SEQ ID NO:194. In some embodiments, a CD30 CAR has an amino acid sequence that is at least 99% identical to SEQ ID NO:194. In some embodiments, a CAR has an amino acid sequence comprising SEQ ID NO:194.

In some embodiments, a CD30 CAR has an amino acid sequence that is at least 80% identical to SEQ ID NO:201. In some embodiments, a CD30 CAR has an amino acid sequence that is at least 85% identical to SEQ ID NO:201. In some embodiments, a CD30 CAR has an amino acid sequence that is at least 90% identical to SEQ ID NO:201. In some embodiments, a CD30 CAR has an amino acid sequence that is at least 95% identical to SEQ ID NO:201. In some embodiments, a CD30 CAR has an amino acid sequence that is at least 97% identical to SEQ ID NO:201. In some embodiments, a CD30 CAR has an amino acid sequence that is at least 99% identical to SEQ ID NO:201. In some embodiments, a CAR has an amino acid sequence comprising SEQ ID NO:201.

In some embodiments, a CD30 CAR has an amino acid sequence that is at least 80% identical to SEQ ID NO:208. In some embodiments, a CD30 CAR has an amino acid sequence that is at least 85% identical to SEQ ID NO:208. In some embodiments, a CD30 CAR has an amino acid sequence that is at least 90% identical to SEQ ID NO:208. In some embodiments, a CD30 CAR has an amino acid sequence that is at least 95% identical to SEQ ID NO:208. In some embodiments, a CD30 CAR has an amino acid sequence that is at least 97% identical to SEQ ID NO:208. In some embodiments, a CD30 CAR has an amino acid sequence that is at least 99% identical to SEQ ID NO:208. In some embodiments, a CAR has an amino acid sequence comprising SEQ ID NO:208.

In some embodiments, a CD30 CAR has an amino acid sequence that is at least 80% identical to SEQ ID NO:209. In some embodiments, a CD30 CAR has an amino acid sequence that is at least 85% identical to SEQ ID NO:209. In some embodiments, a CD30 CAR has an amino acid sequence that is at least 90% identical to SEQ ID NO:209. In some embodiments, a CD30 CAR has an amino acid sequence that is at least 95% identical to SEQ ID NO:209. In some embodiments, a CD30 CAR has an amino acid sequence that is at least 97% identical to SEQ ID NO:209. In some embodiments, a CD30 CAR has an amino acid sequence that is at least 99% identical to SEQ ID NO:209. In some embodiments, a CAR has an amino acid sequence comprising SEQ ID NO:209.

In some embodiments, a CD30 CAR has an amino acid sequence that is at least 80% identical to SEQ ID NO:210. In some embodiments, a CD30 CAR has an amino acid sequence that is at least 85% identical to SEQ ID NO:210. In some embodiments, a CD30 CAR has an amino acid sequence that is at least 90% identical to SEQ ID NO:210. In some embodiments, a CD30 CAR has an amino acid sequence that is at least 95% identical to SEQ ID NO:210. In some embodiments, a CD30 CAR has an amino acid sequence that is at least 97% identical to SEQ ID NO:210. In some embodiments, a CD30 CAR has an amino acid sequence that is at least 99% identical to SEQ ID NO:210. In some embodiments, a CAR has an amino acid sequence comprising SEQ ID NO:210.

In some embodiments, a CD30 CAR has an amino acid sequence that is at least 80% identical to SEQ ID NO:211. In some embodiments, a CD30 CAR has an amino acid sequence that is at least 85% identical to SEQ ID NO:211. In some embodiments, a CD30 CAR has an amino acid sequence that is at least 90% identical to SEQ ID NO:211. In some embodiments, a CD30 CAR has an amino acid sequence that is at least 95% identical to SEQ ID NO:211. In some embodiments, a CD30 CAR has an amino acid sequence that is at least 97% identical to SEQ ID NO:211. In some embodiments, a CD30 CAR has an amino acid sequence that is at least 99% identical to SEQ ID NO:211. In some embodiments, a CAR has an amino acid sequence comprising SEQ ID NO:211.

In some embodiments, a CAR disclosed herein can be of any length. In certain embodiments, the CAR can comprise any number of amino acids, provided that the CAR retain its biological activity (e.g., the ability to specifically bind to antigen, treat a mammal, and/or prevent a condition in a mammal). As a non-limiting example, the CAR can be about 50 to about 5000 amino acids long, such as about 50 to about 500, about 500 to about 1000, about 1000 to about 1500, about 1500 to about 2000, about 2000 to about 2500, about 2500 to about 3000, about 3000 to about 3500, about 3500 to about 4000, about 4000 to about 4500, about 4500 to about 5000, or about 5000 or more amino acids in length.

Further provided herein are variants of the CARs described herein. The variants provided herein are CARs that have substantial sequence identity or similarity to the parent parent CAR, and that retain the biological activities of the parent CAR.

In some embodiments, provided herein are polynucleotides comprising polynucleotides encoding that encode a polypeptide (i.e., a CD30 CAR) described herein. In some embodiments, the polynucleotide comprises a polynucleotide (e.g., a nucleotide sequence) encoding a polypeptide comprising an amino acid sequence selected from the group consisting of: SEQ ID NOs:70-86, 182-198, 201 and 208-211. In some embodiments, the polynucleotide comprises a polynucleotide encoding a polypeptide comprising an amino acid sequence of SEQ ID NO:70. In some embodiments, the polynucleotide comprises a polynucleotide encoding a polypeptide comprising an amino acid sequence of SEQ ID NO:71. In some embodiments, the polynucleotide comprises a polynucleotide encoding a polypeptide comprising an amino acid sequence of SEQ ID NO:72. In some embodiments, the polynucleotide comprises a polynucleotide encoding a polypeptide comprising an amino acid sequence of SEQ ID NO:73. In some embodiments, the polynucleotide comprises a polynucleotide encoding a polypeptide comprising an amino acid sequence of SEQ ID NO:74. In some embodiments, the polynucleotide comprises a polynucleotide encoding a polypeptide comprising an amino acid sequence of SEQ ID NO:75. In some embodiments, the polynucleotide comprises a polynucleotide encoding a polypeptide comprising an amino acid sequence of SEQ ID NO:76. In some embodiments, the polynucleotide comprises a polynucleotide encoding a polypeptide comprising an amino acid sequence of SEQ ID NO:77. In some embodiments, the polynucleotide comprises a polynucleotide encoding a polypeptide comprising an amino acid sequence of SEQ ID NO:78. In some embodiments, the polynucleotide comprises a polynucleotide encoding a polypeptide comprising an amino acid sequence of SEQ ID NO:79. In some embodiments, the polynucleotide comprises a polynucleotide encoding a polypeptide comprising an amino acid sequence of SEQ ID NO:80. In some embodiments, the polynucleotide comprises a polynucleotide encoding a polypeptide comprising an amino acid sequence of SEQ ID NO:81. In some embodiments, the polynucleotide comprises a polynucleotide encoding a polypeptide comprising an amino acid sequence of SEQ ID NO:82. In some embodiments, the polynucleotide comprises a polynucleotide encoding a polypeptide comprising an amino acid sequence of SEQ ID NO:83. In some embodiments, the polynucleotide comprises a polynucleotide encoding a polypeptide comprising an amino acid sequence of SEQ ID NO:84. In some embodiments, the polynucleotide comprises a polynucleotide encoding a polypeptide comprising an amino acid sequence of SEQ ID NO:85. In some embodiments, the polynucleotide comprises a polynucleotide encoding a polypeptide comprising an amino acid sequence of SEQ ID NO:86. In some embodiments, the polynucleotide comprises a polynucleotide encoding a polypeptide comprising an amino acid sequence of SEQ ID NO:182. In some embodiments, the polynucleotide comprises a polynucleotide encoding a polypeptide comprising an amino acid sequence of SEQ ID NO:183. In some embodiments, the polynucleotide comprises a polynucleotide encoding a polypeptide comprising an amino acid sequence of SEQ ID NO:184. In some embodiments, the polynucleotide comprises a polynucleotide encoding a polypeptide comprising an amino acid sequence of SEQ ID NO:185. In some embodiments, the polynucleotide comprises a polynucleotide encoding a polypeptide comprising an amino acid sequence of SEQ ID NO:186. In some embodiments, the polynucleotide comprises a polynucleotide encoding a polypeptide comprising an amino acid sequence of SEQ ID NO:187. In some embodiments, the polynucleotide comprises a polynucleotide encoding a polypeptide comprising an amino acid sequence of SEQ ID NO:188. In some embodiments, the polynucleotide comprises a polynucleotide encoding a polypeptide comprising an amino acid sequence of SEQ ID NO:189. In some embodiments, the polynucleotide comprises a polynucleotide encoding a polypeptide comprising an amino acid sequence of SEQ ID NO:190. In some embodiments, the polynucleotide comprises a polynucleotide encoding a polypeptide comprising an amino acid sequence of SEQ ID NO:191. In some embodiments, the polynucleotide comprises a polynucleotide encoding a polypeptide comprising an amino acid sequence of SEQ ID NO:192. In some embodiments, the polynucleotide comprises a polynucleotide encoding a polypeptide comprising an amino acid sequence of SEQ ID NO:193. In some embodiments, the polynucleotide comprises a polynucleotide encoding a polypeptide comprising an amino acid sequence of SEQ ID NO:194. In some embodiments, the polynucleotide comprises a polynucleotide encoding a polypeptide comprising an amino acid sequence of SEQ ID NO:195. In some embodiments, the polynucleotide comprises a polynucleotide encoding a polypeptide comprising an amino acid sequence of SEQ ID NO:196. In some embodiments, the polynucleotide comprises a polynucleotide encoding a polypeptide comprising an amino acid sequence of SEQ ID NO:197. In some embodiments, the polynucleotide comprises a polynucleotide encoding a polypeptide comprising an amino acid sequence of SEQ ID NO:198. In some embodiments, the polynucleotide comprises a polynucleotide encoding a polypeptide comprising an amino acid sequence of SEQ ID NO:201. In some embodiments, the polynucleotide comprises a polynucleotide encoding a polypeptide comprising an amino acid sequence of SEQ ID NO:208. In some embodiments, the polynucleotide comprises a polynucleotide encoding a polypeptide comprising an amino acid sequence of SEQ ID NO:209. In some embodiments, the polynucleotide comprises a polynucleotide encoding a polypeptide comprising an amino acid sequence of SEQ ID NO:210. In some embodiments, the polynucleotide comprises a polynucleotide encoding a polypeptide comprising an amino acid sequence of SEQ ID NO:211.

The present disclosure also provides variants of the polynucleotides described herein, wherein the variant encodes, for example, fragments, analogs, and/or derivatives of a CD30 CAR described herein. In some embodiments, the present disclosure provides a polynucleotide comprising a polynucleotide having a nucleotide sequence at least about 80% identical, at least about 85% identical, at least about 90% identical, at least about 95% identical, at least about 96% identical, at least 97% identical, at least 98% identical, or at least 99% identical to a polynucleotide sequence encoding a polypeptide described herein.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises a polynucleotide having a nucleotide sequence at least about 80% identical, at least about 85% identical, at least about 90% identical, at least about 95% identical, at least about 96% identical, at least 97% identical, at least 98% identical, or at least 99% identical to a polynucleotide encoding an amino acid sequence selected from the group consisting of: SEQ ID NOs:70-86, 182-198, 201 and 208-211. Also provided is a polynucleotide that comprises a polynucleotide that hybridizes to a polynucleotide encoding an amino acid sequence selected from the group consisting of: SEQ ID NOs: SEQ ID NOs:70-86, 182-198, 201 and 208-211. In some embodiments, the hybridization is under conditions of high stringency as is known to those skilled in the art.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker may be used in conjunction with other markers or tags.

In some embodiments, a polynucleotide is isolated. In some embodiments, a polynucleotide is substantially pure.

Vectors and cells comprising the polynucleotides described herein are also provided. In some embodiments, an expression vector comprises a polynucleotide encoding a CD30 CAR described herein. In some embodiments, an expression vector comprises a polynucleotide molecule encoding a polypeptide that is part of a CD30 CAR described herein. In certain embodiments, the vector is a viral vector. In some embodiments, a host cell comprises an expression vector comprising a polynucleotide molecule encoding a CD30 CAR described herein.

In certain embodiments, a vector can include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the vector is a lentiviral vector. Lentiviruses are one of the most efficient methods of a gene delivery. Lentiviruses can infect non-dividing cells and they can deliver a significant amount of genetic information into a host cell. A lentiviral vector can be a vector derived from at least a portion of a lentivirus genome, including especially a self-inactivating lentiviral vector as provided in Milone et al., Mol. Ther. 17(8): 1453-1464 (2009). In some embodiments, any lentiviral vector known in the art may be used.

The CARs provided herein can be obtained by methods known in the art. Once assembled, the polynucleotide sequences encoding a polypeptide sequence (e.g. a CD30 CAR) disclosed herein can be inserted into an expression vector and operatively linked to an expression control sequence appropriate for expression of the protein in the desired host. The proper assembly can be confirmed by nucleotide sequencing, restriction enzyme mapping, and/or expression of a biologically active polypeptide in a suitable host.

As is well-known in the art, in order to obtain high expression levels of a transfected gene in a host, the gene must be operatively linked to transcriptional and translational expression control sequences that are functional in the chosen expression host. In some embodiments, a recombinant expression vector is used to amplify and express DNA encoding a polypeptide or molecule described herein. Appropriate cloning and expression vectors for use with bacterial, fungal, yeast, and mammalian cellular hosts are well known by those skilled in the art.

The nucleic acid can be cloned into a number of types of vectors including, but not limited to a plasmid, a phagemid, a phage derivative, an animal virus, and a cosmid. Vectors of particular interest include expression vectors, replication vectors, probe generation vectors, and sequencing vectors. In certain embodiments, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York). Viruses, which can useful as vectors include, but are not limited to, retroviruses, adenoviruses, adeno-associated viruses, herpes viruses, and lentiviruses. In a specific embodiment, a lentiviral vector can be used to express a polynucleotide sequence encoding a polypeptide sequence disclosed herein.

In some embodiments, a host cell comprises an expression vector comprising a polynucleotide molecule encoding a polypeptide that is part of a CD30 CAR described herein. In some embodiments, a host cell comprises a polynucleotide encoding a CD30-binding moiety described herein. As a non-limiting example, suitable host cells for expression of a polypeptide disclosed herein include prokaryotes, yeast cells, insect cells, or higher eukaryotic cells under the control of appropriate promoters. In certain embodiments, prokaryotic host cells can include E. coli. and eukaryotic cells can include established cell lines of mammalian origin, such as simian COS cells or Chinese hamster ovary (CHO) cells. Cell-free translation systems can also be employed. Expression of recombinant proteins in mammalian cells are generally appropriately modified, correctly folded, and biologically functional. In other embodiments, recombinant proteins, or fragments thereof, can be isolated from phage display libraries or using other cell surface display techniques.

The CARs disclosed herein that specifically bind CD30 can be made by any suitable method of making polypeptides or proteins. Suitable methods of de novo synthesizing polypeptides and proteins are known in the art. Also, the CARs can be recombinantly produced using the nucleic acids described herein using standard recombinant methods as described in, for example, Green and Sambrook, Molecular Cloning: A Laboratory Manual (4th Ed.), Cold Spring Harbor Laboratory Press (2012). Alternatively, the CARs described herein can be commercially synthesized by companies, such as, for example, Synpep (Dublin, Calif.) and Multiple Peptide Systems (San Diego, Calif.). In this respect, the CARs provided herein can be synthetic and/or recombinant.

Any method disclosed herein or known in the art can be used to introduce a nucleic acids disclosed herein into a host cell. In order to confirm the presence of the recombinant DNA sequence in the host cell, a variety of assays may be performed. As a non-limiting example, such assays include molecular biological assays well known to those of skill in the art, such as Southern blotting, northern blotting, RT-PCR and PCR; biochemical assays, such as detecting the presence or absence of a particular peptide, e.g., by immunological means (ELISAs and western blots).

### 4. CD30 CAR-EXPRESSING CELLS

Provided in the present disclosure is a cell that recombinantly expresses a CD30 CAR disclosed herein. In some embodiments, the cell is an immune cell. In some embodiments, the cells are derived from a human (are of human origin prior to being made recombinant). The immune cells can be cells of the lymphoid lineage. Non-limiting examples of cells of the lymphoid lineage include T cells and Natural Killer (NK) cells. T cells express the T cell receptor (TCR), with most cells expressing α and β chains and a smaller population expressing γ and δ chains (the “γδ T cells”). T cells useful as immune cells of the present disclosures can be CD4+ or CD8+ and can include, but are not limited to, T helper cells (CD4+), cytotoxic T cells (CD8+), natural killer T cells, γδT cells, mucosal associated invariant T cells (MAIT), and memory T cells, including central memory T cells, stem-cell-like memory T cells (or stem-like memory T cells), and effector memory T cells, for example, TEM cells and TEMRA (CD45RA+) cells. Precursor cells of immune cells that can be used in present disclosure, which recombinantly express a CAR as described above, are, by way of example, hematopoietic stem and/or progenitor cells. Hematopoietic stem and/or progenitor cells can be derived from bone marrow, umbilical cord blood, adult peripheral blood after cytokine mobilization, and the like, by methods known in the art, and then are genetically engineered to recombinantly express a CD30 CAR disclosed herein. Particularly useful precursor cells are those that can differentiate into the lymphoid lineage, for example, hematopoietic stem cells or progenitor cells of the lymphoid lineage.

In some embodiments, the cell is a T cell. Provided herein is a T cell that recombinantly expresses a CAR (“CAR T”) that specifically binds CD30 disclosed herein. In some embodiments, the T cell is selected from the group consisting of a cytotoxic T cell, a helper T cell, a natural killer T (NKT) cell, and a γδT cell. Provided herein is a cytotoxic T cell that recombinantly expresses a CD30 CAR disclosed herein. Provided herein is a helper T cell that recombinantly expresses a CD30 CAR disclosed herein. Provided herein is a cytotoxic T cell that recombinantly expresses a CD30 CAR disclosed herein. Provided herein is a helper T cell that recombinantly expresses a CD30 CAR disclosed herein. Provided herein is a NKT cell that recombinantly expresses a CD30 CAR disclosed herein. In some embodiments, the cell is a Vα24-invariant NKT cells. Provided herein is a γδT cell that recombinantly expresses a CD30 CAR disclosed herein.

Immune cells and precursor cells thereof can be isolated by methods well known in the art, including commercially available isolation methods (see, for example, Rowland-Jones et al., Lymphocytes: A Practical Approach, Oxford University Press, New York (1999)). Sources for the immune cells or precursor cells thereof include, but are not limited to, peripheral blood, umbilical cord blood, bone marrow, or other sources of hematopoietic cells. Various techniques can be employed to separate the cells to isolate or enrich for desired immune cells. For instance, negative selection methods can be used to remove cells that are not the desired immune cells. Additionally, positive selection methods can be used to isolate or enrich for desired immune cells or precursor cells thereof, or a combination of positive and negative selection methods can be employed. Monoclonal antibodies (MAbs) are particularly useful for identifying markers associated with particular cell lineages and/or stages of differentiation for both positive and negative selections. If a particular type of cell is to be isolated, for example, a particular type of T cell, various cell surface markers or combinations of markers, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009)).

Procedures for separation of cells include, but are not limited to, density gradient centrifugation, coupling to particles that modify cell density, magnetic separation with antibody-coated magnetic beads, affinity chromatography; cytotoxic agents joined to or used in conjunction with a monoclonal antibody (mAb), including, but not limited to, complement and cytotoxins, and panning with an antibody attached to a solid matrix, for example, a plate or chip, elutriation, flow cytometry, or any other convenient technique (see, for example, Recktenwald et al., Cell Separation Methods and Applications, Marcel Dekker, Inc., New York (1998)).

The immune cells or precursor cells thereof can be autologous or non-autologous to the subject to which they are administered in the methods of treatment disclosed herein. Autologous cells are isolated from the subject to which the engineered cells recombinantly expressing a CD30 CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine an appropriate level of compatibility, as is well known in the art. For both autologous and non-autologous cells, the cells can optionally be cryopreserved until ready to be used for genetic manipulation and/or administration to a subject using methods well known in the art.

Various methods for isolating immune cells that can be used for recombinant expression of a CAR have been described previously, and can be used, including but not limited to, using peripheral donor lymphocytes (Sadelain et al., Nat. Rev. Cancer 3:35-45 (2003); Morgan et al., Science 314: 126-129 (2006), using lymphocyte cultures derived from tumor infiltrating lymphocytes (TILs) in tumor biopsies (Panelli et al., J. Immunol. 164:495-504 (2000); Panelli et al., J Immunol. 164:4382-4392 (2000)), and using selectively in vitro-expanded antigen-specific peripheral blood leukocytes employing artificial antigen-presenting cells (AAPCs) or dendritic cells (Dupont et al., Cancer Res. 65:5417-5427 (2005); Papanicolaou et al., Blood 102:2498-2505 (2003)). In the case of using stem cells, the cells can be isolated by methods well known in the art (see, for example, Klug et al., Hematopoietic Stem Cell Protocols, Humana Press, New Jersey (2002); Freshney et al., Culture of Human Stem Cells, John Wiley & Sons (2007)).

A CAR-expressing cell (e.g. CAR T) disclosed herein can further recombinantly express one or more additional factors (e.g., polynucleotides or polypeptides) that can enhance the survival, proliferation or functionality (e.g., anti-cancer activity) of the CAR-expressing cell. In some embodiments, the additional factor is conjugated to the CD30 CAR. In some embodiments, isolated immune cells and precursor cells are genetically engineered ex vivo for recombinant expression of a CAR. The cells can be genetically engineered for recombinant expression by methods well known in the art.

In certain embodiments, the additional factor is conjugated to the C-terminus of the CAR. In some embodiments, the factor is conjugated to the N-terminus of the CAR. In some embodiments, the CAR is conjugated directly to the factor. In some embodiments, the CAR is conjugated to the factor via a linker. Any linker known in the art appropriate for connecting two polypeptides can be used to connect the CAR and the distinct factor. In some embodiments, the linker is a cleavable linker. In some embodiments, the cleavable linker is a self-cleaving peptide.

In some embodiments, the linker is a “2A” peptide. A 2A peptide is about 18-22 amino-acid long viral oligopeptides that mediates cleavage of polypeptides during translation in eukaryotic cells. The designation “2A” refers to a specific region of the viral genome and different viral 2As have generally been named after the virus they were derived from. In some embodiments, the CAR is conjugated to the additional factor via a 2A linker. In some embodiments the 2A linker is selected from the group consisting of 2A porcine teschovirus-1 (P2A), thosea asigna virus 2A (T2A), equine rhinitis A virus 2A (E2A), foot-and-mouth disease virus (F2A), cytoplasmic polyhedrosis virus (BmCPV 2A). In some embodiments, the CAR is conjugated to the additional factor via a P2A linker. In some embodiments, the CAR is conjugated to the additional factor via a T2A linker. In some embodiments, the CAR is conjugated to the additional factor via a E2A linker. In some embodiments, the CAR is conjugated to the additional factor via a F2A linker. In some embodiments, the linker comprises or consists of the amino acid sequence of SEQ ID NO:66.

In some embodiments, the additional factor can promote the trafficking and infiltration of the CAR-expressing cells into tumor sites. In some embodiments, the additional factor can promote the proliferation of the CAR-expressing cells. In some embodiments, the additional factor can promote the cytotoxicity of the CAR-expressing cells. In some embodiments, the additional factor can promote the cytokine production of the CAR-expressing cells. In some embodiments, the additional factor can release the immune-suppressive effects induced by an inhibitory molecule (i.e., an immune inhibitory molecule). As a non-limiting example, in some embodiments, the immune inhibitory molecule is PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta. In some embodiments, the additional factor can have one or more of the above-mentioned function.

In certain embodiments, a CAR-expressing cell (e.g. CAR T) further comprises one additional factor. In certain embodiments, a CAR-expressing cell (e.g. CAR T) further comprises two additional factors. In certain embodiments, a CAR-expressing cell (e.g. CAR T) further comprises three additional factors. In certain embodiments, a CAR-expressing cell (e.g. CAR T) further comprises four or more additional factors. In certain embodiments, a CAR-expressing cell (e.g. CAR T) further comprises at least one additional factor(s) selected from the group consisting of C—C chemokine receptor type 4 (CCR4), dominant negative transforming growth factor beta receptor II (dnTGFβRII), a chimeric switch programmed death 1 receptor (PD1CD28), and any combination thereof.

In some embodiments, a CAR-expressing cell (e.g. CAR T) disclosed herein further comprises CCR4. The chemokine receptor CCR4 (also known as CD194) is a seven trans-membrane G protein-coupled cell surface receptor molecule with selective expression on cells of the hematopoietic system. In some embodiments, incorporation and expression of chemokine receptor genes such as CCR4 in CAR T cells can promote their trafficking and infiltration into tumor sites, and facilitate effective T-cell mediated killing of tumor cells. In some embodiments, the expression of CCR4 can improve effector function of the CAR-expressing cell. In certain embodiments, CCR4 expression improves the cytotoxicity of T cells to CD30+ tumor cells.

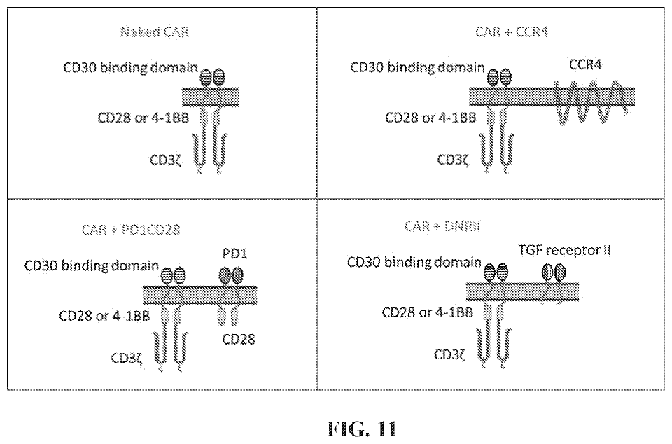

In some embodiments, a CAR-expressing cell (e.g. CAR T) disclosed herein further expresses CCR4. In some embodiments, the CAR disclosed herein is conjugated to CCR4. In some embodiments, CCR4 is conjugated to the N-terminus of the CAR disclosed herein, designated as “4C-[CAR].” In some embodiments, CCR4 is conjugated to the C-terminus of the CAR disclosed herein, designated as “KARI-4C.” (FIG. 11, top right.) In some embodiments, CCR4 has an amino acid sequence comprising SEQ ID NO:67. The CAR can be any CAR disclosed herein. In some constructs, CCR4 molecule and CAR are connected via P2A (SEQ ID NO: 66).

For example, provided herein is a CD30 CAR conjugate designated AS48542VH5bbz-4C (SEQ ID NO:198), which comprises, from N-terminus to C-terminus, a CAR designated AS48542VH5bbz and CCR4. In some embodiments, provided herein is a CD30 CAR conjugate having an amino acid sequence that is at least 80% identical to SEQ ID NO:198. In some embodiments, provided herein is a CD30 CAR conjugate having an amino acid sequence that is at least 85% identical to SEQ ID NO:198. In some embodiments, provided herein is a CD30 CAR conjugate having an amino acid sequence that is at least 90% identical to SEQ ID NO:198. In some embodiments, provided herein is a CD30 CAR conjugate having an amino acid sequence that is at least 95% identical to SEQ ID NO:198. In some embodiments, provided herein is a CD30 CAR conjugate having an amino acid sequence that is at least 97% identical to SEQ ID NO:198. In some embodiments, provided herein is a CD30 CAR conjugate having an amino acid sequence that is at least 99% identical to SEQ ID NO:198. In some embodiments, provided herein is a CD30 CAR conjugate having an amino acid sequence comprising SEQ ID NO:198. In some embodiments, provided herein are polynucleotides encoding a polypeptide comprising the CD30 CAR conjugate described herein. In some embodiments, provided herein are polynucleotides encoding a polypeptide comprising the CD30 CAR conjugate having an amino acid sequence that is at least 80%, 85%, 90%, 95%, 97%, or 99% identical to SEQ ID NO:198. In some embodiments, provided herein are polynucleotides encoding a polypeptide comprising an amino acid sequence of SEQ ID NO:198.

In some embodiments, a CAR-expressing cell (e.g. CAR T) disclosed herein further comprises a factor that can antagonize the activity of transforming growth factor beta (TGFβ). TGFβ is a cytokine with pleiotropic functions including regulation of cell growth, differentiation and immunoregulation. As a potent suppressor of the immune system, it is secreted by many human tumors as part of an immune evasion strategy. TGFβ markedly inhibits tumor-specific cellular immunity, suppressing the activity of cytotoxic lymphocytes in the tumor microenvironment. Release from TGFβ-mediated immune suppression can restore anti-tumor immunity. In some embodiments, expression of a dominant negative TGFβRII (dnTGFβRII) can block TGFβ signaling in T cells, thereby increasing their ability to infiltrate, proliferate, and mediate anti-tumor responses. In certain embodiments, expression of to dnTGFβRII can enhance production and/or secretion of one or more cytokines (e.g., IL-4, IL-5, IL-13, IL-2, IFN-γ, MIP1-α, MIP1-β, GM-CSF and/or RANTES). In certain embodiments, expression of to dnTGFβRII can enhance production and/or secretion of, IFN-γ and GM-CSF. In some embodiments, expression of to dnTGFβRII can enhance infiltration and/or proliferation of the CAR-expressing cell disclosed herein.

In some embodiments, a CAR-expressing cell (e.g. CAR T) disclosed herein further comprises dnTGFβRII. In some embodiments, expression of a dominant negative TGFβRII can block TGFβ signal transduction in a T cell by, for example, inhibition of Smad2 phosphorylation. In some embodiments, a CAR-expressing cell (e.g. CAR T) disclosed herein further expresses dnTGFβRII. In some embodiments, the CAR disclosed herein is conjugated to dnTGFβRII. In some embodiments, dnTGFβRII is conjugated to the N-terminus of the CAR disclosed herein, designated as “TR2D-[CAR].” In some embodiments, dnTGFβRII is conjugated to the C-terminus of the CAR disclosed herein, designated as “[CAR]-TR2D.” In some embodiments, dnTGFβRII has an amino acid sequence comprising SEQ ID NO:68. The CAR can be any CAR disclosed herein.

For example, provided herein is a CD30 CAR conjugate designated TR2D-AS48542VH5bbz (SEQ ID NO:196), which comprises, from N-terminus to C-terminus, dnTGFβRII and a CAR designated AS48542VH5bbz. In some embodiments, provided herein is a CD30 CAR conjugate having an amino acid sequence that is at least 80% identical to SEQ ID NO:196. In some embodiments, provided herein is a CD30 CAR conjugate having an amino acid sequence that is at least 85% identical to SEQ ID NO:196. In some embodiments, provided herein is a CD30 CAR conjugate having an amino acid sequence that is at least 90% identical to SEQ ID NO:196. In some embodiments, provided herein is a CD30 CAR conjugate having an amino acid sequence that is at least 95% identical to SEQ ID NO:196. In some embodiments, provided herein is a CD30 CAR conjugate having an amino acid sequence that is at least 97% identical to SEQ ID NO:196. In some embodiments, provided herein is a CD30 CAR conjugate having an amino acid sequence that is at least 99% identical to SEQ ID NO:196. In some embodiments, provided herein is a CD30 CAR conjugate having an amino acid sequence comprising SEQ ID NO:196. In some embodiments, provided herein are polynucleotides encoding a polypeptide comprising the CD30 CAR conjugate described herein. In some embodiments, provided herein are polynucleotides encoding a polypeptide comprising the CD30 CAR conjugate having an amino acid sequence that is at least 80%, 85%, 90%, 95%, 97%, or 99% identical to SEQ ID NO:196. In some embodiments, provided herein are polynucleotides encoding a polypeptide comprising an amino acid sequence of SEQ ID NO:196. The present disclosure also provides CD30 CAR conjugates designated TR2D-AS47863VH4bbz (SEQ ID NO:206), which comprises, from N-terminus to C-terminus, dnTGFβRII and a CAR designated AS47863VH4bbz. In some embodiments, provided herein is a CD30 CAR conjugate having an amino acid sequence that is at least 80%, 85%, 90%, 95%, 97%, or 99% identical to SEQ ID NO:206. In some embodiments, provided herein is a CD30 CAR conjugate having an amino acid sequence of SEQ ID NO:206. The present disclosure provides CD30 CAR conjugates designated TR2D-AS48542VH5dil-bbz (SEQ ID NO:205), which comprises, from N-terminus to C-terminus, dnTGFβRII and a CAR designated AS48542VH5dil-bbz. In some embodiments, provided herein is a CD30 CAR conjugate having an amino acid sequence that is at least 80%, 85%, 90%, 95%, 97%, or 99% identical to SEQ ID NO:205. In some embodiments, provided herein is a CD30 CAR conjugate having an amino acid sequence of SEQ ID NO:205. The present disclosure also provides CD30 CAR conjugates designated TR2D-AS47863VH4dil-bbz (SEQ ID NO:207), which comprises, from N-terminus to C-terminus, dnTGFβRII and a CAR designated AS47863VH4dil-bbz. In some embodiments, provided herein is a CD30 CAR conjugate having an amino acid sequence that is at least 80%, 85%, 90%, 95%, 97%, or 99% identical to SEQ ID NO:207. In some embodiments, provided herein is a CD30 CAR conjugate having an amino acid sequence of SEQ ID NO:207. The present disclosure provides CD30 CAR conjugates designated TR2D-AS48542VH5-28z (SEQ ID NO:212), which comprises, from N-terminus to C-terminus, dnTGFβRII and a CAR designated AS48542VH5-28z. In some embodiments, provided herein is a CD30 CAR conjugate having an amino acid sequence that is at least 80%, 85%, 90%, 95%, 97%, or 99% identical to SEQ ID NO:212. In some embodiments, provided herein is a CD30 CAR conjugate having an amino acid sequence of SEQ ID NO:212. The present disclosure provides CD30 CAR conjugates designated TR2D-AS47863VH4-28z (SEQ ID NO:214), which comprises, from N-terminus to C-terminus, dnTGFβRII and a CAR designated AS47863VH4-28z. In some embodiments, provided herein is a CD30 CAR conjugate having an amino acid sequence that is at least 80%, 85%, 90%, 95%, 97%, or 99% identical to SEQ ID NO:214. In some embodiments, provided herein is a CD30 CAR conjugate having an amino acid sequence of SEQ ID NO:214. The present also disclosure provides CD30 CAR conjugates designated TR2D-AS48542VH5dil-28z (SEQ ID NO: 213), which comprises, from N-terminus to C-terminus, dnTGFβRII and a CAR designated AS48542VH5dil-28z. In some embodiments, provided herein is a CD30 CAR conjugate having an amino acid sequence that is at least 80%, 85%, 90%, 95%, 97%, or 99% identical to SEQ ID NO:213. In some embodiments, provided herein is a CD30 CAR conjugate having an amino acid sequence of SEQ ID NO:213. The present also disclosure provides CD30 CAR conjugates designated TR2D-AS47863VH4dil-28z (SEQ ID NO:215), which comprises, from N-terminus to C-terminus, dnTGFβRII and a CAR designated AS47863VH4dil-28z. In some embodiments, provided herein is a CD30 CAR conjugate having an amino acid sequence that is at least 80%, 85%, 90%, 95%, 97%, or 99% identical to SEQ ID NO:215. In some embodiments, provided herein is a CD30 CAR conjugate having an amino acid sequence of SEQ ID NO:215.

In some embodiments, a CAR-expressing cell (e.g. CAR T) disclosed herein further comprises a factor that is an inhibitor of PD-1 signaling PD-1 (also known as CD279) is cell surface receptor that belongs to the immunoglobulin superfamily and is expressed at the cell surface of activated T cells, NK cells, B cells, macrophages and several subsets of DCs. Its expression is upregulated after antigen- and ligand-receptor engagement by its currently known ligands, PD-L1 (also known as B7-H1 or CD274) and PD-L2 (also known as B7-DC or CD273). In some embodiments, inhibition of PD-1 signaling enhance the anti-tumor activities of the CAR-expressing cells.

PD is a chimeric switch-receptor containing the extracellular domain of PD-1 fused to the transmembrane and cytoplasmic domain of the co-stimulatory molecule CD28. PD1CD28 switch receptor can antagonize the immune suppressive activity of PD-1 via at least two mechanisms. First, when the PD-1 portion of this switch-receptor engages its ligand (e.g., PD-L1), rather than transmitting the inhibitory signal, it can transmit an activating signal via the CD28 cytoplasmic domain. Second, the receptor can function as a dominant negative receptor, engaging the PD-L1 present on tumor and myeloid cells to sequester it from the intact inhibitory PD-1, thereby reducing inhibitory signaling. In certain embodiments, expression of PD1CD28 in CAR-expressing cells disclosed herein has increased production and/or secretion of cytokine (e.g., IFNγ and IL2). In certain embodiments, expression of PD1CD28 in CAR-expressing cells disclosed herein can enhance the anti-tumor activity of the CAR-expressing cells. In certain embodiments, expression of PD1CD28 in CAR T cells disclosed herein can enhance the cytotoxic activity of the CAR T cells.

In some embodiments, a CAR-expressing cell (e.g. CAR T) disclosed herein further comprises PD1CD28. In some embodiments, a CAR-expressing cell (e.g. CAR T) disclosed herein further expresses PD1CD28. In some embodiments, the CAR disclosed herein is conjugated to PD1CD28. In some embodiments, PD1CD28 is conjugated to the N-terminus of the CAR disclosed herein, designated as “PD1CD28-[CAR].” In some embodiments, PD1CD28 is conjugated to the C-terminus of the CAR disclosed herein, designated as “[CAR]-PD1CD28.” In some embodiments, PD1CD28 has an amino acid sequence comprising SEQ ID NO:69. The CAR can be any CAR disclosed herein.

For example, provided herein is a CD30 CAR conjugate designated PD1CD28-AS48542VH5bil-bbz (SEQ ID NO:197), which comprises, from N-terminus to C-terminus, PD1CD28 and a CAR designated AS48542VH5bbz. In some embodiments, provided herein is a CD30 CAR conjugate having an amino acid sequence that is at least 80% identical to SEQ ID NO:197. In some embodiments, provided herein is a CD30 CAR conjugate having an amino acid sequence that is at least 85% identical to SEQ ID NO:197. In some embodiments, provided herein is a CD30 CAR conjugate having an amino acid sequence that is at least 90% identical to SEQ ID NO:197. In some embodiments, provided herein is a CD30 CAR conjugate having an amino acid sequence that is at least 95% identical to SEQ ID NO:197. In some embodiments, provided herein is a CD30 CAR conjugate having an amino acid sequence that is at least 97% identical to SEQ ID NO:197. In some embodiments, provided herein is a CD30 CAR conjugate having an amino acid sequence that is at least 99% identical to SEQ ID NO:197. In some embodiments, provided herein is a CD30 CAR conjugate having an amino acid sequence comprising SEQ ID NO:197. In some embodiments, provided herein are polynucleotides encoding a polypeptide comprising the CD30 CAR conjugate described herein. In some embodiments, provided herein are polynucleotides encoding a polypeptide comprising the CD30 CAR conjugate having an amino acid sequence that is at least 80%, 85%, 90%, 95%, 97%, or 99% identical to SEQ ID NO:197. In some embodiments, provided herein are polynucleotides encoding a polypeptide comprising an amino acid sequence of SEQ ID NO:197.

In some embodiments, a CAR-expressing cell (e.g. CAR T) disclosed herein further comprises CCR4 and dnTGFβRII. In some embodiments, a CAR-expressing cell (e.g. CAR T) disclosed herein further comprises CCR4 and PD1CD28. In some embodiments, a CAR-expressing cell (e.g. CAR T) disclosed herein further comprises dnTGFβRII and PD1CD28. In some embodiments, a CAR-expressing cell (e.g. CAR T) disclosed herein further comprises CCR4, dnTGFβRII and PD1CD28. The CAR can be any CAR disclosed herein.

In some embodiments, a CAR disclosed herein is conjugated to CCR4 and dnTGFβRII. In some embodiments, the conjugates provided herein comprises, from N-terminus to C-terminus, CAR, CCR4, and dnTGFβRII, designated as “[CAR]-4C-TR2D.” In some embodiments, the CD30 CAR conjugates provided herein comprises, from N-terminus to C-terminus, CAR, dnTGFβRII, and CCR4, designated as “[CAR]-TR2D-4C.” In some embodiments, the CD30 CAR conjugates provided herein comprises, from N-terminus to C-terminus, dnTGFβRII, CAR, and CCR4, designated as “TR2D-[CAR]-4C.” In some embodiments, the CD30 CAR conjugates provided herein comprises, from N-terminus to C-terminus, CCR4, CAR, and dnTGFβRII designated as “4C-[CAR]-TR2D.” In some embodiments, the CD30 CAR conjugates provided herein comprises, from N-terminus to C-terminus, dnTGFβRII, CCR4, and CAR, designated as “TR2D-4C-[CAR].” In some embodiments, the CD30 CAR conjugates provided herein comprises, from N-terminus to C-terminus, CCR4, dnTGFβRII, and CAR, designated as “4C-TR2D-[CAR].” The CAR can be any CAR disclosed herein.

For example, provided herein is a CD30 CAR conjugate designated TR2D-AS48542VH5bbz-4C (SEQ ID NO:195). TR2D-AS48542VH5bbz-4C comprises, from N-terminus to C-terminus, dnTGFβRII, the CAR designated AS48542VH5bbz, and CCR4. In some embodiments, provided herein is a CD30 CAR conjugate having an amino acid sequence that is at least 80% identical to SEQ ID NO:195. In some embodiments, provided herein is a CD30 CAR conjugate having an amino acid sequence that is at least 85% identical to SEQ ID NO:195. In some embodiments, provided herein is a CD30 CAR conjugate having an amino acid sequence that is at least 90% identical to SEQ ID NO:195. In some embodiments, provided herein is a CD30 CAR conjugate having an amino acid sequence that is at least 95% identical to SEQ ID NO:195. In some embodiments, provided herein is a CD30 CAR conjugate having an amino acid sequence that is at least 97% identical to SEQ ID NO:195. In some embodiments, provided herein is a CD30 CAR conjugate having an amino acid sequence that is at least 99% identical to SEQ ID NO:195. In some embodiments, provided herein is a CD30 CAR conjugate having an amino acid sequence comprising SEQ ID NO:195. In some embodiments, provided herein are polynucleotides encoding a polypeptide comprising the CD30 CAR conjugate described herein. In some embodiments, provided herein are polynucleotides encoding a polypeptide comprising the CD30 CAR conjugate having an amino acid sequence that is at least 80%, 85%, 90%, 95%, 97%, or 99% identical to SEQ ID NO:195. In some embodiments, provided herein are polynucleotides encoding a polypeptide comprising an amino acid sequence of SEQ ID NO:195.

The immune cells or precursor cells thereof can be subjected to conditions that favor maintenance or expansion of the immune cells or precursor cells thereof (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009); Parente-Pereira et al., J. Biol. Methods 1(2) e7 (doi 10.14440/jbm.2014.30) (2014); Movassagh et al., Hum. Gene Ther. 11: 1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1999); Quinn et al., Hum. Gene Ther. 9: 1457-1467 (1998); see also commercially available methods such as Dynabeads™ human T cell activator products, Thermo Fisher Scientific, Waltham, Mass.)). In some embodiments, the immune cells or precursor cells thereof can be expanded prior to or after ex vivo genetic engineering. Expansion of the cells is particularly useful to increase the number of cells for administration to a subject. Such methods for expansion of immune cells are well known in the art (see Kaiser et al., Cancer Gene Therapy 22:72-78 (2015); Wolfl et al., Nat. Protocols 9:950-966 (2014)). Furthermore, the cells can optionally be cryopreserved after isolation and/or genetic engineering, and/or expansion of genetically engineered cells (see Kaiser et al., supra, 2015)). Methods for cyropreserving cells are well known in the art (see, for example, Freshney, Culture of Animal Cells: A Manual of Basic Techniques, 4th ed., Wiley-Liss, New York (2000); Harrison and Rae, General Techniques of Cell Culture, Cambridge University Press (1997)).

With respect to generating cells recombinantly expressing a CD30 CAR disclosed herein and optionally with at least one additional factor, one or more nucleic acids encoding the CD30 CAR and optionally the at least additional factor is introduced into the immune cell or precursor cell thereof using a suitable expression vector. The immune cells (for example, T cells) or precursor cells thereof are preferably transduced with one or more nucleic acids encoding a CD30 CAR and optionally an additional factor. In the case of expressing both a CAR and an additional factor, the CAR and additional factor encoding nucleic acids can be on separate vectors or on the same vector, as desired. For example, a polynucleotide encoding a CD30 CAR or an additional factor can be cloned into a suitable vector, such as a retroviral vector, and introduced into the immune cell using well known molecular biology techniques (see Ausubel et al., Current Protocols in Molecular Biology, John Wiley and Sons, Baltimore, Md. (1999)). Any vector suitable for expression in a cell described herein, particularly a human immune cell or a precursor cell thereof, can be employed. The vectors contain suitable expression elements such as promoters that provide for expression of the encoded nucleic acids in the immune cell. In the case of a retroviral vector, cells can optionally be activated to increase transduction efficiency (see Parente-Pereira et al., J. Biol Methods 1(2) e7 (doi 10.14440/jbm.2014.30) (2014); Movassagh et al., Hum. Gene Ther. 11: 1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1998); Quinn et al., Hum. Gene Ther. 9: 1457-1467 (1998); see also commercially available methods such as Dynabeads™ human T cell activator products, Thermo Fisher Scientific, Waltham, Mass.).

In one embodiment, the vector is a retroviral vector, for example, a gamma retroviral or lentiviral vector, which is employed for the introduction of a CD30 CAR and optionally an additional factor into the immune cell or precursor cell thereof. For genetic modification of the cells to express a CD30 CAR and optionally an additional factor, a retroviral vector is generally employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al., Mol. Cell. Biol. 5:431-437 (1985)); PA317 (Miller et al., Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD1 14 or GALV envelope and any other known in the art (Relander et al., Mol. Therap. 11:452-459 (2005)). Possible methods of transduction also include direct co-culture of the cells with producer cells (for example, Bregni et al., Blood 80: 1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89: 1817-1824 (1992)).

Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J. Virol. 71:6641-6649 (1997); Naldini et al., Science 272:263 267 (1996); and Miyoshi et al., Proc. Natl. Acad. Sci. USA. 94: 10319-10323 (1997)). Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene Ther. 1(1):5-14 (1990); Friedman, Science 244: 1275-1281 (1989); Eglitis et al., BioTechniques 6:608-614 (1988); Tolstoshev et al., Current Opin. Biotechnol. 1: 55-61 (1990); Sharp, Lancet 337: 1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med. 323: 370 (1990); Anderson et al., U.S. Pat. No. 5,399,346).

Particularly useful vectors for expressing a CD30 CAR and optionally an additional factor as described herein include vectors that have been used in human gene therapy. In one non-limiting embodiment, a vector is a retroviral vector. The use of retroviral vectors for expression in T cells or other immune cells, including engineered CAR T cells, has been described (see Scholler et al., Sci. Transl. Med. 4: 132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15: 1454-1459 (2008)).

The vectors described herein include suitable promoters for expression in a particular host cell. The promoter can be an inducible promoter or a constitutive promoter. In a particular embodiment, the promoter of an expression vector provides expression in an immune cell, such as a T cell, or precursor cell thereof. Non-viral vectors can be used as well, so long as the vector contains suitable expression elements for expression in the immune cell or precursor cell thereof. Some vectors, such as retroviral vectors, can integrate into the host genome. If desired, targeted integration can be implemented using technologies such as a nuclease, transcription activator-like effector nucleases (TALENs), Zinc-finger nucleases (ZFNs), and/or clustered regularly interspaced short palindromic repeats (CRISPRs), by homologous recombination, and the like (Gersbach et al., Nucl. Acids Res. 39:7868-7878 (2011); Vasileva, et al. Cell Death Dis. 6:e1831. (Jul. 23 2015); Sontheimer, Hum. Gene Ther. 26(7):413-424 (2015)).

The vectors and constructs can optionally be designed to include a reporter. For example, the vector can be designed to express a reporter protein, which can be useful to identify cells comprising the vector or nucleic acids provided on the vector, such as nucleic acids that have integrated into the host chromosome. In one embodiment, the reporter can be expressed as a bicistronic or multicistronic expression construct with the CD30 CAR and the at least one additional factor. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. In an additional embodiment, a vector construct can comprise a P2A sequence, which provides for optional co-expression of a reporter molecule. P2A is a self-cleaving peptide sequence, which can be used for bicistronic or multicistronic expression of protein sequences (see Szymczak et al., Expert Opin. Biol. Therapy 5(5):627-638 (2005)).

Assays can be used to determine the transduction efficiency of a CD30 CAR and optionally an additional factor using routine molecular biology techniques. If a marker has been included in the construct, such as a fluorescent protein, gene transfer efficiency can be monitored by FACS analysis to quantify the fraction of transduced (for example, GFP+) immune cells, such as T cells, or precursor cells thereof, and/or by quantitative PCR. Using a well-established cocultivation system (Gade et al., Cancer Res. 65:9080-9088 (2005); Gong et al., Neoplasia 1: 123-127 (1999); Latouche et al., Nat. Biotechnol. 18:405-409 (2000)) it can be determined whether fibroblast AAPCs expressing cancer antigen (vs. controls) direct cytokine release from transduced immune cells, such as T cells, expressing a CAR (cell supernatant LUMINEX (Austin Tex.) assay for IL-2, IL-4, IL-10, IFN-γ, TNF-α, and GM-CSF), T cell proliferation (by carboxyfluorescein succinimidyl ester (CFSE) labeling), and T cell survival (by Annexin V staining). The influence of CD80 and/or 4-1BBL on T cell survival, proliferation, and efficacy can be evaluated. T cells can be exposed to repeated stimulation by cancer antigen positive target cells, and it can be determined whether T cell proliferation and cytokine response remain similar or diminished with repeated stimulation. The CD30 CAR constructs can be compared side by side under equivalent assay conditions. Cytotoxicity assays with multiple E:T ratios can be conducted using chromium-release assays.

In addition to providing a nucleic acid encoding a CD30 CAR and optionally an additional factor in a vector for expression in an immune cell or precursor cell thereof, a nucleic acid encoding the polypeptide can also be provided in other types of vectors more suitable for genetic manipulation, such as for expression of various constructs in a bacterial cell such as E. coli. Such vectors can be any of the well known expression vectors, including commercially available expression vectors (see in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

If desired, a nucleic acid encoding a polypeptide for genetic engineering of a cell of the invention, such a CD30 CAR and optionally an additional factor, can be codon optimized to increase efficiency of expression in an immune cell or precursor cell thereof. Codon optimization can be used to achieve higher levels of expression in a given cell. Factors that are involved in different stages of protein expression include codon adaptability, mRNA structure, and various cis-elements in transcription and translation. Any suitable codon optimization methods or technologies that are known to one skilled in the art can be used to modify the polynucleotides encoding the polypeptides. Such codon optimization methods are well known, including commercially available codon optimization services, for example, OptimumGene™ (GenScript; Piscataway, N.J.), Encor optimization (EnCor Biotechnology; Gainseville Fla.), Blue Heron (Blue Heron Biotech; Bothell, Wash.), and the like. Optionally, multiple codon optimizations can be performed based on different algorithms, and the optimization results blended to generate a codon optimized nucleic acid encoding a polypeptide.

Further modification can be introduced to the immune cells or precursor cells thereof of the invention. For example, the cells can be modified to address immunological complications and/or targeting by the CD30 CAR to healthy tissues that express the same target antigens as the tumor cells. For example, a suicide gene can be introduced into the cells to provide for depletion of the cells when desired. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. Agents are administered to the subject to which the cells containing the suicide genes have been administered, including but not limited to, gancilovir (GCV) for hsv-tk (Greco et al., Frontiers Pharmacol. 6:95 (2015); Barese et al., Mol. Therapy 20: 1932-1943 (2012)), AP1903 for iCasp-9 (Di Stasi et al., N. Engl. J. Med. 365: 1673-1683 (2011), and cetuximab for EGFRt (U.S. Pat. No. 8,802,374), to promote cell death. In one embodiment, administration of a prodrug designed to activate the suicide gene, for example, a prodrug such as API 903 that can activate iCasp-9, triggers apoptosis in the suicide gene-activated cells. In one embodiment, iCasp9 consists of the sequence of the human FK506-binding protein (FKBP12; GenBank number, AH002818 (AH002818.1, M92422.1, GL 182645; AH002818.2, GI: 1036032368)) with an F36V mutation, connected through a Ser-Gly-Gly-Gly-Ser linker (SEQ ID NO:204) to the gene encoding human caspase 9 (CASP9; GenBank number, NM001229 (NM_001229.4, GL493798577)), which has had its endogenous caspase activation and recruitment domain deleted. FKBP12-F36V binds with high affinity to an otherwise bioinert small-molecule dimerizing agent, AP1903. In the presence of AP1903, the iCasp9 promolecule dimerizes and activates the intrinsic apoptotic pathway, leading to cell death (Di Stasi et al., N. Engl. J. Med. 365: 1673-1683 (2011)). In another embodiment, the suicide gene is an EGFRt polypeptide. The EGFRt polypeptide can provide for cell elimination by administering anti-EGFR monoclonal antibody, for example, cetuximab. The suicide gene can be expressed on a separate vector or, optionally, expressed within the vector encoding a CD30 CAR and optionally an additional factor, and can be a bicistronic or multicistronic construct joined to a CD30 CAR and optionally an additional factor-encoding nucleic acid.

### 5. COMPOSITIONS

The present disclosures also provide pharmaceutical compositions comprising the CAR-expressing cells disclosed herein. The pharmaceutical composition comprises an effective amount of the CAR-expressing cells disclosed herein and a pharmaceutically acceptable carrier. The CAR-expressing cells can be engineered immune cells. In some embodiments, the engineered immune cell is a T cell selected from the group consisting of a cytotoxic T cell, a helper T cell, a γδT cell, and a NKT cell. In some embodiments, a pharmaceutical composition comprises a therapeutically effective population of the CAR T cells disclosed herein and a pharmaceutically acceptable carrier. The CAR-expressing cells disclosed herein and compositions comprising these cells can be conveniently provided in sterile liquid preparations, for example, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating the CAR-expressing cells disclosed herein in a suitable amount of the appropriate solvent with various amounts of the other ingredients, as desired. Such compositions can include a pharmaceutically acceptable carrier, diluent, or excipient such as sterile water, physiological saline, glucose, dextrose, or the like, that are suitable for use with a cell composition and for administration to a subject such as a human. Suitable buffers for providing a cell composition are well known in the art. Any vehicle, diluent, or additive used is compatible with preserving the integrity and viability of the cells of the invention.

The compositions will generally be isotonic, that is, they have the same osmotic pressure as blood and lacrimal fluid. The desired isotonicity of the cell compositions of the invention can be accomplished using sodium chloride, or other pharmaceutically acceptable agents such as dextrose, boric acid, sodium tartrate, or other inorganic or organic solutes. Sodium chloride is preferred particularly for buffers containing sodium ions. One particularly useful buffer is saline, for example, normal saline. Those skilled in the art will recognize that the components of the compositions should be selected to be chemically inert and will not affect the viability or efficacy of the cells of the invention and will be compatible for administration to a subject, such as a human. The skilled artisan can readily determine the amount of cells and optional additives, vehicles, and/or carrier in compositions to be administered in methods of the invention.

In certain embodiments, a pharmaceutical compositions of the present invention comprises a population of CAR-expressing cells disclosed herein, in combination with one or more pharmaceutically acceptable carriers. The pharmaceutically acceptable carriers can be any acceptable carriers, diluents and/or excipients known in the art. In some embodiments, pharmaceutical compositions disclosed herein can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The CAR-expressing cells disclosed herein can be administered in any physiologically acceptable vehicle. Suitable doses for administration are described herein. A cell population comprising the CAR-expressing cells disclosed herein can comprise a purified population of cells. Those skilled in the art can readily determine the percentage of cells in a cell population using various well-known methods, as described herein. The ranges of purity in cell populations comprising the CAR-expressing cells disclosed herein can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art; for example, a decrease in purity may require an increase in dosage.

The present disclosure also provides kits for preparation of cells of the invention. In one embodiment, the kit comprises one or more vectors for generating a genetically engineered immune cell, such as a T cell, or precursor cell thereof, that expresses the CD30 CARs disclosed herein. The kits can be used to generate genetically engineered immune cells from autologous cells derived from a subject or from non-autologous cells to be administered to a compatible subject. In another embodiment, the kits can comprise the CAR-expressing cells disclosed herein, for example, autologous or non-autologous cells, for administration to a subject. In specific embodiments, the kits comprise the CAR-expressing cells disclosed herein in one or more containers.

In some embodiments, provided herein is a population of cells comprising at least two of the CAR-expressing cells disclosed herein. In some embodiments, a population of cells is a homogenous population of cells. In some embodiments, a population of cells is a heterogenous population of cells. In some embodiments, a population of cells comprises a mixture of cells expressing different CARs (e.g., one or more CARs disclosed herein and, optionally, one or more CARs not disclosed herein). As an example, in some embodiments, a population of cells comprises a first cell expressing a CD30 CAR as disclosed herein and a second cell expressing a CAR that specifically binds an antigen other than CD30. In one embodiment, a population of cells comprises a first cell expressing a CAR that specifically binds a first CD30 epitope and a second cell expressing a CAR that specifically binds a second CD30 epitope. The second CD30 epitope can be different from the first CD30 epitope. In some embodiments, a population of cells comprises a first cell expressing a CAR having a CD30-binding moiety described herein and a second cell expressing a CAR having a second CD30-binding moiety described herein. The second CD30-binding moiety can be different from the first CD30-binding moiety.

In some embodiments, a population of cells comprises a homogenous population of the CAR-expressing cells disclosed herein described herein. The population of cells can include 0.1-500 million cells. In some embodiments, a population of cells comprises a homogenous population of 0.2 million cells described herein. In some embodiments, a population of cells comprises a homogenous population of 0.5 million cells described herein. In some embodiments, a population of cells comprises a homogenous population of 1 million cells described herein. In some embodiments, a population of cells comprises a homogenous population of 2 million cells described herein. In some embodiments, a population of cells comprises a homogenous population of 5 million cells described herein. In some embodiments, a population of cells comprises a homogenous population of 10 million cells described herein. In some embodiments, a population of cells comprises a homogenous population of 20 million cells described herein. In some embodiments, a population of cells comprises a homogenous population of 50 million cells described herein. In some embodiments, a population of cells comprises a homogenous population of 100 million cells described herein. In some embodiments, a population of cells comprises a homogenous population of 200 million cells described herein. In some embodiments, a population of cells comprises a homogenous population of 300 million cells described herein. In some embodiments, a population of cells comprises a homogenous population of 500 million cells described herein.

### 6. METHODS AND USES

The present disclosure also provides methods of uses of the CD30-binding moieties, CD30 CARs, polynucleotides encoding such CD30-binding moieties and CD30 CARs, recombinant expression vectors comprising such polynucleotides, CD30 CAR-expressing cells or pharmaceutical compositions having such cells disclosed herein in treating CD30-expressing cancer or tumor. Without being bound by theory, the CD30 CAR-expressing cells disclosed herein can specifically target CD30-expressing cancer cells in vivo, thereby delivering their therapeutic effect of eliminating, lysing and/or killing cancer cells. In one embodiment, the methods include administering a therapeutically effective amount of CD30 CAR-expressing immune cell or precursor cell disclosed herein to a subject in need thereof. In one embodiment, the methods can include administering a therapeutically effective amount of a CD30 CAR T cells disclosed herein to a subject in need thereof. The CD30 CAR-expressing cells should be administered in a sufficient amount to effect a therapeutic or prophylactic response in the subject or animal over a reasonable time frame.

In some embodiments, provided herein is a method of treating a CD30-expressing tumor or cancer in a subject in need thereof, comprising administering to the subject a therapeutically effective amount of the CD30 CAR-expressing cells or pharmaceutical compositions disclosed herein. CD30-expressing cancers or tumors that can be treated include non-solid tumors (such as hematological tumors, for example, leukemias and lymphomas) and solid tumors. In some embodiments, the CD30-expressing cancer or tumor can be lymphoma. The lymphoma can be B-cell lymphoma or T-cell lymphoma. The B-cell lymphoma can be diffuse large B cell lymphoma (DLBCL), primary mediastinal B-cell lymphoma (PMBL), Hodgkin lymphoma (HL), non-Hodgkin lymphoma (NHL), mediastinal gray zone lymphoma, or nodular sclerosis HL. The T-cell lymphoma can be anaplastic large cell lymphoma (ALCL), peripheral T cell lymphoma (PTCL), peripheral T cell lymphoma not otherwise specified (PTCL-NOS), or angioimmunoblastic T cell lymphoma (AITL).

In some embodiments, provided herein is a method of to treating a lymphoma in a subject in need thereof, comprising administering to the subject a therapeutically effective amount of the CD30 CAR-expressing cells or pharmaceutical compositions disclosed herein. In some embodiments, the lymphoma is B-cell lymphoma. In some embodiments, the lymphoma is DLBCL. In some embodiments, the lymphoma is PMBL. In some embodiments, the lymphoma is HL. In some embodiments, the lymphoma is NHL. In some embodiments, the lymphoma is mediastinal gray zone lymphoma. In some embodiments, the lymphoma is nodular sclerosis HL. In some embodiments, the lymphoma is extra-nodal NK-T-cell lymphoma. In some embodiments, the lymphoma is diffuse large B-cell lymphoma. In some embodiments, the lymphoma is EBV-positive diffuse large B-cell lymphoma. In some embodiments, the lymphoma is T-cell lymphoma. In some embodiments, the lymphoma is ALCL. In some embodiments, the lymphoma is PTCL. In some embodiments, the lymphoma is PTCL-NOS. In some embodiments, the lymphoma is CTCL. For example, in some embodiments, provided herein is a method of to treating a HL in a subject in need thereof, comprising administering to the subject a therapeutically effective amount of the CD30 CAR-expressing cells or pharmaceutical compositions disclosed herein. In particular embodiments, the CD30 CAR-expressing cells are CAR T cells.

Neoplastic mast cells in advanced systemic mastocytosis have been shown to express CD30. In some embodiments, provided herein is a method of to treating a CD30-expressing solid cancer or tumor in a subject in need thereof, comprising administering to the subject a therapeutically effective amount of the CD30 CAR-expressing cells or pharmaceutical compositions disclosed herein. In some embodiments, the CD30-expressing cancer or tumor is a germ cell tumor. In some embodiments, the CD30-expressing cancer or tumor is an embryonal carcinoma (EC). In some embodiments, the CD30-expressing cancer or tumor is testicular embryonal carcinomas. In some embodiments, the CD30-expressing cancer or tumor is a testicular germ cell tumor (TGCT).

In some embodiments, the methods disclosed herein can increase the levels of TNFα and/or IL12p70 in peripheral blood of the subject having a CD30-expressing cancer. In some embodiments, the methods disclosed herein can decrease the number of CD30 positive tumor cells. In some embodiments, the methods disclosed herein can result in a shrinkage of lymph node masses. In some embodiments, the methods disclosed herein can result in a shrinkage of lymph node masses. In some embodiments, a pharmaceutical composition can decrease measurable lymph nodes and/or extranodal burdens. In some embodiments, the methods disclosed herein can decrease tumor burden in the subject.

Methods for monitoring patient response to administration of a pharmaceutical composition disclosed herein are known in the art and can be employed in accordance with methods disclosed herein. In some embodiments, methods known in the art can be employed to monitor the patient for response to administration of a pharmaceutical composition disclosed herein. In some embodiments, methods known in the art can be used to monitor size of lesions, and/or size of lymph nodes.

As a non-limiting example, in some embodiments, contrast-enhanced CT scans can detect and/or monitor lesions and/or lymph nodes in a patient. In some embodiments, administration of a pharmaceutical composition disclosed herein can reduce the size of lesions detected by CT scans in a patient. In some embodiments, administration of a pharmaceutical composition disclosed herein can cause shrinkage of abnormal lymph nodes.

In some embodiments assays can be used to detect infiltration of a pharmaceutical composition disclosed herein (e.g., CAR T cells) in tumor cells and/or lymph nodes. In some embodiments, the persistence of CD30 CAR-expressing cells and/or cell populations at a specific site can be detected using methods known in the art. As a non-limiting example, immunohistochemistry and/or qPCR can be used to detect infiltration of cells and/or cell populations of the invention at a specific site (e.g., in tumor cells and/or lymph nodes). In some embodiments, copy numbers of CD30 CAR transgenes in peripheral blood can be detected using methods known in the art.

In some embodiments, the CAR-expressing cells to be administered can be purified or enriched. For example, the methods provided herein can be used to treat cancer or reduce tumor burden in a subject, wherein the cancer or tumor is CD30-expressing cancer or tumor. In one embodiment, the methods provided herein are used to treat cancer. It is understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers. In one non-limiting example, the methods provided herein can reduce tumor burden. Thus, administration of the cells of the invention can reduce the number of tumor cells, reduce tumor size, and/or eradicate the tumor in the subject. The tumor can be a solid tumor. The methods of the invention can also provide for increased or lengthened survival of a subject having cancer. Additionally, methods of the invention can provide for an increased immune response in the subject against the cancer.

In the methods of the invention, the immune cells or precursor cells thereof are administered to a subject in need of cancer treatment. The subject can be a mammal. The subject is a human A pharmaceutical composition comprising a cell of the invention is administered to a subject to elicit an anti-cancer response, with the objective of palliating the subject's condition. Eliminating cancer or tumor cells in a subject can occur, but any clinical improvement constitutes a benefit. Clinical improvement comprises decreased risk or rate of progression or reduction in pathological consequences of the cancer or tumor.

Another group of suitable subjects can be a subject who has a history of cancer, but has been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for different types of cancers. Features typical of high-risk subgroups are those in which the tumor has invaded neighboring tissues, or who show involvement of lymph nodes. Optionally, a cell of the invention can be administered for treatment prophylactically to prevent the occurrence of cancer in a subject suspected of having a predisposition to a cancer, for example, based on family history and/or genetic testing.

The subject can have an advanced form of disease, in which case the treatment objective can include mitigation or reversal of disease progression, and/or amelioration of side effects. The subjects can have a history of the condition, for which they have already been treated, in which case the therapeutic objective can be to decrease or delay the risk of recurrence. Additionally, refractory or recurrent malignancies can be treated using the cells or pharmaceutical compositions disclosed herein. In some embodiments, provided herein is a method of to treating a HL in a subject in need thereof, comprising administering to the subject a therapeutically effective amount of the CD30 CAR-expressing cells or pharmaceutical compositions disclosed herein, wherein the subject with HL is refractory to or relapse after the first-line treatment (e.g. chemotherapy) or autologous stem cell transplantation (ASCT).

Any CD30 CAR-expressing cells can be used in the methods disclosed herein. As described above, in some embodiments, the cells are T cells including but are not limited to, T helper cells (CD4+), cytotoxic T cells (CD8+), natural killer T cells, γδT cells, mucosal associated invariant T cells (MAIT), and memory T cells, including central memory T cells, stem-cell-like memory T cells (or stem-like memory T cells), and effector memory T cells, for example, TEM cells and TEMRA (CD45RA+) cells. In some embodiments, CD30 CAR-expressing cells used in methods described herein are cytotoxic T cells. In some embodiments, CD30 CAR-expressing cells used in methods described herein are T helper cells. In some embodiments, the CD30 CAR expressing cells that are administered to the subject comprise both cytotoxic T cells and T helper cells, thus generating both helper and cytotoxic T cell responses in the subject.

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the cells of the invention being administered.

The cells of the invention are generally administered as a dose based on cells per kilogram (cells/kg) of body weight of the subject to which the cells are administered.

Generally the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to 10⁶, depending on the mode and location of administration. In general, in the case of systemic administration, a higher dose is used than in regional administration, where the immune cells of the invention are administered in the region of a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 3×10⁵ to 3×10⁶ cells/kg, and the like. Such dose ranges can be particularly useful for regional administration. In a particular embodiment, cells disclosed herein are provided in a dose of 1×10⁵ to 1×10⁸, for example 1×10⁵ to 1×10⁷, 1×10⁵ to 1×10⁶, 1×10⁶ to 1×10⁸, 1×10⁶ to 1×10⁷, 1×10⁷ to 1×10⁸, 1×10⁵ to 5×10⁶, in particular 1×10⁵ to 3×10⁶ or 3×10⁵ to 3×10⁶ cells/kg for regional administration, for example, intrapleural administration. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷ cells/kg, and the like. Such does can be particularly useful for systemic administration. In a particular embodiment, cells are provided in a dose of 1×10⁶ to 3×10⁷ cells/kg for systemic administration.

Exemplary cell doses include, but are not limited to, a dose of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹ and so forth in the range of about 10⁴ to about 10¹⁰ cells/kg. In addition, the dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The cells or pharmaceutical compositions provided herein can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus. In some embodiments, the cells or pharmaceutical compositions provided herein can be administered intravenously. In one embodiment, the cells provided herein can be delivered regionally to a tumor using well known methods, including but not limited to, hepatic or aortic pump; limb, lung or liver perfusion; in the portal vein; through a venous shunt; in a cavity or in a vein that is nearby a tumor, and the like. In another embodiment, the cells provided herein can be administered systemically. In a preferred embodiment, the cells are administered regionally at the site of a tumor. The cells can also be administered intratumorally, for example, by direct injection of the cells at the site of a tumor and/or into the tumor vasculature. One skilled in the art can select a suitable mode of administration based on the type of cancer and/or location of a tumor to be treated. The cells can be introduced by injection or catheter. In one embodiment, the cells are pleurally administered to the subject in need, for example, using an intrapleural catheter. Optionally, expansion and/or differentiation agents can be administered to the subject prior to, during or after administration of cells to increase production of the cells of the invention in vivo.

Proliferation of the cells of the invention is generally done ex vivo, prior to administration to a subject, and can be desirable in vivo after administration to a subject (see Kaiser et al., Cancer Gene Therapy 22:72-78 (2015)). Cell proliferation should be accompanied by cell survival to permit cell expansion and persistence, such as with T cells.

The methods provided herein can further comprise adjuvant therapy in combination with, either prior to, during, or after treatment with the cells of the invention. Thus, the cell therapy methods of the invention can be used with other standard cancer care and/or therapies that are compatible with administration of the cells of the invention.

In certain embodiments, the CD30-expressing cells or pharmaceutical compositions disclosed herein are administered more than once to the subject in need thereof. In some embodiment, a subject receives the second administration less than about 15 days after the first administration. In some embodiment, a subject receives the second administration less than about 14, less than about 13, less than about 12, less than about 11, less than about 10, less than about 9, less than about 8, less than about 7, less than about 6, less than about 5, less than about 4, less than about 3, or less than about 2 days after the first administration. In one embodiment, the CD30-expressing cells or pharmaceutical compositions disclosed herein are administered biweekly, weekly, every two weeks, or monthly to the subject in need thereof.

In some embodiments, the CD30 CAR expressing cells used in methods described herein are autologous to the subject to which they are administered. In some embodiments, the methods disclosed herein further comprise obtaining the parent immune or precursor cells from the subject. The parent immune or precursor cells can be obtained from a number of sources known in the art. In some embodiments, cells can be obtained from peripheral blood mononuclear cells, bone marrow, lymph node tissue, cord blood, thymus tissue, tissue from a site of infection, ascites, pleural effusion, spleen tissue, and tumors. In some embodiments, the cells can be obtained by leukapheresis. In some embodiments, the CD30 CAR expressing cells used in methods described herein are non-autologous to the subject to which they are administered. In some embodiments, the methods disclosed herein further comprise HLA-matching to determine an appropriate level of compatibility. Method of HLA-matching are also well known in the art.

In some embodiments, the CD30 CAR-expressing cells used in methods described herein are CAR T cells. Prior to expansion and genetic modification of the parent T cells to produce CAR T cells using methods disclosed herein, the parent T cells can be obtained from a subject using methods known in the art. In some embodiments, T cells can be obtained from blood collected from a subject using any number of techniques known to the skilled artisan (e.g., Ficoll separation, apheresis). In some embodiments, T cells can be isolated from peripheral blood lymphocytes by lysing the red blood cells and depleting the monocytes using methods known in the art (e.g., by centrifugation through a gradient or by counterflow centrifugal elutriation). In certain embodiments, a specific subpopulation of T cells can be further isolated by positive or negative selection techniques known in the art.

The methods described herein relate to generating cancer-targeted immune cells, or precursor cells thereof, for adoptive therapy to enhance immune cell function through the design of improved antigen receptors and inclusion of cell intrinsic inhibition of immune checkpoint pathways. In some embodiments, the methods provided herein can further comprise administering an additional therapy to the subject. In some embodiments, the additional therapy is chemotherapy, radiation therapy, combined-modality therapy (CMT), autologous stem cell transplantation (ASCT). Optionally, the methods of administering cells provided herein can additionally include immunomodulation of the host to facilitate the effectiveness of the administered cells of the invention in combination therapy. In some embodiments, the additional therapy comprises administering one immunomodulatory agent. Non-limiting examples of immunomodulatory agents include immunostimulatory agents and checkpoint immune blockade agents.

Combination therapy using agents with different mechanisms of action can result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that resistant cancer cells will develop. In some embodiments, the additional therapy results in an increase in the therapeutic index of the cells or pharmaceutical compositions described herein. In some embodiments, the additional therapy results in a decrease in the toxicity and/or side effects of cells or pharmaceutical compositions described herein.

The additional therapy can be administered prior to, concurrently with, or subsequent to administration of the cells or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering cells described herein and an additional therapy in combination, including the timing and dosing of an additional agent to be used in a combination therapy, based on the needs of the subject being treated.

It is understood that modifications which do not substantially affect the activity of the various embodiments of this invention are also provided within the definition of the invention provided herein. Accordingly, the following examples are intended to illustrate but not limit the present invention.

### EXAMPLES

The examples provided below are for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1 Animal Immunization and Library Construction

### Animal Immunization

One camel was immunized with recombinant human CD30 protein under all current animal welfare regulations. For immunization, the antigen was formulated as an emulsion with CFA (primary immunization) or IFA (boost immunizations). The antigen was administered by double-spot injections intramuscularly at the neck. The animal received two injections of the emulsion, containing 100 μg of recombinant human CD30 extra cellular domain (ECD) protein (Accession # P28908, F19-K379, SEQ ID NO: 1) from R&D systems (Cat. #6126-CD) and 4 subsequent injections containing 50 μg of human CD30 ECD protein at weekly intervals. At different time points during immunization, 10 ml blood samples were collected from the animal and sera were prepared. Conventional IgG (IgG1) and heavy chain antibodies (HCAbs, IgG2 and IgG3) were fractioned from pre-immune and immunized sera. The antigen specific humoral immune response was verified using the fractioned IgG1, IgG2 and IgG3 in an enzyme-linked immune sorbent assay (ELISA)-based experiment with immobilized human CD30 ECD (R&D systems) and rhesus CD30 ECD (Accession # EHH14336, F19-K379, SEQ ID NO: 2) produced in house.

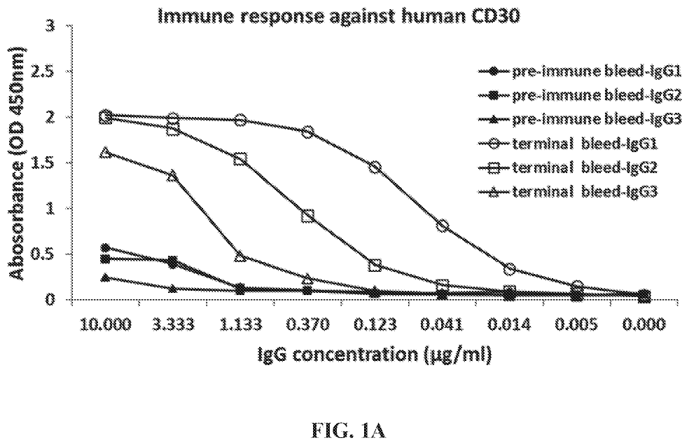

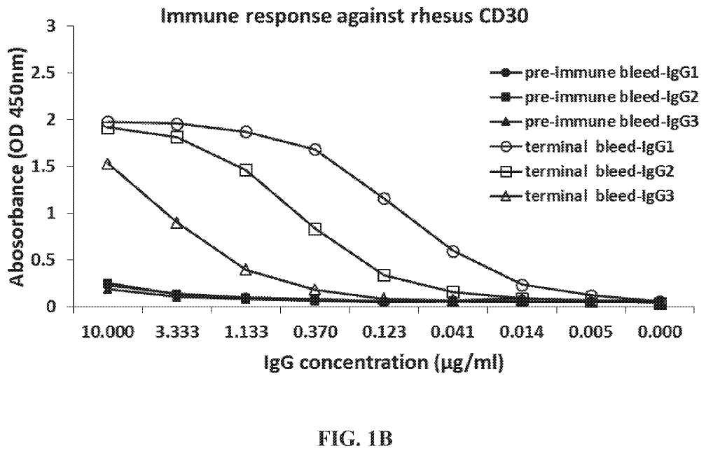

The immune response of the sixth immunizations was high (FIGS. 1A and 1B). Five days afterwards, 150 ml blood sample was collected from the camel (terminal bleed). About 1×10⁹ peripheral blood lymphocytes (PBLs), as the genetic source of the conventional and heavy chain immunoglobulins, were isolated from the blood. The maximal diversity of antibodies is expected to be equal to the number of B-lymphocytes, which is about 10% of total PBLs. The fraction of HCAb-producing B-lymphocytes in a camel is about 20% of total B-lymphocytes. Therefore, the maximal diversity of HCAbs in the blood sample is estimated to be approximately 2×10⁷.

### Phage Display Library Construction

Total RNA was extracted from lymphocytes of the immunized camel using TRIZOL® Reagent. cDNA was synthesized based on RNA template using PRIMESCRIPT™ 1^{st} Strand cDNA Synthesis Kit with an oligo(dT)20 primer. V_{H}Hs (V_{H}H referring to the variable region of a heavy chain antibody) were amplified from camel cDNA, purified and ligated in an in house phagemid vector. The ligation product was used to transform SS320 electrocompetent cells. The resulting library was supplemented with 20% glycerol and stored at −80° C.

The size of the library is estimated to be larger than 10⁹. More than 100 randomly picked clones were sequenced. The insert rate, i.e. the percentage of clones with sdAb inserts, was 98.7%. The in-frame rate, i.e. the percentage of clones with sdAb DNA inserted that could be correctly translated into a sdAb amino acid sequence, was 96.6%.

### Example 2 Anti-CD30 Antibody Generation and Characterization

Anti-CD30 antibodies provided herein include single domain antibodies (sdAbs) generated from an immunized camel or human Fab isolated from synthetic human Fab library.

### Phage Display

Both immunized sdAb and synthetic human Fab phage libraries were rescued and stored after filter sterilization at 4° C. for further use. Binders were isolated with the above-mentioned phage libraries using protein-based panning as well as cell-based panning. At least one round of panning was carried for both protein- and cell-based panning approaches using both libraries until the percentage of CD30-specific phage clones reached 30%. Output phage of each round were assessed for the number of total output clones, percentage of CD30 positive clones by ELISA and sequence diversity of CD30-specific clones. Based on these results the best panning outputs were selected for high-throughput screening.

### High-Throughput Screening

The selected output phages were used to infect exponentially growing E. coli cells. The double-strand DNA of the output was extracted. The sdAb/Fab inserts were cut from the phagemid vector and inserted into an antibody fragment expression vector for high-throughput screening. The resulting plasmid was used to transform exponentially growing E. coli cells, which were subsequently plated and grown overnight at 37° C. Thousands of colonies were picked individually and grown in 96 deep well plates containing 1 ml 2YT medium. The expression of antibody fragment was induced by adding 1.0 mM IPTG.

The sdAb/Fab proteins in the supernatant were analyzed for their ability to bind to human and rhesus CD30 ECD proteins by ELISA and CD30 expressing HH cell lines (cutaneous T cell lymphoma (CTL), American Type Culture Collection (ATCC)® CRL2105™) by FACS. All binders were sequenced. The redundant sequences were removed. All together, 38 camel sdAb and 74 human Fab binders that bound both human and rhesus CD30 proteins and cell lines were obtained. All these binders have unique sequences.

Some of these unique binders were subjected to for further characterization by surface plasmon resonance (SPR) on a BIAcore T200 instrument (GE Healthcare). The experiment was carried out as follows. The crude sdAb/Fab proteins were captured through an affinity tag (6×Histidine tag) onto the sensorchip. High-concentration (100.0 nM) of human CD30 (R&D systems, Cat. #6126-CD) flowed over the sensorchip surface, and were allowed to bind the antibody fragments for 300 s followed by injection of the running buffer to allow dissociation of the complex. On-rate (10 and off-rate (k_{d}) were roughly calculated based on one association and dissociation curve, and were used to estimate the equilibrium dissociation constant (K_{D}). 5F11 scFv (SEQ ID NO:216, prepared according to WO2017066122) was used as a positive control. Data were summarized in Table 3 below. The first 10 antibodies in Table 3 were sdAbs with amino acid sequence ID numbers summarized in Table 1, the last antibody was 5F11 scFv, and the remainings were human Fabs with CDR and scFv amino acid sequence ID numbers in Table 2.

The binding affinities of these binders were not high, ranging from 3.0 nM to 170.0 nM, compared to that of 5F11 scFv, which was below 100.0 pM (Table 3).

TABLE 3
Affinity ranking of camel sdAb and human Fab binders

| | Antibody | rhCD30 | | | |
|---|---|---|---|---|---|
| Antibody | capture | Binding | | | |
| ID | (RU) | (RU) | kₐ (1/Ms) | k_{d} (1/s) | K_{D} (M) |
| AS47863 | 446.9 | 118.6 | 1.4E+05 | 4.2E−03 | 2.9E−08 |
| AS48433 | 362.1 | 83.5 | 9.3E+04 | 2.3E−03 | 2.5E−08 |
| AS48463 | 289.3 | 106 | 3.4E+05 | 1.0E−02 | 3.0E−08 |
| AS48481 | 216.1 | 26.3 | 8.6E+04 | 8.1E−03 | 9.5E−08 |
| AS48508 | 214 | 69.8 | 2.0E+05 | 6.1E−03 | 3.1E−08 |
| AS48542 | 278.9 | 60.8 | 1.2E+05 | 2.4E−03 | 1.9E−08 |
| AS53750 | 807.1 | 284.2 | 5.6E+04 | 6.9E−03 | 1.3E−07 |
| AS54233 | 502.5 | 59.2 | 6.1E+04 | 4.6E−03 | 7.6E−08 |
| AS53445 | 481.9 | 100.7 | 1.1E+05 | 8.5E−03 | 8.0E−08 |
| AS53574 | 946.2 | 568.9 | 6.9E+04 | 2.1E−04 | 3.0E−09 |
| AS57911 | 1213 | 44.6 | 9.2E+04 | 1.5E−02 | 1.7E−07 |
| AS57659 | 1120.2 | 83.6 | 8.2E+04 | 2.2E−03 | 2.7E−08 |
| AS57765 | 908.8 | 37.7 | 9.8E+04 | 1.1E−03 | 1.1E−08 |
| 5F11-scFv | 382.1 | 25.8 | 1.2E+05 | <1.0E−05* | <8.62E−11 |

*beyond off-rate detection limit of Biacore T200 which is 1.0E−05.

### Epitope Determination

Cysteine rich domains (CRDs) of human CD30 and the flanking residues, i.e., CRD1 (F19-Q68, SEQ ID NO: 3), CRD2 (R66-E107, SEQ ID NO: 4), CRD3 (E107-5153, SEQ ID NO: 5), CRD4 (E150-Q243, SEQ ID NO: 6), CRD5 (R241-E282, SEQ ID NO: 7) and CRD6 (E282-K379, SEQ ID NO: 8), were produced as Fc fusion proteins with human IgG1 Fc fragment (SEQ ID NO: 217) at the C terminus and used for epitope determination using SPR following the protocol similar to what was described above, only the analytes used were human IgG1 Fc-fused CRD proteins.

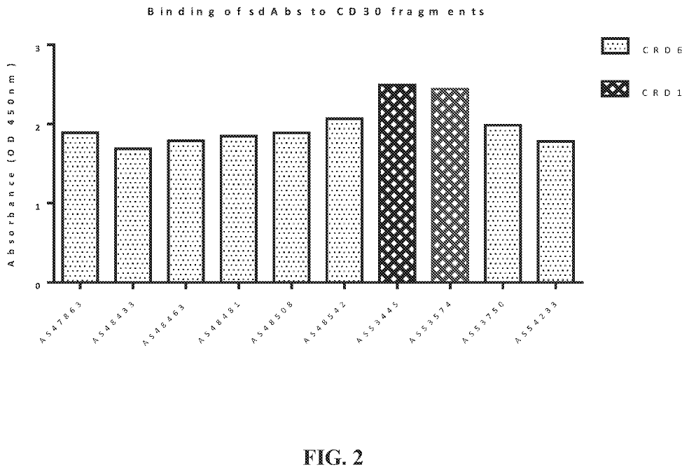

Of all binders, 8 sdAb binders, i.e., AS47863 (SEQ ID NO: 9), AS48433 (SEQ ID NO: 10), AS48463 (SEQ ID NO: 11), AS48481 (SEQ ID NO: 12), AS48508 (SEQ ID NO: 13), AS48542 (SEQ ID NO: 14), AS53750 (SEQ ID NO: 17), and AS54233 (SEQ ID NO: 18), were confirmed to bind CRD6 (membrane-proximal binders), same as the positive control 5F11 scFv (SEQ ID NO: 216); 2 sdAb binders, i.e., AS53445 (SEQ ID NO: 15) and AS53574 (SEQ ID NO: 16), were confirmed to bind CRD1 (membrane-distal binders) (FIG. 2). The 3 human Fabs did not seem to specifically bind any of the CRD constructs.

### Example 3 In Vitro Efficacy of Camel sdAb and Human scFv Single-CD30 CAR Constructs

### Cloning of Anti-CD30 CAR Constructs

The 10 single-domain antibodies of Example 2 and human scFvs prepared from the 3 human Fabs of Example 2 (amino acid sequences set forth in SEQ ID NOs: 58, 59 and 60) were used as CD30-binding moieties for CAR constructs, with the 5F11 scFv used as the binding moiety for CAR positive control.

The anti-CD30 CAR constructs were designed in the format of a conventional 2^{nd} generation CAR, henceforth called naked CARs. As provided above, the sequences of these CARs contained from the N-terminus to the C-terminus: leader sequence (SEQ ID NO: 61), target binding moiety (i.e. anti-CD30 sdAb or scFv), CD8α hinge (SEQ ID NO: 62), CD8α transmembrane (TM) region (SEQ ID NO: 63), the cytoplasmic portion of the 4-1BB (CD137) molecule (SEQ ID NO: 64), and the cytoplasmic portion of the CD3ζ molecule (SEQ ID NO: 65). These constructs were designated “[CD30-binding moiety]bbz”. For example, the naked CAR construct using AS48542 sdAb was designated AS48542bbz.

### Generation of Anti-CD30 CAR T Cells

Lentiviral production: The lentivirus packaging plasmids including pCMV-ΔR-8.47 and pMD2.G (Addgene, Cat #12259) were mixed with Legend's in house CAR-encoding lentiviral transfer plasmid at approximately equal molar ratio with polyethylenimine (PEI) at plasmids: PEI ratio of 1:4.5. HEK293 cells were transfected with the mixture and were cultured overnight. The culture supernatant was collected and centrifuged to remove cell debris. The supernatant was filtered through a 0.45 μm PES filter. The virus particles were pelleted, and rinsed with pre-chilled Dulbecco's Phosphate Buffered Saline (DPBS). The virus was aliquoted and stored at −80° C. immediately. The virus titer was determined by measuring supT1 cell line transduction efficiency by flow cytometric assay.

T cell transduction: Leukocytes were collected from healthy donors by apheresis. Peripheral blood mononuclear cells (PBMCs) were isolated using Ficoll-Paque™ PLUS Media. Human T cells were purified from PMBCs using Pan T cell isolation kit (Miltenyi, Cat #130-096-535). The purified T cells were subsequently pre-activated for 48 hours with human T cell activation/expansion kit (Miltenyi, Cat #130-091-441), where anti-CD3/CD28 MACSiBead particles were added at a bead-to-cell ratio of 1:2. The pre-activated T cells were transduced with lentivirus stock at multiplicity of infection (MOI) between 2:1 and 10:1 in the presence of 8 μg/ml polybrene. The cells were cultured in AIM V™ medium (Thermofisher, Cat #31035025) supplemented with 5% FBS (GIBCO, Cat #10099-141) and 200 U/mL rIL2 in 6-well tissue culture plates (Corning, Corning, N.Y.) at 4.0×10⁶ T cells/well density. The cells were cultured for approximately 48 hours at 37° C. The transduced cells were centrifuged and resuspended at 0.5×10⁶ cells/ml density in fresh medium supplemented with 5% FBS and 200 U/mL IL-2. This process was repeated every 2 to 3 days until enough cells were obtained.

For CAR expression on T cells, protein L and rabbit-anti-sdAb (GenScript, Piscataway, N.J.) were added to detect the cell surface scFvs and sdAbs, respectively. The CAR expression levels were shown in Table 4.

### Cytotoxicity by LDH Assay

On day 5 of transduction, transduced T cells were harvested and co-incubated with CD30-expressing tumor cell line HH (at cell density of approximately 1×10⁵ cells/mL), at effector cell: target cell ratio of 0.5:1 for 20 hours. 5F11 scFv CAR T cells were used as a positive control in all assays. Untransduced T cells were used as a negative control.

Lactate dehydrogenase (LDH) level was measured at endpoint. The cytotoxicity was defined as follows:

$$cytotoxicity = \frac{{[ LDH ]}_{e + T} - {[ LDH ]}_{E} - {[ LDH ]}_{T}}{{[ LDH ]}_{max}}$$

where [LDH]_{E+T} is the LDH level of effector and target cell mixture, [LDH]_{E} is the LDH level of effector cells alone, [LDH]_{T} is the LDH level of target cells alone and [LDH]ₘₐₓ is the LDH level when target cells are completely lysed by 1% Triton X-100 (JK chemical, Cat #993361).

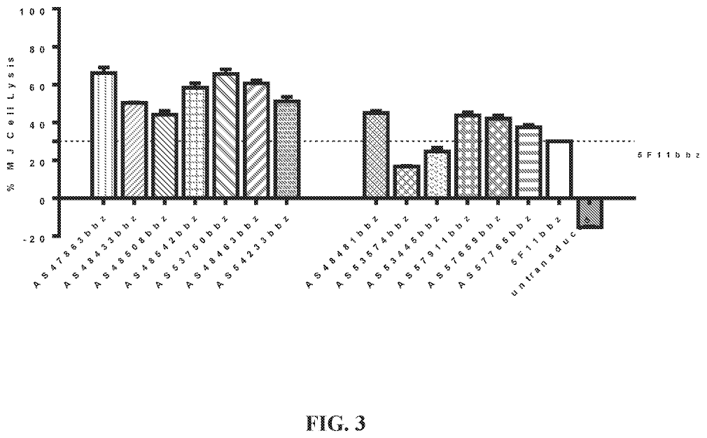

According to the assay result, the in vitro cytotoxicity of T cells transduced with 8 membrane-proximal-binder CAR constructs (i.e. AS47863bbz, AS48433bbz, AS48508bbz, AS48542bbz, AS53750bbz, AS48463bbz, AS54233bbz and AS48481bbz) were superior to that of the 5F11bbz CAR T cells (Table 4, FIG. 3), whereas T cells with the membrane-distal-binder CAR T constructs (i.e. AS53574bbz and AS53445bbz) showed inferior in vitro cytotoxicity. The position of the targeted epitope within the molecule can have major impact on the efficacy of T cell activation (Hombach et al. J Immunol., 178: 4650-4657(2007)). The fact that 5F11 antibody is a CRD6 binder with extremely high binding affinity toward the target CD30 and yet 5F11bbz CAR T cells did not show better cytotoxicity than the abovementioned 8 CAR constructs which used lower-affinity anti-CRD6 sdAbs as CD30 binding moiety indicated that T cell activation may be independent of the binding affinity of the immunoreceptor. These data support that the affinity thresholds for CAR immunoreceptors exist, above which T cell function cannot be improved, or is rather reduced (Chmielewski et al. J Immunol., 173(12):7647-53(2004)). The T cells transduced with 3 scFv CARs (i.e., AS57911bbz, AS57659bbz and AS57765bbz) also showed higher cytotoxicity than 5F11bbz CAR T cells.

TABLE 4
Expression level and Cytotoxicity
of selected CAR T cells

| | % CAR+ | % MJ |
|---|---|---|
| | Day 5 of | cell lysis |
| CAR construct | transduction | E:T = 0.5:1 |
| AS47863bbz (SEQ ID NO:70) | 83.5% | 65.0% |
| AS48433bbz (SEQ ID NO:71) | 68.9% | 50.5% |
| AS48508bbz (SEQ ID NO:74) | 54.6% | 44.1% |
| AS48542bbz (SEQ ID NO:75) | 82.0% | 58.4% |
| AS53750bbz (SEQ ID NO:76) | 88.7% | 65.7% |
| AS48463bbz (SEQ ID NO:72) | 78.6% | 60.8% |
| AS54233bbz (SEQ ID NO:77) | 66.0% | 51.0% |
| AS48481bbz (SEQ ID NO:73) | 59.7% | 45.1% |
| AS53574bbz (SEQ ID NO:79) | 72.9% | 16.6% |
| AS53445bbz (SEQ ID NO:78) | 73.2% | 24.6% |
| AS57911bbz (SEQ ID NO:80) | 79.4% | 43.9% |
| AS57659bbz (SEQ ID NO:81) | 69.3% | 42.0% |
| AS57765bbz (SEQ ID NO:82) | 64.4% | 37.3% |
| 5F11bbz | 65.3% | 29.9% |
| Untransduced | N/A | −15.2% |

This example demonstrated that most of the CAR T cells which used the selected camel sdAb and human scFv as the CD30-binding moiety had similar or superior in vitro cytotoxicity to CD30-expressing cell lines, compared to the positive control 5F11bbz CAR T cells.

### Example 4 Evaluation of Anti-CD30 CAR T Cells in In Vivo Mouse Model

Anti-tumor activity of anti-CD30 CAR T cells was assessed in vivo in an HH xenograft model. Five million (5.0×10⁶) HH cells were implanted subcutaneously on day 0 in NSG mice. Once tumors size reached 150-250 mm³, mice were randomized into treatment groups (3-4 mice in each group). CAR+ T cells (2.0×10⁶ or 5.0×10⁶ per mouse) in 200 μl volume were administered intravenously. Mice and tumor size were monitored for more than 2 months after tumor cell implantation. Mice were euthanized when the tumor size reached 2000 mm³.

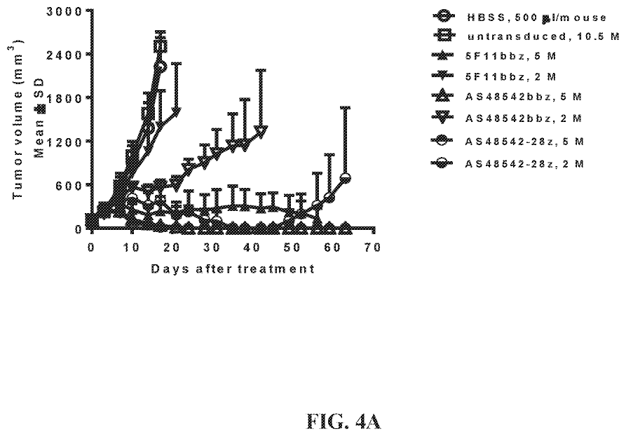

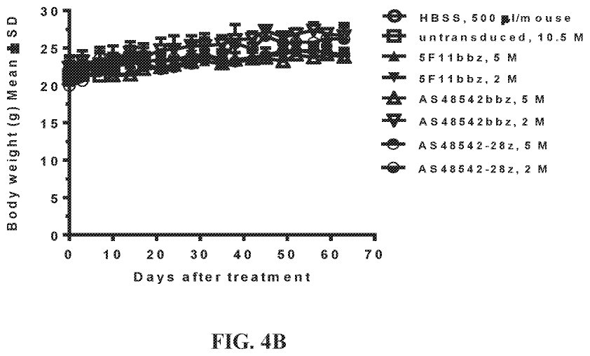

As shown in FIG. 4A, AS48542bbz T cells and AS48542-28z CAR T cells at 5×10⁶ dose showed similar excellent tumor growth inhibition efficacy. All animals in these two groups became tumor free 20 days post CAR T cell administration. On the other hand, only one out of three animals became tumor free after treatment by 5×10⁶ 5 F11bbz CAR T cells. At 2×10⁶ dosage, AS48542-28z CAR T cells showed better efficacy than AS48542bbz CAR T cells, while 5F11bbz CAR T cells showed the worse efficacy. The body weight of all animals remained at about the same level, as shown in FIG. 4B. The result indicated that AS48542bbz is a better CAR than the positive control 5F11bbz, which is consistent with the in vitro assay result shown in Example 3.

CAR designated “AS48542-28z”, as used above, contained, from N-terminus to the C-terminus, a leader sequence (SEQ ID NO: 61), AS48542 sdAb, CD28 hinge (SEQ ID NO: 127), CD28 transmembrane (TM) region (SEQ ID NO: 128), the cytoplasmic portion of CD28 molecule (SEQ ID NO: 129), and the cytoplasmic portion of the CD3ζ molecule (SEQ ID NO: 65).

Considering that 6 membrane-proximal CRD6 binders (i.e. AS47863, AS48433, AS48542, AS53750, AS48463 and AS54233) are highly homologous in amino acid sequence, coupled with the fact that they showed similar in vitro efficacy, the in vivo efficacy of T cells with these sdAb CD30-binding moieties constructed CARs are expected to be comparable and all superior to that of 5F11bbz CAR T cells.

Although the in vivo efficacy of [CD30 binding moiety]-28z CAR T cells are proved to be more efficacious, [CD30 binding moiety]-bbz CAR T cells are generally believed to be more persistent than [CD30 binding moiety]-28z CAR T cells in clinical trials. Therefore, [CD30 binding moiety]-bbz constructs were further tested in the following assays.

### Example 5 In Vitro Cytotoxicity of Tandem-Repeat and Biparatopic CAR T Cells

This example demonstrated that using the tandem repeat of a membrane-proximal sdAb CD30-binding moiety could improve CAR T cell's cytotoxicity towards cell lines with relatively lower CD30 expression like H9, independently of linker length, whereas using biparatopic CD30-binding moieties showed comparable efficacy as using a single CD30 binding moiety.

For tandem-repeat CD30-binding moieties, two identical anti-CD30 sdAb molecules were linked by a short G4S linker or a long (G4S)₃ linker. The tandem-repeat AS48542 CAR construct with the short G45 linker was designated AS48542dis-bbz (SEQ ID NO: 83), and the one with the long (G45)₃ linker was designated AS48542dil-bbz (SEQ ID NO: 84). For biparatopic CD30-binding moieties, two anti-CD30 sdAbs which recognized different epitopes (e.g. CRD1 binder AS53574 and CRD6 binder AS48542) were linked by a (G45)₃ linker. The CAR construct with the N-terminal AS48542 and C-terminal AS53574 was designated AS48542-AS53574bil-bbz (SEQ ID NO:85), and the construct with the N-terminal AS53574 and C-terminal AS48542 was designated AS53574-AS48542bil-bbz (SEQ ID NO:86). CAR construction, lentivirus preparation and CAR T cell production were carried out as described in Example 3.

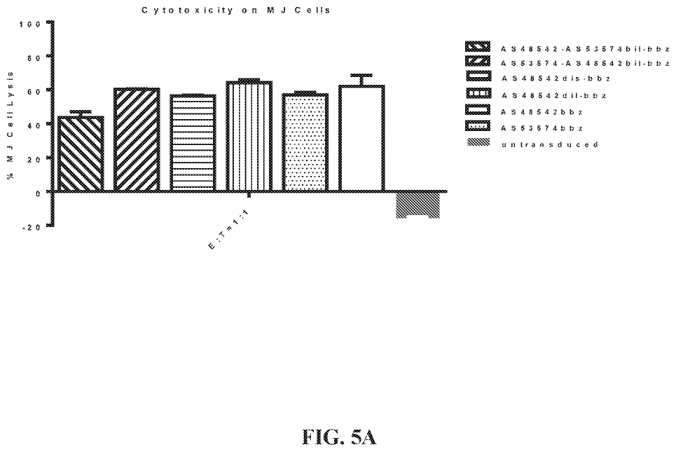

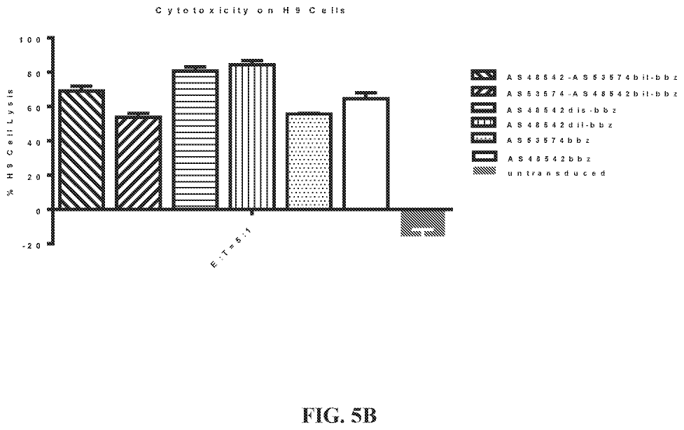

Nine days after transduction, in vitro cytotoxicity assay (LDH method) on CD30 high-expression MJ and CD30 low-expression H9 cell lines was carried out using tandem-repeat and biparatopic CAR T cells following the protocol described in Example 3. CAR expression level and cytotoxicity level were shown in Table 5, FIGS. 5A and 5B. The percentage of sdAb-based CAR-positive cells were all extremely high, >90%. All CAR constructs, whether they used a single sdAb CD30-binding moiety or tandem-repeat CD30-binding moiety or biparatopic CD30-binding moiety, showed similar cytotoxicity on MJ cell line. On the other hand, when H9 was used as the target cells, both tandem repeat CAR constructs showed superior cytotoxicity to AS48542bbz CAR T cells with single CD30-binding moiety, independently of the linker length, whereas the biparatopic CAR showed similar cytotoxicity compared with AS48542bbz CAR T cells.

TABLE 5
In vitro cytotoxicity of biparatopic and tandem
repeat CAR T on MJ and H9 cell lines.

| | % CAR T+ | % MJ | % H9 |
|---|---|---|---|
| | Day 10 of | cell lysis | cell lysis |
| Construct | transduction | E:T = 1:1 | E:T = 5:1 |
| AS48542-AS53574bil-bbz | 96.5% | 43.6% | 69% |
| AS53574-AS48542bil-bbz | 98.1% | 60.5% | 54% |
| AS48542dis-bbz | 95.1% | 56.4% | 81% |
| AS48542dil-bbz | 96.8% | 64.3% | 84% |
| AS53574bbz | 96.2% | 57.0% | 56% |
| AS48542bbz | 93.5% | 58.5% | 64% |
| Untransduced | NA | −14.9% | −15% |

### Example 6 Humanization of sdAbs and CARs

Selected camel sdAbs (SEQ ID NOs. 9-18) were humanized using CDR grafting technology (see, e.g., U.S. Pat. No. 5,225,539). The camel sdAb sequence was searched in NCBI human germline V gene database so the human VH germline sequence with highest identity to the sdAb (i e human acceptor) was identified (Foote and Winter, J. Mol. Biol. 224:487-499 (1992); Morea V. et al., Methods 20:267-279 (2000); Chothia C. et al., J. Mol. Biol. 186:651-663 (1985).). The most appropriate human frameworks on which to build the CDR grafted VH (henceforth called human acceptor) were listed in Table 6.

TABLE 6
Human acceptors selected for camel sdAbs

| Human acceptor | |
|---|---|
| accession # | Camel sdAb clones |
| AEX29643 | AS48433, AS54233 |
| AXA12214 | AS48508, AS53750 |
| CAE45450 | AS48463 |
| BAA36306 | AS48481 |
| AGP01450 | AS47863 |
| AEX29678 | AS53445 |
| AKU38584 | AS53574 |
| ABF83229 | AS48542 |

In CDR grafting approach, CDRs of the human acceptor were replaced by those of the camel sdAb, which made the straight-graft sequence. Straight-graft antibody usually loses binding activity, which can be restored by replacing the framework residues that are critical for the activity of the antibody with non-human residues. To identify these residues, a homology model of the camel sdAb was built. Briefly, the camel sdAb sequence was compared to those available in the Research Collaboratory for Structural Bioinformatics (RCSB) protein databank. The closest VH structure was used as a template based on which the homology model of camel sdAb was generated. From the model structure, residues that were in the proximity of CDRs or buried inside the molecule (i.e. with sidechain solvent accessible surface area less than 15%) or both were identified. These residues are usually important for the activity and structure of the antibody, therefore were considered potential back-mutation sites. Human residues in the potential back mutation sites were introduced to the straight-graft sequences step by step, resulting in humanized sdAbs with varying degrees of humanness (SEQ ID NOs. 19-54 and 199).

The camel and humanized sdAb sequences were fused with human CD8α hinge (SEQ ID NO: 62) and human IgG1 Fc fragment (SEQ ID NO:217), making the humanized HCAb sequences. The DNAs encoding these HCAbs were synthesized and inserted into pTT5 vector. HCAb expression plasmids were used to transfect HEK293 cells. Crude HCAb proteins secreted to the medium were subjected to SPR affinity measurement. Briefly, capturing antibody anti-human Fc pAb (GE healthcare) was immobilized on a Biacore™ CMS chip to approximately 6,000 RU using EDC-activated amine coupling chemistry. HCAbs of interest were captured for 300 seconds onto the sensorchip surface. Human CD30 (R&D systems, Cat. #6126-CD) flowed over the sensorchip surface at a series of increasing concentrations. Association and dissociation were monitored. Captured antibody and antigen were removed between cycles using 10 mM Glycine-HCl, pH 2.0 buffer in order to ensure a fresh binding surface for the next round. The resulting sensorgram was fit globally using a 1:1 binding model in order to calculate on- and off-rates (kₐ and k_{d}, respectively), as well as the binding affinities (K_{D}).

The binding affinities of some humanized HCAbs were compared with their chimeric counterparts. Most of the humanized HCAbs retained their binding affinities (Table 7).

TABLE 7
Monovalent binding affinity of camel and humanized antibodies.

| | kₐ | | | Rₘₐₓ | Chi² | U- |
|---|---|---|---|---|---|---|
| Antibody ID | (1/Ms) | k_{d} (1/s) | K_{D} (M) | (RU) | (RU²) | value |
| AS48542 | 2.2E+04 | 2.7E−03 | 1.2E−07 | 111.1 | 0.361 | 1 |
| AS48542VH5 | 2.9E+04 | 7.6E−03 | 2.6E−07 | 105.1 | 1.06 | 1 |
| AS48542VH12 | 2.7E+04 | 6.8E−03 | 2.5E−07 | 80.6 | 0.446 | 1 |
| AS48463 | 7.5E+04 | 2.0E−02 | 2.7E−07 | 46.31 | 0.082 | 1 |
| AS48463VH4 | 1.0E+05 | 4.9E−02 | 4.8E−07 | 30.40 | 0.026 | 2 |
| AS48463VH11 | 8.2E+04 | 4.2E−02 | 5.1E−07 | 40.76 | 0.048 | 1 |
| AS53445 | 2.4E+05 | 1.9E−02 | 7.8E−08 | 53.25 | 0.372 | 1 |
| AS53445VH4 | 1.5E+05 | 3.0E−02 | 2.0E−07 | 34.81 | 0.095 | 1 |
| AS53445VH5 | 1.4E+05 | 3.4E−02 | 2.4E−07 | 40.44 | 0.311 | 2 |
| AS53445VH11 | 2.3E+05 | 4.9E−02 | 2.1E−07 | 48.30 | 0.145 | 2 |
| AS53574 | 2.1E+05 | 3.1E−04 | 1.5E−09 | 54.71 | 0.345 | 3 |
| AS53574VH4 | 1.6E+05 | 4.4E−04 | 2.8E−09 | 54.23 | 0.328 | 2 |
| AS53574VH5 | 1.9E+05 | 4.2E−04 | 2.2E−09 | 48.04 | 0.306 | 2 |
| AS53574VH6 | 1.6E+05 | 3.9E−04 | 2.4E−09 | 53.62 | 0.301 | 2 |
| AS53574VH7 | 1.8E+05 | 4.3E−04 | 2.4E−09 | 55.67 | 0.542 | 4 |
| AS47863 | 3.2E+04 | 3.7E−03 | 1.2E−07 | 66.48 | 0.272 | 1 |
| AS47863VH4 | 4.4E+04 | 2.0E−02 | 4.5E−07 | 43.25 | 0.112 | 1 |
| AS47863VH11 | 5.2E+04 | 2.4E−02 | 4.6E−07 | 59.01 | 0.16 | 1 |
| AS53750 | 4.4E+04 | 3.7E−03 | 8.5E−08 | 168.9 | 0.351 | 1 |
| AS53750VH4 | 5.7E+04 | 2.4E−02 | 4.3E−07 | 225.6 | 0.341 | 1 |
| AS53750VH5 | 4.6E+04 | 1.1E−02 | 2.4E−07 | 229.6 | 0.378 | 1 |
| AS53750VH11 | 5.4E+04 | 2.4E−02 | 4.5E−07 | 220.7 | 0.471 | 1 |

CAR constructs using four humanized sdAbs, i.e., AS48542VH5bbz (SEQ ID NO: 182), AS48463VH4bbz (SEQ ID NO: 183), AS47863VH4bbz (SEQ ID NO: 184) and AS53574VH7bbz (SEQ ID NO: 185) were constructed and tested for cytotoxicity as described in Example 3.

TABLE 8
In vitro cytotoxicity of humanized
CAR T cells on MJ and H9 cell lines

| | % CAR T+ | % MJ | % H9 |
|---|---|---|---|
| | Day 6 of | cell lysis | cell lysis |
| CAR construct | transduction | E:T = 1:1 | E:T = 2:1 |
| AS48542VH5bbz | 89.9% | 65% | 62% |
| AS48463VH4bbz | 94.0% | 67% | 64% |
| AS47863VH4bbz | 97.5% | 57% | 63% |
| AS53574VH7bbz | 64.1% | 49% | 67% |
| 5F11bbz | 82.1% | 41% | 45% |
| AS48542bbz | 93% | 55% | 50% |
| Untransduced | N/A | −24% | −17% |

Compared to positive control 5F11bbz, T cells with all four humanized CARs showed higher cytotoxicity on both MJ and H9 cell lines. The humanized CAR AS48542VH5bbz had similar or slightly better cytotoxicity than camelid AS48542bbz CAR (Table 8 and FIG. 6). The result suggested that humanization of CAR constructs was successful.

### Example 7 In Vitro Cytotoxicity of Humanized Tandem-Repeat and Biparatopic CAR T Cells

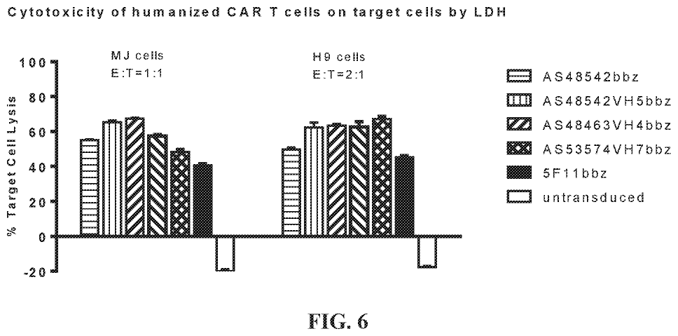

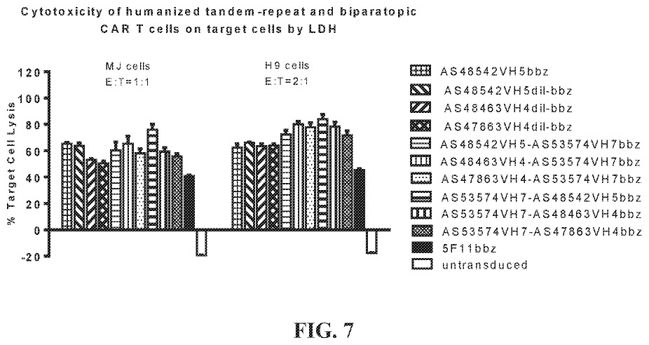

In this example, humanized sdAb sequences were used to construct humanized tandem-repeat and biparatopic CAR constructs (SEQ ID NOs: 186-194). Lentivirus preparation, CAR T cell production and in vitro cytotoxic assay by LDH method were carried out as described in Example 3. According to the result of this assay, all CAR T cells using humanized sdAb (single-binder, tandem-repeat or biparatopic) showed similar cytotoxicity to target cell lines, and they all were superior to the positive control 5F11bbz T cells. Swapping the humanized membrane-proximal and membrane-distal sdAb binders of the biparatopic CARs did not make much difference to the cytotoxic activity of CAR T cells (Table 9 and FIG. 7).

TABLE 9
In vitro cytotoxicity of humanized tandem-repeat
and biparatopic CAR T cells by LDH method.

| | % CAR T+ | % MJ | % H9 |
|---|---|---|---|
| | Day 6 of | cell lysis | cell lysis |
| CAR construct | transduction | E:T = 1:1 | E:T = 2:1 |
| AS48542VH5bbz | 89.9% | 65% | 62% |
| AS48542VH5dil-bbz | 70.5% | 64% | 66% |
| AS48463VH4dil-bbz | 93.5% | 53% | 63% |
| AS47863VH4dil-bbz | 96.1% | 50% | 64% |
| AS48542VH5-AS53574VH7bil-bbz | 87.1% | 60% | 72% |
| AS48463VH4-AS53574VH7bil-bbz | 88.1% | 66% | 80% |
| AS47863VH4-AS53574VH7bil-bbz | 90.6% | 58% | 78% |
| AS53574VH7-AS48542VH5bil-bbz | 87.3% | 76% | 84% |
| AS53574VH7-AS48463VH4bil-bbz | 88.0% | 59% | 78% |
| AS53574VH7-AS47863VH4bil-bbz | 88.1% | 56% | 72% |
| 5F11bbz | 82.1% | 41% | 45% |
| untransduced | N/A | −24% | −17% |

To differentiate these CARs, in vitro cytotoxic study was carried out at lower effector-to-target ratio of 0.2:1 using FACS method as follows.

Target cells (MJ or HH cell lines) were labelled with the fluorescent dye, carboxyfluourescein diacetate succinimidyl ester (CFSE (SIGMA-ALDRICH, Cat #21888)). The target cells were mixed with effector T cells that were either un-transduced or transduced with anti-CD30 CARs at cell density of approximately 1×10⁵ cells/mL, and incubated at 37° C., 5% CO2 for 48 hours. Flow cytometry was performed with a BD FACs Calibur machine. Analysis of flow cytometric data was performed using FlowJo software. The percentage of viable target cells of each samples was recorded. The ratio of viable target cell percentage in the presence of CAR transduced T cell sample over that in the presence of un-transduced T cell sample was calculated and subtracted from unity as follows:

$$cytotoxicity = 100 \% \times ( 1 - \frac{target cell percentage in CAR T well}{target cell percentage in untransduced T well} ) .$$

Such a value incorporates both target cell killing and T cell proliferation, and is a good indicator of the functional activity of CAR T cells.

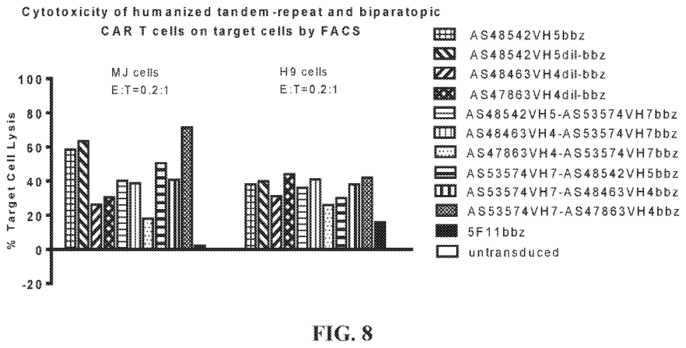

CAR expression level and cytotoxicity level were shown in Table 10 and FIG. 8, and the in vitro cytotoxicity of CAR T cells could be ranked according to the assay result. In consistence with the LDH assay result, all CAR T cells using humanized sdAb CARs showed superior cytotoxicity to that of T cells using the positive control 5F11bbz. T cells transduced with the single-binder CAR construct AS48542VH5bbz were highly toxic to both MJ and H9 cell lines. Of all tandem-repeat CAR constructs, AS48542VH5dil-bbz was the most toxic to MJ cells and AS47863VH4dil-bbz was the most toxic to H9 cells. Of all biparatopic CAR constructs, AS53574VH7-AS47863VH4bil-bbz was the most toxic to both MJ and H9 cells. All four constructs were further evaluated using in vivo mouse model.

TABLE 10
In vitro cytotoxicity of humanized tandem-repeat
and biparatopic CAR T by FACS.

| | % CAR T+ | % MJ | % H9 |
|---|---|---|---|
| | Day 6 of | cell lysis | cell lysis |
| CAR construct | transduction | E:T = 0.2:1 | |
| AS48542VH5bbz | 70.5% | 58.6% | 38.0% |
| AS48542VH5dil-bbz | 70.5% | 63.4% | 40.4% |
| AS48463VH4dil-bbz | 93.5% | 26.3% | 30.8% |
| AS47863VH4dil-bbz | 96.1% | 30.4% | 44.2% |
| AS48542VH5-AS53574VH7bil-bbz | 87.1% | 40.3% | 35.6% |
| AS48463VH4-AS53574VH7bil-bbz | 88.1% | 38.7% | 40.8% |
| AS47863VH4-AS53574VH7bil-bbz | 90.6% | 17.9% | 25.7% |
| AS53574VH7-AS48542VH5bil-bbz | 87.3% | 50.5% | 29.8% |
| AS53574VH7-AS48463VH4bil-bbz | 88.0% | 40.7% | 38.3% |
| AS53574VH7-AS47863VH4bil-bbz | 88.1% | 71.3% | 41.6% |
| 5F11bbz | 82.1% | 2.3% | 16.0% |
| untransduced | N/A | 0.0% | 0.0% |

### Example 8 Evaluation of Humanized Anti-CD30 CAR T Cells in In Vivo Mouse Model

Anti-tumor activities of AS48542VH5bbz, AS48542VH5dil-bbz, AS47863VH4dil-bbz and AS53574VH7-AS47863VH4 bil-bbz CAR T cells were assessed in vivo in an HH xenograft model as described in Example 4, except that CAR T cells were administered at a dose of 1×10⁶ or 2×10⁶ cells per mouse.

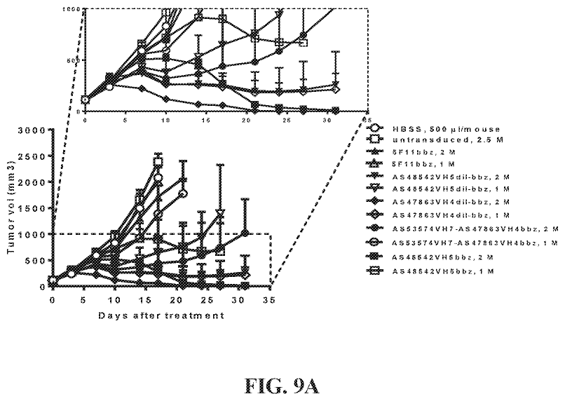

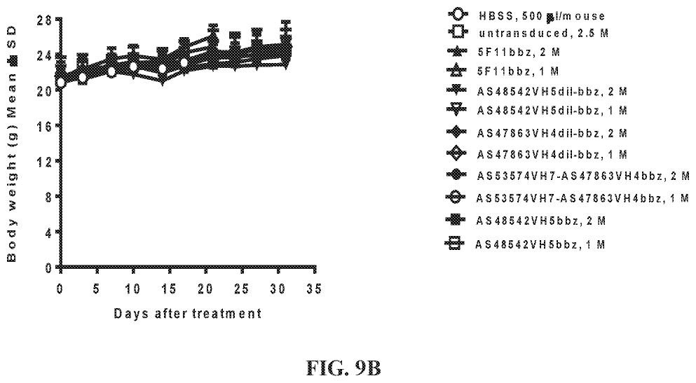

As shown in FIGS. 9A and 9B, at 2×10⁶ dosage, both AS48542VH5bbz and AS47863VH4dil-bbz could completely suppress tumor growth, and all animals were tumor-free at the end of the study. AS47863VH4dil-bbz CAR T cells started to suppress tumor growth on fifth day after the administration, whereas AS48542VH5bbz started to suppress tumor growth on tenth day after administration. Three out of 4 animals treated by 1×10⁶ AS47863VH4dil-bbz CAR T cells were tumor-free and one experienced tumor progression (tumor size˜766 mm³) at the end of the study. Two out of 4 animals treated by 1×10⁶ AS48542VH5bbz CAR T cells experienced partial tumor growth inhibition (tumor sizes reduced to ˜200 mm³). One animal experienced tumor progression (tumor size˜600 mm³) and one was euthanized due to large tumor burden. The result indicated that the in vivo efficacy of AS47863VH4dil-bbz is better than AS48542VH5bbz. All four animals treated by 2×10⁶ AS48542VH5dil-bbz CAR T cells experienced slow tumor progression. The rest of the animals either experienced rapid tumor progression or were euthanized due to large tumor burdens. The positive control 5F11bbz CAR T cells had little tumor growth inhibition activity even at 2×10⁶ cells dosage. So the in vivo tumor growth inhibition efficacy of CAR T cells are ranked from high to low: AS47863VH4dil-bbz, AS48542VH5bbz, AS48542VH5dil-bbz, AS53574VH7-AS47863VH4bil-bbz and 5F11bbz.

### Example 9 In Vitro Cytotoxicity of Humanized Armored CAR T Cells

This example demonstrated that incorporating dominant negative TGFβ receptor II (dnTGFβRII) in the CAR constructs improved the in vitro efficacy of CAR T cells.

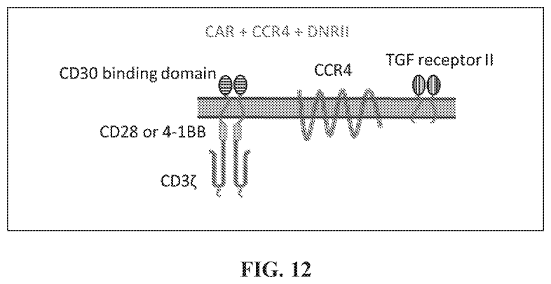

In this example, CAR constructs were designed so other molecules were co-expressed with the conventional 2^{nd} generation CARs. In some constructs, CCR4 molecule was co-expressed with the conventional 2^{nd} generation CARs in the pattern from the N-terminus to the C-terminus: 2nd generation CAR, P2A (SEQ ID NO: 66), and full-length CCR4 (SEQ ID NO: 67). These constructs were designated “[CD30-binding moiety]bbz-4C” (e.g. AS48542VH5bbz-4C, SEQ ID NO: 198) (FIG. 11, top right). In some constructs, dnTGFβRII molecule was co-expressed with the conventional 2nd generation CARs in the pattern from the N-terminus to the C-terminus: dnTGFβRII (SEQ ID NO: 68), P2A (SEQ ID NO: 66), and 2nd generation CAR. These constructs were designated “TR2D-[CD30-binding moiety]bbz” (e.g. TR2D-AS48542VH5bbz, SEQ ID NO: 196 (FIG. 11, bottom right). The dnTGFβRII sequence (M1-S199) lacking the intracellular kinase domain was originally reported by Wieser R, et al. (Wieser R, et al., Molecular and cellular biology 13:7239-7247 (1993)). In some constructs, a chimeric switch PD-1 receptor (PD1CD28) was co-expressed with the conventional 2^{nd} generation CARs in the pattern from the N-terminus to the C-terminus: PD1CD28 (SEQ ID NO: 69), P2A (SEQ ID NO: 66), and 2^{nd} generation CAR. These constructs were designated “PD1CD28-[CD30-binding moiety]bbz” (e.g. PD1CD28-AS48542VH5bbz, SEQ ID NO: 197) (FIG. 11, bottom left). The chimeric switch PD-1 receptor is the PD-1 ECD (M1-H155) fused at the N-terminus of the hinge, transmembrane and cytoplasmic portion of CD28 (C141-S220). In some constructs, both dnTGFβRII and full-length CCR4 molecules were co-expressed with the conventional 2nd generation CARs in the pattern from the N-terminus to the C-terminus: dnTGFβRII (SEQ ID NO: 68), P2A (SEQ ID NO: 66), 2^{nd} generation CAR, and full-length CCR4 (SEQ ID NO: 67). These constructs were designated TR2D-[CD30-binding moiety]bbz-4C (e.g. TR2D-AS48542VH5bbz-4C, SEQ ID NO: 195) (FIG. 12). DNAs encoding all the CAR sequences with the co-expressed molecules were codon optimized, synthesized, and ligated into a lentiviral vector plasmid with human EF1 alpha promoter for expression.

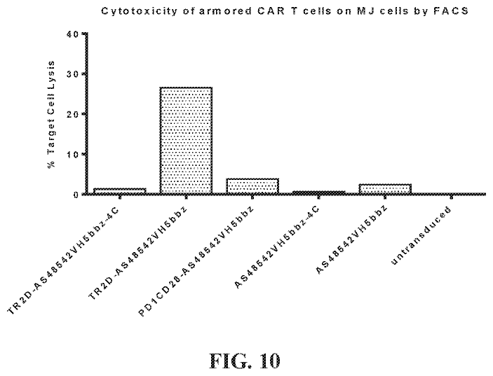

In vitro cytotoxicity assay was carried out using FACS method as described in Example 7 on AS48542VH5bbz CARs co-expressed with the above-mentioned molecules and combination thereof. In this assay transduced and untransduced CAR T cells were incubated with MJ cells at E:T ratio of 0.1:1 for 96 hours (at cell density of approximately 1×10⁵ cells/mL). According to the result, AS48542VH5bbz CAR co-expressed with dnTGFβRII improved the cytotoxicity of CAR T cells significantly (Table 11 and FIG. 10).

TABLE 11
In vitro cytotoxicity of armored
CAR T cells on MJ cells by FACS.

| | % CAR T+ | % MJ |
|---|---|---|
| | Day 8 of | cell lysis |
| CAR construct | transduction | E:T = 0.1:1 |
| TR2D-AS48542VH5bbz-4C | 77.6% | 1.3% |
| TR2D-AS48542VH5bbz | 76.3% | 26.5% |
| PD1CD28-AS48542VH5bbz | 80.1% | 3.8% |
| AS48542VH5bbz-4C | 60.7% | 0.6% |
| AS48542VH5bbz | 94.0% | 2.4% |
| untransduced | N/A | 0.0% |

New CAR constructs using a single or a tandem-repeat (‘dil’) of AS47863VH4 or AS48542VH5 CD30-binding moieties, with 4-1BB (‘bbz’) or CD28 (‘28z’) costimulatory signals, with or without dnTGFβRII armor were constructed as described in Example 3 (SEQ ID NOs: 205-215), and were were subjected to long-term repeated stimulation assays.

Viral vectors of 16 anti-CD30 CAR (SEQ ID NOs: 182, 186, 184, 188, 196 and 205-215), as well as 5F11bbz were used to transduce activated T cells as described in Example 3. Six days after transduction, CAR T cells were co-incubated with cHL line L540 cells at E:T ratio of 1:3 (i.e. 10⁵ CAR T cells+3×10⁵ L540 cells) in 1.5 ml of AIM V™ medium supplemented with 5% FBS. After 2 days of co-incubation, cells were counted and cell mixtures were analyzed by FACS. About 10⁵ CAR T cells from the cell mixture of the first round of killing were again co-incubated with 3×10⁵ fresh L540 cells (i.e. E:T ratio=1:3). Co-incubation of CAR T cells and tumor cells at E:T ratio of 1:3 were repeated every 2 days until CAR T cells could no longer kill tumor cells. CAR T cell expansion was calculated as the number of T cells after each round of killing over the number of T cells before killing (i.e. 10⁵ T cells).

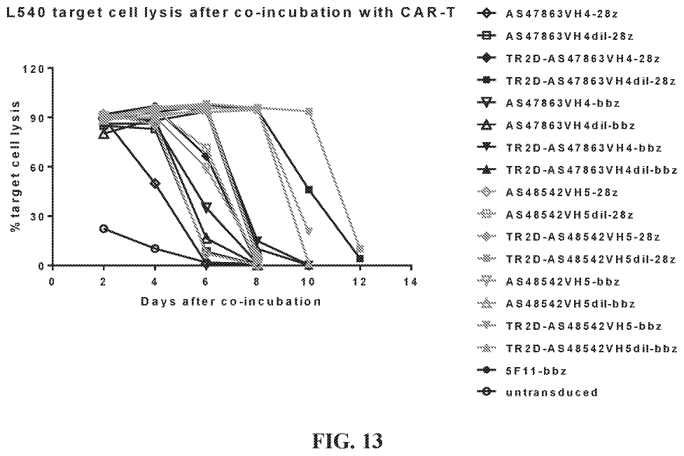

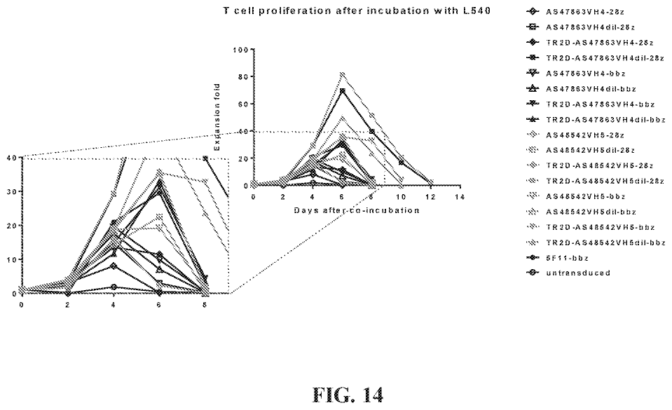

According to the result of target cell lysis (Table 12 and FIG. 13), untransduced T cells failed to eliminate L540 cells even at the first round of co-incubation. 5F11bbz CAR T cells eliminated almost all tumor cells at the first three round of co-incubation, but failed at the forth round. Most armored CAR constructs showed superior long-term killing potential than their unarmored counterparts, except TR2D-AS47863VH4-28z, which showed similar potential to that of the unarmored CAR. This suggests that co-expression of TGFβ DNRII indeed improved the in vitro efficacy of CAR T cells. Of all constructs, TR2D-AS47863VH4dil-28z and TR2D-AS48542VH5dil-28z showed highest potential of long-term killing activity. These two constructs also showed the best T cell expansion upon stimulation by target cells (Table 13 and FIG. 14).

TABLE 12
Percentage of lyzed L540 cells after every round of killing by CAR T cells.

| | % Target cell lysis | | | | | |
|---|---|---|---|---|---|---|
| CAR constructs | Day 2 | Day 4 | Day 6 | Day 8 | Day 10 | Day 12 |
| AS47863VH4-28z | 89.6 | 49.8 | 1.0 | 0.2 | — | — |
| AS47863VH4dil-28z | 85 | 82.9 | 8.4 | 0.4 | — | — |
| TR2D-AS47863VH4-28z | 90 | 95.7 | 66.6 | 0.3 | — | — |
| TR2D-AS47863VH4dil-28z | 90 | 95.3 | 97.7 | 94.5 | 46.1 | 4.1 |
| AS47863VH4-bbz | 86.1 | 86.1 | 35 | 1.2 | — | — |
| AS47863VH4dil-bbz | 80.1 | 89.1 | 16.5 | 0.3 | — | — |
| TR2D-AS47863VH4-bbz | 91.1 | 88.1 | 93.5 | 14.8 | 0.3 | — |
| TR2D-AS47863VH4dil-bbz | 88 | 93.1 | 97.2 | 10 | 0.1 | — |
| AS48542VH5-28z | 92 | 85.8 | 7.2 | 0.2 | — | — |
| AS48542VH5dil-28z | 90.8 | 94.4 | 71.3 | 0.7 | — | — |
| TR2D-AS48542VH5-28z | 90.7 | 95.1 | 97 | 4.8 | — | — |
| TR2D-AS48542VH5dil-28z | 90.1 | 96.2 | 98.2 | 95.9 | 93.7 | 10.1 |
| AS48542VH5-bbz | 89.2 | 90.2 | 59.1 | 4.3 | — | — |
| AS48542VH5dil-bbz | 88.7 | 90.2 | 95 | 6.8 | — | — |
| TR2D-AS48542VH5-bbz | 90.1 | 92.3 | 93.2 | 94.5 | 20.2 | — |
| TR2D-AS48542VH5dil-bbz | 90.2 | 95.1 | 95.6 | 95.1 | 1.2 | — |
| 5F11bbz | 91.9 | 96.9 | 94.3 | 4.5 | — | — |
| untransduced | 22.3 | 10.2 | 1.8 | 1.0 | — | — |

TABLE 13
T cell expansion fold after every round of killing.

| | T cell expansion fold | | | | | |
|---|---|---|---|---|---|---|
| CAR constructs | Day 2 | Day 4 | Day 6 | Day 8 | Day 10 | Day 12 |
| AS47863VH4-28z | 3.1 | 8.0 | 0.2 | 0.1 | — | — |
| AS47863VH4dil-28z | 2.7 | 14.7 | 3.0 | 0.2 | — | — |
| TR2D-AS47863VH4-28z | 3.5 | 13.5 | 11.5 | 0.1 | — | — |
| TR2D-AS47863VH4dil-28z | 2.7 | 29.2 | 69.6 | 39.7 | 16.8 | 1.4 |
| AS47863VH4-bbz | 2.2 | 19.5 | 9.8 | 0.4 | — | — |
| AS47863VH4dil-bbz | 3.2 | 17.6 | 7.1 | 0.1 | — | — |
| TR2D-AS47863VH4-bbz | 3.2 | 14.8 | 31.3 | 4.5 | — | — |
| TR2D-AS47863VH4dil-bbz | 2.5 | 11.7 | 33.0 | 3.4 | — | — |
| AS48542VH5-28z | 1.6 | 16.0 | 2.3 | 0.1 | — | — |
| AS48542VH5dil-28z | 2.7 | 14.3 | 22.5 | 0.2 | — | — |
| TR2D-AS48542VH5-28z | 3.8 | 19.2 | 35.2 | 1.8 | — | — |
| TR2D-AS48542VH5dil-28z | 4.0 | 29.4 | 81.3 | 51.4 | 20.5 | 2.4 |
| AS48542VH5-bbz | 3.7 | 18.8 | 19.1 | 1.7 | — | — |
| AS48542VH5dil-bbz | 4.0 | 18.4 | 35.6 | 1.5 | — | — |
| TR2D-AS48542VH5-bbz | 2.6 | 19.0 | 35.5 | 32.7 | 4.0 | — |
| TR2D-AS48542VH5dil-bbz | 2.6 | 18.8 | 49.7 | 23.6 | 0.1 | — |
| 5F11bbz | 0.1 | 20.9 | 29.5 | 1.9 | — | — |
| untransduced | 0.0 | 1.8 | 0.4 | 0.2 | — | — |

### Example 10 Evaluation of Armored Humanized CAR T Cells in In Vivo Mouse Model

This example demonstrated that incorporating dominant negative TGFβ receptor II (dnTGFβRII) in the CAR constructs improved the in vivo efficacy of CAR T cells in HH mouse model.

Anti-tumor activities of AS48542VH5bbz, TR2D-AS48542VH5bbz, AS47863VH4dil-bbz and TR2D-AS47863VH4dil-bbz CAR T cells were assessed in vivo in an HH xenograft model as described in Example 4, only the dose of CAR T cells was reduced to 1×10⁶ cells per mouse.

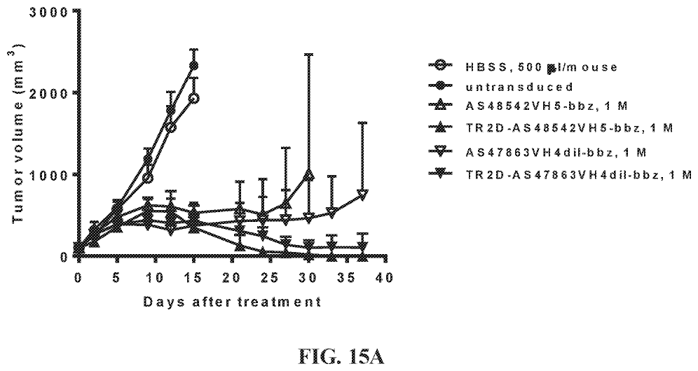

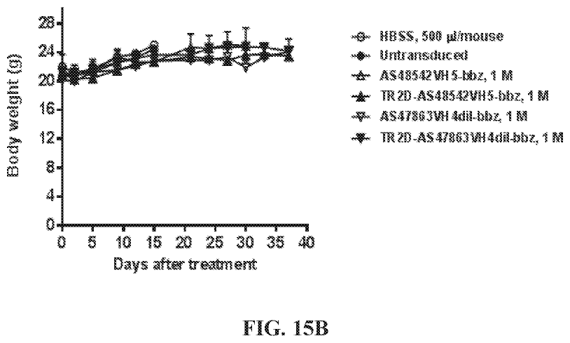

As shown in FIG. 15A, at 10⁶ dosage unarmored AS48542VH5bbz and AS47863VH4dil-bbz CAR T cells could inhibit tumor growth to some extent compared to HBSS and untransduced T cell controls, whereas both TR2D-AS48542VH5bbz and TR2D-AS47863VH4dil-bbz CAR T cells could completely suppress tumor growth. The result suggests by co-expressing dnTGFβRII T cells gained more anti-tumor activity. The body weights of mice did not show significant change (FIG. 15B), suggesting little or no toxicity associated with the expression of dnTGFβRII to mice.

SEQUENCES

| SEQ | | |
|---|---|---|
| ID NO. | Description | Sequence |
| 1 | human CD30 | FPQDRPFEDTCHGNPSHYYDKAVRRCCYRCPMGLFPTQQCPQRPTDCRKQC |
| | ECD | EPDYYLDEADRCTACVTCSRDDLVEKTPCAWNSSRVCECRPGMFCSTSAVN |
| | | SCARCFFHSVCPAGMIVKFPGTAQKNTVCEPASPGVSPACASPENCKEPSS |
| | | GTIPQAKPTPVSPATSSASTMPVRGGTRLAQEAASKLTRAPDSPSSVGRPS |
| | | SDPGLSPTQPCPEGSGDCRKQCEPDYYLDEAGRCTACVSCSRDDLVEKTPC |
| | | AWNSSRTCECRPGMICATSATNSCARCVPYPICAAETVTKPQDMAEKDTTF |
| | | EAPPLGTQPDCNPTPENGEAPASTSPTQSLLVDSQASKTLPIPTSAPVALS |
| | | STGK |
| 2 | rhesus CD30 | FPQDRPFEDTCRGNPGHYYDKAVRRCCYRCPTGLFPTQQCPQRPADCRKQC |
| | ECD | EPDYYLDEAGRCTACVSCSRDDLVEKMPCAWNSSRVCECQPGMFCAVSVVN |
| | | SCARCFFHSVCPAGMIVKFPGTAQKNTVCEPASPGVSPACASPENCKEPSS |
| | | GTIPQAKPTPVSPATSNASTMPLRGGTRLAQEAASKLTRAPGSPSSVGRPS |
| | | SDPGLSPTQPCPQGSGDCRKQCEPDYYLDEAGRCTACVSCSRDDLVEKTPC |
| | | AWNSSRICECRPGMICATSATNSCARCVPYPICAAETGTKPQDMAEKDTTF |
| | | EAPPVGTQPDCSPTPENGEAPASTSPTLSSLVDSQASKTLPIPTSAPIALS |
| | | STGK |
| 3 | CRD1 | FPQDRPFEDTCHGNPSHYYDKAVRRCCYRCPMGLFPTQQCPQRPTDCRKQ |
| 4 | CRD2 | RKQCEPDYYLDEADRCTACVTCSRDDLVEKTPCAWNSSRVCE |
| 5 | CRD3 | ECRPGMTFCSTSAVNSCARCFFHSVCPAGMIVKFPGTAQKNTVCEPAS |
| 6 | CRD4 | EPASPGVSPACASPENCKEPSSGTIPQAKPTPVSPATSSASTMPVRGGTRL |
| | | AQEAASKLTRAPDSPSSVGRPSSDPGLSPTQPCPEGSGDCRKQ |
| 7 | CRD5 | RKQCEPDYYLDEAGRCTACVSCSRDDLVEKTPCAWNSSRTCE |
| 8 | CRD6 | ECRPGMICATSATNSCARCVPYPICAAETVTKPQDMAEKDTTFEAPPLGTQ |
| | | PDCNPTPENGEAPASTSPTQSLLVDSQASKTLPIPTSAPVALSSTGK |
| 9 | AS47863 | QVQLEESGGGSVQAGETLRLSCTASGSTFGDSDMGWYRQAPGNACELVSII |
| | | SSDGRTYYVDSVKGRFTISQDNAVSTVYLQMNSLKPEDTGVYYCAADLRQY |
| | | CRDGRCCGYWGQGTQVTVSS |
| 10 | AS48433 | QIQLVESGGGSVQAGETLRLSCTASGSTFGDSDMGWYRQAPGNACELVSII |
| | | SSDGRTYYVDSVKGRFTISQDNAVSTVYLQMNSLNPEDTGVYYCAADLRLN |
| | | CRDGRCCGYWGQGTQVTVSS |
| 11 | AS48463 | QVHLMESGGGSVQAGETLRLSCTASGFTFANSDMGWYRQAPGNACELVSII |
| | | SSHGGTTYYVDSVKGRFTISRHNAENTVYLRMTSLKPEDTALYYCVADPRS |
| | | NCRGGYCCGYWGPGTQVTVSS |
| 12 | AS48481 | EVQLVASGGGSVQAGETLRLSCTASGFTFADSAMGWYRKGPGNVCDLVAII |
| | | RTDGTTYYGDSAKGRFTISRDNAKSTLYLQMNSLKPEDTAVYFCAADRETS |
| | | FIGGSWCVAKYWDQGTQVTVSS |
| 13 | AS48508 | EVQLVESGGGSVQAGGSLRLSCTASRFTFDGPDMAWYRQAPGNACELVSII |
| | | SADGRTYYTDSVKGRFTISRDNAKNTVFLYLNSLQPEDTAVYYCAPDPRRN |
| | | CRGGYCCGNWGPGTQVTVSS |
| 14 | AS48542 | QMQLVESGGGSVQAGETLRLSCTTSAFTFDGPDMAWYRQAPGNECVLVSII |
| | | SADGRTYYADSVKGRFTISRDNAKNTVFLNLNSLQPEDTAVYYCALDPRKN |
| | | CRGGYCCANWGPGTQVTVSS |
| 15 | AS53445 | QVQLVESGGGSVQAGGSLRLSCTASGYIFCMGWFRQAPGKAREGIATIYTG |
| | | GDSTYYDDSVKGRFTISRDNAKNTVYLQMNSLKPEDTAMYYCAAGGQECYL |
| | | TNWVSYWGQGTQVTVSS |
| 16 | AS53574 | QVKLVESGGGSVQAGGSLRLSCAASGYIYSSNCMGWFRQAPGKEREWVARI |
| | | HTGSGSTYYADSVKGRFTISQDNAKNTVYLQMNSLRPEDTAMYDCAAGRVV |
| | | LGAVVCTNEYWGQGTQVTVSS |
| 17 | AS53750 | EVQLVESGGGLVQPGGSLRLSCTASGFTDDGPDMAWYRRAPGNECELVSII |
| | | SADGRTYYTDSVKGRFTISRDNAKNTVFLYLNSLQPEDTAVYYCAPDPRRN |
| | | CRGGDCCGNWGPGTQVTVSS |
| 18 | AS54233 | QVQLVESGGGSVQAGETLRLSCTASGFTFDGPDMAWYRQAPGNECELVSII |
| | | SADGRTYYTDSVKGRFTASQDNAKNTVSLYLKSLQPEDTAVYYCAADPRRN |
| | | CRGNCCGNWGPGTQVTVSS |
| 19 | AS47863VH4 | EVQLVESGGGLVQPGGSLRLSCAASGSTFGDSDMGWYRQAPGKGCELVSII |
| | | SSDGRTYYVDSVKGRFTISQDNSKNTLYLQMNSLRAEDTAVYYCAADLRQY |
| | | CRDGRCCGYWGQGTLVTVSS |
| 20 | AS47863VH5 | EVQLVESGGGLVQPGGSLRLSCAASGSTFGDSDMGWYRQAPGKGCELVSII |
| | | SSDGRTYYVDSVKGRFTISQDNSKNTVYLQMNSLRAEDTAVYYCAADLRQY |
| | | CRDGRCCGYWGQGTLVTVSS |
| 21 | AS47863VH11 | EVQLVESGGGLVQPGGSLRLSCAASGSTFGDSDMGWYRQAPGKGCELVSII |
| | | SSDGRTYYVDSVKGRFTISQDNAKNTLYLQMNSLRPEDTAVYYCAADLRQY |
| | | CRDGRCCGYWGQGTLVTVSS |
| 22 | AS47863VH12 | EVQLVESGGGLVQPGGSLRLSCAASGSTFGDSDMGWYRQAPGKGCELVSII |
| | | SSDGRTYYVDSVKGRFTISQDNAKNTVYLQMNSLRPEDTAVYYCAADLRQY |
| | | CRDGRCCGYWGQGTLVTVSS |
| 23 | AS48433VH4 | EVQLVESGGGLVQPGGSLRLSCAASGSTFGDSDMGWYRQAPGKGCELVSII |
| | | SSDGRTYYVDSVKGRFTISQDNSKNTLYLQMNSLRAEDTAVYYCAADLRLN |
| | | CRDGRCCGYWGQGTLVTVSS |
| 24 | AS48433VH5 | EVQLVESGGGLVQPGGSLRLSCAASGSTFGDSDMGWYRQAPGKGCELVSII |
| | | SSDGRTYYVDSVKGRFTISQDNSKNTVYLQMNSLRAEDTAVYYCAADLRLN |
| | | CRDGRCCGYWGQGTLVTVSS |
| 25 | AS48433VH11 | EVQLVESGGGLVQPGGSLRLSCAASGSTFGDSDMGWYRQAPGKGCELVSII |
| | | SSDGRTYYVDSVKGRFTISQDNAKNTLYLQMNSLRPEDTAVYYCAADLRLN |
| | | CRDGRCCGYWGQGTLVTVSS |
| 26 | AS48433VH12 | EVQLVESGGGLVQPGGSLRLSCAASGSTFGDSDMGWYRQAPGKGCELVSII |
| | | SSDGRTYYVDSVKGRFTISQDNAKNTVYLQMNSLRPEDTAVYYCAADLRLN |
| | | CRDGRCCGYWGQGTLVTVSS |
| 27 | AS48463VH4 | EVQLLESGGGLVQPGGSLRLSCAASGFTFANSDMGWYRQAPGKGCELVSII |
| | | SSHGGTTYYVDSVKGRFTISRDNSKNTLYLQMNSLRAEDTAVYYCVADPRS |
| | | NCRGGYCCGYWGQGTLVTVSS |
| 28 | AS48463VH11 | EVQLLESGGGLVQPGGSLRLSCAASGFTFANSDMGWYRQAPGKGCELVSII |
| | | SSHGGTTYYVDSVKGRFTISRDNAKNTLYLQMNSLRPEDTAVYYCVADPRS |
| | | NCRGGYCCGYWGQGTLVTVSS |
| 29 | AS48481VH5 | QVQLVESGGGVVQPGRSLRLSCAASGFTFADSAMGWYRQAPGKGCELVAII |
| | | RTDGTTYYGDSAKGRFTISRDNSKNTLYLQMNSLRAEDTAVYFCAADRETS |
| | | FIGGSWCVAKYWDQGTLVTVSS |
| 30 | AS48481VH6 | QVQLVESGGGVVQPGRSLRLSCAASGFTFADSAMGWYRQAPGKVCELVAII |
| | | RTDGTTYYGDSAKGRFTISRDNSKNTLYLQMNSLRAEDTAVYFCAADRETS |
| | | FIGGSWCVAKYWDQGTLVTVSS |
| 31 | AS48481VH13 | QVQLVESGGGVVQPGRSLRLSCAASGFTFADSAMGWYRQAPGKGCELVAII |
| | | RTDGTTYYGDSAKGRFTISRDNAKNTLYLQMNSLRPEDTAVYFCAADRETS |
| | | FIGGSWCVAKYWDQGTLVTVSS |
| 32 | AS48481VH14 | QVQLVESGGGVVQPGRSLRLSCAASGFTFADSAMGWYRQAPGKVCELVAII |
| | | RTDGTTYYGDSAKGRFTISRDNAKNTLYLQMNSLRPEDTAVYFCAADRETS |
| | | FIGGSWCVAKYWDQGTLVTVSS |
| 33 | AS48508VH4 | EVQLVESGGGLVQPGGSLRLSCAASRFTFDGPDMAWYRQAPGKGCELVSII |
| | | SADGRTYYTDSVKGRFTISRDNSKNTLYLQMNSLRAEDTAVYYCAPDPRRN |
| | | CRGGYCCGNWGQGTTVTVSS |
| 34 | AS48508VH5 | EVQLVESGGGLVQPGGSLRLSCAASRFTFDGPDMAWYRQAPGKGCELVSII |
| | | SADGRTYYTDSVKGRFTISRDNSKNTVYLQMNSLRAEDTAVYYCAPDPRRN |
| | | CRGGYCCGNWGQGTTVTVSS |
| 35 | AS48508VH11 | EVQLVESGGGLVQPGGSLRLSCAASRFTFDGPDMAWYRQAPGKGCELVSII |
| | | SADGRTYYTDSVKGRFTISRDNAKNTLYLQMNSLRPEDTAVYYCAPDPRRN |
| | | CRGGYCCGNWGQGTTVTVSS |
| 36 | AS48508VH12 | EVQLVESGGGLVQPGGSLRLSCAASRFTFDGPDMAWYRQAPGKGCELVSII |
| | | SADGRTYYTDSVKGRFTISRDNAKNTVYLQMNSLRPEDTAVYYCAPDPRRN |
| | | CRGGYCCGNWGQGTTVTVSS |
| 37 | AS48542VH5 | EVQLVESGGGLVQPGGSLRLSCATSAFTFDGPDMAWYRQAPGKGCELVSII |
| | | SADGRTYYADSVKGRFTISRDNSKNTVYLQMNSLRAEDTAVYYCALDPRKN |
| | | CRGGYCCANWGQGTLVTVSS |
| 38 | AS48542VH12 | EVQLVESGGGLVQPGGSLRLSCATSAFTFDGPDMAWYRQAPGKGCELVSII |
| | | SADGRTYYADSVKGRFTISRDNAKNTVYLQMNSLRPEDTAVYYCALDPRKN |
| | | CRGGYCCANWGQGTLVTVSS |
| 39 | AS53445VH4 | QVQLVESGGGVVQPGGSLRLSCAASGYIFCMGWFRQAPGKGLEGIATIYTG |
| | | GDSTYYDDSVKGRFTISRDNSKNTLYLQMNSLRAEDTAVYYCAAGGQECYL |
| | | TNWVSYWGQGTLVTVSS |
| 40 | AS53445VH11 | QVQLVESGGGVVQPGGSLRLSCAASGYIFCMGWFRQAPGKGREGIATIYTG |
| | | GDSTYYDDSVKGRFTISRDNAKNTLYLQMNSLRPEDTAVYYCAAGGQECYL |
| | | TNWVSYWGQGTLVTVSS |
| 41 | AS53574VH4 | EVQLVESGGGLVQPGGSLRLSCAASGYIYSSNCMGWFRQAPGKGLEWVSRI |
| | | HTGSGSTYYADSVKGRFTISRDNSKNTLYLQMNSLRAEDTAVYYCAAGRVV |
| | | LGAVVCTNEYWGQGTLVTVSS |
| 42 | AS53574VH5 | EVQLVESGGGLVQPGGSLRLSCAASGYIYSSNCMGWFRQAPGKGLEWVSRI |
| | | HTGSGSTYYADSVKGRFTISQDNSKNTLYLQMNSLRAEDTAVYYCAAGRVV |
| | | LGAVVCTNEYWGQGTLVTVSS |
| 43 | AS53574VH6 | EVQLVESGGGLVQPGGSLRLSCAASGYIYSSNCMGWFRQAPGKGLEWVARI |
| | | HTGSGSTYYADSVKGRFTISQDNSKNTLYLQMNSLRAEDTAVYYCAAGRVV |
| | | LGAVVCTNEYWGQGTLVTVSS |
| 44 | AS53574VH11 | EVQLVESGGGLVQPGGSLRLSCAASGYIYSSNCMGWFRQAPGKGREWVSRI |
| | | HTGSGSTYYADSVKGRFTISRDNAKNTLYLQMNSLRPEDTAVYYCAAGRVV |
| | | LGAVVCTNEYWGQGTLVTVSS |
| 45 | AS53574VH12 | EVQLVESGGGLVQPGGSLRLSCAASGYIYSSNCMGWFRQAPGKGREWVSRI |
| | | HTGSGSTYYADSVKGRFTISQDNAKNTLYLQMNSLRPEDTAVYYCAAGRVV |
| | | LGAVVCTNEYWGQGTLVTVSS |
| 46 | AS53574VH13 | EVQLVESGGGLVQPGGSLRLSCAASGYIYSSNCMGWFRQAPGKGREWVARI |
| | | HTGSGSTYYADSVKGRFTISQDNAKNTLYLQMNSLRPEDTAVYYCAAGRVV |
| | | LGAVVCTNEYWGQGTLVTVSS |
| 47 | AS53750VH4 | EVQLVESGGGLVQPGGSLRLSCAASGFTDDGPDMAWYRQAPGKGCELVSII |
| | | SADGRTYYTDSVKGRFTISRDNSKNTLYLQMNSLRAEDTAVYYCAPDPRRN |
| | | CRGGDCCGNWGQGTTVTVSS |
| 48 | AS53750VH5 | EVQLVESGGGLVQPGGSLRLSCAASGFTDDGPDMAWYRQAPGKGCELVSII |
| | | SADGRTYYTDSVKGRFTISRDNSKNTVYLQMNSLRAEDTAVYYCAPDPRRN |
| | | CRGGDCCGNWGQGTTVTVSS |
| 49 | AS53750VH11 | EVQLVESGGGLVQPGGSLRLSCAASGFTDDGPDMAWYRQAPGKGCELVSII |
| | | SADGRTYYTDSVKGRFTISRDNAKNTLYLQMNSLRPEDTAVYYCAPDPRRN |
| | | CRGGDCCGNWGQGTTVTVSS |
| 50 | AS53750VH12 | EVQLVESGGGLVQPGGSLRLSCAASGFTDDGPDMAWYRQAPGKGCELVSII |
| | | SADGRTYYTDSVKGRFTISRDNAKNTVYLQMNSLRPEDTAVYYCAPDPRRN |
| | | CRGGDCCGNWGQGTTVTVSS |
| 51 | AS54233VH4 | EVQLVESGGGLVQPGGSLRLSCAASGFTFDGPDMAWYRQAPGKGCELVSII |
| | | SADGRTYYTDSVKGRFTASQDNSKNTLYLQMNSLRAEDTAVYYCAADPRRN |
| | | CRGNCCGNWGQGTLVTVSS |
| 52 | AS54233VH5 | EVQLVESGGGLVQPGGSLRLSCAASGFTFDGPDMAWYRQAPGKGCELVSII |
| | | SADGRTYYTDSVKGRFTASQDNSKNTVYLQMNSLRAEDTAVYYCAADPRRN |
| | | CRGNCCGNWGQGTLVTVSS |
| 53 | AS54233VH11 | EVQLVESGGGLVQPGGSLRLSCAASGFTFDGPDMAWYRQAPGKGCELVSII |
| | | SADGRTYYTDSVKGRFTASQDNAKNTLYLQMNSLRPEDTAVYYCAADPRRN |
| | | CRGNCCGNWGQGTLVTVSS |
| 54 | AS54233VH12 | EVQLVESGGGLVQPGGSLRLSCAASGFTFDGPDMAWYRQAPGKGCELVSII |
| | | SADGRTYYTDSVKGRFTASQDNAKNTVYLQMNSLRPEDTAVYYCAADPRRN |
| | | CRGNCCGNWGQGTLVTVSS |
| 55 | 5F11 scFv | GSTSGSGKPGSGEGSTKG |
| | linker | |
| 56 | (G4S)₃ linker | GGGGSGGGGSGGGGS |
| 57 | G4S linker | GGGGS |
| 58 | AS57911 | DIQMTQSPSSLSASVGDRVTITCRASQSVSSAVAWYQQKPGKAPKLLIYSA |
| | scFv | SSLYSGVPSRFSGSRSGTDFTLTISSLQPEDFATYYCQQSHALITFGQGTK |
| | | VEIKGGGGSGGGGSGGGGSEVQLVESGGGLVQPGGSLRLSCAASGFNISSS |
| | | YIHWVRQAPGKGLEWVAYISSYYSYTYYADSVKGRFTISADTSKNTAYLQM |
| | | NSLRAEDTAVYYCARGYPYGMDYWGQGTLVTVSS |
| 59 | AS57659 | DIQMTQSPSSLSASVGDRVTITCRASQSVSSAVAWYQQKPGKAPKLLIYSA |
| | scFv | SSLYSGVPSRFSGSRSGTDFTLTISSLQPEDFATYYCQQPYYLITFGQGTK |
| | | VEIKGGGGSGGGGSGGGGSEVQLVESGGGLVQPGGSLRLSCAASGFNIYSY |
| | | YIHWVRQAPGKGLEWVASIYSSYSSTYYADSVKGRFTISADTSKNTAYLQM |
| | | NSLRAEDTAVYYCARSWFSYPGLDYWGQGTLVTVSS |
| 60 | AS57765 | DIQMTQSPSSLSASVGDRVTITCRASQSVSSAVAWYQQKPGKAPKLLIYSA |
| | scFv | SSLYSGVPSRFSGSRSGTDFTLTISSLQPEDFATYYCQQAYYSLITFGQGT |
| | | KVEIKGGGGSGGGGSGGGGSEVQLVESGGGLVQPGGSLRLSCAASGFNIYY |
| | | SYMHWVRQAPGKGLEWVAYIYPYSGSTSYADSVKGRFTISADTSKNTAYLQ |
| | | MNSLRAEDTAVYYCARPAVHWHGYGGGYYYGLDYWGQGTLVTVSS |
| 61 | leader | MALPVTALLLPLALLLHAARP |
| | sequence | |
| 62 | CD8α hinge | TTTPAPRPPTPAPTIASQPLSLRPEACRPAAGGAVHTRGLDFACD |
| 63 | CD8α TM | IYIWAPLAGTCGVLLLSLVITLYC |
| 64 | cytoplasmic | KRGRKKLLYIFKQPFMRPVQTTQEEDGCSCRFPEEEEGGCEL |
| | portion of the | |
| | 4-1BB | |
| | (CD137) | |
| 65 | cytoplasmic | RVKFSRSADAPAYKQGQNQLYNELNLGRREEYDVLDKRRGRDPEMGGKPRR |
| | portion of the | KNPQEGLYNELQKDKMAEAYSEIGMKGERRRGKGHDGLYQGLSTATKDTYD |
| | CD3ζ | ALHMQALPPR |
| 66 | P2A | GSGATNFSLLKQAGDVEENPGP |
| 67 | full-length | MNPTDIADTTLDESIYSNYYLYESIPKPCTKEGIKAFGELFLPPLYSLVFV |
| | CCR4 | FGLLGNSVVVLVLFKYKRLRSMTDVYLLNLAISDLLFVFSLPFWGYYAADQ |
| | | WVFGLGLCKMISWMYLVGFYSGIFFVMLMSIDRYLAIVHAVFSLRARTLTY |
| | | GVITSLATWSVAVFASLPGFLFSTCYTERNHTYCKTKYSLNSTTWKVLSSL |
| | | EINILGLVIPLGIMLFCYSMIIRTLQHCKNEKKNKAVKMIFAVVVLFLGFW |
| | | TPYNIVLFLETLVELEVLQDCTFERYLDYAIQATETLAFVHCCLNPIIYFF |
| | | LGEKFRKYILQLFKTCRGLFVLCQYCGLLQIYSADTPSSSYTQSTMDHDLH |
| | | DAL |
| 68 | dnTGFβRII | MGRGLLRGLWPLHIVLWTRIASTIPPHVQKSVNNDMIVTDNNGAVKFPQLC |
| | | KFCDVRFSTCDNQKSCMSNCSITSICEKPQEVCVAVWRKNDENITLETVCH |
| | | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSS |
| 69 | PD1CD28 | MQIPQAPWPVVWAVLQLGWRPGWFLDSPDRPWNPPTFSPALLVVTEGDNAT |
| | | FTCSFSNTSESFVLNWYRMSPSNQTDKLAAFPEDRSQPGQDCRFRVTQLPN |
| | | GRDFHMSVVRARRNDSGTYLCGAISLAPKAQIKESLRAELRVTERRAEVPT |
| | | AHCPSPLFPGPSKPFWVLVVVGGVLACYSLLVTVAFIIFWVRSKRSRLLHS |
| | | DYMNMTPRRPGPTRKHYQPYAPPRDFAAYRS |
| 70 | AS47863bbz | MALPVTALLLPLALLLHAARPQVQLEESGGGSVQAGETLRLSCTASGSTFG |
| | | DSDMGWYRQAPGNACELVSIISSDGRTYYVDSVKGRFTISQDNAVSTVYLQ |
| | | MNSLKPEDTGVYYCAADLRQYCRDGRCCGYWGQGTQVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 71 | AS48433bbz | MALPVTALLLPLALLLHAARPQVQLEESGGGSVQAGETLRLSCTASGSTFG |
| | | DSDMGWYRQAPGNACELVSIISSDGRTYYVDSVKGRFTISQDNAVSTVYLQ |
| | | MNSLKPEDTGVYYCAADLRQYCRDGRCCGYWGQGTQVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 72 | AS48463bbz | MALPVTALLLPLALLLHAARPQVHLMESGGGSVQAGETLRLSCTASGFTFA |
| | | NSDMGWYRQAPGNACELVSIISSHGGTTYYVDSVKGRFTISRHNAENTVYL |
| | | RMTSLKPEDTALYYCVADPRSNCRGGYCCGYWGPGTQVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 73 | AS48481bbz | MALPVTALLLPLALLLHAARPEVQLVASGGGSVQAGETLRLSCTASGFTFA |
| | | DSAMGWYRKGPGNVCDLVAIIRTDGTTYYGDSAKGRFTISRDNAKSTLYLQ |
| | | MNSLKPEDTAVYFCAADRETSFIGGSWCVAKYWDQGTQVTVSSTTTPAPRP |
| | | PTPAPTIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEEGGCE |
| | | LRVKFSRSADAPAYKQGQNQLYNELNLGRREEYDVLDKRRGRDPEMGGKPR |
| | | RKNPQEGLYNELQKDKMAEAYSEIGMKGERRRGKGHDGLYQGLSTATKDTY |
| | | DALHMQALPPR |
| 74 | AS48508bbz | MALPVTALLLPLALLLHAARPEVQLVESGGGSVQAGGSLRLSCTASRFTFD |
| | | GPDMAWYRQAPGNACELVSIISADGRTYYTDSVKGRFTISRDNAKNTVFLY |
| | | LNSLQPEDTAVYYCAPDPRRNCRGGYCCGNWGPGTQVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 75 | AS48542bbz | MALPVTALLLPLALLLHAARPQMQLVESGGGSVQAGETLRLSCTTSAFTFD |
| | | GPDMAWYRQAPGNECVLVSIISADGRTYYADSVKGRFTISRDNAKNTVFLN |
| | | LNSLQPEDTAVYYCALDPRKNCRGGYCCANWGPGTQVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 76 | AS53750bbz | MALPVTALLLPLALLLHAARPEVQLVESGGGLVQPGGSLRLSCTASGFTDD |
| | | GPDMAWYRRAPGNECELVSIISADGRTYYTDSVKGRFTISRDNAKNTVFLY |
| | | LNSLQPEDTAVYYCAPDPRRNCRGGDCCGNWGPGTQVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 77 | AS54233bbz | MALPVTALLLPLALLLHAARPQVQLVESGGGSVQAGETLRLSCTASGFTFD |
| | | GPDMAWYRQAPGNECELVSIISADGRTYYTDSVKGRFTASQDNAKNTVSLY |
| | | LKSLQPEDTAVYYCAADPRRNCRGNCCGNWGPGTQVTVSSTTTPAPRPPTP |
| | | APTIASQPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGGCELRV |
| | | KFSRSADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKDTYDAL |
| | | HMQALPPR |
| 78 | AS53445bbz | MALPVTALLLPLALLLHAARPQVQLVESGGGSVQAGGSLRLSCTASGYIFC |
| | | MGWFRQAPGKAREGIATIYTGGDSTYYDDSVKGRFTISRDNAKNTVYLQMN |
| | | SLKPEDTAMYYCAAGGQECYLTNWVSYWGQGTQVTVSSTTTPAPRPPTPAP |
| | | TIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALHM |
| | | QALPPR |
| 79 | AS53574bbz | MALPVTALLLPLALLLHAARPQVKLVESGGGSVQAGGSLRLSCAASGYIYS |
| | | SNCMGWFRQAPGKEREWVARIHTGSGSTYYADSVKGRFTISQDNAKNTVYL |
| | | QMNSLRPEDTAMYDCAAGRVVLGAVVCTNEYWGQGTQVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 80 | AS57911bbz | MALPVTALLLPLALLLHAARPDIQMTQSPSSLSASVGDRVTITCRASQSVS |
| | | SAVAWYQQKPGKAPKLLIYSASSLYSGVPSRFSGSRSGTDFTLTISSLQPE |
| | | DFATYYCQQSHALITFGQGTKVEIKGGGGSGGGGSGGGGSEVQLVESGGGL |
| | | VQPGGSLRLSCAASGFNISSSYIHWVRQAPGKGLEWVAYISSYYSYTYYAD |
| | | SVKGRFTISADTSKNTAYLQMNSLRAEDTAVYYCARGYPYGMDYWGQGTLV |
| | | TVSSTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCS |
| | | CRFPEEEEGGCELRVKFSRSADAPAYKQGQNQLYNELNLGRREEYDVLDKR |
| | | RGRDPEMGGKPRRKNPQEGLYNELQKDKMAEAYSEIGMKGERRRGKGHDGL |
| | | YQGLSTATKDTYDALHMQALPPR |
| 81 | AS57659bbz | MALPVTALLLPLALLLHAARPDIQMTQSPSSLSASVGDRVTITCRASQSVS |
| | | SAVAWYQQKPGKAPKLLIYSASSLYSGVPSRFSGSRSGTDFTLTISSLQPE |
| | | DFATYYCQQPYYLITFGQGTKVEIKGGGGSGGGGSGGGGSEVQLVESGGGL |
| | | VQPGGSLRLSCAASGFNIYSYYIHWVRQAPGKGLEWVASIYSSYSSTYYAD |
| | | SVKGRFTISADTSKNTAYLQMNSLRAEDTAVYYCARSWFSYPGLDYWGQGT |
| | | LVTVSSTTTPAPRPPTPAPTIASQPLSLRPEACRPAAGGAVHTRGLDFACD |
| | | IYIWAPLAGTCGVLLLSLVITLYCKRGRKKLLYIFKQPFMRPVQTTQEEDG |
| | | CSCRFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRRGKGHD |
| | | GLYQGLSTATKDTYDALHMQALPPR |
| 82 | AS57765bbz | MALPVTALLLPLALLLHAARPDIQMTQSPSSLSASVGDRVTITCRASQSVS |
| | | SAVAWYQQKPGKAPKLLIYSASSLYSGVPSRFSGSRSGTDFTLTISSLQPE |
| | | DFATYYCQQAYYSLITFGQGTKVEIKGGGGSGGGGSGGGGSEVQLVESGGG |
| | | LVQPGGSLRLSCAASGFNIYYSYMHWVRQAPGKGLEWVAYIYPYSGSTSYA |
| | | DSVKGRFTISADTSKNTAYLQMNSLRAEDTAVYYCARPAVHWHGYGGGYYY |
| | | GLDYWGQGTLVTVSSTTTPAPRPPTPAPTIASQPLSLRPEACRPAAGGAVH |
| | | TRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGMKG |
| | | ERRRGKGHDGLYQGLSTATKDTYDALHMQALPPR |
| 83 | AS48542dis- | MALPVTALLLPLALLLHAARPQMQLVESGGGSVQAGETLRLSCTTSAFTFD |
| | bbz | GPDMAWYRQAPGNECVLVSIISADGRTYYADSVKGRFTISRDNAKNTVFLN |
| | | LNSLQPEDTAVYYCALDPRKNCRGGYCCANWGPGTQVTVSSGGGGSQMQLV |
| | | ESGGGSVQAGETLRLSCTTSAFTFDGPDMAWYRQAPGNECVLVSIISADGR |
| | | TYYADSVKGRFTISRDNAKNTVFLNLNSLQPEDTAVYYCALDPRKNCRGGY |
| | | CCANWGPGTQVTVSSTTTPAPRPPTPAPTIASQPLSLRPEACRPAAGGAVH |
| | | TRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGMKG |
| | | ERRRGKGHDGLYQGLSTATKDTYDALHMQALPPR |
| 84 | AS48542dil- | MALPVTALLLPLALLLHAARPQMQLVESGGGSVQAGETLRLSCTTSAFTFD |
| | bbz | GPDMAWYRQAPGNECVLVSIISADGRTYYADSVKGRFTISRDNAKNTVFLN |
| | | LNSLQPEDTAVYYCALDPRKNCRGGYCCANWGPGTQVTVSSGGGGSGGGGS |
| | | GGGGSQMQLVESGGGSVQAGETLRLSCTTSAFTFDGPDMAWYRQAPGNECV |
| | | LVSIISADGRTYYADSVKGRFTISRDNAKNTVFLNLNSLQPEDTAVYYCAL |
| | | DPRKNCRGGYCCANWGPGTQVTVSSTTTPAPRPPTPAPTIASQPLSLRPEA |
| | | CRPAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 85 | AS48542- | MALPVTALLLPLALLLHAARPQMQLVESGGGSVQAGETLRLSCTTSAFTFD |
| | AS53574bil- | GPDMAWYRQAPGNECVLVSIISADGRTYYADSVKGRFTISRDNAKNTVFLN |
| | bbz | LNSLQPEDTAVYYCALDPRKNCRGGYCCANWGPGTQVTVSSGGGGSGGGGS |
| | | GGGGSQVKLVESGGGSVQAGGSLRLSCAASGYIYSSNCMGWFRQAPGKERE |
| | | WVARIHTGSGSTYYADSVKGRFTISQDNAKNTVYLQMNSLRPEDTAM86YD |
| | | CAAGRVVLGAVVCTNEYWGQGTQVTVSSTTTPAPRPPTPAPTIASQPLSLR |
| | | PEACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 86 | AS53574- | MALPVTALLLPLALLLHAARPQVKLVESGGGSVQAGGSLRLSCAASGYIYS |
| | AS48542bil- | SNCMGWFRQAPGKEREWVARIHTGSGSTYYADSVKGRFTISQDNAKNTVYL |
| | bbz | QMNSLRPEDTAMYDCAAGRVVLGAVVCTNEYWGQGTQVTVSSGGGGSGGGG |
| | | SGGGGSQMQLVESGGGSVQAGETLRLSCTTSAFTFDGPDMAWYRQAPGNEC |
| | | VLVSIISADGRTYYADSVKGRFTISRDNAKNTVFLNLNSLQPEDTAVYYCA |
| | | LDPRKNCRGGYCCANWGPGTQVTVSSTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 87 | AS47863-CDR1 | GSTFGDSDMG |
| 87 | AS48433-CDR1 | GSTFGDSDMG |
| 88 | AS48463-CDR1 | GFTFANSDMG |
| 89 | AS48481-CDR1 | GFTFADSAMG |
| 90 | AS48508-CDR1 | RFTFDGPDMA |
| 91 | AS48542-CDR1 | AFTFDGPDMA |
| 92 | AS53445-CDR1 | GYIFCMG |
| 93 | AS53574-CDR1 | GYIYSSNCMG |
| 94 | AS53750-CDR1 | GFTDDGPDMA |
| 95 | AS54233-CDR1 | GFTFDGPDMA |
| 96 | AS57659VH-CDR1 | GFNIYSYYIH |
| 97 | AS57765VH-CDR1 | GFNIYYSYMH |
| 98 | AS57911VH-CDR1 | GFNISSSYIH |
| 99 | AS57659VL-CDR1 | RASQSVSSAVA |
| 99 | AS57765VL-CDR1 | RASQSVSSAVA |
| 99 | AS57911VL-CDR1 | RASQSVSSAVA |
| 100 | AS47863-CDR2 | IISSDGRTYYVDSVKG |
| 100 | AS48433-CDR2 | IISSDGRTYYVDSVKG |
| 101 | AS48463-CDR2 | IISSHGGTTYYVDSVKG |
| 102 | AS48481-CDR2 | IIRTDGTTYYGDSAKG |
| 103 | AS48508-CDR2 | IISADGRTYYTDSVKG |
| 104 | AS48542-CDR2 | IISADGRTYYADSVKG |
| 105 | AS53445-CDR2 | TIYTGGDSTYYDDSVKG |
| 106 | AS53574-CDR2 | RIHTGSGSTYYADSVKG |
| 103 | AS53750-CDR2 | IISADGRTYYTDSVKG |
| 103 | AS54233-CDR2 | IISADGRTYYTDSVKG |
| 107 | AS57659VH-CDR2 | SIYSSYSSTYYADSVKG |
| 108 | AS57765VH-CDR2 | YIYPYSGSTSYADSVKG |
| 109 | AS57911VH-CDR2 | YISSYYSYTYYADSVKG |
| 110 | AS57659VL-CDR2 | SASSLYS |
| 110 | AS57765VL-CDR2 | SASSLYS |
| 110 | AS57911VL-CDR2 | SASSLYS |
| 111 | AS47863-CDR3 | DLRQYCRDGRCCGY |
| 112 | AS48433-CDR3 | DLRLNCRDGRCCGY |
| 113 | AS48463-CDR3 | DPRSNCRGGYCCGY |
| 114 | AS48481-CDR3 | DRETSFIGGSWCVAKY |
| 115 | AS48508-CDR3 | DPRRNCRGGYCCGN |
| 116 | AS48542-CDR3 | DPRKNCRGGYCCAN |
| 117 | AS53445-CDR3 | GGQECYLTNWVSY |
| 118 | AS53574-CDR3 | GRVVLGAVVCTNEY |
| 119 | AS53750-CDR3 | DPRRNCRGGDCCGN |
| 120 | AS54233-CDR3 | DPRRNCRGNCCGN |
| 121 | AS57659VH-CDR3 | SWFSYPGLDY |
| 122 | AS57765VH-CDR3 | PAVHWHGYGGGYYYGLDY |
| 123 | AS57911VH-CDR3 | GYPYGMDY |
| 124 | AS57659VL-CDR3 | QQPYYLIT |
| 125 | AS57765VL-CDR3 | QQAYYSLIT |
| 126 | AS57911VL-CDR3 | QQSHALIT |
| 127 | CD28 hinge | IEVMYPPPYLDNEKSNGTIIHVKGKHLCPSPLFPGPSKP |
| 128 | CD28 TM | FWVLVVVGGVLACYSLLVTVAFIIFWV |
| 129 | cytoplasmic | RSKRSRLLHSDYMNMTPRRPGPTRKHYQPYAPPRDFAAYRS |
| | portion of | |
| | CD28 | |
| 130 | AS47863 | CAGGTGCAATTGGAGGAGTCTGGGGGAGGCTCGGTGCAGGCTGGAGAGA |
| | sdAb | CTCTGAGACTCTCCTGTACAGCCTCTGGATCCACTTTTGGTGATTCTGAC |
| | | ATGGGCTGGTACCGCCAGGCTCCAGGAAATGCGTGCGAGTTGGTATCAA |
| | | TTATTAGTAGTGACGGTAGGACATACTATGTGGACTCCGTGAAGGGCCG |
| | | ATTCACCATCTCCCAAGACAACGCCGTGAGCACGGTGTATCTGCAAATG |
| | | AACAGCCTGAAACCTGAGGACACAGGCGTGTATTACTGTGCGGCAGACC |
| | | TCCGCCAATATTGTAGGGATGGTCGCTGCTGCGGTTATTGGGGCCAGGGG |
| | | ACCCAGGTCACCGTCTCCTCA |
| 131 | AS48433 | CAGATTCAGCTGGTGGAGTCTGGGGGAGGCTCGGTGCAGGCTGGAGAGA |
| | sdAb | CTCTGAGACTCTCCTGTACAGCCTCTGGATCCACTTTTGGTGATTCTGAC |
| | | ATGGGCTGGTACCGCCAGGCTCCAGGGAATGCGTGCGAGTTGGTGTCAA |
| | | TTATTAGTAGTGACGGGCGGACATACTATGTGGACTCCGTGAAGGGCCG |
| | | ATTCACCATCTCCCAAGACAACGCCGTGAGCACGGTGTATCTGCAAATG |
| | | AACAGCCTGAATCCTGAGGACACAGGCGTGTATTACTGTGCGGCAGACC |
| | | TCCGCCTCAATTGTAGGGATGGTCGCTGCTGCGGTTATTGGGGCCAGGGG |
| | | ACCCAGGTCACCGTCTCCTCA |
| 132 | AS48463 | CAGGTGCACCTGATGGAGTCTGGGGGAGGCTCGGTGCAGGCTGGAGAGA |
| | sdAb | CTCTGAGACTCTCCTGTACAGCCTCTGGATTCACTTTTGCTAATTCTGACA |
| | | TGGGCTGGTACCGCCAGGCTCCAGGAAATGCGTGCGAGTTGGTCTCAAT |
| | | TATTAGTAGTCATGGTGGTACGACATACTATGTAGACTCCGTGAAGGGCC |
| | | GATTCACCATCTCCCGGCACAACGCCGAGAACACGGTGTATCTGCGAAT |
| | | GACTAGCCTGAAACCTGAGGACACAGCCCTATATTACTGTGTCGCAGAC |
| | | CCGAGGTCAAATTGTCGTGGTGGTTACTGCTGTGGTTACTGGGGCCCGGG |
| | | GACCCAGGTCACCGTCTCCTCA |
| 133 | AS48481 | GAGGTGCAACTGGTGGCGTCTGGGGGAGGCTCGGTGCAGGCTGGAGAGA |
| | sdAb | CTCTGAGACTCTCCTGTACAGCCTCTGGATTCACTTTTGCTGATTCTGCCA |
| | | TGGGCTGGTACCGAAAGGGTCCAGGGAATGTGTGCGACTTGGTAGCAAT |
| | | TATTAGGACAGATGGTACCACATACTATGGCGACTCCGCGAAGGGCCGA |
| | | TTCACCATCTCCCGAGACAACGCCAAGAGCACGCTGTATCTGCAAATGA |
| | | ACAGCCTGAAACCTGAGGATACAGCCGTGTATTTCTGTGCGGCAGACCG |
| | | GGAGACGTCTTTTATCGGTGGTAGCTGGTGTGTTGCTAAGTACTGGGACC |
| | | AGGGGACCCAGGTCACCGTCTCCTCA |
| 134 | AS48508 | GAGGTGCAGCTGGTGGAGTCTGGGGGAGGCTCGGTGCAGGCTGGAGGGT |
| | sdAb | CTCTGAGACTCTCATGTACAGCCTCTAGATTCACTTTTGATGGTCCCGAC |
| | | ATGGCCTGGTACCGCCAGGCTCCAGGGAATGCGTGCGAGTTGGTCTCAA |
| | | TTATTAGTGCTGATGGTAGAACCTACTATACAGACTCCGTGAAGGGCCGA |
| | | TTCACCATCTCCCGAGACAACGCCAAGAACACGGTGTTCCTGTATTTGAA |
| | | CAGCCTGCAACCTGAGGACACAGCCGTATATTACTGTGCGCCAGATCCC |
| | | CGTAGAAATTGTAGAGGTGGTTATTGCTGTGGCAACTGGGGCCCGGGGA |
| | | CCCAGGTCACCGTCTCCTCA |
| 135 | AS48542 | CAGATGCAGCTGGTGGAGTCTGGGGGAGGCTCGGTGCAGGCTGGAGAGA |
| | sdAb | CTCTGAGACTCTCATGTACAACCTCTGCCTTCACTTTTGATGGTCCCGAC |
| | | ATGGCCTGGTACCGCCAGGCTCCAGGGAATGAGTGCGTGTTGGTCTCAA |
| | | TTATTAGTGCTGATGGTAGAACCTACTATGCAGACTCCGTGAAGGGCCGA |
| | | TTCACCATCTCCCGAGACAACGCCAAGAACACGGTGTTCCTGAATTTGAA |
| | | CAGCCTGCAACCTGAGGACACAGCCGTATATTACTGTGCGTTAGATCCCC |
| | | GTAAAAATTGTAGAGGTGGTTATTGCTGTGCCAACTGGGGCCCGGGGAC |
| | | CCAGGTCACCGTCTCCTCA |
| 136 | AS53445 | CAGGTGCAGCTGGTGGAGTCTGGGGGAGGCTCGGTACAGGCTGGAGGGT |
| | sdAb | CTCTGAGACTCTCCTGTACAGCCTCTGGATACATTTTTTGCATGGGCTGGT |
| | | TCCGCCAGGCTCCAGGGAAGGCCCGCGAGGGGATCGCAACTATTTATAC |
| | | GGGTGGTGATAGCACATATTATGACGACTCCGTGAAGGGCCGATTCACC |
| | | ATCTCCCGGGACAACGCCAAGAACACGGTGTATCTGCAAATGAACAGCC |
| | | TGAAACCTGAGGACACTGCCATGTACTACTGTGCGGCAGGGGGCCAAGA |
| | | GTGCTATTTAACGAACTGGGTTAGCTACTGGGGCCAGGGGACCCAGGTC |
| | | ACCGTCTCCTCA |
| 137 | AS53574 | CAGGTGAAGTTAGTGGAGTCTGGGGGAGGCTCGGTGCAGGCTGGAGGGT |
| | sdAb | CTCTGAGACTCTCCTGTGCAGCCTCTGGATACATCTACAGTAGTAACTGC |
| | | ATGGGCTGGTTCCGCCAGGCTCCAGGGAAGGAGCGCGAGTGGGTCGCAC |
| | | GTATTCATACTGGTAGTGGTAGCACATACTATGCCGACTCCGTGAAGGGC |
| | | CGATTCACCATCTCCCAAGACAACGCCAAGAACACGGTGTACCTGCAAA |
| | | TGAACAGCCTGAGACCTGAGGACACTGCCATGTACGACTGTGCGGCAGG |
| | | CCGAGTGGTACTTGGTGCGGTGGTCTGCACGAATGAGTACTGGGGCCAG |
| | | GGGACCCAGGTCACCGTCTCCTCA |
| 138 | AS53750 | GAGGTGCAGCTGGTGGAGTCTGGGGGAGGCTTGGTGCAGCCTGGGGGGT |
| | sdAb | CTCTGAGACTCTCATGTACAGCCTCTGGATTCACTGATGATGGTCCCGAC |
| | | ATGGCCTGGTACCGCCGGGCTCCAGGGAATGAGTGCGAGTTGGTCTCAA |
| | | TTATTAGTGCTGATGGTAGAACCTACTATACAGACTCCGTGAAGGGGCG |
| | | ATTCACCATCTCCCGAGACAACGCCAAAAACACGGTGTTCCTGTATTTGA |
| | | ACAGCCTGCAACCTGAGGACACAGCCGTATATTACTGTGCGCCAGATCC |
| | | CCGTAGAAATTGTAGAGGTGGTGATTGCTGTGGCAACTGGGGCCCGGGG |
| | | ACCCAGGTCACCGTCTCCTCA |
| 139 | AS54233 | CAGGTGCAGCTGGTGGAGTCTGGGGGAGGCTCGGTGCAGGCTGGAGAGA |
| | sdAb | CTCTGAGACTCTCATGTACAGCCTCTGGATTCACTTTTGATGGTCCCGAC |
| | | ATGGCCTGGTACCGCCAGGCTCCAGGGAATGAGTGCGAGTTGGTCTCAA |
| | | TTATTAGTGCTGATGGTAGAACCTACTATACAGACTCCGTGAAGGGCCGA |
| | | TTCACCGCCTCCCAAGACAACGCCAAGAACACGGTGTCTCTATATTTGAA |
| | | AAGCCTGCAACCTGAGGACACAGCCGTATATTACTGTGCGGCAGATCCC |
| | | CGTAGAAATTGTAGAGGTAATTGCTGTGGCAACTGGGGCCCGGGGACCC |
| | | AGGTCACCGTCTCCTCA |
| 140 | AS47863VH4 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCC |
| | | ATCATCAGCTCCGACGGCAGGACATACTATGTGGATTCTGTGAAGGGCC |
| | | GCTTTACCATCTCTCAGGACAACAGCAAGAATACACTGTATCTGCAGATG |
| | | AACTCTCTGCGGGCCGAGGATACCGCCGTGTACTATTGCGCCGCCGACCT |
| | | GAGACAGTACTGTCGGGATGGCAGATGCTGTGGCTATTGGGGCCAGGGC |
| | | ACCCTGGTGACAGTGTCTAGC |
| 141 | AS47863VH5 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCC |
| | | ATCATCAGCTCCGACGGCAGGACATACTATGTGGATTCTGTGAAGGGCC |
| | | GCTTTACCATCTCTCAGGACAACAGCAAGAATACAGTGTATCTGCAGAT |
| | | GAACTCTCTGCGGGCCGAGGATACCGCCGTGTACTATTGCGCCGCCGAC |
| | | CTGAGACAGTACTGTCGGGATGGCAGATGCTGTGGCTATTGGGGCCAGG |
| | | GCACCCTGGTGACAGTGTCTAGC |
| 142 | AS47863VH11 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCT |
| | | ATCATCAGCTCCGACGGCAGGACATACTATGTGGATTCTGTGAAGGGCC |
| | | GCTTTACCATCAGCCAGGACAACGCCAAGAATACACTGTATCTGCAGAT |
| | | GAACTCCCTGCGGCCCGAGGATACCGCCGTGTACTATTGCGCCGCCGAC |
| | | CTGAGACAGTACTGTCGGGATGGCAGATGCTGTGGCTATTGGGGCCAGG |
| | | GCACCCTGGTGACAGTGTCTAGC |
| 143 | AS47863VH12 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCT |
| | | ATCATCAGCTCCGACGGCAGGACATACTATGTGGATTCTGTGAAGGGCC |
| | | GCTTTACCATCAGCCAGGACAACGCCAAGAATACAGTGTATCTGCAGAT |
| | | GAACTCCCTGCGGCCCGAGGATACCGCCGTGTACTATTGCGCCGCCGAC |
| | | CTGAGACAGTACTGTCGGGATGGCAGATGCTGTGGCTATTGGGGCCAGG |
| | | GCACCCTGGTGACAGTGTCTAGC |
| 144 | AS48433VH4 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCC |
| | | ATCATCAGCTCCGACGGCAGGACATACTATGTGGATTCTGTGAAGGGCC |
| | | GCTTTACCATCTCTCAGGACAACAGCAAGAATACACTGTACCTGCAGAT |
| | | GAACTCTCTGCGGGCCGAGGATACCGCCGTGTACTATTGCGCCGCCGAC |
| | | CTGAGACTGAATTGTCGGGATGGCAGATGCTGTGGCTATTGGGGCCAGG |
| | | GCACCCTGGTGACAGTGTCTAGC |
| 145 | AS48433VH5 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCC |
| | | ATCATCAGCTCCGACGGCAGGACATACTATGTGGATTCTGTGAAGGGCC |
| | | GCTTTACCATCTCTCAGGACAACAGCAAGAATACAGTGTACCTGCAGAT |
| | | GAACTCTCTGCGGGCCGAGGATACCGCCGTGTACTATTGCGCCGCCGAC |
| | | CTGAGACTGAATTGTCGGGATGGCAGATGCTGTGGCTATTGGGGCCAGG |
| | | GCACCCTGGTGACAGTGTCTAGC |
| 146 | AS48433VH11 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCT |
| | | ATCATCAGCTCCGACGGCAGGACATACTATGTGGATTCTGTGAAGGGCC |
| | | GCTTTACCATCAGCCAGGACAACGCCAAGAATACACTGTACCTGCAGAT |
| | | GAACTCCCTGCGGCCCGAGGATACCGCCGTGTACTATTGCGCCGCCGAC |
| | | CTGAGACTGAATTGTCGGGATGGCAGATGCTGTGGCTATTGGGGCCAGG |
| | | GCACCCTGGTGACAGTGTCTAGC |
| 147 | AS48433VH12 | GAGGTGCAGCTGGTGGAGAGCGGAGGAGGACTGGTGCAGCCAGGAGGC |
| | sdAb | AGCCTGAGGCTGTCCTGCGCAGCATCCGGATCTACCTTCGGCGACTCCGA |
| | | TATGGGCTGGTACAGACAGGCCCCTGGCAAGGGCTGTGAGCTGGTGTCT |
| | | ATCATCAGCTCCGACGGCAGGACATACTATGTGGATTCTGTGAAGGGCC |
| | | GCTTTACCATCAGCCAGGACAACGCCAAGAATACAGTGTACCTGCAGAT |
| | | GAACTCCCTGCGGCCCGAGGATACCGCCGTGTACTATTGCGCCGCCGAC |
| | | CTGAGACTGAATTGTCGGGATGGCAGATGCTGTGGCTATTGGGGCCAGG |
| | | GCACCCTGGTGACAGTGTCTAGC |
| 148 | AS48463VH4 | GAGGTGCAGCTGCTGGAGTCCGGAGGAGGACTGGTGCAGCCAGGAGGCT |
| | sdAb | CCCTGAGGCTGTCTTGCGCAGCAAGCGGCTTCACCTTTGCCAACTCTGAC |
| | | ATGGGATGGTACAGGCAGGCACCTGGCAAGGGATGTGAGCTGGTGAGCA |
| | | TCATCAGCTCCCACGGCGGCACCACATACTATGTGGACTCCGTGAAGGG |
| | | CAGGTTCACCATCTCCCGCGATAACTCTAAGAATACACTGTATCTGCAGA |
| | | TGAACTCTCTGCGGGCCGAGGACACAGCCGTGTACTATTGCGTGGCCGA |
| | | TCCCCGGAGCAATTGTAGAGGCGGCTACTGCTGTGGCTATTGGGGCCAG |
| | | GGCACCCTGGTGACAGTGTCTAGC |
| 149 | AS48463VH11 | GAGGTGCAGCTGCTGGAGTCCGGAGGAGGACTGGTGCAGCCAGGAGGCT |
| | sdAb | CCCTGAGGCTGTCTTGCGCAGCAAGCGGCTTCACCTTTGCCAACAGCGAC |
| | | ATGGGATGGTACAGGCAGGCACCAGGCAAGGGATGTGAGCTGGTGTCCA |
| | | TCATCAGCTCCCACGGCGGCACCACATACTATGTGGACTCCGTGAAGGG |
| | | CAGGTTCACCATCTCTCGCGATAACGCCAAGAATACACTGTATCTGCAGA |
| | | TGAACTCTCTGCGGCCCGAGGACACAGCCGTGTACTATTGCGTGGCCGAT |
| | | CCTCGGAGCAATTGTAGAGGCGGCTACTGCTGTGGCTATTGGGGCCAGG |
| | | GCACCCTGGTGACAGTGTCTAGC |
| 150 | AS48481VH5 | CAGGTGCAGCTGGTGGAGTCTGGAGGAGGAGTGGTGCAGCCAGGCCGGT |
| | sdAb | CTCTGAGACTGAGCTGCGCAGCATCCGGCTTCACCTTTGCCGACAGCGCC |
| | | ATGGGATGGTACAGGCAGGCACCTGGCAAGGGATGTGAGCTGGTGGCCA |
| | | TCATCAGAACAGACGGCACCACATACTATGGCGATAGCGCCAAGGGCAG |
| | | GTTCACCATCTCTCGCGATAACAGCAAGAATACACTGTACCTGCAGATG |
| | | AACTCCCTGAGGGCAGAGGACACCGCCGTGTATTTCTGCGCCGCCGATA |
| | | GAGAGACATCCTTTATCGGCGGCTCTTGGTGCGTGGCCAAGTATTGGGAC |
| | | CAGGGCACCCTGGTGACAGTGAGCTCC |
| 151 | AS48481VH6 | CAGGTGCAGCTGGTGGAGTCTGGAGGAGGAGTGGTGCAGCCAGGCCGGT |
| | sdAb | CTCTGAGACTGAGCTGCGCAGCATCCGGCTTCACCTTTGCCGACAGCGCC |
| | | ATGGGATGGTACAGGCAGGCACCTGGCAAGGTATGTGAGCTGGTGGCCA |
| | | TCATCAGAACAGACGGCACCACATACTATGGCGATAGCGCCAAGGGCAG |
| | | GTTCACCATCTCTCGCGATAACAGCAAGAATACACTGTACCTGCAGATG |
| | | AACTCCCTGAGGGCAGAGGACACCGCCGTGTATTTCTGCGCCGCCGATA |
| | | GAGAGACATCCTTTATCGGCGGCTCTTGGTGCGTGGCCAAGTATTGGGAC |
| | | CAGGGCACCCTGGTGACAGTGAGCTCC |
| 152 | AS48481VH13 | CAGGTGCAGCTGGTGGAGAGCGGAGGAGGAGTGGTGCAGCCAGGACGG |
| | sdAb | TCTCTGAGACTGAGCTGCGCAGCATCCGGCTTCACCTTTGCAGACTCCGC |
| | | AATGGGATGGTACAGGCAGGCACCTGGCAAGGGATGTGAGCTGGTGGCC |
| | | ATCATCAGAACAGACGGCACCACATACTATGGCGATTCCGCCAAGGGCA |
| | | GGTTCACCATCTCTCGCGATAACGCCAAGAATACACTGTACCTGCAGATG |
| | | AACTCTCTGCGGCCCGAGGACACCGCCGTGTATTTCTGCGCCGCCGATAG |
| | | AGAGACATCTTTTATCGGCGGCAGCTGGTGTGTGGCCAAGTATTGGGACC |
| | | AGGGCACCCTGGTGACAGTGAGCTCC |
| 153 | AS48481VH14 | CAGGTGCAGCTGGTGGAGAGCGGAGGAGGAGTGGTGCAGCCAGGACGG |
| | sdAb | TCTCTGAGACTGAGCTGCGCAGCATCCGGCTTCACCTTTGCAGACTCCGC |
| | | AATGGGATGGTACAGGCAGGCACCTGGCAAGGTCTGTGAGCTGGTGGCC |
| | | ATCATCAGAACAGACGGCACCACATACTATGGCGATTCCGCCAAGGGCA |
| | | GGTTCACCATCTCTCGCGATAACGCCAAGAATACACTGTACCTGCAGATG |
| | | AACTCTCTGCGGCCCGAGGACACCGCCGTGTATTTCTGCGCCGCCGATAG |
| | | AGAGACATCTTTTATCGGCGGCAGCTGGTGTGTGGCCAAGTATTGGGACC |
| | | AGGGCACCCTGGTGACAGTGAGCTCC |
| 154 | AS48508VH4 | GAGGTGCAGCTGGTGGAGTCTGGAGGAGGACTGGTGCAGCCAGGAGGCT |
| | sdAb | CCCTGCGGCTGTCTTGCGCCGCCAGCAGATTCACCTTTGACGGCCCAGAT |
| | | ATGGCATGGTACAGGCAGGCACCAGGCAAGGGATGTGAGCTGGTGTCTA |
| | | TCATCAGCGCCGACGGCCGCACCTACTATACAGATAGCGTGAAGGGCAG |
| | | GTTCACCATCTCCCGCGACAACTCTAAGAATACACTGTACCTGCAGATGA |
| | | ACTCCCTGAGGGCAGAGGACACCGCAGTGTACTATTGCGCCCCCGATCC |
| | | TCGGAGAAACTGTCGGGGCGGCTATTGCTGTGGCAATTGGGGCCAGGGC |
| | | ACCACAGTGACAGTGAGCTCC |
| 155 | AS48508VH5 | GAGGTGCAGCTGGTGGAGTCTGGAGGAGGACTGGTGCAGCCAGGAGGCT |
| | sdAb | CCCTGCGGCTGTCTTGCGCCGCCAGCAGATTCACCTTTGACGGCCCAGAT |
| | | ATGGCATGGTACAGGCAGGCACCAGGCAAGGGATGTGAGCTGGTGTCTA |
| | | TCATCAGCGCCGACGGCCGCACCTACTATACAGATAGCGTGAAGGGCAG |
| | | GTTCACCATCTCCCGCGACAACTCTAAGAATACAGTGTACCTGCAGATGA |
| | | ACTCCCTGAGGGCAGAGGACACCGCAGTGTACTATTGCGCCCCCGATCC |
| | | TCGGAGAAACTGTCGGGGCGGCTATTGCTGTGGCAATTGGGGCCAGGGC |
| | | ACCACAGTGACAGTGAGCTCC |
| 156 | AS48508VH11 | GAGGTGCAGCTGGTGGAGAGCGGAGGAGGACTGGTGCAGCCTGGAGGC |
| | sdAb | TCCCTGAGGCTGTCTTGCGCAGCAAGCAGATTCACCTTTGACGGCCCAGA |
| | | TATGGCATGGTACAGGCAGGCACCAGGCAAGGGATGTGAGCTGGTGTCT |
| | | ATCATCAGCGCCGACGGCCGCACCTACTATACAGATTCCGTGAAGGGCA |
| | | GGTTCACCATCTCTCGCGACAACGCCAAGAATACACTGTACCTGCAGAT |
| | | GAACTCCCTGAGGCCAGAGGACACCGCAGTGTACTATTGCGCCCCCGAT |
| | | CCTCGGAGAAACTGTCGGGGCGGCTATTGCTGTGGCAATTGGGGCCAGG |
| | | GCACCACAGTGACAGTGAGCTCC |
| 157 | AS48508VH12 | GAGGTGCAGCTGGTGGAGAGCGGAGGAGGACTGGTGCAGCCTGGAGGC |
| | sdAb | TCCCTGAGGCTGTCTTGCGCAGCAAGCAGATTCACCTTTGACGGCCCAGA |
| | | TATGGCATGGTACAGGCAGGCACCAGGCAAGGGATGTGAGCTGGTGTCT |
| | | ATCATCAGCGCCGACGGCCGCACCTACTATACAGATTCCGTGAAGGGCA |
| | | GGTTCACCATCTCTCGCGACAACGCCAAGAATACAGTGTACCTGCAGAT |
| | | GAACTCCCTGAGGCCAGAGGACACCGCAGTGTACTATTGCGCCCCCGAT |
| | | CCTCGGAGAAACTGTCGGGGCGGCTATTGCTGTGGCAATTGGGGCCAGG |
| | | GCACCACAGTGACAGTGAGCTCC |
| 158 | AS48542VH5 | GAGGTGCAGCTGGTGGAGTCCGGAGGAGGACTGGTGCAGCCAGGAGGC |
| | sdAb | TCCCTGAGGCTGTCTTGCGCCACAAGCGCCTTCACCTTTGACGGCCCCGA |
| | | TATGGCATGGTACAGGCAGGCACCTGGCAAGGGATGTGAGCTGGTGTCT |
| | | ATCATCAGCGCCGACGGCCGCACATACTATGCCGATTCTGTGAAGGGCA |
| | | GGTTCACAATCTCCCGCGACAACTCTAAGAATACCGTGTACCTGCAGATG |
| | | AACAGCCTGAGGGCAGAGGACACCGCCGTGTACTATTGCGCCCTGGATC |
| | | CCCGGAAGAACTGTAGAGGCGGCTATTGCTGTGCCAATTGGGGCCAGGG |
| | | CACACTGGTGACCGTGAGCTCC |
| 159 | AS48542VH12 | GAGGTGCAGCTGGTGGAGTCTGGAGGAGGACTGGTGCAGCCTGGAGGCT |
| | sdAb | CCCTGAGGCTGTCTTGCGCCACAAGCGCCTTCACCTTTGACGGCCCCGAT |
| | | ATGGCATGGTACAGGCAGGCACCTGGCAAGGGATGTGAGCTGGTGTCTA |
| | | TCATCAGCGCCGACGGCCGCACATACTATGCCGATAGCGTGAAGGGCAG |
| | | GTTCACAATCTCCCGCGACAACGCCAAGAATACCGTGTACCTGCAGATG |
| | | AACAGCCTGCGGCCAGAGGACACCGCCGTGTACTATTGCGCCCTGGATC |
| | | CCCGGAAGAACTGTAGAGGCGGCTATTGCTGTGCCAATTGGGGCCAGGG |
| | | CACACTGGTGACCGTGAGCTCC |
| 160 | AS53445VH4 | CAGGTGCAGCTGGTGGAGTCTGGAGGAGGAGTGGTGCAGCCAGGAGGCT |
| | sdAb | CTCTGAGGCTGAGCTGCGCAGCATCCGGATACATCTTCTGTATGGGATGG |
| | | TTTAGGCAGGCACCTGGCAAGGGACTGGAGGGAATCGCCACCATCTATA |
| | | CAGGCGGCGACTCCACCTACTATGACGATTCTGTGAAGGGCCGGTTCAC |
| | | CATCTCTAGAGATAACAGCAAGAATACACTGTACCTGCAGATGAACAGC |
| | | CTGAGGGCAGAGGACACCGCAGTGTACTATTGCGCAGCAGGAGGACAG |
| | | GAGTGTTACCTGACAAATTGGGTGTCCTATTGGGGCCAGGGCACCCTGGT |
| | | GACAGTGAGCTCC |
| 161 | AS53445VH11 | CAGGTGCAGCTGGTGGAGAGCGGAGGAGGAGTGGTGCAGCCAGGAGGC |
| | sdAb | TCTCTGCGGCTGAGCTGCGCCGCCTCCGGCTACATCTTCTGTATGGGCTG |
| | | GTTTAGGCAGGCACCTGGCAAGGGAAGGGAGGGAATCGCAACCATCTAT |
| | | ACAGGCGGCGACTCTACCTACTATGACGATAGCGTGAAGGGCCGGTTCA |
| | | CCATCTCCAGAGATAACGCCAAGAATACACTGTACCTGCAGATGAACTC |
| | | TCTGAGGCCCGAGGACACCGCAGTGTACTATTGCGCAGCAGGAGGACAG |
| | | GAGTGTTACCTGACAAATTGGGTGTCCTATTGGGGCCAGGGCACCCTGGT |
| | | GACAGTGAGCTCC |
| 162 | AS53574VH4 | GAGGTGCAGCTGGTGGAGTCCGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGATGGTTCAGGCAGGCACCTGGCAAGGGACTGGAGTGGGTGTCT |
| | | CGCATCCACACCGGCTCCGGCTCTACATACTATGCCGACAGCGTGAAGG |
| | | GCCGGTTTACCATCAGCAGAGATAACTCCAAGAATACACTGTACCTGCA |
| | | GATGAACTCTCTGCGGGCCGAGGACACCGCAGTGTACTATTGCGCAGCA |
| | | GGAAGGGTGGTGCTGGGAGCAGTGGTGTGTACAAATGAGTATTGGGGCC |
| | | AGGGCACCCTGGTGACAGTGTCTAGC |
| 163 | AS53574VH5 | GAGGTGCAGCTGGTGGAGTCCGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGATGGTTCAGGCAGGCACCTGGCAAGGGACTGGAGTGGGTGTCT |
| | | AGAATCCACACCGGCTCCGGCTCTACATACTATGCCGACAGCGTGAAGG |
| | | GCAGGTTTACCATCAGCCAGGATAACTCCAAGAATACACTGTACCTGCA |
| | | GATGAACTCTCTGAGGGCCGAGGACACCGCAGTGTACTATTGCGCAGCA |
| | | GGAAGGGTGGTGCTGGGAGCAGTGGTGTGTACAAATGAGTATTGGGGCC |
| | | AGGGCACCCTGGTGACAGTGTCTAGC |
| 164 | AS53574VH6 | GAGGTGCAGCTGGTGGAGTCCGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGATGGTTCAGGCAGGCACCTGGCAAGGGCCTGGAGTGGGTGGCC |
| | | AGAATCCACACCGGCTCCGGCTCTACATACTATGCCGACTCTGTGAAGG |
| | | GCAGGTTTACCATCAGCCAGGATAACTCCAAGAATACACTGTACCTGCA |
| | | GATGAACAGCCTGAGGGCCGAGGACACCGCAGTGTACTATTGCGCAGCA |
| | | GGAAGGGTGGTGCTGGGAGCAGTGGTGTGTACAAATGAGTATTGGGGCC |
| | | AGGGCACCCTGGTGACAGTGTCTAGC |
| 165 | AS53574VH11 | GAGGTGCAGCTGGTGGAGTCCGGAGGAGGACTGGTGCAGCCAGGAGGC |
| | sdAb | AGCCTGAGGCTGTCCTGCGCCGCCTCTGGCTACATCTATAGCTCCAACTG |
| | | TATGGGCTGGTTCAGACAGGCACCTGGCAAGGGAAGGGAGTGGGTGTCT |
| | | AGAATCCACACCGGCTCCGGCTCTACATACTATGCCGACAGCGTGAAGG |
| | | GCAGGTTTACCATCTCCCGCGATAACGCCAAGAATACACTGTACCTGCA |
| | | GATGAACAGCCTGAGGCCAGAGGACACCGCAGTGTACTATTGCGCAGCA |
| | | GGAAGAGTGGTGCTGGGAGCAGTGGTGTGTACAAATGAGTATTGGGGCC |
| | | AGGGCACCCTGGTGACAGTGTCTAGC |
| 166 | AS53574VH12 | GAGGTGCAGCTGGTGGAGTCCGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGCTGGTTCAGGCAGGCACCTGGCAAGGGAAGGGAGTGGGTGTCT |
| | | AGAATCCACACCGGCTCCGGCTCTACATACTATGCCGACAGCGTGAAGG |
| | | GCCGGTTTACCATCTCCCAGGATAACGCCAAGAATACACTGTACCTGCA |
| | | GATGAACAGCCTGAGGCCCGAGGACACCGCAGTGTACTATTGCGCAGCA |
| | | GGAAGGGTGGTGCTGGGAGCAGTGGTGTGTACAAATGAGTATTGGGGCC |
| | | AGGGCACCCTGGTGACAGTGTCTAGC |
| 167 | AS53574VH13 | GAGGTGCAGCTGGTGGAGTCTGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGCTGGTTCAGGCAGGCACCTGGCAAGGGAAGGGAGTGGGTGGCC |
| | | AGAATCCACACCGGCTCCGGCTCTACATACTATGCCGACAGCGTGAAGG |
| | | GCCGGTTTACCATCTCCCAGGATAACGCCAAGAATACACTGTACCTGCA |
| | | GATGAACAGCCTGAGGCCCGAGGACACCGCAGTGTACTATTGCGCAGCA |
| | | GGAAGGGTGGTGCTGGGAGCAGTGGTGTGTACAAATGAGTATTGGGGCC |
| | | AGGGCACCCTGGTGACAGTGTCTAGC |
| 168 | AS53750VH4 | GAGGTGCAGCTGGTGGAGTCTGGAGGAGGACTGGTGCAGCCAGGAGGCT |
| | sdAb | CCCTGAGGCTGTCTTGCGCAGCAAGCGGCTTCACCGACGATGGACCAGA |
| | | TATGGCATGGTACAGGCAGGCACCAGGCAAGGGATGTGAGCTGGTGTCT |
| | | ATCATCAGCGCCGACGGCAGAACCTACTATACAGATAGCGTGAAGGGCA |
| | | GGTTTACCATCTCCCGCGACAACTCTAAGAATACACTGTATCTGCAGATG |
| | | AACTCCCTGAGGGCCGAGGACACCGCCGTGTACTATTGCGCCCCCGATC |
| | | CTCGGAGAAACTGTAGGGGAGGCGACTGCTGTGGAAATTGGGGACAGGG |
| | | CACCACAGTGACAGTGAGCTCC |
| 169 | AS53750VH5 | GAGGTGCAGCTGGTGGAGTCTGGAGGAGGACTGGTGCAGCCAGGAGGCT |
| | sdAb | CCCTGAGGCTGTCTTGCGCAGCAAGCGGCTTCACCGACGATGGACCAGA |
| | | TATGGCATGGTACAGGCAGGCACCAGGCAAGGGATGTGAGCTGGTGTCT |
| | | ATCATCAGCGCCGACGGCAGAACCTACTATACAGATAGCGTGAAGGGCA |
| | | GGTTTACCATCTCCCGCGACAACTCTAAGAATACAGTGTATCTGCAGATG |
| | | AACTCCCTGAGGGCCGAGGACACCGCCGTGTACTATTGCGCCCCCGATC |
| | | CTCGGAGAAACTGTAGGGGAGGCGACTGCTGTGGAAATTGGGGACAGGG |
| | | CACCACAGTGACAGTGAGCTCC |
| 170 | AS53750VH11 | GAGGTGCAGCTGGTGGAGAGCGGAGGAGGACTGGTGCAGCCTGGAGGC |
| | sdAb | TCCCTGAGGCTGTCTTGCGCAGCAAGCGGCTTCACCGACGATGGACCAG |
| | | ATATGGCATGGTACAGGCAGGCACCAGGCAAGGGATGTGAGCTGGTGTC |
| | | TATCATCAGCGCCGACGGCAGAACCTACTATACAGATTCCGTGAAGGGC |
| | | AGGTTTACCATCTCTCGCGACAACGCCAAGAATACACTGTATCTGCAGAT |
| | | GAACTCCCTGAGGCCCGAGGACACCGCCGTGTACTATTGCGCCCCCGAT |
| | | CCTCGGAGAAACTGTAGGGGAGGCGACTGCTGTGGAAATTGGGGACAGG |
| | | GCACCACAGTGACAGTGAGCTCC |
| 171 | AS53750VH12 | GAGGTGCAGCTGGTGGAGAGCGGAGGAGGACTGGTGCAGCCTGGAGGC |
| | sdAb | TCCCTGAGGCTGTCTTGCGCAGCAAGCGGCTTCACCGACGATGGACCAG |
| | | ATATGGCATGGTACAGGCAGGCACCAGGCAAGGGATGTGAGCTGGTGTC |
| | | TATCATCAGCGCCGACGGCAGAACCTACTATACAGATTCCGTGAAGGGC |
| | | AGGTTTACCATCTCTCGCGACAACGCCAAGAATACAGTGTATCTGCAGAT |
| | | GAACTCCCTGAGGCCCGAGGACACCGCCGTGTACTATTGCGCCCCCGAT |
| | | CCTCGGAGAAACTGTAGGGGAGGCGACTGCTGTGGAAATTGGGGACAGG |
| | | GCACCACAGTGACAGTGAGCTCC |
| 172 | AS54233VH4 | GAGGTGCAGCTGGTGGAGTCTGGAGGAGGACTGGTGCAGCCAGGAGGCT |
| | sdAb | CCCTGAGGCTGTCTTGCGCAGCAAGCGGCTTCACCTTTGACGGACCCGAT |
| | | ATGGCCTGGTACAGACAGGCCCCTGGCAAGGGCTGTGAGCTGGTGTCTA |
| | | TCATCAGCGCCGACGGCAGGACCTACTATACAGATAGCGTGAAGGGACG |
| | | CTTCACCGCATCCCAGGACAACTCTAAGAATACACTGTATCTGCAGATGA |
| | | ACAGCCTGCGGGCCGAGGACACAGCCGTGTACTATTGCGCCGCCGATCC |
| | | CCGGAGAAACTGTAGAGGCAATTGCTGTGGAAACTGGGGACAGGGAAC |
| | | CCTGGTGACAGTGAGCTCC |
| 173 | AS54233VH5 | GAGGTGCAGCTGGTGGAGTCTGGAGGAGGACTGGTGCAGCCAGGAGGCT |
| | sdAb | CCCTGAGGCTGTCTTGCGCAGCAAGCGGCTTCACCTTTGACGGACCCGAT |
| | | ATGGCCTGGTACAGACAGGCCCCTGGCAAGGGCTGTGAGCTGGTGTCTA |
| | | TCATCAGCGCCGACGGCAGGACCTACTATACAGATAGCGTGAAGGGACG |
| | | CTTCACCGCATCCCAGGACAACTCTAAGAATACAGTGTATCTGCAGATG |
| | | AACAGCCTGCGGGCCGAGGACACAGCCGTGTACTATTGCGCCGCCGATC |
| | | CCCGGAGAAACTGTAGAGGCAATTGCTGTGGAAACTGGGGACAGGGAA |
| | | CCCTGGTGACAGTGAGCTCC |
| 174 | AS54233VH11 | GAGGTGCAGCTGGTGGAGAGCGGAGGAGGACTGGTGCAGCCAGGAGGC |
| | sdAb | TCCCTGAGGCTGTCTTGCGCAGCAAGCGGCTTCACCTTTGACGGACCCGA |
| | | TATGGCCTGGTACAGACAGGCCCCTGGCAAGGGCTGTGAGCTGGTGTCT |
| | | ATCATCAGCGCCGACGGCAGGACCTACTATACAGATTCCGTGAAGGGCC |
| | | GCTTCACCGCCTCTCAGGACAACGCCAAGAATACACTGTATCTGCAGAT |
| | | GAACAGCCTGCGGCCAGAGGACACAGCCGTGTACTATTGCGCCGCCGAT |
| | | CCCCGGAGAAACTGTAGAGGCAATTGCTGTGGAAACTGGGGACAGGGA |
| | | ACCCTGGTGACAGTGAGCTCC |
| 175 | AS54233VH12 | GAGGTGCAGCTGGTGGAGAGCGGAGGAGGACTGGTGCAGCCAGGAGGC |
| | sdAb | TCCCTGAGGCTGTCTTGCGCAGCAAGCGGCTTCACCTTTGACGGACCCGA |
| | | TATGGCCTGGTACAGACAGGCCCCTGGCAAGGGCTGTGAGCTGGTGTCT |
| | | ATCATCAGCGCCGACGGCAGGACCTACTATACAGATTCCGTGAAGGGCC |
| | | GCTTCACCGCCTCTCAGGACAACGCCAAGAATACAGTGTATCTGCAGAT |
| | | GAACAGCCTGCGGCCAGAGGACACAGCCGTGTACTATTGCGCCGCCGAT |
| | | CCCCGGAGAAACTGTAGAGGCAATTGCTGTGGAAACTGGGGACAGGGA |
| | | ACCCTGGTGACAGTGAGCTCC |
| 176 | 5F11 scFv | GGCAGCACCTCCGGATCTGGCAAGCCAGGAAGCGGAGAGGGCAGCACA |
| | linker | AAGGGC |
| 177 | (G4S)₃ linker | GGAGGAGGAGGAAGCGGAGGAGGAGGATCCGGCGGCGGCGGCTCT |
| 178 | G4S linker | GGAGGAGGAGGAAGC |
| 179 | AS57911 scFv | GACATCCAGATGACCCAGAGCCCGAGCAGCCTGAGCGCGAGCGTTGGTG |
| | | ACCGTGTTACCATTACCTGCCGTGCGAGCCAGAGCGTTAGCAGCGCGGT |
| | | GGCGTGGTACCAGCAAAAGCCGGGTAAAGCGCCGAAGCTGCTGATCTAT |
| | | AGCGCGAGCAGCCTGTATAGCGGCGTTCCGAGCCGTTTCAGCGGTAGCC |
| | | GTAGCGGCACCGACTTTACCCTGACCATTAGCAGCCTGCAGCCGGAAGA |
| | | TTTCGCAACTTATTACTGTCAGCAATCTCATGCTCTGATCACGTTCGGAC |
| | | AGGGCACCAAAGTTGAGATTAAAGGAGGAGGAGGAAGCGGAGGAGGAG |
| | | GATCCGGCGGCGGCGGCTCTGAGGTTCAACTGGTGGAGAGCGGTGGTGG |
| | | TCTGGTTCAGCCGGGTGGTAGCCTGCGTCTGAGCTGCGCAGCTTCTGGCT |
| | | TCAACATCTCTTCTTCTTATATCCACTGGGTGCGTCAGGCGCCGGGTAAA |
| | | GGCCTGGAATGGGTTGCATATATTTCTTCTTATTATAGCTATACTTATTAT |
| | | GCCGATAGCGTCAAGGGCCGTTTCACCATCAGCGCGGATACCAGCAAAA |
| | | ACACCGCATACCTGCAAATGAACAGCCTGCGTGCGGAAGATACCGCCGT |
| | | CTATTATTGTGCTCGCGGTTACCCGTACGGTATGGACTACTGGGGTCAAG |
| | | GCACCCTGGTTACCGTGAGCAGC |
| 180 | AS57659 scFv | GACATCCAGATGACCCAGAGCCCGAGCAGCCTGAGCGCGAGCGTTGGTG |
| | | ACCGTGTTACCATTACCTGCCGTGCGAGCCAGAGCGTTAGCAGCGCGGT |
| | | GGCGTGGTACCAGCAAAAGCCGGGTAAAGCGCCGAAGCTGCTGATCTAT |
| | | AGCGCGAGCAGCCTGTATAGCGGCGTTCCGAGCCGTTTCAGCGGTAGCC |
| | | GTAGCGGCACCGACTTTACCCTGACCATTAGCAGCCTGCAGCCGGAAGA |
| | | TTTCGCAACTTATTACTGTCAGCAACCGTACTACCTGATCACGTTCGGAC |
| | | AGGGCACCAAAGTTGAGATTAAAGGAGGAGGAGGAAGCGGAGGAGGAG |
| | | GATCCGGCGGCGGCGGCTCTGAGGTTCAACTGGTGGAGAGCGGTGGTGG |
| | | TCTGGTTCAGCCGGGTGGTAGCCTGCGTCTGAGCTGCGCAGCTTCTGGCT |
| | | TCAACATCTATTCTTATTATATCCACTGGGTGCGTCAGGCGCCGGGTAAA |
| | | GGCCTGGAATGGGTTGCATCTATTTATTCTTCTTATAGCTCTACTTATTAT |
| | | GCCGATAGCGTCAAGGGCCGTTTCACCATCAGCGCGGATACCAGCAAAA |
| | | ACACCGCATACCTGCAAATGAACAGCCTGCGTGCGGAAGATACCGCCGT |
| | | CTATTATTGTGCTCGCTCTTGGTTCTCTTACCCGGGTTTGGACTACTGGGG |
| | | TCAAGGCACCCTGGTTACCGTGAGCAGC |
| 181 | AS57765 scFv | GACATCCAGATGACCCAGAGCCCGAGCAGCCTGAGCGCGAGCGTTGGTG |
| | | ACCGTGTTACCATTACCTGCCGTGCGAGCCAGAGCGTTAGCAGCGCGGT |
| | | GGCGTGGTACCAGCAAAAGCCGGGTAAAGCGCCGAAGCTGCTGATCTAT |
| | | AGCGCGAGCAGCCTGTATAGCGGCGTTCCGAGCCGTTTCAGCGGTAGCC |
| | | GTAGCGGCACCGACTTTACCCTGACCATTAGCAGCCTGCAGCCGGAAGA |
| | | TTTCGCAACTTATTACTGTCAGCAAGCTTACTACTCTCTGATCACGTTCGG |
| | | ACAGGGCACCAAAGTTGAGATTAAAGGAGGAGGAGGAAGCGGAGGAGG |
| | | AGGATCCGGCGGCGGCGGCTCTGAGGTTCAACTGGTGGAGAGCGGTGGT |
| | | GGTCTGGTTCAGCCGGGTGGTAGCCTGCGTCTGAGCTGCGCAGCTTCTGG |
| | | CTTCAACATCTATTATTCTTATATGCACTGGGTGCGTCAGGCGCCGGGTA |
| | | AAGGCCTGGAATGGGTTGCATATATTTATCCTTATTCTGGCTCTACTTCTT |
| | | ATGCCGATAGCGTCAAGGGCCGTTTCACCATCAGCGCGGATACCAGCAA |
| | | AAACACCGCATACCTGCAAATGAACAGCCTGCGTGCGGAAGATACCGCC |
| | | GTCTATTATTGTGCTCGCCCGGCTGTTCATTGGCATGGTTACGGTGGTGG |
| | | TTACTACTACGGTTTGGACTACTGGGGTCAAGGCACCCTGGTTACCGTGA |
| | | GCAGC |
| 182 | AS48542VH5 | MALPVTALLLPLALLLHAARPEVQLVESGGGLVQPGGSLRLSCATSAFTFD |
| | bbz | GPDMAWYRQAPGKGCELVSIISADGRTYYADSVKGRFTISRDNSKNTVYLQ |
| | | MNSLRAEDTAVYYCALDPRKNCRGGYCCANWGQGTLVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 183 | AS48463VH4 | MALPVTALLLPLALLLHAARPEVQLLESGGGLVQPGGSLRLSCAASGFTFA |
| | bbz | NSDMGWYRQAPGKGCELVSIISSHGGTTYYVDSVKGRFTISRDNSKNTLYL |
| | | QMNSLRAEDTAVYYCVADPRSNCRGGYCCGYWGQGTLVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 184 | AS47863VH4 | MALPVTALLLPLALLLHAARPEVQLVESGGGLVQPGGSLRLSCAASGSTFG |
| | bbz | DSDMGWYRQAPGKGCELVSIISSDGRTYYVDSVKGRFTISQDNSKNTLYLQ |
| | | MNSLRAEDTAVYYCAADLRQYCRDGRCCGYWGQGTLVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPR |
| 185 | AS53574VH7 | MALPVTALLLPLALLLHAARPEVQLVESGGGLVQPGGSLRLSCAASGYIYS |
| | bbz | SNCMGWFRQAPGKGREWVARIHTGSGSTYYADSVKGRFTISQDNSKNTLYL |
| | | QMNSLRAEDTAVYDCAAGRVVLGAVVCTNEYWGQGTLVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 186 | AS48542VH5 | MALPVTALLLPLALLLHAARPEVQLVESGGGLVQPGGSLRLSCATSAFTFD |
| | dil-bbz | GPDMAWYRQAPGKGCELVSIISADGRTYYADSVKGRFTISRDNSKNTVYLQ |
| | | MNSLRAEDTAVYYCALDPRKNCRGGYCCANWGQGTLVTVSSGGGGSGGGGS |
| | | GGGGSEVQLVESGGGLVQPGGSLRLSCATSAFTFDGPDMAWYRQAPGKGCE |
| | | LVSIISADGRTYYADSVKGRFTISRDNSKNTVYLQMNSLRAEDTAVYYCAL |
| | | DPRKNCRGGYCCANWGQGTLVTVSSTTTPAPRPPTPAPTIASQPLSLRPEA |
| | | CRPAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 187 | AS48463VH4 | MALPVTALLLPLALLLHAARPEVQLLESGGGLVQPGGSLRLSCAASGFTFA |
| | dil-bbz | NSDMGWYRQAPGKGCELVSIISSHGGTTYYVDSVKGRFTISRDNSKNTLYL |
| | | QMNSLRAEDTAVYYCVADPRSNCRGGYCCGYWGQGTLVTVSSGGGGSGGGG |
| | | SGGGGSEVQLLESGGGLVQPGGSLRLSCAASGFTFANSDMGWYRQAPGKGC |
| | | ELVSIISSHGGTTYYVDSVKGRFTISRDNSKNTLYLQMNSLRAEDTAVYYC |
| | | VADPRSNCRGGYCCGYWGQGTLVTVSSTTTPAPRPPTPAPTIASQPLSLRP |
| | | EACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 188 | AS47863VH4 | MALPVTALLLPLALLLHAARPEVQLVESGGGLVQPGGSLRLSCAASGSTFG |
| | dil-bbz | DSDMGWYRQAPGKGCELVSIISSDGRTYYVDSVKGRFTISQDNSKNTLYLQ |
| | | MNSLRAEDTAVYYCAADLRQYCRDGRCCGYWGQGTLVTVSSGGGGSGGGGS |
| | | GGGGSEVQLVESGGGLVQPGGSLRLSCAASGSTFGDSDMGWYRQAPGKGCE |
| | | LVSIISSDGRTYYVDSVKGRFTISQDNSKNTLYLQMNSLRAEDTAVYYCAA |
| | | DLRQYCRDGRCCGYWGQGTLVTVSSTTTPAPRPPTPAPTIASQPLSLRPEA |
| | | CRPAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 189 | AS48542VH5- | MALPVTALLLPLALLLHAARPEVQLVESGGGLVQPGGSLRLSCATSAFTFD |
| | AS53574VH7 | GPDMAWYRQAPGKGCELVSIISADGRTYYADSVKGRFTISRDNSKNTVYLQ |
| | bil-bbz | MNSLRAEDTAVYYCALDPRKNCRGGYCCANWGQGTLVTVSSGGGGSGGGGS |
| | | GGGGSEVQLVESGGGLVQPGGSLRLSCAASGYIYSSNCMGWFRQAPGKGRE |
| | | WVARIHTGSGSTYYADSVKGRFTISQDNSKNTLYLQMNSLRAEDTAVYDCA |
| | | AGRVVLGAVVCTNEYWGQGTLVTVSSTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 190 | AS48463VH4- | MALPVTALLLPLALLLHAARPEVQLLESGGGLVQPGGSLRLSCAASGFTFA |
| | AS53574VH7 | NSDMGWYRQAPGKGCELVSIISSHGGTTYYVDSVKGRFTISRDNSKNTLYL |
| | bil-bbz | QMNSLRAEDTAVYYCVADPRSNCRGGYCCGYWGQGTLVTVSSGGGGSGGGG |
| | | SGGGGSEVQLVESGGGLVQPGGSLRLSCAASGYIYSSNCMGWFRQAPGKGR |
| | | EWVARIHTGSGSTYYADSVKGRFTISQDNSKNTLYLQMNSLRAEDTAVYDC |
| | | AAGRVVLGAVVCTNEYWGQGTLVTVSSTTTPAPRPPTPAPTIASQPLSLRP |
| | | EACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 191 | AS47863VH4- | MALPVTALLLPLALLLHAARPEVQLVESGGGLVQPGGSLRLSCAASGSTFG |
| | AS53574VH7 | DSDMGWYRQAPGKGCELVSIISSDGRTYYVDSVKGRFTISQDNSKNTLYLQ |
| | bil-bbz | MNSLRAEDTAVYYCAADLRQYCRDGRCCGYWGQGTLVTVSSGGGGSGGGGS |
| | | GGGGSEVQLVESGGGLVQPGGSLRLSCAASGYIYSSNCMGWFRQAPGKGRE |
| | | WVARIHTGSGSTYYADSVKGRFTISQDNSKNTLYLQMNSLRAEDTAVYDCA |
| | | AGRVVLGAVVCTNEYWGQGTLVTVSSTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 192 | AS53574VH7- | MALPVTALLLPLALLLHAARPEVQLVESGGGLVQPGGSLRLSCAASGYIYS |
| | AS48542VH5 | SNCMGWFRQAPGKGREWVARIHTGSGSTYYADSVKGRFTISQDNSKNTLYL |
| | bil-bbz | QMNSLRAEDTAVYDCAAGRVVLGAVVCTNEYWGQGTLVTVSSGGGGSGGGG |
| | | SGGGGSEVQLVESGGGLVQPGGSLRLSCATSAFTFDGPDMAWYRQAPGKGC |
| | | ELVSIISADGRTYYADSVKGRFTISRDNSKNTVYLQMNSLRAEDTAVYYCA |
| | | LDPRKNCRGGYCCANWGQGTLVTVSSTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 193 | AS53574VH7- | MALPVTALLLPLALLLHAARPEVQLVESGGGLVQPGGSLRLSCAASGYIYS |
| | AS48463VH4 | SNCMGWFRQAPGKGREWVARIHTGSGSTYYADSVKGRFTISQDNSKNTLYL |
| | bil-bbz | QMNSLRAEDTAVYDCAAGRVVLGAVVCTNEYWGQGTLVTVSSGGGGSGGGG |
| | | SGGGGSEVQLLESGGGLVQPGGSLRLSCAASGFTFANSDMGWYRQAPGKGC |
| | | ELVSIISSHGGTTYYVDSVKGRFTISRDNSKNTLYLQMNSLRAEDTAVYYC |
| | | VADPRSNCRGGYCCGYWGQGTLVTVSSTTTPAPRPPTPAPTIASQPLSLRP |
| | | EACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 194 | AS53574VH7- | MALPVTALLLPLALLLHAARPEVQLVESGGGLVQPGGSLRLSCAASGYIYS |
| | AS47863VH4 | SNCMGWFRQAPGKGREWVARIHTGSGSTYYADSVKGRFTISQDNSKNTLYL |
| | bil-bbz | QMNSLRAEDTAVYDCAAGRVVLGAVVCTNEYWGQGTLVTVSSGGGGSGGGG |
| | | SGGGGSEVQLVESGGGLVQPGGSLRLSCAASGSTFGDSDMGWYRQAPGKGC |
| | | ELVSIISSDGRTYYVDSVKGRFTISQDNSKNTLYLQMNSLRAEDTAVYYCA |
| | | ADLRQYCRDGRCCGYWGQGTLVTVSSTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 195 | TR2D- | MGRGLLRGLWPLHIVLWTRIASTIPPHVQKSVNNDMIVTDNNGAVKFPQLC |
| | AS48542VH5 | KFCDVRFSTCDNQKSCMSNCSITSICEKPQEVCVAVWRKNDENITLETVCH |
| | bbz-4C | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCATSAFTFDGPDMAWYRQAPGKGCELVSIISADGRTYYADSVK |
| | | GRFTISRDNSKNTVYLQMNSLRAEDTAVYYCALDPRKNCRGGYCCANWGQG |
| | | TLVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEED |
| | | GCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGH |
| | | DGLYQGLSTATKDTYDALHMQALPPRGSGATNFSLLKQAGDVEENPGPMNP |
| | | TDIADTTLDESIYSNYYLYESIPKPCTKEGIKAFGELFLPPLYSLVFVFGL |
| | | LGNSVVVLVLFKYKRLRSMTDVYLLNLAISDLLFVFSLPFWGYYAADQWVF |
| | | GLGLCKMISWMYLVGFYSGIFFVMLMSIDRYLAIVHAVFSLRARTLTYGVI |
| | | TSLATWSVAVFASLPGFLFSTCYTERNHTYCKTKYSLNSTTWKVLSSLEIN |
| | | ILGLVIPLGIMLFCYSMIIRTLQHCKNEKKNKAVKMIFAVVVLFLGFWTPY |
| | | NIVLFLETLVELEVLQDCTFERYLDYAIQATETLAFVHCCLNPIIYFFLGE |
| | | KFRKYILQLFKTCRGLFVLCQYCGLLQIYSADTPSSSYTQSTMDHDLHDAL |
| 196 | TR2D- | MGRGLLRGLWPLHIVLWTRIASTIPPHVQKSVNNDMIVTDNNGAVKFPQLC |
| | AS48542VH5 | KFCDVRFSTCDNQKSCMSNCSITSICEKPQEVCVAVWRKNDENITLETVCH |
| | bbz | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCATSAFTFDGPDMAWYRQAPGKGCELVSIISADGRTYYADSVK |
| | | GRFTISRDNSKNTVYLQMNSLRAEDTAVYYCALDPRKNCRGGYCCANWGQG |
| | | TLVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEED |
| | | GCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGH |
| | | DGLYQGLSTATKDTYDALHMQALPPR |
| 197 | PD1CD28- | MQIPQAPWPVVWAVLQLGWRPGWFLDSPDRPWNPPTFSPALLVVTEGDNAT |
| | AS48542VH5 | FTCSFSNTSESFVLNWYRMSPSNQTDKLAAFPEDRSQPGQDCRFRVTQLPN |
| | bbz | GRDFHMSVVRARRNDSGTYLCGAISLAPKAQIKESLRAELRVTERRAEVPT |
| | | AHCPSPLFPGPSKPFWVLVVVGGVLACYSLLVTVAFIIFWVRSKRSRLLHS |
| | | DYMNMTPRRPGPTRKHYQPYAPPRDFAAYRSGSGATNFSLLKQAGDVEENP |
| | | GPMALPVTALLLPLALLLHAARPEVQLVESGGGLVQPGGSLRLSCATSAFT |
| | | FDGPDMAWYRQAPGKGCELVSIISADGRTYYADSVKGRFTISRDNSKNTVY |
| | | LQMNSLRAEDTAVYYCALDPRKNCRGGYCCANWGQGTLVTVSSTTTPAPRP |
| | | PTPAPTIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEEGGCE |
| | | LRVKFSRSADAPAYKQGQNQLYNELNLGRREEYDVLDKRRGRDPEMGGKPR |
| | | RKNPQEGLYNELQKDKMAEAYSEIGMKGERRRGKGHDGLYQGLSTATKDTY |
| | | DALHMQALPPR |
| 198 | AS48542VH5 | MALPVTALLLPLALLLHAARPEVQLVESGGGLVQPGGSLRLSCATSAFTFD |
| | bbz-4C | GPDMAWYRQAPGKGCELVSIISADGRTYYADSVKGRFTISRDNSKNTVYLQ |
| | | MNSLRAEDTAVYYCALDPRKNCRGGYCCANWGQGTLVTVSSTTTPAPRPPT |
| | | PAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTYDA |
| | | LHMQALPPRGSGATNFSLLKQAGDVEENPGPMNPTDIADTTLDESIYSNYY |
| | | LYESIPKPCTKEGIKAFGELFLPPLYSLVFVFGLLGNSVVVLVLFKYKRLR |
| | | SMTDVYLLNLAISDLLFVFSLPFWGYYAADQWVFGLGLCKMISWMYLVGFY |
| | | SGIFFVMLMSIDRYLAIVHAVFSLRARTLTYGVITSLATWSVAVFASLPGF |
| | | LFSTCYTERNHTYCKTKYSLNSTTWKVLSSLEINILGLVIPLGIMLFCYSM |
| | | IIRTLQHCKNEKKNKAVKMIFAVVVLFLGFWTPYNIVLFLETLVELEVLQD |
| | | CTFERYLDYAIQATETLAFVHCCLNPIIYFFLGEKFRKYILQLFKTCRGLF |
| | | VLCQYCGLLQIYSADTPSSSYTQSTMDHDLHDAL |
| 199 | AS53574VH7 | EVQLVESGGGLVQPGGSLRLSCAASGYIYSSNCMGWFRQAPGKGREWVARI |
| | | HTGSGSTYYADSVKGRFTISQDNSKNTLYLQMNSLRAEDTAVYDCAAGRVV |
| | | LGAVVCTNEYWGQGTLVTVSS |
| 200 | AS53574VH7 | GAGGTGCAGCTGGTGGAGTCCGGAGGAGGACTGGTGCAGCCAGGAGGC |
| | sdAb | AGCCTGCGGCTGTCCTGCGCCGCCTCTGGCTACATCTATAGCTCCAACTG |
| | | TATGGGCTGGTTCAGGCAGGCACCTGGCAAGGGAAGGGAGTGGGTGGCC |
| | | AGAATCCACACCGGCTCCGGCTCTACATACTATGCCGACTCTGTGAAGG |
| | | GCCGGTTTACCATCAGCCAGGATAACTCCAAGAATACACTGTACCTGCA |
| | | GATGAACAGCCTGAGGGCCGAGGACACCGCCGTGTATGATTGCGCAGCA |
| | | GGAAGGGTGGTGCTGGGAGCAGTGGTGTGCACAAATGAGTACTGGGGCC |
| | | AGGGCACCCTGGTGACAGTGTCTAGC |
| 201 | AS48542-28z | MALPVTALLLPLALLLHAARPQMQLVESGGGSVQAGETLRLSCTTSAFTFD |
| | | GPDMAWYRQAPGNECVLVSIISADGRTYYADSVKGRFTISRDNAKNTVFLN |
| | | LNSLQPEDTAVYYCALDPRKNCRGGYCCANWGPGTQVTVSSIEVMYPPPYL |
| | | DNEKSNGTIIHVKGKHLCPSPLFPGPSKPFWVLVVVGGVLACYSLLVTVAF |
| | | IIFWVRSKRSRLLHSDYMNMTPRRPGPTRKHYQPYAPPRDFAAYRSRVKFS |
| | | RSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQ |
| | | ALPPR |
| 202 | GS linker | GGGGSGGGS |
| 203 | GS linker | (GGGGS)n |
| 204 | GS linker | SGGGS |
| 205 | TR2D- | MGRGLLRGLWPLHIVLWTRIASTIPPHVQKSVNNDMIVTDNNGAVKFPQLC |
| | AS48542VH5 | KFCDVRFSTCDNQKSCMSNCSITSICEKPQEVCVAVWRKNDENITLETVCH |
| | dil-bbz | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCATSAFTFDGPDMAWYRQAPGKGCELVSIISADGRTYYADSVK |
| | | GRFTISRDNSKNTVYLQMNSLRAEDTAVYYCALDPRKNCRGGYCCANWGQG |
| | | TLVTVSSGGGGSGGGGSGGGGSEVQLVESGGGLVQPGGSLRLSCATSAFTF |
| | | DGPDMAWYRQAPGKGCELVSIISADGRTYYADSVKGRFTISRDNSKNTVYL |
| | | QMNSLRAEDTAVYYCALDPRKNCRGGYCCANWGQGTLVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 206 | TR2D- | MGRGLLRGLWPLHIVLWTRIASTIPPHVQKSVNNDMIVTDNNGAVKFPQLC |
| | AS47863VH4 | KFCDVRFSTCDNQKSCMSNCSITSICEKPQEVCVAVWRKNDENITLETVCH |
| | bbz | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCAASGSTFGDSDMGWYRQAPGKGCELVSIISSDGRTYYVDSVK |
| | | GRFTISQDNSKNTLYLQMNSLRAEDTAVYYCAADLRQYCRDGRCCGYWGQG |
| | | TLVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEED |
| | | GCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGH |
| | | DGLYQGLSTATKDTYDALHMQALPPR |
| 207 | TR2D- | MGRGLLRGLWPLHIVLWTRIASTIPPHVQKSVNNDMIVTDNNGAVKFPQLC |
| | AS47863VH4 | KFCDVRFSTCDNQKSCMSNCSITSICEKPQEVCVAVWRKNDENITLETVCH |
| | dil-bbz | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCAASGSTFGDSDMGWYRQAPGKGCELVSIISSDGRTYYVDSVK |
| | | GRFTISQDNSKNTLYLQMNSLRAEDTAVYYCAADLRQYCRDGRCCGYWGQG |
| | | TLVTVSSGGGGSGGGGSGGGGSEVQLVESGGGLVQPGGSLRLSCAASGSTF |
| | | GDSDMGWYRQAPGKGCELVSIISSDGRTYYVDSVKGRFTISQDNSKNTLYL |
| | | QMNSLRAEDTAVYYCAADLRQYCRDGRCCGYWGQGTLVTVSSTTTPAPRPP |
| | | TPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDTYD |
| | | ALHMQALPPR |
| 208 | AS48542VH5- | MALPVTALLLPLALLLHAARPEVQLVESGGGLVQPGGSLRLSCATSAFTFD |
| | 28z | GPDMAWYRQAPGKGCELVSIISADGRTYYADSVKGRFTISRDNSKNTVYLQ |
| | | MNSLRAEDTAVYYCALDPRKNCRGGYCCANWGQGTLVTVSSIEVMYPPPYL |
| | | DNEKSNGTIIHVKGKHLCPSPLFPGPSKPFWVLVWGGVLACYSLLVTVAFI |
| | | IFWVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQA |
| | | LPPR |
| 209 | AS48542VH5 | MALPVTALLLPLALLLHAARPEVQLVESGGGLVQPGGSLRLSCATSAFTFD |
| | dil-28z | GPDMAWYRQAPGKGCELVSIISADGRTYYADSVKGRFTISRDNSKNTVYLQ |
| | | MNSLRAEDTAVYYCALDPRKNCRGGYCCANWGQGTLVTVSSGGGGSGGGGS |
| | | GGGGSEVQLVESGGGLVQPGGSLRLSCATSAFTFDGPDMAWYRQAPGKGCE |
| | | LVSIISADGRTYYADSVKGRFTISRDNSKNTVYLQMNSLRAEDTAVYYCAL |
| | | DPRKNCRGGYCCANWGQGTLVTVSSIEVMYPPPYLDNEKSNGTIIHVKGKH |
| | | LCPSPLFPGPSKPFWVLVVVGGVLACYSLLVTVAFIIFWVRSKRSRLLHSD |
| | | YMNMTPRRPGPTRKHYQPYAPPRDFAAYRSRVKFSRSADAPAYQQGQNQLY |
| | | NELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 210 | AS47863VH4- | MALPVTALLLPLALLLHAARPEVQLVESGGGLVQPGGSLRLSCAASGSTFG |
| | 28z | DSDMGWYRQAPGKGCELVSIISSDGRTYYVDSVKGRFTISQDNSKNTLYLQ |
| | | MNSLRAEDTAVYYCAADLRQYCRDGRCCGYWGQGTLVTVSSIEVMYPPPYL |
| | | DNEKSNGTIIHVKGKHLCPSPLFPGPSKPFWVLVVVGGVLACYSLLVTVAF |
| | | IIFWVRSKRSRLLHSDYMNMTPRRPGPTRKHYQPYAPPRDFAAYRSRVKFS |
| | | RSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQ |
| | | ALPPR |
| 211 | AS47863VH4 | MALPVTALLLPLALLLHAARPEVQLVESGGGLVQPGGSLRLSCAASGSTFG |
| | dil-28z | DSDMGWYRQAPGKGCELVSIISSDGRTYYVDSVKGRFTISQDNSKNTLYLQ |
| | | MNSLRAEDTAVYYCAADLRQYCRDGRCCGYWGQGTLVTVSSGGGGSGGGGS |
| | | GGGGSEVQLVESGGGLVQPGGSLRLSCAASGSTFGDSDMGWYRQAPGKGCE |
| | | LVSIISSDGRTYYVDSVKGRFTISQDNSKNTLYLQMNSLRAEDTAVYYCAA |
| | | DLRQYCRDGRCCGYWGQGTLVTVSSIEVMYPPPYLDNEKSNGTIIHVKGKH |
| | | LCPSPLFPGPSKPFWVLVVVGGVLACYSLLVTVAFIIFWVRSKRSRLLHSD |
| | | YMNMTPRRPGPTRKHYQPYAPPRDFAAYRSRVKFSRSADAPAYQQGQNQLY |
| | | NELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 212 | TR2D- | MGRGLLRGLWPLHIVLWTRIASTIPPHVQKSVNNDMIVTDNNGAVKFPQLC |
| | AS48542VH5- | KFCDVRFSTCDNQKSCMSNCSITSICEKPQEVCVAVWRKNDENITLETVCH |
| | 28z | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCATSAFTFDGPDMAWYRQAPGKGCELVSIISADGRTYYADSVK |
| | | GRFTISRDNSKNTVYLQMNSLRAEDTAVYYCALDPRKNCRGGYCCANWGQG |
| | | TLVTVSSIEVMYPPPYLDNEKSNGTIIHVKGKHLCPSPLFPGPSKPFWVLV |
| | | VVGGVLACYSLLVTVAFIIFWVRSKRSRLLHSDYMNMTPRRPGPTRKHYQP |
| | | YAPPRDFAAYRSRVKFSRSADAPAYQQGQNQLYNELNLGRREEYDVLDKRR |
| | | GRDPEMGGKPRRKNPQEGLYNELQKDKMAEAYSEIGMKGERRRGKGHDGLY |
| | | QGLSTATKDTYDALHMQALPPR |
| 213 | TR2D- | MGRGLLRGLWPLHIVLWTRIASTIPPHVQKSVNNDMIVTDNNGAVKFPQLC |
| | AS48542VH5 | KFCDVRFSTCDNQKSCMSNCSITSICEKPQEVCVAVWRKNDENITLETVCH |
| | dil-28z | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCATSAFTFDGPDMAWYRQAPGKGCELVSIISADGRTYYADSVK |
| | | GRFTISRDNSKNTVYLQMNSLRAEDTAVYYCALDPRKNCRGGYCCANWGQG |
| | | TLVTVSSGGGGSGGGGSGGGGSEVQLVESGGGLVQPGGSLRLSCATSAFTF |
| | | DGPDMAWYRQAPGKGCELVSIISADGRTYYADSVKGRFTISRDNSKNTVYL |
| | | QMNSLRAEDTAVYYCALDPRKNCRGGYCCANWGQGTLVTVSSIEVMYPPPY |
| | | LDNEKSNGTIIHVKGKHLCPSPLFPGPSKPFWVLVVVGGVLACYSLLVTVA |
| | | FIIFWVRSKRSRLLHSDYMNMTPRRPGPTRKHYQPYAPPRDFAAYRSRVKF |
| | | SRSADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALHM |
| | | QALPPR |
| 214 | TR2D- | MGRGLLRGLWPLHIVLWTRIASTIPPHVQKSVNNDMIVTDNNGAVKFPQLC |
| | AS47863VH4- | KFCDVRFSTCDNQKSCMSNCSITSICEKPQEVCVAVWRKNDENITLETVCH |
| | 28z | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCAASGSTFGDSDMGWYRQAPGKGCELVSIISSDGRTYYVDSVK |
| | | GRFTISQDNSKNTLYLQMNSLRAEDTAVYYCAADLRQYCRDGRCCGYWGQG |
| | | TLVTVSSIEVMYPPPYLDNEKSNGTIIHVKGKHLCPSPLFPGPSKPFWVLV |
| | | VVGGVLACYSLLVTVAFIIFWVRSKRSRLLHSDYMNMTPRRPGPTRKHYQP |
| | | YAPPRDFAAYRSRVKFSRSADAPAYQQGQNQLYNELNLGRREEYDVLDKRR |
| | | GRDPEMGGKPRRKNPQEGLYNELQKDKMAEAYSEIGMKGERRRGKGHDGLY |
| | | QGLSTATKDTYDALHMQALPPR |
| 215 | TR2D- | MGRGLLRGLWPLHIVLWTRIASTIPPHVQKSVNNDMIVTDNNGAVKFPQLC |
| | AS47863VH4 | KFCDVRFSTCDNQKSCMSNCSITSICEKPQEVCVAVWRKNDENITLETVCH |
| | dil-28z | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSSGSGAT |
| | | NFSLLKQAGDVEENPGPMALPVTALLLPLALLLHAARPEVQLVESGGGLVQ |
| | | PGGSLRLSCAASGSTFGDSDMGWYRQAPGKGCELVSIISSDGRTYYVDSVK |
| | | GRFTISQDNSKNTLYLQMNSLRAEDTAVYYCAADLRQYCRDGRCCGYWGQG |
| | | TLVTVSSGGGGSGGGGSGGGGSEVQLVESGGGLVQPGGSLRLSCAASGSTF |
| | | GDSDMGWYRQAPGKGCELVSIISSDGRTYYVDSVKGRFTISQDNSKNTLYL |
| | | QMNSLRAEDTAVYYCAADLRQYCRDGRCCGYWGQGTLVTVSSIEVMYPPPY |
| | | LDNEKSNGTIIHVKGKHLCPSPLFPGPSKPFWVLVVVGGVLACYSLLVTVA |
| | | FIIFWVRSKRSRLLHSDYMNMTPRRPGPTRKHYQPYAPPRDFAAYRSRVKF |
| | | SRSADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALHM |
| | | QALPPR |
| 216 | 5F11 scFv | DIQMTQSPTSLSASVGDRVTITCRASQGISSWLTWYQQKPEKAPKSLIYAA |
| | | SSLQSGVPSRFSGSGSGTDFTLTISSLQPEDFATYYCQQYDSYPITFGQGT |
| | | RLEIKGSTSGSGKPGSGEGSTKGQVQLQQWGAGLLKPSETLSLTCAVYGGS |
| | | FSAYYWSWIRQPPGKGLEWIGDINHGGGTNYNPSLKSRVTISVDTSKNQFS |
| | | LKLNSVTAADTAVYYCASLTAYWGQGSLVTVSS |
| 217 | Human IgG1 | PKSCDKTHTCPPCPAPELLGGPSVFLFPPKPKDTLMISRTPEVTCVVVDVS |
| | Fc fragment | HEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQQG |
| | | NVFSCSVMHEALHNHYTQKSLSLSPGK |
| 218 | AS57911VH | DIQMTQSPSSLSASVGDRVTITCRASQSVSSAVAWYQQKPGKAPKLLIYSA |
| | | SSLYSGVPSRFSGSRSGTDFTLTISSLQPEDFATYYCQQSHALITFGQGTK |
| | | VEIK |
| 219 | AS57911VL | EVQLVESGGGLVQPGGSLRLSCAASGFNISSSYIHWVRQAPGKGLEWVAYI |
| | | SSYYSYTYYADSVKGRFTISADTSKNTAYLQMNSLRAEDTAVYYCARGYPY |
| | | GMDYWGQGTLVTVSS |
| 220 | AS57659VH | DIQMTQSPSSLSASVGDRVTITCRASQSVSSAVAWYQQKPGKAPKLLIYSA |
| | | SSLYSGVPSRFSGSRSGTDFTLTISSLQPEDFATYYCQQPYYLITFGQGTK |
| | | VEIK |
| 221 | AS57659VL | EVQLVESGGGLVQPGGSLRLSCAASGFNIYSYYIHWVRQAPGKGLEWVASI |
| | | YSSYSSTYYADSVKGRFTISADTSKNTAYLQMNSLRAEDTAVYYCARSWFS |
| | | YPGLDYWGQGTLVTVSS |
| 222 | AS57765VH | DIQMTQSPSSLSASVGDRVTITCRASQSVSSAVAWYQQKPGKAPKLLIYSA |
| | | SSLYSGVPSRFSGSRSGTDFTLTISSLQPEDFATYYCQQAYYSLITFGQGT |
| | | KVEIK |
| 223 | AS57765VL | EVQLVESGGGLVQPGGSLRLSCAASGFNIYYSYMHWVRQAPGKGLEWVAYI |
| | | YPYSGSTSYADSVKGRFTISADTSKNTAYLQMNSLRAEDTAVYYCARPAVH |
| | | WHGYGGGYYYGLDYWGQGTLVTVSS |

1. A binding moiety that specifically binds CD30, comprising a single domain antibody comprising:
(1) a CDR1 comprising SEQ ID NO:87; a CDR2 comprising SEQ ID NO:100; and a CDR3 comprising SEQ ID NO:111;
(2) a CDR1 comprising SEQ ID NO:87; a CDR2 comprising SEQ ID NO:100; and a CDR3 comprising SEQ ID NO:112;
(3) a CDR1 comprising SEQ ID NO:88; a CDR2 comprising SEQ ID NO:101; and a CDR3 comprising SEQ ID NO:113;
(4) a CDR1 comprising SEQ ID NO:89; a CDR2 comprising SEQ ID NO:102; and a CDR3 comprising SEQ ID NO:114;
(5) a CDR1 comprising SEQ ID NO:90; a CDR2 comprising SEQ ID NO:103; and a CDR3 comprising SEQ ID NO:115;
(6) a CDR1 comprising SEQ ID NO:91; a CDR2 comprising SEQ ID NO:104; and a CDR3 comprising SEQ ID NO:116;
(7) a CDR1 comprising SEQ ID NO:92; a CDR2 comprising SEQ ID NO:105; and a CDR3 comprising SEQ ID NO:117;
(8) a CDR1 comprising SEQ ID NO:93; a CDR2 comprising SEQ ID NO:106; and a CDR3 comprising SEQ ID NO:118;
(9) a CDR1 comprising SEQ ID NO:94; a CDR2 comprising SEQ ID NO:103; and a CDR3 comprising SEQ ID NO:119; or
(10) a CDR1 comprising SEQ ID NO:95; a CDR2 comprising SEQ ID NO:103; and a CDR3 comprising SEQ ID NO:120;
or a variant of the single domain antibody comprising up to about 5 amino acid substitutions in the CDRs.

2. The binding moiety of claim 1, wherein the single domain antibody has an amino acid sequence that is at least 90%, 95%, or 99% identical to an amino acid sequence selected from the group consisting of SEQ ID NOs:9-54 and 199.

3. The binding moiety of claim 1, wherein the single domain antibody has an amino acid sequence selected from the group consisting of SEQ ID NOs:9-54 and 199.

4. A binding moiety that specifically binds CD30, comprising a single domain antibody comprising a CDR1, CDR2, and CDR3 from a binding moiety comprising a single domain antibody having an amino acid sequence selected from the group consisting of SEQ ID NOs:9-54 and 199.

5. The binding moiety of any one of claims 1 to 4, wherein the binding moiety specifically binds human CD30, rhesus CD30, or both.

6. The binding moiety of any one of claims 1 to 5, wherein the binding moiety specifically binds the cysteine rich domain 6 (CRD6) of human CD30 (SEQ ID NO:8), or the cysteine rich domain 1 (CRD1) of human CD30 (SEQ ID NO:3).

7. The binding moiety of any one of claims 1 to 6, wherein the single domain antibody is a camel, chimeric, humanized or human antibody.

8. The binding moiety of any one of claims 1 to 7, further comprising a human IgG1 hinge and Fc region linked to the single domain antibody.

9. A binding moiety that specifically binds CD30, comprising from N-terminus to C-terminus a first single domain antibody, a linker, and a second single domain antibody, wherein each of the first and second single domain antibodies is the single domain antibody of claim 1.

10. The binding moiety of claim 9, wherein each of the first and second single domain antibodies has an amino acid sequence selected from the group consisting of SEQ ID NOs:9-54 and 199.

11. The binding moiety of claim 9, wherein the first and second single domain antibodies recognize different epitopes on CD30.

12. The binding moiety of claim 9, wherein the first and second single domain antibodies recognize the same epitope on CD30.

13. The binding moiety of claim 11, wherein the second single domain antibody is a tandem repeat of the first single domain antibody.

14. The binding moiety of any one of claims 9 to 13, wherein the linker has an amino acid sequence comprising or consisting of SEQ ID NO:55, 56, 57, 202 or 203.

15. A binding moiety that specifically binds CD30, comprising an antibody or an antigen-binding fragment thereof comprising:
(a) a heavy chain variable region (VH) comprising
(i) a VH CDR1 comprising SEQ ID NO:96, 97, or 98;
(ii) a VH CDR2 comprising SEQ ID NO:107, 108, or 109; and
(iii) a VH CDR3 comprising SEQ ID NO:121, 122, or 123; and/or
(b) a light chain variable region (VL) comprising
(i) a VL CDR1 comprising SEQ ID NO:99;
(ii) a VL CDR2 comprising SEQ ID NO:110; and
(iii) a VL CDR3 comprising SEQ ID NO:124, 125, or 126;
or a variant thereof comprising up to 3 amino acid substitutions in each of VH CDR1, VH CDR2, VH CDR3, VL CDR1, VL CDR2, and VL CDR3.

16. The binding moiety of claim 15, comprising an antibody or antigen-binding fragment thereof comprising:
(i) (a) a VH comprising a VH CDR1 comprising SEQ ID NO:96, a VH CDR2 comprising SEQ ID NO:107, and a VH CDR3 comprising SEQ ID NO:121; and/or (b) a VL comprising a VL CDR1 comprising SEQ ID NO:99, a VL CDR2 comprising SEQ ID NO:110, and a VL CDR3 comprising SEQ ID NO:124;
or a variant thereof comprising up to 5 amino acid substitutions in VH CDRs and/or up to 5 amino acid substitutions in VL CDRs;
(ii) (a) a VH comprising a VH CDR1 comprising SEQ ID NO:97, a VH CDR2 comprising SEQ ID NO:108, and a VH CDR3 comprising SEQ ID NO:122; and/or (b) a VL comprising a VL CDR1 comprising SEQ ID NO:99, a VL CDR2 comprising SEQ ID NO:110, and a VL CDR3 comprising SEQ ID NO:125;
or a variant thereof comprising up to 5 amino acid substitutions in VH CDRs and/or up to 5 amino acid substitutions in VL CDRs;
(iii) (a) a VH comprising a VH CDR1 comprising SEQ ID NO:98, a VH CDR2 comprising SEQ ID NO:109, and a VH CDR3 comprising SEQ ID NO:123; and/or (b) a VL comprising a VL CDR1 comprising SEQ ID NO:99, a VL CDR2 comprising SEQ ID NO:110, and a VL CDR3 comprising SEQ ID NO:126;
or a variant thereof comprising up to 5 amino acid substitutions in VH CDRs and/or up to 5 amino acid substitutions in VL CDRs.

17. The binding moiety of claim 16, wherein the antibody or antigen-binding fragment thereof comprises:
(i) (a) a VH comprising a VH CDR1 comprising SEQ ID NO:96, a VH CDR2 comprising SEQ ID NO:107, and a VH CDR3 comprising SEQ ID NO:121; and/or (b) a VL comprising a VL CDR1 comprising SEQ ID NO:99, a VL CDR2 comprising SEQ ID NO:110, and a VL CDR3 comprising SEQ ID NO:124;
(ii) (a) a VH comprising a VH CDR1 comprising SEQ ID NO:97, a VH CDR2 comprising SEQ ID NO:108, and a VH CDR3 comprising SEQ ID NO:122; and/or (b) a VL comprising a VL CDR1 comprising SEQ ID NO:99, a VL CDR2 comprising SEQ ID NO:110, and a VL CDR3 comprising SEQ ID NO:125; or
(iii) (a) a VH comprising a VH CDR1 comprising SEQ ID NO:98, a VH CDR2 comprising SEQ ID NO:109, and a VH CDR3 comprising SEQ ID NO:123; and/or (b) a VL comprising a VL CDR1 comprising SEQ ID NO:99, a VL CDR2 comprising SEQ ID NO:110, and a VL CDR3 comprising SEQ ID NO:126.

18. The binding moiety of claim 17, wherein the antibody or antigen-binding fragment thereof comprises
(i) a VH comprising an amino acid sequence having 90%, 95%, 99% or 100% identity to SEQ ID NO: 218, and/or a VL comprising an amino acid sequence having 90%, 95%, 99% or 100% identity to SEQ ID NO: 219;
(ii) a VH comprising an amino acid sequence having 90%, 95%, 99% or 100% identity to SEQ ID NO:220, and/or a VL comprising an amino acid sequence having 90%, 95%, 99% or 100% identity to SEQ ID NO:221; or
(iii) a VH comprising an amino acid sequence having 90%, 95%, 99% or 100% identity to SEQ ID NO: 222, and/or a VL comprising an amino acid sequence having 90%, 95%, 99% or 100% identity to SEQ ID NO:223.

19. The binding moiety of any one of claims 15 to 18, wherein the antibody or antigen-binding fragment thereof is a single chain variable fragment containing the VH and the VL connected by a linker.

20. The binding moiety of claim 19, wherein the linker has an amino acid sequence comprising or consisting of SEQ ID NO:55, 56, 57, 202 or 203.

21. The binding moiety of claim 20, wherein the single chain variable fragment has an amino acid sequence that is at least 90%, 95%, 99% identical to SEQ ID NO:58, 59, or 60.

22. The binding moiety of claim 20, wherein the single chain variable fragment has an amino acid sequence of SEQ ID NO:58, 59, or 60.

23. A binding moiety that specifically binds CD30, comprising an antibody or an antigen-binding fragment thereof comprising a VH CDR1, VH CDR2, VH CDR3, VL CDR1, VL CDR2, and VL CDR3 from a binding moiety comprising the antibody or antigen-binding fragment thereof having an amino acid sequence comprising SEQ ID: 58, 59, or 60.

24. The binding moiety of any one of claims 15 to 23, wherein the binding moiety specifically binds human CD30, rhesus CD30, or both.

25. The binding moiety of any one of claims 15 to 20, wherein the antibody or antigen-binding fragment thereof is selected from the group consisting of a single domain antibody (sdAb), a heavy chain antibody (HCAb), a Fab, a Fab′, a F(ab′)₂, a Fv, a (scFv)₂, an IgG1 antibody, an IgG2 antibody, an IgG3 antibody, and an IgG4 antibody.

26. The binding moiety of any one of claims 15 to 25, wherein the antibody is a camel, chimeric, humanized or human antibody.

27. The binding moiety of any one of claims 1 to 26, having a binding affinity (K_{D}) to CD30 that is between 10 pM and 500 nM, 100 pM and 200 nM, or 1 nM and 200 nM.

28. The binding moiety of claim 27, wherein the K_{D} is between 3 nM and 170 nM.

29. A polynucleotide encoding the binding moiety of any one of claims 1 to 28.

30. A vector comprising the polynucleotide of claim 29, wherein optionally the vector is a viral vector.

31. A CAR that specifically binds CD30, comprising, from N-terminus to C-terminus:
(a) a bivalent binding moiety comprising a first anti-CD30 sdAb and a second anti-CD30 sdAb;
(b) a transmembrane domain; and
(c) a cytoplasmic domain.

32. A CAR that specifically binds CD30, comprising, from N-terminus to C-terminus:
(a) an extracellular antigen binding domain comprising a binding moiety of any one of claims 1 to 28;
(b) a transmembrane domain; and
(c) a cytoplasmic domain.

33. The CAR of claim 31 or 32, wherein the transmembrane domain comprises CD8α transmembrane region or CD28 transmembrane region.

34. The CAR of any one of claims 31 to 33, wherein the cytoplasmic domain comprises at least one signaling domain selected from the group consisting of CD3ζ FcRγ, FcRβ, CD3γ, CD3δ, CD3ε, CDS, CD22, CD79a, CD79b, and CD66d.

35. The CAR of any one of claims 31 to 34, wherein the cytoplasmic domain comprises at least one costimulatory domains selected from the group consisting of CD28, 4-1BB (CD137), CD27, OX40, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

36. The CAR of any one of claims 31 to 35, wherein the cytoplasmic domain comprises a CD3ζ signaling domain and a 4-1BB costimulatory domain.

37. The CAR of any one of claims 31 to 36, wherein the cytoplasmic domain comprises a CD3ζ signaling domain and a CD28 costimulatory domain.

38. The CAR of any one of claims 31 to 37, further comprising a CD8α hinge or CD28 hinge between the CD30-binding moiety and the transmembrane domain.

39. The CAR of any one of claims 31 to 38, further comprising a leader sequence at the N-terminus.

40. The CAR of any one of claims 31 to 39, having an amino acid sequence selected from the group consisting of SEQ ID NOs: 70-86, 182-194, 201 and 208-211.

41. The CAR of any one of claims 31 to 40, wherein the CAR is conjugated to a factor selected from the group consisting of: (i) C—C chemokine receptor type 4 (CCR4), (ii) dominant negative transforming growth factor beta receptor II (dnTGFβRII), and (iii) a chimeric switch programmed death 1 receptor (PD1CD28).

42. The CAR of claim 41, wherein CCR4 comprises SEQ ID NO:67, wherein dnTGFβRII comprises SEQ ID NO:68, or wherein PD1CD28 comprises SEQ ID NO:69.

43. The CAR of claim 41 or 42, wherein the CAR is conjugated to the C-terminus of the factor.

44. The CAR of claim 41 or 42, wherein the CAR is conjugated to the N-terminus of the factor.

45. The CAR of any one of claims 41 to 44, wherein the CAR is conjugated to the factor via a 2A linker selected from the group consisting of P2A, T2A, E2A and F2A.

46. The CAR of any one of claims 31 to 40, wherein the CAR is conjugated to a first factor and a second factor, each selected from the group consisting of: CCR4, PD1CD28 and dnTGFβRII.

47. The CAR of claim 41 or 42, wherein the CAR is conjugated to dnTGFβRII.

48. The CAR of any one of claims 41 to 47, comprising an amino acid sequence selected from the group consisting of SEQ ID NO:195-198, 205-207, and 212-215.

49. The CAR of claim 48, comprising an amino acid sequence selected from the group consisting of SEQ ID NO:195, 196, 205-207, and 212-215.

50. The CAR of claim 46, wherein the CAR is conjugated to the C-terminus of the first factor, and the N-terminus of the second factor.

51. A polynucleotide encoding the CAR of any one of claims 31 to 50.

52. A vector comprising the polynucleotide of claim 51 wherein optionally the vector is a viral vector.

53. A host cell comprising the polynucleotide of claim 51 or the vector of claim 52.

54. A cell that recombinantly expresses the CAR of any one of claims 31 to 50.

55. The cell of claim 54, wherein the cell is a T cell.

56. The cell of claim 55, wherein the T cell is selected from the group consisting of a cytotoxic T cell, a helper T cell, a natural killer T cell, and a γδT cell.

57. A population of cells comprising at least two of the cells of any one of claims 54 to 56.

58. A pharmaceutical composition comprising a therapeutically effective amount of the population of cells of claim 57, and a pharmaceutically acceptable carrier.

59. A method of treating CD30-expressing tumor or cancer in a subject in need thereof, comprising administering to the subject a therapeutically effective amount of the population of cells of claim 57.

60. A method of treating CD30-expressing tumor or cancer in a subject in need thereof, comprising administering to the subject a therapeutically effective amount of the pharmaceutical composition of claim 58.

61. The method of claim 59 or 60, wherein the CD30-expressing tumor is a lymphoma, an embryonal carcinoma (EC) or a testicular germ cell tumor (TGCT).

62. The method of claim 61, wherein the CD30-expressing tumor is a lymphoma.

63. The method of claim 62, wherein the lymphoma is a B-cell lymphoma.

64. The method of claim 63, wherein the B-cell lymphoma is diffuse large B cell lymphoma (DLBCL), primary mediastinal B-cell lymphoma (PMBL), Hodgkin lymphoma (HL), non-Hodgkin lymphoma, mediastinal gray zone lymphoma, or nodular sclerosis HL.

65. The method of claim 62, wherein the lymphoma is T-cell lymphoma.

66. The method of claim 65, wherein the T-cell lymphoma is anaplastic large cell lymphoma (ALCL), peripheral T cell lymphoma not otherwise specified (PTCL-NOS), or angioimmunoblastic T cell lymphoma (AITL).

67. The method of any one of claims 58 to 66, wherein the population of cells is autologous to the subject.

68. The method of claim 67, further comprising obtaining T cells from the subject.

69. The method of any one of claims 58 to 68, further comprising administering an additional therapy to the subject.

70. The method of any one of claims 58 to 68, wherein the subject is a human.


SEQUENCE LISTING (223 sequences)

SEQ ID NO: 1 - protein, 361 aa, homo sapiens
  human CD30 ECD
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 2 - protein, 361 aa, rhesus
  rhesus CD30 ECD
FPQDRPFEDTCRGNPGHYYDKAVRRCCYRCPTGLFPTQQCPQRPADCRKQCEPDYYLDEAGRCTACVSCSRDDLVEKMPCAWNSSRVCECQPGMFCAVSVVNSCARCFFHSVCPAGMIVKFPGTAQKNTVCEPASPGVSPACASPENCKEPSSGTIPQAKPTPVSPATSNASTMPLRGGTRLAQEAASKLTRAPGSPSSVGRPSSDPGLSPTQPCPQGSGDCRKQCEPDYYLDEAGRCTACVSCSRDDLVEKTPCAWNSSRICECRPGMICATSATNSCARCVPYPICAAETGTKPQDMAEKDTTFEAPPVGTQPDCSPTPENGEAPASTSPTLSSLVDSQASKTLPIPTSAPIALSSTGK

SEQ ID NO: 3 - protein, 50 aa, Artificial Sequence
  CRD1
FPQDRPFEDTCHGNPSHYYDKAVRRCCYRCPMGLFPTQQCPQRPTDCRKQ

SEQ ID NO: 4 - protein, 42 aa, Artificial Sequence
  CRD2
RKQCEPDYYLDEADRCTACVTCSRDDLVEKTPCAWNSSRVCE

SEQ ID NO: 5 - protein, 47 aa, Artificial Sequence
  CRD3
ECRPGMFCSTSAVNSCARCFFHSVCPAGMIVKFPGTAQKNTVCEPAS

SEQ ID NO: 6 - protein, 94 aa, Artificial Sequence
  CRD4
EPASPGVSPACASPENCKEPSSGTIPQAKPTPVSPATSSASTMPVRGGTRLAQEAASKLTRAPDSPSSVGRPSSDPGLSPTQPCPEGSGDCRKQ

SEQ ID NO: 7 - protein, 42 aa, Artificial Sequence
  CRD5
RKQCEPDYYLDEAGRCTACVSCSRDDLVEKTPCAWNSSRTCE

SEQ ID NO: 8 - protein, 98 aa, Artificial Sequence
  CRD6
ECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 9 - protein, 122 aa, Artificial Sequence
  AS47863
QVQLEESGGGSVQAGETLRLSCTASGSTFGDSDMGWYRQAPGNACELVSIISSDGRTYYVDSVKGRFTISQDNAVSTVYLQMNSLKPEDTGVYYCAADLRQYCRDGRCCGYWGQGTQVTVSS

SEQ ID NO: 10 - protein, 122 aa, Artificial Sequence
  AS48433
QIQLVESGGGSVQAGETLRLSCTASGSTFGDSDMGWYRQAPGNACELVSIISSDGRTYYVDSVKGRFTISQDNAVSTVYLQMNSLNPEDTGVYYCAADLRLNCRDGRCCGYWGQGTQVTVSS

SEQ ID NO: 11 - protein, 123 aa, Artificial Sequence
  AS48463
QVHLMESGGGSVQAGETLRLSCTASGFTFANSDMGWYRQAPGNACELVSIISSHGGTTYYVDSVKGRFTISRHNAENTVYLRMTSLKPEDTALYYCVADPRSNCRGGYCCGYWGPGTQVTVSS

SEQ ID NO: 12 - protein, 124 aa, Artificial Sequence
  AS48481
EVQLVASGGGSVQAGETLRLSCTASGFTFADSAMGWYRKGPGNVCDLVAIIRTDGTTYYGDSAKGRFTISRDNAKSTLYLQMNSLKPEDTAVYFCAADRETSFIGGSWCVAKYWDQGTQVTVSS

SEQ ID NO: 13 - protein, 122 aa, Artificial Sequence
  AS48508
EVQLVESGGGSVQAGGSLRLSCTASRFTFDGPDMAWYRQAPGNACELVSIISADGRTYYTDSVKGRFTISRDNAKNTVFLYLNSLQPEDTAVYYCAPDPRRNCRGGYCCGNWGPGTQVTVSS

SEQ ID NO: 14 - protein, 122 aa, Artificial Sequence
  AS48542
QMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSS

SEQ ID NO: 15 - protein, 119 aa, Artificial Sequence
  AS53445
QVQLVESGGGSVQAGGSLRLSCTASGYIFCMGWFRQAPGKAREGIATIYTGGDSTYYDDSVKGRFTISRDNAKNTVYLQMNSLKPEDTAMYYCAAGGQECYLTNWVSYWGQGTQVTVSS

SEQ ID NO: 16 - protein, 123 aa, Artificial Sequence
  AS53574
QVKLVESGGGSVQAGGSLRLSCAASGYIYSSNCMGWFRQAPGKEREWVARIHTGSGSTYYADSVKGRFTISQDNAKNTVYLQMNSLRPEDTAMYDCAAGRVVLGAVVCTNEYWGQGTQVTVSS

SEQ ID NO: 17 - protein, 122 aa, Artificial Sequence
  AS53750
EVQLVESGGGLVQPGGSLRLSCTASGFTDDGPDMAWYRRAPGNECELVSIISADGRTYYTDSVKGRFTISRDNAKNTVFLYLNSLQPEDTAVYYCAPDPRRNCRGGDCCGNWGPGTQVTVSS

SEQ ID NO: 18 - protein, 121 aa, Artificial Sequence
  AS54233
QVQLVESGGGSVQAGETLRLSCTASGFTFDGPDMAWYRQAPGNECELVSIISADGRTYYTDSVKGRFTASQDNAKNTVSLYLKSLQPEDTAVYYCAADPRRNCRGNCCGNWGPGTQVTVSS

SEQ ID NO: 19 - protein, 122 aa, Artificial Sequence
  AS47863VH4
EVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSS

SEQ ID NO: 20 - protein, 122 aa, Artificial Sequence
  AS47863VH5
EVQLVESGGGLVQPGGSLRLSCAASGSTFGDSDMGWYRQAPGKGCELVSIISSDGRTYYVDSVKGRFTISQDNSKNTVYLQMNSLRAEDTAVYYCAADLRQYCRDGRCCGYWGQGTLVTVSS

SEQ ID NO: 21 - protein, 122 aa, Artificial Sequence
  AS47863VH11
EVQLVESGGGLVQPGGSLRLSCAASGSTFGDSDMGWYRQAPGKGCELVSIISSDGRTYYVDSVKGRFTISQDNAKNTLYLQMNSLRPEDTAVYYCAADLRQYCRDGRCCGYWGQGTLVTVSS

SEQ ID NO: 22 - protein, 122 aa, Artificial Sequence
  AS47863VH12
EVQLVESGGGLVQPGGSLRLSCAASGSTFGDSDMGWYRQAPGKGCELVSIISSDGRTYYVDSVKGRFTISQDNAKNTVYLQMNSLRPEDTAVYYCAADLRQYCRDGRCCGYWGQGTLVTVSS

SEQ ID NO: 23 - protein, 122 aa, Artificial Sequence
  AS48433VH4
EVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRLNCRDGRCCGYWGQGTLVTVSS

SEQ ID NO: 24 - protein, 122 aa, Artificial Sequence
  AS48433VH5
EVQLVESGGGLVQPGGSLRLSCAASGSTFGDSDMGWYRQAPGKGCELVSIISSDGRTYYVDSVKGRFTISQDNSKNTVYLQMNSLRAEDTAVYYCAADLRLNCRDGRCCGYWGQGTLVTVSS

SEQ ID NO: 25 - protein, 122 aa, Artificial Sequence
  AS48433VH11
EVQLVESGGGLVQPGGSLRLSCAASGSTFGDSDMGWYRQAPGKGCELVSIISSDGRTYYVDSVKGRFTISQDNAKNTLYLQMNSLRPEDTAVYYCAADLRLNCRDGRCCGYWGQGTLVTVSS

SEQ ID NO: 26 - protein, 122 aa, Artificial Sequence
  AS48433VH12
EVQLVESGGGLVQPGGSLRLSCAASGSTFGDSDMGWYRQAPGKGCELVSIISSDGRTYYVDSVKGRFTISQDNAKNTVYLQMNSLRPEDTAVYYCAADLRLNCRDGRCCGYWGQGTLVTVSS

SEQ ID NO: 27 - protein, 123 aa, Artificial Sequence
  AS48463VH4
EVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSS

SEQ ID NO: 28 - protein, 123 aa, Artificial Sequence
  AS48463VH11
EVQLLESGGGLVQPGGSLRLSCAASGFTFANSDMGWYRQAPGKGCELVSIISSHGGTTYYVDSVKGRFTISRDNAKNTLYLQMNSLRPEDTAVYYCVADPRSNCRGGYCCGYWGQGTLVTVSS

SEQ ID NO: 29 - protein, 124 aa, Artificial Sequence
  AS48481VH5
QVQLVESGGGVVQPGRSLRLSCAASGFTFADSAMGWYRQAPGKGCELVAIIRTDGTTYYGDSAKGRFTISRDNSKNTLYLQMNSLRAEDTAVYFCAADRETSFIGGSWCVAKYWDQGTLVTVSS

SEQ ID NO: 30 - protein, 124 aa, Artificial Sequence
  AS48481VH6
QVQLVESGGGVVQPGRSLRLSCAASGFTFADSAMGWYRQAPGKVCELVAIIRTDGTTYYGDSAKGRFTISRDNSKNTLYLQMNSLRAEDTAVYFCAADRETSFIGGSWCVAKYWDQGTLVTVSS

SEQ ID NO: 31 - protein, 124 aa, Artificial Sequence
  AS48481VH13
QVQLVESGGGVVQPGRSLRLSCAASGFTFADSAMGWYRQAPGKGCELVAIIRTDGTTYYGDSAKGRFTISRDNAKNTLYLQMNSLRPEDTAVYFCAADRETSFIGGSWCVAKYWDQGTLVTVSS

SEQ ID NO: 32 - protein, 124 aa, Artificial Sequence
  AS48481VH14
QVQLVESGGGVVQPGRSLRLSCAASGFTFADSAMGWYRQAPGKVCELVAIIRTDGTTYYGDSAKGRFTISRDNAKNTLYLQMNSLRPEDTAVYFCAADRETSFIGGSWCVAKYWDQGTLVTVSS

SEQ ID NO: 33 - protein, 122 aa, Artificial Sequence
  AS48508VH4
EVQLVESGGGLVQPGGSLRLSCAASRFTFDGPDMAWYRQAPGKGCELVSIISADGRTYYTDSVKGRFTISRDNSKNTLYLQMNSLRAEDTAVYYCAPDPRRNCRGGYCCGNWGQGTTVTVSS

SEQ ID NO: 34 - protein, 122 aa, Artificial Sequence
  AS48508VH5
EVQLVESGGGLVQPGGSLRLSCAASRFTFDGPDMAWYRQAPGKGCELVSIISADGRTYYTDSVKGRFTISRDNSKNTVYLQMNSLRAEDTAVYYCAPDPRRNCRGGYCCGNWGQGTTVTVSS

SEQ ID NO: 35 - protein, 122 aa, Artificial Sequence
  AS48508VH11
EVQLVESGGGLVQPGGSLRLSCAASRFTFDGPDMAWYRQAPGKGCELVSIISADGRTYYTDSVKGRFTISRDNAKNTLYLQMNSLRPEDTAVYYCAPDPRRNCRGGYCCGNWGQGTTVTVSS

SEQ ID NO: 36 - protein, 122 aa, Artificial Sequence
  AS48508VH12
EVQLVESGGGLVQPGGSLRLSCAASRFTFDGPDMAWYRQAPGKGCELVSIISADGRTYYTDSVKGRFTISRDNAKNTVYLQMNSLRPEDTAVYYCAPDPRRNCRGGYCCGNWGQGTTVTVSS

SEQ ID NO: 37 - protein, 122 aa, Artificial Sequence
  AS48542VH5
EVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSS

SEQ ID NO: 38 - protein, 122 aa, Artificial Sequence
  AS48542VH12
EVQLVESGGGLVQPGGSLRLSCATSAFTFDGPDMAWYRQAPGKGCELVSIISADGRTYYADSVKGRFTISRDNAKNTVYLQMNSLRPEDTAVYYCALDPRKNCRGGYCCANWGQGTLVTVSS

SEQ ID NO: 39 - protein, 119 aa, Artificial Sequence
  AS53445VH4
QVQLVESGGGVVQPGGSLRLSCAASGYIFCMGWFRQAPGKGLEGIATIYTGGDSTYYDDSVKGRFTISRDNSKNTLYLQMNSLRAEDTAVYYCAAGGQECYLTNWVSYWGQGTLVTVSS

SEQ ID NO: 40 - protein, 119 aa, Artificial Sequence
  AS53445VH11
QVQLVESGGGVVQPGGSLRLSCAASGYIFCMGWFRQAPGKGREGIATIYTGGDSTYYDDSVKGRFTISRDNAKNTLYLQMNSLRPEDTAVYYCAAGGQECYLTNWVSYWGQGTLVTVSS

SEQ ID NO: 41 - protein, 123 aa, Artificial Sequence
  AS53574VH4
EVQLVESGGGLVQPGGSLRLSCAASGYIYSSNCMGWFRQAPGKGLEWVSRIHTGSGSTYYADSVKGRFTISRDNSKNTLYLQMNSLRAEDTAVYYCAAGRVVLGAVVCTNEYWGQGTLVTVSS

SEQ ID NO: 42 - protein, 123 aa, Artificial Sequence
  AS53574VH5
EVQLVESGGGLVQPGGSLRLSCAASGYIYSSNCMGWFRQAPGKGLEWVSRIHTGSGSTYYADSVKGRFTISQDNSKNTLYLQMNSLRAEDTAVYYCAAGRVVLGAVVCTNEYWGQGTLVTVSS

SEQ ID NO: 43 - protein, 123 aa, Artificial Sequence
  AS53574VH6
EVQLVESGGGLVQPGGSLRLSCAASGYIYSSNCMGWFRQAPGKGLEWVARIHTGSGSTYYADSVKGRFTISQDNSKNTLYLQMNSLRAEDTAVYYCAAGRVVLGAVVCTNEYWGQGTLVTVSS

SEQ ID NO: 44 - protein, 123 aa, Artificial Sequence
  AS53574VH11
EVQLVESGGGLVQPGGSLRLSCAASGYIYSSNCMGWFRQAPGKGREWVSRIHTGSGSTYYADSVKGRFTISRDNAKNTLYLQMNSLRPEDTAVYYCAAGRVVLGAVVCTNEYWGQGTLVTVSS

SEQ ID NO: 45 - protein, 123 aa, Artificial Sequence
  AS53574VH12
EVQLVESGGGLVQPGGSLRLSCAASGYIYSSNCMGWFRQAPGKGREWVSRIHTGSGSTYYADSVKGRFTISQDNAKNTLYLQMNSLRPEDTAVYYCAAGRVVLGAVVCTNEYWGQGTLVTVSS

SEQ ID NO: 46 - protein, 123 aa, Artificial Sequence
  AS53574VH13
EVQLVESGGGLVQPGGSLRLSCAASGYIYSSNCMGWFRQAPGKGREWVARIHTGSGSTYYADSVKGRFTISQDNAKNTLYLQMNSLRPEDTAVYYCAAGRVVLGAVVCTNEYWGQGTLVTVSS

SEQ ID NO: 47 - protein, 122 aa, Artificial Sequence
  AS53750VH4
EVQLVESGGGLVQPGGSLRLSCAASGFTDDGPDMAWYRQAPGKGCELVSIISADGRTYYTDSVKGRFTISRDNSKNTLYLQMNSLRAEDTAVYYCAPDPRRNCRGGDCCGNWGQGTTVTVSS

SEQ ID NO: 48 - protein, 122 aa, Artificial Sequence
  AS53750VH5
EVQLVESGGGLVQPGGSLRLSCAASGFTDDGPDMAWYRQAPGKGCELVSIISADGRTYYTDSVKGRFTISRDNSKNTVYLQMNSLRAEDTAVYYCAPDPRRNCRGGDCCGNWGQGTTVTVSS

SEQ ID NO: 49 - protein, 122 aa, Artificial Sequence
  AS53750VH11
EVQLVESGGGLVQPGGSLRLSCAASGFTDDGPDMAWYRQAPGKGCELVSIISADGRTYYTDSVKGRFTISRDNAKNTLYLQMNSLRPEDTAVYYCAPDPRRNCRGGDCCGNWGQGTTVTVSS

SEQ ID NO: 50 - protein, 122 aa, Artificial Sequence
  AS53750VH12
EVQLVESGGGLVQPGGSLRLSCAASGFTDDGPDMAWYRQAPGKGCELVSIISADGRTYYTDSVKGRFTISRDNAKNTVYLQMNSLRPEDTAVYYCAPDPRRNCRGGDCCGNWGQGTTVTVSS

SEQ ID NO: 51 - protein, 121 aa, Artificial Sequence
  AS54233VH4
EVQLVESGGGLVQPGGSLRLSCAASGFTFDGPDMAWYRQAPGKGCELVSIISADGRTYYTDSVKGRFTASQDNSKNTLYLQMNSLRAEDTAVYYCAADPRRNCRGNCCGNWGQGTLVTVSS

SEQ ID NO: 52 - protein, 121 aa, Artificial Sequence
  AS54233VH5
EVQLVESGGGLVQPGGSLRLSCAASGFTFDGPDMAWYRQAPGKGCELVSIISADGRTYYTDSVKGRFTASQDNSKNTVYLQMNSLRAEDTAVYYCAADPRRNCRGNCCGNWGQGTLVTVSS

SEQ ID NO: 53 - protein, 121 aa, Artificial Sequence
  AS54233VH11
EVQLVESGGGLVQPGGSLRLSCAASGFTFDGPDMAWYRQAPGKGCELVSIISADGRTYYTDSVKGRFTASQDNAKNTLYLQMNSLRPEDTAVYYCAADPRRNCRGNCCGNWGQGTLVTVSS

SEQ ID NO: 54 - protein, 121 aa, Artificial Sequence
  AS54233VH12
EVQLVESGGGLVQPGGSLRLSCAASGFTFDGPDMAWYRQAPGKGCELVSIISADGRTYYTDSVKGRFTASQDNAKNTVYLQMNSLRPEDTAVYYCAADPRRNCRGNCCGNWGQGTLVTVSS

SEQ ID NO: 55 - protein, 18 aa, Artificial Sequence
  5F11 scFv linker
GSTSGSGKPGSGEGSTKG

SEQ ID NO: 56 - protein, 15 aa, Artificial Sequence
  (G4S)3 linker
GGGGSGGGGSGGGGS

SEQ ID NO: 57 - protein, 5 aa, Artificial Sequence
  G4S linker
GGGGS

SEQ ID NO: 58 - protein, 238 aa, Artificial Sequence
  AS57911 scFv
DIQMTQSPSSLSASVGDRVTITCRASQSVSSAVAWYQQKPGKAPKLLIYSASSLYSGVPSRFSGSRSGTDFTLTISSLQPEDFATYYCQQSHALITFGQGTKVEIKGGGGSGGGGSGGGGSEVQLVESGGGLVQPGGSLRLSCAASGFNISSSYIHWVRQAPGKGLEWVAYISSYYSYTYYADSVKGRFTISADTSKNTAYLQMNSLRAEDTAVYYCARGYPYGMDYWGQGTLVTVSS

SEQ ID NO: 59 - protein, 240 aa, Artificial Sequence
  AS57659 scFv
DIQMTQSPSSLSASVGDRVTITCRASQSVSSAVAWYQQKPGKAPKLLIYSASSLYSGVPSRFSGSRSGTDFTLTISSLQPEDFATYYCQQPYYLITFGQGTKVEIKGGGGSGGGGSGGGGSEVQLVESGGGLVQPGGSLRLSCAASGFNIYSYYIHWVRQAPGKGLEWVASIYSSYSSTYYADSVKGRFTISADTSKNTAYLQMNSLRAEDTAVYYCARSWFSYPGLDYWGQGTLVTVSS

SEQ ID NO: 60 - protein, 249 aa, Artificial Sequence
  AS57765 scFv
DIQMTQSPSSLSASVGDRVTITCRASQSVSSAVAWYQQKPGKAPKLLIYSASSLYSGVPSRFSGSRSGTDFTLTISSLQPEDFATYYCQQAYYSLITFGQGTKVEIKGGGGSGGGGSGGGGSEVQLVESGGGLVQPGGSLRLSCAASGFNIYYSYMHWVRQAPGKGLEWVAYIYPYSGSTSYADSVKGRFTISADTSKNTAYLQMNSLRAEDTAVYYCARPAVHWHGYGGGYYYGLDYWGQGTLVTVSS

SEQ ID NO: 61 - protein, 21 aa, Artificial Sequence
  leader sequence
MALPVTALLLPLALLLHAARP

SEQ ID NO: 62 - protein, 45 aa, Artificial Sequence
  CD8-alpha hinge
TTTPAPRPPTPAPTIASQPLSLRPEACRPAAGGAVHTRGLDFACD

SEQ ID NO: 63 - protein, 24 aa, Artificial Sequence
  CD8-alpha TM
IYIWAPLAGTCGVLLLSLVITLYC

SEQ ID NO: 64 - protein, 42 aa, Artificial Sequence
  cytoplasmic portion of the 4-1BB (CD137)
KRGRKKLLYIFKQPFMRPVQTTQEEDGCSCRFPEEEEGGCEL

SEQ ID NO: 65 - protein, 112 aa, Artificial Sequence
  cytoplasmic portion of the CD3-zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 22 aa, Artificial Sequence
  P2A
GSGATNFSLLKQAGDVEENPGP

SEQ ID NO: 67 - protein, 360 aa, Artificial Sequence
  full-length CCR4
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 68 - protein, 199 aa, Artificial Sequence
  dnTGF-beta-RII
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 69 - protein, 235 aa, Artificial Sequence
  PD1CD28
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 70 - protein, 366 aa, Artificial Sequence
  AS47863bbz
MALPVTALLLPLALLLHAARPQVQLEESGGGSVQAGETLRLSCTASGSTFGDSDMGWYRQAPGNACELVSIISSDGRTYYVDSVKGRFTISQDNAVSTVYLQMNSLKPEDTGVYYCAADLRQYCRDGRCCGYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 366 aa, Artificial Sequence; identical to SEQ ID NO: 70
  AS48433bbz

SEQ ID NO: 72 - protein, 367 aa, Artificial Sequence
  AS48463bbz
MALPVTALLLPLALLLHAARPQVHLMESGGGSVQAGETLRLSCTASGFTFANSDMGWYRQAPGNACELVSIISSHGGTTYYVDSVKGRFTISRHNAENTVYLRMTSLKPEDTALYYCVADPRSNCRGGYCCGYWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 368 aa, Artificial Sequence
  AS48481bbz
MALPVTALLLPLALLLHAARPEVQLVASGGGSVQAGETLRLSCTASGFTFADSAMGWYRKGPGNVCDLVAIIRTDGTTYYGDSAKGRFTISRDNAKSTLYLQMNSLKPEDTAVYFCAADRETSFIGGSWCVAKYWDQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 366 aa, Artificial Sequence
  AS48508bbz
MALPVTALLLPLALLLHAARPEVQLVESGGGSVQAGGSLRLSCTASRFTFDGPDMAWYRQAPGNACELVSIISADGRTYYTDSVKGRFTISRDNAKNTVFLYLNSLQPEDTAVYYCAPDPRRNCRGGYCCGNWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 366 aa, Artificial Sequence
  AS48542bbz
MALPVTALLLPLALLLHAARPQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 366 aa, Artificial Sequence
  AS53750bbz
MALPVTALLLPLALLLHAARPEVQLVESGGGLVQPGGSLRLSCTASGFTDDGPDMAWYRRAPGNECELVSIISADGRTYYTDSVKGRFTISRDNAKNTVFLYLNSLQPEDTAVYYCAPDPRRNCRGGDCCGNWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 365 aa, Artificial Sequence
  AS54233bbz
MALPVTALLLPLALLLHAARPQVQLVESGGGSVQAGETLRLSCTASGFTFDGPDMAWYRQAPGNECELVSIISADGRTYYTDSVKGRFTASQDNAKNTVSLYLKSLQPEDTAVYYCAADPRRNCRGNCCGNWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 363 aa, Artificial Sequence
  AS53445bbz
MALPVTALLLPLALLLHAARPQVQLVESGGGSVQAGGSLRLSCTASGYIFCMGWFRQAPGKAREGIATIYTGGDSTYYDDSVKGRFTISRDNAKNTVYLQMNSLKPEDTAMYYCAAGGQECYLTNWVSYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 367 aa, Artificial Sequence
  AS53574bbz
MALPVTALLLPLALLLHAARPQVKLVESGGGSVQAGGSLRLSCAASGYIYSSNCMGWFRQAPGKEREWVARIHTGSGSTYYADSVKGRFTISQDNAKNTVYLQMNSLRPEDTAMYDCAAGRVVLGAVVCTNEYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 482 aa, Artificial Sequence
  AS57911bbz
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQSHALITFGQGTKVEIKGGGGSGGGGSGGGGSEVQLVESGGGLVQPGGSLRLSCAASGFNISSSYIHWVRQAPGKGLEWVAYISSYYSYTYYADSVKGRFTISADTSKNTAYLQMNSLRAEDTAVYYCARGYPYGMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 484 aa, Artificial Sequence
  AS57659bbz
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQPYYLITFGQGTKVEIKGGGGSGGGGSGGGGSEVQLVESGGGLVQPGGSLRLSCAASGFNIYSYYIHWVRQAPGKGLEWVASIYSSYSSTYYADSVKGRFTISADTSKNTAYLQMNSLRAEDTAVYYCARSWFSYPGLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 493 aa, Artificial Sequence
  AS57765bbz
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQAYYSLITFGQGTKVEIKGGGGSGGGGSGGGGSEVQLVESGGGLVQPGGSLRLSCAASGFNIYYSYMHWVRQAPGKGLEWVAYIYPYSGSTSYADSVKGRFTISADTSKNTAYLQMNSLRAEDTAVYYCARPAVHWHGYGGGYYYGLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 493 aa, Artificial Sequence
  AS48542dis-bbz
MALPVTALLLPLALLLHAARPQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSGGGGSQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 503 aa, Artificial Sequence
  AS48542dil-bbz
MALPVTALLLPLALLLHAARPQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSGGGGSGGGGSGGGGSQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 504 aa, Artificial Sequence
  AS48542-AS53574bil-bbz
MALPVTALLLPLALLLHAARPQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSGGGGSGGGGSGGGGSQVKLVESGGGSVQAGGSLRLSCAASGYIYSSNCMGWFRQAPGKEREWVARIHTGSGSTYYADSVKGRFTISQDNAKNTVYLQMNSLRPEDTAMYDCAAGRVVLGAVVCTNEYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 504 aa, Artificial Sequence
  AS53574-AS48542bil-bbz
MALPVTALLLPLALLLHAARPQVKLVESGGGSVQAGGSLRLSCAASGYIYSSNCMGWFRQAPGKEREWVARIHTGSGSTYYADSVKGRFTISQDNAKNTVYLQMNSLRPEDTAMYDCAAGRVVLGAVVCTNEYWGQGTQVTVSSGGGGSGGGGSGGGGSQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 10 aa, Artificial Sequence
  AS47863-CDR1
GSTFGDSDMG

SEQ ID NO: 88 - protein, 10 aa, Artificial Sequence
  AS48463-CDR1
GFTFANSDMG

SEQ ID NO: 89 - protein, 10 aa, Artificial Sequence
  AS48481-CDR1
GFTFADSAMG

SEQ ID NO: 90 - protein, 10 aa, Artificial Sequence
  AS48508-CDR1
RFTFDGPDMA

SEQ ID NO: 91 - protein, 10 aa, Artificial Sequence
  AS48542-CDR1
AFTFDGPDMA

SEQ ID NO: 92 - protein, 7 aa, Artificial Sequence
  AS53445-CDR1
GYIFCMG

SEQ ID NO: 93 - protein, 10 aa, Artificial Sequence
  AS53574-CDR1
GYIYSSNCMG

SEQ ID NO: 94 - protein, 10 aa, Artificial Sequence
  AS53750-CDR1
GFTDDGPDMA

SEQ ID NO: 95 - protein, 10 aa, Artificial Sequence
  AS54233-CDR1
GFTFDGPDMA

SEQ ID NO: 96 - protein, 10 aa, Artificial Sequence
  AS57659VH-CDR1
GFNIYSYYIH

SEQ ID NO: 97 - protein, 10 aa, Artificial Sequence
  AS57765VH-CDR1
GFNIYYSYMH

SEQ ID NO: 98 - protein, 10 aa, Artificial Sequence
  AS57911VH-CDR1
GFNISSSYIH

SEQ ID NO: 99 - protein, 11 aa, Artificial Sequence
  AS57659VL-CDR1
RASQSVSSAVA

SEQ ID NO: 100 - protein, 16 aa, Artificial Sequence
  AS47863-CDR2
IISSDGRTYYVDSVKG

SEQ ID NO: 101 - protein, 17 aa, Artificial Sequence
  AS48463-CDR2
IISSHGGTTYYVDSVKG

SEQ ID NO: 102 - protein, 16 aa, Artificial Sequence
  AS48481-CDR2
IIRTDGTTYYGDSAKG

SEQ ID NO: 103 - protein, 16 aa, Artificial Sequence
  AS48508-CDR2
IISADGRTYYTDSVKG

SEQ ID NO: 104 - protein, 16 aa, Artificial Sequence
  AS48542-CDR2
IISADGRTYYADSVKG

SEQ ID NO: 105 - protein, 17 aa, Artificial Sequence
  AS53445-CDR2
TIYTGGDSTYYDDSVKG

SEQ ID NO: 106 - protein, 17 aa, Artificial Sequence
  AS53574-CDR2
RIHTGSGSTYYADSVKG

SEQ ID NO: 107 - protein, 17 aa, Artificial Sequence
  AS57659VH-CDR2
SIYSSYSSTYYADSVKG

SEQ ID NO: 108 - protein, 17 aa, Artificial Sequence
  AS57765VH-CDR2
YIYPYSGSTSYADSVKG

SEQ ID NO: 109 - protein, 17 aa, Artificial Sequence
  AS57911VH-CDR2
YISSYYSYTYYADSVKG

SEQ ID NO: 110 - protein, 7 aa, Artificial Sequence
  AS57765VL-CDR2
SASSLYS

SEQ ID NO: 111 - protein, 14 aa, Artificial Sequence
  AS47863-CDR3
DLRQYCRDGRCCGY

SEQ ID NO: 112 - protein, 14 aa, Artificial Sequence
  AS48433-CDR3
DLRLNCRDGRCCGY

SEQ ID NO: 113 - protein, 14 aa, Artificial Sequence
  AS48463-CDR3
DPRSNCRGGYCCGY

SEQ ID NO: 114 - protein, 16 aa, Artificial Sequence
  AS48481-CDR3
DRETSFIGGSWCVAKY

SEQ ID NO: 115 - protein, 14 aa, Artificial Sequence
  AS48508-CDR3
DPRRNCRGGYCCGN

SEQ ID NO: 116 - protein, 14 aa, Artificial Sequence
  AS48542-CDR3
DPRKNCRGGYCCAN

SEQ ID NO: 117 - protein, 13 aa, Artificial Sequence
  AS53445-CDR3
GGQECYLTNWVSY

SEQ ID NO: 118 - protein, 14 aa, Artificial Sequence
  AS53574-CDR3
GRVVLGAVVCTNEY

SEQ ID NO: 119 - protein, 14 aa, Artificial Sequence
  AS53750-CDR3
DPRRNCRGGDCCGN

SEQ ID NO: 120 - protein, 13 aa, Artificial Sequence
  AS54233-CDR3
DPRRNCRGNCCGN

SEQ ID NO: 121 - protein, 10 aa, Artificial Sequence
  AS57659VH-CDR3
SWFSYPGLDY

SEQ ID NO: 122 - protein, 18 aa, Artificial Sequence
  AS57765VH-CDR3
PAVHWHGYGGGYYYGLDY

SEQ ID NO: 123 - protein, 8 aa, Artificial Sequence
  AS57911VH-CDR3
GYPYGMDY

SEQ ID NO: 124 - protein, 8 aa, Artificial Sequence
  AS57659VL-CDR3
QQPYYLIT

SEQ ID NO: 125 - protein, 9 aa, Artificial Sequence
  AS57765VL-CDR3
QQAYYSLIT

SEQ ID NO: 126 - protein, 8 aa, Artificial Sequence
  AS57911VL-CDR3
QQSHALIT

SEQ ID NO: 127 - protein, 39 aa, Artificial Sequence
  CD28 hinge
IEVMYPPPYLDNEKSNGTIIHVKGKHLCPSPLFPGPSKP

SEQ ID NO: 128 - protein, 27 aa, Artificial Sequence
  CD28 TM
FWVLVVVGGVLACYSLLVTVAFIIFWV

SEQ ID NO: 129 - protein, 41 aa, Artificial Sequence
  cytoplasmic portion of CD28
RSKRSRLLHSDYMNMTPRRPGPTRKHYQPYAPPRDFAAYRS

SEQ ID NO: 130 - DNA, 366 bp, Artificial Sequence
  AS47863 sdAb
caggtgcaattggaggagtctgggggaggctcggtgcaggctggagagactctgagactctcctgtacagcctctggatccacttttggtgattctgacatgggctggtaccgccaggctccaggaaatgcgtgcgagttggtatcaattattagtagtgacggtaggacatactatgtggactccgtgaagggccgattcaccatctcccaagacaacgccgtgagcacggtgtatctgcaaatgaacagcctgaaacctgaggacacaggcgtgtattactgtgcggcagacctccgccaatattgtagggatggtcgctgctgcggttattggggccaggggacccaggtcaccgtctcctca

SEQ ID NO: 131 - DNA, 366 bp, Artificial Sequence
  AS48433 sdAb
cagattcagctggtggagtctgggggaggctcggtgcaggctggagagactctgagactctcctgtacagcctctggatccacttttggtgattctgacatgggctggtaccgccaggctccagggaatgcgtgcgagttggtgtcaattattagtagtgacgggcggacatactatgtggactccgtgaagggccgattcaccatctcccaagacaacgccgtgagcacggtgtatctgcaaatgaacagcctgaatcctgaggacacaggcgtgtattactgtgcggcagacctccgcctcaattgtagggatggtcgctgctgcggttattggggccaggggacccaggtcaccgtctcctca

SEQ ID NO: 132 - DNA, 369 bp, Artificial Sequence
  AS48463 sdAb
caggtgcacctgatggagtctgggggaggctcggtgcaggctggagagactctgagactctcctgtacagcctctggattcacttttgctaattctgacatgggctggtaccgccaggctccaggaaatgcgtgcgagttggtctcaattattagtagtcatggtggtacgacatactatgtagactccgtgaagggccgattcaccatctcccggcacaacgccgagaacacggtgtatctgcgaatgactagcctgaaacctgaggacacagccctatattactgtgtcgcagacccgaggtcaaattgtcgtggtggttactgctgtggttactggggcccggggacccaggtcaccgtctcctca

SEQ ID NO: 133 - DNA, 372 bp, Artificial Sequence
  AS48481 sdAb
gaggtgcaactggtggcgtctgggggaggctcggtgcaggctggagagactctgagactctcctgtacagcctctggattcacttttgctgattctgccatgggctggtaccgaaagggtccagggaatgtgtgcgacttggtagcaattattaggacagatggtaccacatactatggcgactccgcgaagggccgattcaccatctcccgagacaacgccaagagcacgctgtatctgcaaatgaacagcctgaaacctgaggatacagccgtgtatttctgtgcggcagaccgggagacgtcttttatcggtggtagctggtgtgttgctaagtactgggaccaggggacccaggtcaccgtctcctca

SEQ ID NO: 134 - DNA, 366 bp, Artificial Sequence
  AS48508 sdAb
gaggtgcagctggtggagtctgggggaggctcggtgcaggctggagggtctctgagactctcatgtacagcctctagattcacttttgatggtcccgacatggcctggtaccgccaggctccagggaatgcgtgcgagttggtctcaattattagtgctgatggtagaacctactatacagactccgtgaagggccgattcaccatctcccgagacaacgccaagaacacggtgttcctgtatttgaacagcctgcaacctgaggacacagccgtatattactgtgcgccagatccccgtagaaattgtagaggtggttattgctgtggcaactggggcccggggacccaggtcaccgtctcctca

SEQ ID NO: 135 - DNA, 366 bp, Artificial Sequence
  AS48542 sdAb
cagatgcagctggtggagtctgggggaggctcggtgcaggctggagagactctgagactctcatgtacaacctctgccttcacttttgatggtcccgacatggcctggtaccgccaggctccagggaatgagtgcgtgttggtctcaattattagtgctgatggtagaacctactatgcagactccgtgaagggccgattcaccatctcccgagacaacgccaagaacacggtgttcctgaatttgaacagcctgcaacctgaggacacagccgtatattactgtgcgttagatccccgtaaaaattgtagaggtggttattgctgtgccaactggggcccggggacccaggtcaccgtctcctca

SEQ ID NO: 136 - DNA, 357 bp, Artificial Sequence
  AS53445 sdAb
caggtgcagctggtggagtctgggggaggctcggtacaggctggagggtctctgagactctcctgtacagcctctggatacattttttgcatgggctggttccgccaggctccagggaaggcccgcgaggggatcgcaactatttatacgggtggtgatagcacatattatgacgactccgtgaagggccgattcaccatctcccgggacaacgccaagaacacggtgtatctgcaaatgaacagcctgaaacctgaggacactgccatgtactactgtgcggcagggggccaagagtgctatttaacgaactgggttagctactggggccaggggacccaggtcaccgtctcctca

SEQ ID NO: 137 - DNA, 369 bp, Artificial Sequence
  AS53574 sdAb
caggtgaagttagtggagtctgggggaggctcggtgcaggctggagggtctctgagactctcctgtgcagcctctggatacatctacagtagtaactgcatgggctggttccgccaggctccagggaaggagcgcgagtgggtcgcacgtattcatactggtagtggtagcacatactatgccgactccgtgaagggccgattcaccatctcccaagacaacgccaagaacacggtgtacctgcaaatgaacagcctgagacctgaggacactgccatgtacgactgtgcggcaggccgagtggtacttggtgcggtggtctgcacgaatgagtactggggccaggggacccaggtcaccgtctcctca

SEQ ID NO: 138 - DNA, 366 bp, Artificial Sequence
  AS53750 sdAb
gaggtgcagctggtggagtctgggggaggcttggtgcagcctggggggtctctgagactctcatgtacagcctctggattcactgatgatggtcccgacatggcctggtaccgccgggctccagggaatgagtgcgagttggtctcaattattagtgctgatggtagaacctactatacagactccgtgaaggggcgattcaccatctcccgagacaacgccaaaaacacggtgttcctgtatttgaacagcctgcaacctgaggacacagccgtatattactgtgcgccagatccccgtagaaattgtagaggtggtgattgctgtggcaactggggcccggggacccaggtcaccgtctcctca

SEQ ID NO: 139 - DNA, 363 bp, Artificial Sequence
  AS54233 sdAb
caggtgcagctggtggagtctgggggaggctcggtgcaggctggagagactctgagactctcatgtacagcctctggattcacttttgatggtcccgacatggcctggtaccgccaggctccagggaatgagtgcgagttggtctcaattattagtgctgatggtagaacctactatacagactccgtgaagggccgattcaccgcctcccaagacaacgccaagaacacggtgtctctatatttgaaaagcctgcaacctgaggacacagccgtatattactgtgcggcagatccccgtagaaattgtagaggtaattgctgtggcaactggggcccggggacccaggtcaccgtctcctca

SEQ ID NO: 140 - DNA, 366 bp, Artificial Sequence
  AS47863VH4 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtccatcatcagctccgacggcaggacatactatgtggattctgtgaagggccgctttaccatctctcaggacaacagcaagaatacactgtatctgcagatgaactctctgcgggccgaggataccgccgtgtactattgcgccgccgacctgagacagtactgtcgggatggcagatgctgtggctattggggccagggcaccctggtgacagtgtctagc

SEQ ID NO: 141 - DNA, 366 bp, Artificial Sequence
  AS47863VH5 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtccatcatcagctccgacggcaggacatactatgtggattctgtgaagggccgctttaccatctctcaggacaacagcaagaatacagtgtatctgcagatgaactctctgcgggccgaggataccgccgtgtactattgcgccgccgacctgagacagtactgtcgggatggcagatgctgtggctattggggccagggcaccctggtgacagtgtctagc

SEQ ID NO: 142 - DNA, 366 bp, Artificial Sequence
  AS47863VH11 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtctatcatcagctccgacggcaggacatactatgtggattctgtgaagggccgctttaccatcagccaggacaacgccaagaatacactgtatctgcagatgaactccctgcggcccgaggataccgccgtgtactattgcgccgccgacctgagacagtactgtcgggatggcagatgctgtggctattggggccagggcaccctggtgacagtgtctagc

SEQ ID NO: 143 - DNA, 366 bp, Artificial Sequence
  AS47863VH12 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtctatcatcagctccgacggcaggacatactatgtggattctgtgaagggccgctttaccatcagccaggacaacgccaagaatacagtgtatctgcagatgaactccctgcggcccgaggataccgccgtgtactattgcgccgccgacctgagacagtactgtcgggatggcagatgctgtggctattggggccagggcaccctggtgacagtgtctagc

SEQ ID NO: 144 - DNA, 366 bp, Artificial Sequence
  AS48433VH4 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtccatcatcagctccgacggcaggacatactatgtggattctgtgaagggccgctttaccatctctcaggacaacagcaagaatacactgtacctgcagatgaactctctgcgggccgaggataccgccgtgtactattgcgccgccgacctgagactgaattgtcgggatggcagatgctgtggctattggggccagggcaccctggtgacagtgtctagc

SEQ ID NO: 145 - DNA, 366 bp, Artificial Sequence
  AS48433VH5 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtccatcatcagctccgacggcaggacatactatgtggattctgtgaagggccgctttaccatctctcaggacaacagcaagaatacagtgtacctgcagatgaactctctgcgggccgaggataccgccgtgtactattgcgccgccgacctgagactgaattgtcgggatggcagatgctgtggctattggggccagggcaccctggtgacagtgtctagc

SEQ ID NO: 146 - DNA, 366 bp, Artificial Sequence
  AS48433VH11 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtctatcatcagctccgacggcaggacatactatgtggattctgtgaagggccgctttaccatcagccaggacaacgccaagaatacactgtacctgcagatgaactccctgcggcccgaggataccgccgtgtactattgcgccgccgacctgagactgaattgtcgggatggcagatgctgtggctattggggccagggcaccctggtgacagtgtctagc

SEQ ID NO: 147 - DNA, 366 bp, Artificial Sequence
  AS48433VH12 sdAb
gaggtgcagctggtggagagcggaggaggactggtgcagccaggaggcagcctgaggctgtcctgcgcagcatccggatctaccttcggcgactccgatatgggctggtacagacaggcccctggcaagggctgtgagctggtgtctatcatcagctccgacggcaggacatactatgtggattctgtgaagggccgctttaccatcagccaggacaacgccaagaatacagtgtacctgcagatgaactccctgcggcccgaggataccgccgtgtactattgcgccgccgacctgagactgaattgtcgggatggcagatgctgtggctattggggccagggcaccctggtgacagtgtctagc

SEQ ID NO: 148 - DNA, 369 bp, Artificial Sequence
  AS48463VH4 sdAb
gaggtgcagctgctggagtccggaggaggactggtgcagccaggaggctccctgaggctgtcttgcgcagcaagcggcttcacctttgccaactctgacatgggatggtacaggcaggcacctggcaagggatgtgagctggtgagcatcatcagctcccacggcggcaccacatactatgtggactccgtgaagggcaggttcaccatctcccgcgataactctaagaatacactgtatctgcagatgaactctctgcgggccgaggacacagccgtgtactattgcgtggccgatccccggagcaattgtagaggcggctactgctgtggctattggggccagggcaccctggtgacagtgtctagc

SEQ ID NO: 149 - DNA, 369 bp, Artificial Sequence
  AS48463VH11 sdAb
gaggtgcagctgctggagtccggaggaggactggtgcagccaggaggctccctgaggctgtcttgcgcagcaagcggcttcacctttgccaacagcgacatgggatggtacaggcaggcaccaggcaagggatgtgagctggtgtccatcatcagctcccacggcggcaccacatactatgtggactccgtgaagggcaggttcaccatctctcgcgataacgccaagaatacactgtatctgcagatgaactctctgcggcccgaggacacagccgtgtactattgcgtggccgatcctcggagcaattgtagaggcggctactgctgtggctattggggccagggcaccctggtgacagtgtctagc

SEQ ID NO: 150 - DNA, 372 bp, Artificial Sequence
  AS48481VH5 sdAb
caggtgcagctggtggagtctggaggaggagtggtgcagccaggccggtctctgagactgagctgcgcagcatccggcttcacctttgccgacagcgccatgggatggtacaggcaggcacctggcaagggatgtgagctggtggccatcatcagaacagacggcaccacatactatggcgatagcgccaagggcaggttcaccatctctcgcgataacagcaagaatacactgtacctgcagatgaactccctgagggcagaggacaccgccgtgtatttctgcgccgccgatagagagacatcctttatcggcggctcttggtgcgtggccaagtattgggaccagggcaccctggtgacagtgagctcc

SEQ ID NO: 151 - DNA, 372 bp, Artificial Sequence
  AS48481VH6 sdAb DNA seque
caggtgcagctggtggagtctggaggaggagtggtgcagccaggccggtctctgagactgagctgcgcagcatccggcttcacctttgccgacagcgccatgggatggtacaggcaggcacctggcaaggtatgtgagctggtggccatcatcagaacagacggcaccacatactatggcgatagcgccaagggcaggttcaccatctctcgcgataacagcaagaatacactgtacctgcagatgaactccctgagggcagaggacaccgccgtgtatttctgcgccgccgatagagagacatcctttatcggcggctcttggtgcgtggccaagtattgggaccagggcaccctggtgacagtgagctcc

SEQ ID NO: 152 - DNA, 372 bp, Artificial Sequence
  AS48481VH13 sdAb
caggtgcagctggtggagagcggaggaggagtggtgcagccaggacggtctctgagactgagctgcgcagcatccggcttcacctttgcagactccgcaatgggatggtacaggcaggcacctggcaagggatgtgagctggtggccatcatcagaacagacggcaccacatactatggcgattccgccaagggcaggttcaccatctctcgcgataacgccaagaatacactgtacctgcagatgaactctctgcggcccgaggacaccgccgtgtatttctgcgccgccgatagagagacatcttttatcggcggcagctggtgtgtggccaagtattgggaccagggcaccctggtgacagtgagctcc

SEQ ID NO: 153 - DNA, 372 bp, Artificial Sequence
  AS48481VH14 sdAb
caggtgcagctggtggagagcggaggaggagtggtgcagccaggacggtctctgagactgagctgcgcagcatccggcttcacctttgcagactccgcaatgggatggtacaggcaggcacctggcaaggtctgtgagctggtggccatcatcagaacagacggcaccacatactatggcgattccgccaagggcaggttcaccatctctcgcgataacgccaagaatacactgtacctgcagatgaactctctgcggcccgaggacaccgccgtgtatttctgcgccgccgatagagagacatcttttatcggcggcagctggtgtgtggccaagtattgggaccagggcaccctggtgacagtgagctcc

SEQ ID NO: 154 - DNA, 366 bp, Artificial Sequence
  AS48508VH4 sdAb
gaggtgcagctggtggagtctggaggaggactggtgcagccaggaggctccctgcggctgtcttgcgccgccagcagattcacctttgacggcccagatatggcatggtacaggcaggcaccaggcaagggatgtgagctggtgtctatcatcagcgccgacggccgcacctactatacagatagcgtgaagggcaggttcaccatctcccgcgacaactctaagaatacactgtacctgcagatgaactccctgagggcagaggacaccgcagtgtactattgcgcccccgatcctcggagaaactgtcggggcggctattgctgtggcaattggggccagggcaccacagtgacagtgagctcc

SEQ ID NO: 155 - DNA, 366 bp, Artificial Sequence
  AS48508VH5 sdAb
gaggtgcagctggtggagtctggaggaggactggtgcagccaggaggctccctgcggctgtcttgcgccgccagcagattcacctttgacggcccagatatggcatggtacaggcaggcaccaggcaagggatgtgagctggtgtctatcatcagcgccgacggccgcacctactatacagatagcgtgaagggcaggttcaccatctcccgcgacaactctaagaatacagtgtacctgcagatgaactccctgagggcagaggacaccgcagtgtactattgcgcccccgatcctcggagaaactgtcggggcggctattgctgtggcaattggggccagggcaccacagtgacagtgagctcc

SEQ ID NO: 156 - DNA, 366 bp, Artificial Sequence
  AS48508VH11 sdAb
gaggtgcagctggtggagagcggaggaggactggtgcagcctggaggctccctgaggctgtcttgcgcagcaagcagattcacctttgacggcccagatatggcatggtacaggcaggcaccaggcaagggatgtgagctggtgtctatcatcagcgccgacggccgcacctactatacagattccgtgaagggcaggttcaccatctctcgcgacaacgccaagaatacactgtacctgcagatgaactccctgaggccagaggacaccgcagtgtactattgcgcccccgatcctcggagaaactgtcggggcggctattgctgtggcaattggggccagggcaccacagtgacagtgagctcc

SEQ ID NO: 157 - DNA, 366 bp, Artificial Sequence
  AS48508VH12 sdAb
gaggtgcagctggtggagagcggaggaggactggtgcagcctggaggctccctgaggctgtcttgcgcagcaagcagattcacctttgacggcccagatatggcatggtacaggcaggcaccaggcaagggatgtgagctggtgtctatcatcagcgccgacggccgcacctactatacagattccgtgaagggcaggttcaccatctctcgcgacaacgccaagaatacagtgtacctgcagatgaactccctgaggccagaggacaccgcagtgtactattgcgcccccgatcctcggagaaactgtcggggcggctattgctgtggcaattggggccagggcaccacagtgacagtgagctcc

SEQ ID NO: 158 - DNA, 366 bp, Artificial Sequence
  AS48542VH5 sdAb
gaggtgcagctggtggagtccggaggaggactggtgcagccaggaggctccctgaggctgtcttgcgccacaagcgccttcacctttgacggccccgatatggcatggtacaggcaggcacctggcaagggatgtgagctggtgtctatcatcagcgccgacggccgcacatactatgccgattctgtgaagggcaggttcacaatctcccgcgacaactctaagaataccgtgtacctgcagatgaacagcctgagggcagaggacaccgccgtgtactattgcgccctggatccccggaagaactgtagaggcggctattgctgtgccaattggggccagggcacactggtgaccgtgagctcc

SEQ ID NO: 159 - DNA, 366 bp, Artificial Sequence
  AS48542VH12 sdAb
gaggtgcagctggtggagtctggaggaggactggtgcagcctggaggctccctgaggctgtcttgcgccacaagcgccttcacctttgacggccccgatatggcatggtacaggcaggcacctggcaagggatgtgagctggtgtctatcatcagcgccgacggccgcacatactatgccgatagcgtgaagggcaggttcacaatctcccgcgacaacgccaagaataccgtgtacctgcagatgaacagcctgcggccagaggacaccgccgtgtactattgcgccctggatccccggaagaactgtagaggcggctattgctgtgccaattggggccagggcacactggtgaccgtgagctcc

SEQ ID NO: 160 - DNA, 357 bp, Artificial Sequence
  AS53445VH4 sdAb
caggtgcagctggtggagtctggaggaggagtggtgcagccaggaggctctctgaggctgagctgcgcagcatccggatacatcttctgtatgggatggtttaggcaggcacctggcaagggactggagggaatcgccaccatctatacaggcggcgactccacctactatgacgattctgtgaagggccggttcaccatctctagagataacagcaagaatacactgtacctgcagatgaacagcctgagggcagaggacaccgcagtgtactattgcgcagcaggaggacaggagtgttacctgacaaattgggtgtcctattggggccagggcaccctggtgacagtgagctcc

SEQ ID NO: 161 - DNA, 357 bp, Artificial Sequence
  AS53445VH11 sdAb
caggtgcagctggtggagagcggaggaggagtggtgcagccaggaggctctctgcggctgagctgcgccgcctccggctacatcttctgtatgggctggtttaggcaggcacctggcaagggaagggagggaatcgcaaccatctatacaggcggcgactctacctactatgacgatagcgtgaagggccggttcaccatctccagagataacgccaagaatacactgtacctgcagatgaactctctgaggcccgaggacaccgcagtgtactattgcgcagcaggaggacaggagtgttacctgacaaattgggtgtcctattggggccagggcaccctggtgacagtgagctcc

SEQ ID NO: 162 - DNA, 369 bp, Artificial Sequence
  AS53574VH4 sdAb
gaggtgcagctggtggagtccggaggaggactggtgcagccaggaggcagcctgcggctgtcctgcgccgcctctggctacatctatagctccaactgtatgggatggttcaggcaggcacctggcaagggactggagtgggtgtctcgcatccacaccggctccggctctacatactatgccgacagcgtgaagggccggtttaccatcagcagagataactccaagaatacactgtacctgcagatgaactctctgcgggccgaggacaccgcagtgtactattgcgcagcaggaagggtggtgctgggagcagtggtgtgtacaaatgagtattggggccagggcaccctggtgacagtgtctagc

SEQ ID NO: 163 - DNA, 369 bp, Artificial Sequence
  AS53574VH5 sdAb
gaggtgcagctggtggagtccggaggaggactggtgcagccaggaggcagcctgcggctgtcctgcgccgcctctggctacatctatagctccaactgtatgggatggttcaggcaggcacctggcaagggactggagtgggtgtctagaatccacaccggctccggctctacatactatgccgacagcgtgaagggcaggtttaccatcagccaggataactccaagaatacactgtacctgcagatgaactctctgagggccgaggacaccgcagtgtactattgcgcagcaggaagggtggtgctgggagcagtggtgtgtacaaatgagtattggggccagggcaccctggtgacagtgtctagc

SEQ ID NO: 164 - DNA, 369 bp, Artificial Sequence
  AS53574VH6 sdAb
gaggtgcagctggtggagtccggaggaggactggtgcagccaggaggcagcctgcggctgtcctgcgccgcctctggctacatctatagctccaactgtatgggatggttcaggcaggcacctggcaagggcctggagtgggtggccagaatccacaccggctccggctctacatactatgccgactctgtgaagggcaggtttaccatcagccaggataactccaagaatacactgtacctgcagatgaacagcctgagggccgaggacaccgcagtgtactattgcgcagcaggaagggtggtgctgggagcagtggtgtgtacaaatgagtattggggccagggcaccctggtgacagtgtctagc

SEQ ID NO: 165 - DNA, 369 bp, Artificial Sequence
  AS53574VH11 sdAb
gaggtgcagctggtggagtccggaggaggactggtgcagccaggaggcagcctgaggctgtcctgcgccgcctctggctacatctatagctccaactgtatgggctggttcagacaggcacctggcaagggaagggagtgggtgtctagaatccacaccggctccggctctacatactatgccgacagcgtgaagggcaggtttaccatctcccgcgataacgccaagaatacactgtacctgcagatgaacagcctgaggccagaggacaccgcagtgtactattgcgcagcaggaagagtggtgctgggagcagtggtgtgtacaaatgagtattggggccagggcaccctggtgacagtgtctagc

SEQ ID NO: 166 - DNA, 369 bp, Artificial Sequence
  AS53574VH12 sdAb
gaggtgcagctggtggagtccggaggaggactggtgcagccaggaggcagcctgcggctgtcctgcgccgcctctggctacatctatagctccaactgtatgggctggttcaggcaggcacctggcaagggaagggagtgggtgtctagaatccacaccggctccggctctacatactatgccgacagcgtgaagggccggtttaccatctcccaggataacgccaagaatacactgtacctgcagatgaacagcctgaggcccgaggacaccgcagtgtactattgcgcagcaggaagggtggtgctgggagcagtggtgtgtacaaatgagtattggggccagggcaccctggtgacagtgtctagc

SEQ ID NO: 167 - DNA, 369 bp, Artificial Sequence
  AS53574VH13 sdAb
gaggtgcagctggtggagtctggaggaggactggtgcagccaggaggcagcctgcggctgtcctgcgccgcctctggctacatctatagctccaactgtatgggctggttcaggcaggcacctggcaagggaagggagtgggtggccagaatccacaccggctccggctctacatactatgccgacagcgtgaagggccggtttaccatctcccaggataacgccaagaatacactgtacctgcagatgaacagcctgaggcccgaggacaccgcagtgtactattgcgcagcaggaagggtggtgctgggagcagtggtgtgtacaaatgagtattggggccagggcaccctggtgacagtgtctagc

SEQ ID NO: 168 - protein, 366 aa, Artificial Sequence
  AS53750VH4 sdAb
GAGGTGCAGCTGGTGGAGTCTGGAGGAGGACTGGTGCAGCCAGGAGGCTCCCTGAGGCTGTCTTGCGCAGCAAGCGGCTTCACCGACGATGGACCAGATATGGCATGGTACAGGCAGGCACCAGGCAAGGGATGTGAGCTGGTGTCTATCATCAGCGCCGACGGCAGAACCTACTATACAGATAGCGTGAAGGGCAGGTTTACCATCTCCCGCGACAACTCTAAGAATACACTGTATCTGCAGATGAACTCCCTGAGGGCCGAGGACACCGCCGTGTACTATTGCGCCCCCGATCCTCGGAGAAACTGTAGGGGAGGCGACTGCTGTGGAAATTGGGGACAGGGCACCACAGTGACAGTGAGCTCC

SEQ ID NO: 169 - DNA, 366 bp, Artificial Sequence
  AS53750VH5 sdAb
gaggtgcagctggtggagtctggaggaggactggtgcagccaggaggctccctgaggctgtcttgcgcagcaagcggcttcaccgacgatggaccagatatggcatggtacaggcaggcaccaggcaagggatgtgagctggtgtctatcatcagcgccgacggcagaacctactatacagatagcgtgaagggcaggtttaccatctcccgcgacaactctaagaatacagtgtatctgcagatgaactccctgagggccgaggacaccgccgtgtactattgcgcccccgatcctcggagaaactgtaggggaggcgactgctgtggaaattggggacagggcaccacagtgacagtgagctcc

SEQ ID NO: 170 - DNA, 366 bp, Artificial Sequence
  AS53750VH11 sdAb
gaggtgcagctggtggagagcggaggaggactggtgcagcctggaggctccctgaggctgtcttgcgcagcaagcggcttcaccgacgatggaccagatatggcatggtacaggcaggcaccaggcaagggatgtgagctggtgtctatcatcagcgccgacggcagaacctactatacagattccgtgaagggcaggtttaccatctctcgcgacaacgccaagaatacactgtatctgcagatgaactccctgaggcccgaggacaccgccgtgtactattgcgcccccgatcctcggagaaactgtaggggaggcgactgctgtggaaattggggacagggcaccacagtgacagtgagctcc

SEQ ID NO: 171 - DNA, 366 bp, Artificial Sequence
  AS53750VH12 sdAb
gaggtgcagctggtggagagcggaggaggactggtgcagcctggaggctccctgaggctgtcttgcgcagcaagcggcttcaccgacgatggaccagatatggcatggtacaggcaggcaccaggcaagggatgtgagctggtgtctatcatcagcgccgacggcagaacctactatacagattccgtgaagggcaggtttaccatctctcgcgacaacgccaagaatacagtgtatctgcagatgaactccctgaggcccgaggacaccgccgtgtactattgcgcccccgatcctcggagaaactgtaggggaggcgactgctgtggaaattggggacagggcaccacagtgacagtgagctcc

SEQ ID NO: 172 - DNA, 363 bp, Artificial Sequence
  AS54233VH4 sdAb
gaggtgcagctggtggagtctggaggaggactggtgcagccaggaggctccctgaggctgtcttgcgcagcaagcggcttcacctttgacggacccgatatggcctggtacagacaggcccctggcaagggctgtgagctggtgtctatcatcagcgccgacggcaggacctactatacagatagcgtgaagggacgcttcaccgcatcccaggacaactctaagaatacactgtatctgcagatgaacagcctgcgggccgaggacacagccgtgtactattgcgccgccgatccccggagaaactgtagaggcaattgctgtggaaactggggacagggaaccctggtgacagtgagctcc

SEQ ID NO: 173 - DNA, 363 bp, Artificial Sequence
  AS54233VH5 sdAb
gaggtgcagctggtggagtctggaggaggactggtgcagccaggaggctccctgaggctgtcttgcgcagcaagcggcttcacctttgacggacccgatatggcctggtacagacaggcccctggcaagggctgtgagctggtgtctatcatcagcgccgacggcaggacctactatacagatagcgtgaagggacgcttcaccgcatcccaggacaactctaagaatacagtgtatctgcagatgaacagcctgcgggccgaggacacagccgtgtactattgcgccgccgatccccggagaaactgtagaggcaattgctgtggaaactggggacagggaaccctggtgacagtgagctcc

SEQ ID NO: 174 - DNA, 363 bp, Artificial Sequence
  AS54233VH11 sdAb
gaggtgcagctggtggagagcggaggaggactggtgcagccaggaggctccctgaggctgtcttgcgcagcaagcggcttcacctttgacggacccgatatggcctggtacagacaggcccctggcaagggctgtgagctggtgtctatcatcagcgccgacggcaggacctactatacagattccgtgaagggccgcttcaccgcctctcaggacaacgccaagaatacactgtatctgcagatgaacagcctgcggccagaggacacagccgtgtactattgcgccgccgatccccggagaaactgtagaggcaattgctgtggaaactggggacagggaaccctggtgacagtgagctcc

SEQ ID NO: 175 - DNA, 363 bp, Artificial Sequence
  AS54233VH12 sdAb
gaggtgcagctggtggagagcggaggaggactggtgcagccaggaggctccctgaggctgtcttgcgcagcaagcggcttcacctttgacggacccgatatggcctggtacagacaggcccctggcaagggctgtgagctggtgtctatcatcagcgccgacggcaggacctactatacagattccgtgaagggccgcttcaccgcctctcaggacaacgccaagaatacagtgtatctgcagatgaacagcctgcggccagaggacacagccgtgtactattgcgccgccgatccccggagaaactgtagaggcaattgctgtggaaactggggacagggaaccctggtgacagtgagctcc

SEQ ID NO: 176 - DNA, 54 bp, Artificial Sequence
  5F11 scFv linker
ggcagcacctccggatctggcaagccaggaagcggagagggcagcacaaagggc

SEQ ID NO: 177 - DNA, 45 bp, Artificial Sequence
  (G4S)3 linker
ggaggaggaggaagcggaggaggaggatccggcggcggcggctct

SEQ ID NO: 178 - DNA, 15 bp, Artificial Sequence
  G4S linker
ggaggaggaggaagc

SEQ ID NO: 179 - DNA, 714 bp, Artificial Sequence
  AS57911 scFv
gacatccagatgacccagagcccgagcagcctgagcgcgagcgttggtgaccgtgttaccattacctgccgtgcgagccagagcgttagcagcgcggtggcgtggtaccagcaaaagccgggtaaagcgccgaagctgctgatctatagcgcgagcagcctgtatagcggcgttccgagccgtttcagcggtagccgtagcggcaccgactttaccctgaccattagcagcctgcagccggaagatttcgcaacttattactgtcagcaatctcatgctctgatcacgttcggacagggcaccaaagttgagattaaaggaggaggaggaagcggaggaggaggatccggcggcggcggctctgaggttcaactggtggagagcggtggtggtctggttcagccgggtggtagcctgcgtctgagctgcgcagcttctggcttcaacatctcttcttcttatatccactgggtgcgtcaggcgccgggtaaaggcctggaatgggttgcatatatttcttcttattatagctatacttattatgccgatagcgtcaagggccgtttcaccatcagcgcggataccagcaaaaacaccgcatacctgcaaatgaacagcctgcgtgcggaagataccgccgtctattattgtgctcgcggttacccgtacggtatggactactggggtcaaggcaccctggttaccgtgagcagc

SEQ ID NO: 180 - DNA, 720 bp, Artificial Sequence
  AS57659 scFv
gacatccagatgacccagagcccgagcagcctgagcgcgagcgttggtgaccgtgttaccattacctgccgtgcgagccagagcgttagcagcgcggtggcgtggtaccagcaaaagccgggtaaagcgccgaagctgctgatctatagcgcgagcagcctgtatagcggcgttccgagccgtttcagcggtagccgtagcggcaccgactttaccctgaccattagcagcctgcagccggaagatttcgcaacttattactgtcagcaaccgtactacctgatcacgttcggacagggcaccaaagttgagattaaaggaggaggaggaagcggaggaggaggatccggcggcggcggctctgaggttcaactggtggagagcggtggtggtctggttcagccgggtggtagcctgcgtctgagctgcgcagcttctggcttcaacatctattcttattatatccactgggtgcgtcaggcgccgggtaaaggcctggaatgggttgcatctatttattcttcttatagctctacttattatgccgatagcgtcaagggccgtttcaccatcagcgcggataccagcaaaaacaccgcatacctgcaaatgaacagcctgcgtgcggaagataccgccgtctattattgtgctcgctcttggttctcttacccgggtttggactactggggtcaaggcaccctggttaccgtgagcagc

SEQ ID NO: 181 - DNA, 747 bp, Artificial Sequence
  AS57765 scFv
gacatccagatgacccagagcccgagcagcctgagcgcgagcgttggtgaccgtgttaccattacctgccgtgcgagccagagcgttagcagcgcggtggcgtggtaccagcaaaagccgggtaaagcgccgaagctgctgatctatagcgcgagcagcctgtatagcggcgttccgagccgtttcagcggtagccgtagcggcaccgactttaccctgaccattagcagcctgcagccggaagatttcgcaacttattactgtcagcaagcttactactctctgatcacgttcggacagggcaccaaagttgagattaaaggaggaggaggaagcggaggaggaggatccggcggcggcggctctgaggttcaactggtggagagcggtggtggtctggttcagccgggtggtagcctgcgtctgagctgcgcagcttctggcttcaacatctattattcttatatgcactgggtgcgtcaggcgccgggtaaaggcctggaatgggttgcatatatttatccttattctggctctacttcttatgccgatagcgtcaagggccgtttcaccatcagcgcggataccagcaaaaacaccgcatacctgcaaatgaacagcctgcgtgcggaagataccgccgtctattattgtgctcgcccggctgttcattggcatggttacggtggtggttactactacggtttggactactggggtcaaggcaccctggttaccgtgagcagc

SEQ ID NO: 182 - protein, 366 aa, Artificial Sequence
  AS48542VH5bbz
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 367 aa, Artificial Sequence
  AS48463VH4bbz
MALPVTALLLPLALLLHAARPEVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 366 aa, Artificial Sequence
  AS47863VH4bbz
MALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 367 aa, Artificial Sequence
  AS53574VH7bbz
MALPVTALLLPLALLLHAARPEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 503 aa, Artificial Sequence
  AS48542VH5dil-bbz
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSGGGGSGGGGSGGGGSEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 505 aa, Artificial Sequence
  AS48463VH4dil-bbz
MALPVTALLLPLALLLHAARPEVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSSGGGGSGGGGSGGGGSEVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 503 aa, Artificial Sequence
  AS47863VH4dil-bbz
MALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSGGGGSGGGGSGGGGSEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 504 aa, Artificial Sequence
  AS48542VH5-AS53574VH7bil-bbz
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSGGGGSGGGGSGGGGSEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 505 aa, Artificial Sequence
  AS48463VH4-AS53574VH7bil-bbz
MALPVTALLLPLALLLHAARPEVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSSGGGGSGGGGSGGGGSEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 504 aa, Artificial Sequence
  AS47863VH4-AS53574VH7bil-bbz
MALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSGGGGSGGGGSGGGGSEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 504 aa, Artificial Sequence
  AS53574VH7-AS48542VH5bil-bbz
MALPVTALLLPLALLLHAARPEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSGGGGSGGGGSGGGGSEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 505 aa, Artificial Sequence
  AS53574VH7-AS48463VH4bil-bbz
MALPVTALLLPLALLLHAARPEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSGGGGSGGGGSGGGGSEVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 504 aa, Artificial Sequence
  AS53574VH7-AS47863VH4bil-bbz
MALPVTALLLPLALLLHAARPEVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSSGGGGSGGGGSGGGGSEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - protein, 969 aa, Artificial Sequence
  TR2D-AS48542VH5bbz-4C
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 196 - protein, 587 aa, Artificial Sequence
  TR2D-AS48542VH5bbz
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 623 aa, Artificial Sequence
  PD1CD28-AS48542VH5bbz
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHCPSPLFPGPSKPFWVLVVVGGVLACYSLLVTVAFIIFWVRSKRSRLLHSDYMNMTPRRPGPTRKHYQPYAPPRDFAAYRSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 748 aa, Artificial Sequence
  AS48542VH5bbz-4C
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 199 - protein, 123 aa, Artificial Sequence
  AS53574VH7
EVQLVESGGGLVQPGGSLRLSCAASGYIYSSNCMGWFRQAPGKGREWVARIHTGSGSTYYADSVKGRFTISQDNSKNTLYLQMNSLRAEDTAVYDCAAGRVVLGAVVCTNEYWGQGTLVTVSS

SEQ ID NO: 200 - DNA, 369 bp, Artificial Sequence
  AS53574VH7 sdAb
gaggtgcagctggtggagtccggaggaggactggtgcagccaggaggcagcctgcggctgtcctgcgccgcctctggctacatctatagctccaactgtatgggctggttcaggcaggcacctggcaagggaagggagtgggtggccagaatccacaccggctccggctctacatactatgccgactctgtgaagggccggtttaccatcagccaggataactccaagaatacactgtacctgcagatgaacagcctgagggccgaggacaccgccgtgtatgattgcgcagcaggaagggtggtgctgggagcagtggtgtgcacaaatgagtactggggccagggcaccctggtgacagtgtctagc

SEQ ID NO: 201 - protein, 362 aa, Artificial Sequence
  AS48542-28z
MALPVTALLLPLALLLHAARPQMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 9 aa, Artificial Sequence
  linker sequence
GGGGSGGGS

SEQ ID NO: 203 - protein, 5 aa, Artificial Sequence
  linker sequence
  REPEAT (1)..(5): amino acid no.1-5 can be repeated 4-6 times
GGGGS

SEQ ID NO: 204 - protein, 5 aa, Artificial Sequence
  linker sequence
SGGGS

SEQ ID NO: 205 - protein, 724 aa, Artificial Sequence
  TR2D-AS48542VH5dil-bbz
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSGGGGSGGGGSGGGGSEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 587 aa, Artificial Sequence
  TR2D-AS47863VH4bbz
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 207 - protein, 724 aa, Artificial Sequence
  TR2D-AS47863VH4dil-bbz
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSGGGGSGGGGSGGGGSEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 208 - protein, 362 aa, Artificial Sequence
  AS48542VH5-28z
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 209 - protein, 499 aa, Artificial Sequence
  AS48542VH5dil-28z
MALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSGGGGSGGGGSGGGGSEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 362 aa, Artificial Sequence
  AS47863VH4-28z
MALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - protein, 499 aa, Artificial Sequence
  AS47863VH4dil-28z
MALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSGGGGSGGGGSGGGGSEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - protein, 583 aa, Artificial Sequence
  TR2D-AS48542VH5-28z
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - protein, 720 aa, Artificial Sequence
  TR2D-AS48542VH5dil-28z
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSGGGGSGGGGSGGGGSEVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 214 - protein, 583 aa, Artificial Sequence
  TR2D-AS47863VH4-28z
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - protein, 720 aa, Artificial Sequence
  TR2D-AS47863VH4dil-28z
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSGGGGSGGGGSGGGGSEVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 216 - protein, 237 aa, Artificial Sequence
  5F11 scFv
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 217 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 106 aa, Artificial Sequence
  AS57911VH
DIQMTQSPSSLSASVGDRVTITCRASQSVSSAVAWYQQKPGKAPKLLIYSASSLYSGVPSRFSGSRSGTDFTLTISSLQPEDFATYYCQQSHALITFGQGTKVEIK

SEQ ID NO: 219 - protein, 117 aa, Artificial Sequence
  AS57911VL
EVQLVESGGGLVQPGGSLRLSCAASGFNISSSYIHWVRQAPGKGLEWVAYISSYYSYTYYADSVKGRFTISADTSKNTAYLQMNSLRAEDTAVYYCARGYPYGMDYWGQGTLVTVSS

SEQ ID NO: 220 - protein, 106 aa, Artificial Sequence
  AS57659VH
DIQMTQSPSSLSASVGDRVTITCRASQSVSSAVAWYQQKPGKAPKLLIYSASSLYSGVPSRFSGSRSGTDFTLTISSLQPEDFATYYCQQPYYLITFGQGTKVEIK

SEQ ID NO: 221 - protein, 119 aa, Artificial Sequence
  AS57659VL
EVQLVESGGGLVQPGGSLRLSCAASGFNIYSYYIHWVRQAPGKGLEWVASIYSSYSSTYYADSVKGRFTISADTSKNTAYLQMNSLRAEDTAVYYCARSWFSYPGLDYWGQGTLVTVSS

SEQ ID NO: 222 - protein, 107 aa, Artificial Sequence
  AS57765VH
DIQMTQSPSSLSASVGDRVTITCRASQSVSSAVAWYQQKPGKAPKLLIYSASSLYSGVPSRFSGSRSGTDFTLTISSLQPEDFATYYCQQAYYSLITFGQGTKVEIK

SEQ ID NO: 223 - protein, 127 aa, Artificial Sequence
  AS57765VL
EVQLVESGGGLVQPGGSLRLSCAASGFNIYYSYMHWVRQAPGKGLEWVAYIYPYSGSTSYADSVKGRFTISADTSKNTAYLQMNSLRAEDTAVYYCARPAVHWHGYGGGYYYGLDYWGQGTLVTVSS